SEQUENCE LISTING (597 sequences)

SEQ ID NO: 1 - protein, 15 aa, synthetic construct
  REGION 1..15: note = Description of Artificial Sequence: Synthetic peptide
GYSTPPKKKRKVEDP

SEQ ID NO: 2 - protein, 12 aa, synthetic construct
  REGION 1..12: note = Description of Artificial Sequence: Synthetic peptide
GYSTPPKTRRRP

SEQ ID NO: 3 - protein, 10 aa, synthetic construct
  REGION 1..10: note = Description of Artificial Sequence: Synthetic peptide
GYSTPGRKKR

SEQ ID NO: 4 - protein, 13 aa, synthetic construct
  REGION 1..13: note = Description of Artificial Sequence: Synthetic peptide
GYSTPRRNRRRRW

SEQ ID NO: 5 - protein, 15 aa, synthetic construct
  REGION 1..15: note = Description of Artificial Sequence: Synthetic peptide
PDEVKRKKKPPTSYG

SEQ ID NO: 6 - protein, 12 aa, synthetic construct
  REGION 1..12: note = Description of Artificial Sequence: Synthetic peptide
PRRRTKPPTSYG

SEQ ID NO: 7 - protein, 10 aa, synthetic construct
  REGION 1..10: note = Description of Artificial Sequence: Synthetic peptide
RKKRGPTSYG

SEQ ID NO: 8 - protein, 13 aa, synthetic construct
  REGION 1..13: note = Description of Artificial Sequence: Synthetic peptide
WRRRRNRRPTSYG

SEQ ID NO: 9 - protein, 17 aa, synthetic construct
  REGION 1..17: note = Description of Artificial Sequence: Synthetic peptide
GYGPPKKKRKVEAPYKA

SEQ ID NO: 10 - protein, 9 aa, synthetic construct
  REGION 1..9: note = Description of Artificial Sequence: Synthetic peptide
PAAKRVKLD

SEQ ID NO: 11 - protein, 11 aa, synthetic construct
  REGION 1..11: note = Description of Artificial Sequence: Synthetic peptide
RQRRNELKRSP

SEQ ID NO: 12 - protein, 16 aa, synthetic construct
  REGION 1..16: note = Description of Artificial Sequence: Synthetic peptide
KRPAATKKAGQAKKKK

SEQ ID NO: 13 - protein, 24 aa, synthetic construct
  REGION 1..24: note = Description of Artificial Sequence: Synthetic peptide
VRKKRKTEEESPLKDKDAKKSKQE

SEQ ID NO: 14 - protein, 24 aa, synthetic construct
  REGION 1..24: note = Description of Artificial Sequence: Synthetic peptide
RLRRDAGGRGGVYEHLGGAPRRRK

SEQ ID NO: 15 - protein, 19 aa, synthetic construct
  REGION 1..19: note = Description of Artificial Sequence: Synthetic peptide
KRKGDEVDGVDECAKKSKK

SEQ ID NO: 16 - protein, 38 aa, synthetic construct
  REGION 1..38: note = Description of Artificial Sequence: Synthetic polypeptide
NQSSNFGPMKGGNFGGRSSGPYGGGGQYFAKPRNQGGY

SEQ ID NO: 17 - protein, 27 aa, synthetic construct
  REGION 1..27: note = Description of Artificial Sequence: Synthetic peptide
GGKRTADGSEFESPKKARKVEAYPKAW

SEQ ID NO: 18 - protein, 27 aa, synthetic construct
  REGION 1..27: note = Description of Artificial Sequence: Synthetic peptide
GGKRTADGSEFESPKKKRAVEAYPKAW

SEQ ID NO: 19 - protein, 27 aa, synthetic construct
  REGION 1..27: note = Description of Artificial Sequence: Synthetic peptide
GGKRTADGSEFESPKKKAKVEAYPKAW

SEQ ID NO: 20 - protein, 28 aa, synthetic construct
  REGION 1..28: note = Description of Artificial Sequence: Synthetic peptide
GGKRTADGSEFESPKKKRKVEAPYKAWK

SEQ ID NO: 21 - protein, 26 aa, synthetic construct
  REGION 1..26: note = Description of Artificial Sequence: Synthetic peptide
GGKRTADGSEFESPKKKRKVEYKAWK

SEQ ID NO: 22 - protein, 20 aa, synthetic construct
  REGION 1..20: note = Description of Artificial Sequence: Synthetic peptide
GYGPAAKRVKLDEAYPKAWK

SEQ ID NO: 23 - protein, 29 aa, synthetic construct
  REGION 1..29: note = Description of Artificial Sequence: Synthetic peptide
GGKRTADGSEFEPAAKRVKLDEAYPKAWK

SEQ ID NO: 24 - protein, 24 aa, synthetic construct
  REGION 1..24: note = Description of Artificial Sequence: Synthetic peptide
GTGPKKKRKVGGGGYGPKKKRLVG

SEQ ID NO: 25 - protein, 25 aa, synthetic construct
  REGION 1..25: note = Description of Artificial Sequence: Synthetic peptide
KRPAATKKAGQAKKKKLEAYPKAWK

SEQ ID NO: 26 - protein, 16 aa, synthetic construct
  REGION 1..16: note = Description of Artificial Sequence: Synthetic peptide
ATKGTKRSYEQMETGE

SEQ ID NO: 27 - protein, 12 aa, synthetic construct
  REGION 1..12: note = Description of Artificial Sequence: Synthetic peptide
GKWERKPIRCAS

SEQ ID NO: 28 - protein, 29 aa, synthetic construct
  REGION 1..29: note = Description of Artificial Sequence: Synthetic peptide
GYGKRTADSQHSTPPKKKRKVEAPYKAWK

SEQ ID NO: 29 - protein, 26 aa, synthetic construct
  REGION 1..26: note = Description of Artificial Sequence: Synthetic peptide
KRTADSQHSTPPKKKRKVEAPYKAWK

SEQ ID NO: 30 - protein, 46 aa, synthetic construct
  REGION 1..46: note = Description of Artificial Sequence: Synthetic polypeptide
GYGPPKKKRKVEAPYKAWKWAKYPAMRRAHHRRRRASHRRRTTTGT

SEQ ID NO: 31 - protein, 43 aa, synthetic construct
  REGION 1..43: note = Description of Artificial Sequence: Synthetic polypeptide
GYGPPKKKRKVEAPYKAWKRGARRYSKMKRRRRRVARRHRRRP

SEQ ID NO: 32 - protein, 23 aa, synthetic construct
  REGION 1..23: note = Description of Artificial Sequence: Synthetic peptide
FWGYGYGPPKKKRKVEAPYKAWK

SEQ ID NO: 33 - protein, 28 aa, synthetic construct
  REGION 1..28: note = Description of Artificial Sequence: Synthetic peptide
GKPSSDDEATADSQHSTPPKKKERKVED

SEQ ID NO: 34 - protein, 21 aa, synthetic construct
  REGION 1..21: note = Description of Artificial Sequence: Synthetic peptide
GKPTADDQHSTPPKKKRKVED

SEQ ID NO: 35 - protein, 27 aa, synthetic construct
  REGION 1..27: note = Description of Artificial Sequence: Synthetic peptide
GGKRTADGSEFESPKKARKVEAYPKAK

SEQ ID NO: 36 - protein, 18 aa, synthetic construct
  REGION 1..18: note = Description of Artificial Sequence: Synthetic peptide
EKIRLRPGRKKRYRLKHL

SEQ ID NO: 37 - protein, 18 aa, synthetic construct
  REGION 1..18: note = Description of Artificial Sequence: Synthetic peptide
PEGTRQARRNRRRRWRKR

SEQ ID NO: 38 - protein, 18 aa, synthetic construct
  REGION 1..18: note = Description of Artificial Sequence: Synthetic peptide
PEGTRQPRRNRRRRWRKR

SEQ ID NO: 39 - protein, 26 aa, synthetic construct
  REGION 1..26: note = Description of Artificial Sequence: Synthetic peptide
GVKRSYGAARGDDRRRPNVVAPYKAW

SEQ ID NO: 40 - protein, 26 aa, synthetic construct
  REGION 1..26: note = Description of Artificial Sequence: Synthetic peptide
KSVPNRTRTYIKLKRLRFKGAPYKAW

SEQ ID NO: 41 - protein, 27 aa, synthetic construct
  REGION 1..27: note = Description of Artificial Sequence: Synthetic peptide
EMRRRREEEGLQLRKQKREEQLFKRRN

SEQ ID NO: 42 - protein, 14 aa, synthetic construct
  REGION 1..14: note = Description of Artificial Sequence: Synthetic peptide
FEAALAEALAEALA

SEQ ID NO: 43 - protein, 12 aa, synthetic construct
  REGION 1..12: note = Description of Artificial Sequence: Synthetic peptide
LARLLPRLLARL

SEQ ID NO: 44 - protein, 20 aa, synthetic construct
  REGION 1..20: note = Description of Artificial Sequence: Synthetic peptide
GLLEELLELLEELWEELLEG

SEQ ID NO: 45 - protein, 19 aa, synthetic construct
  REGION 1..19: note = Description of Artificial Sequence: Synthetic peptide
GWEGLIEGIEGGWEGLIEG

SEQ ID NO: 46 - protein, 20 aa, synthetic construct
  REGION 1..20: note = Description of Artificial Sequence: Synthetic peptide
GLFEALAEFIEGGWEGLIEG

SEQ ID NO: 47 - protein, 20 aa, synthetic construct
  REGION 1..20: note = Description of Artificial Sequence: Synthetic peptide
GLFEALLELLESLWELLLEA

SEQ ID NO: 48 - protein, 22 aa, synthetic construct
  REGION 1..22: note = Description of Artificial Sequence: Synthetic peptide
GGYCLEKWMIVASELKCFGNTA

SEQ ID NO: 49 - protein, 23 aa, synthetic construct
  REGION 1..23: note = Description of Artificial Sequence: Synthetic peptide
GGYCLTRWMLIEAELKCFGNTAV

SEQ ID NO: 50 - protein, 30 aa, synthetic construct
  REGION 1..30: note = Description of Artificial Sequence: Synthetic polypeptide
WEAALAEALAEALAEHLAEALAEALEALAA

SEQ ID NO: 51 - protein, 23 aa, synthetic construct
  REGION 1..23: note = Description of Artificial Sequence: Synthetic peptide
GLFGAIAGFIENGWEGMIDGWYG

SEQ ID NO: 52 - protein, 26 aa, synthetic construct
  REGION 1..26: note = Description of Artificial Sequence: Synthetic peptide
GIGAVLKVLTTGLPALISWIKRKRQQ

SEQ ID NO: 53 - protein, 13 aa, synthetic construct
  REGION 1..13: note = Description of Artificial Sequence: Synthetic peptide
GRKKRRQRRRPPQ

SEQ ID NO: 54 - protein, 16 aa, synthetic construct
  REGION 1..16: note = Description of Artificial Sequence: Synthetic peptide
RQIKIWFQNRRMKWKK

SEQ ID NO: 55 - protein, 27 aa, synthetic construct
  REGION 1..27: note = Description of Artificial Sequence: Synthetic peptide
GWTLNSAGYLLGKINLKALAALAKKIL

SEQ ID NO: 56 - protein, 30 aa, synthetic construct
  REGION 1..30: note = Description of Artificial Sequence: Synthetic polypeptide
WEAKLAKALAKALAKHLAKALAKALKACEA

SEQ ID NO: 57 - protein, 20 aa, synthetic construct
  REGION 1..20: note = Description of Artificial Sequence: Synthetic peptide
GLFKALLKLLKSLWKLLLKA

SEQ ID NO: 58 - protein, 20 aa, synthetic construct
  REGION 1..20: note = Description of Artificial Sequence: Synthetic peptide
GLFRALLRLLRSLWRLLLRA

SEQ ID NO: 59 - protein, 20 aa, synthetic construct
  REGION 1..20: note = Description of Artificial Sequence: Synthetic peptide
GLFEALLELLESLYELLLEA

SEQ ID NO: 60 - protein, 12 aa, synthetic construct
  REGION 1..12: note = Description of Artificial Sequence: Synthetic peptide
GLFEALEELWEA

SEQ ID NO: 61 - protein, 10 aa, synthetic construct
  REGION 1..10: note = Description of Artificial Sequence: Synthetic peptide
GLFLLEEWLE

SEQ ID NO: 62 - protein, 11 aa, synthetic construct
  REGION 1..11: note = Description of Artificial Sequence: Synthetic peptide
GLFLLEEWLEK

SEQ ID NO: 63 - protein, 21 aa, synthetic construct
  REGION 1..21: note = Description of Artificial Sequence: Synthetic peptide
GLFEALLELLESLWELLLEAK

SEQ ID NO: 64 - protein, 11 aa, synthetic construct
  REGION 1..11: note = Description of Artificial Sequence: Synthetic peptide
GLFKLLEEWLE

SEQ ID NO: 65 - protein, 12 aa, synthetic construct
  REGION 1..12: note = Description of Artificial Sequence: Synthetic peptide
GLFKLLEEWLEK

SEQ ID NO: 66 - protein, 20 aa, synthetic construct
  REGION 1..20: note = Description of Artificial Sequence: Synthetic peptide
GLFEAIAEFIEGGWEGLIEG

SEQ ID NO: 67 - protein, 20 aa, synthetic construct
  REGION 1..20: note = Description of Artificial Sequence: Synthetic peptide
GLFKAIAKFIKGGWKGLIKG

SEQ ID NO: 68 - protein, 17 aa, synthetic construct
  REGION 1..17: note = Description of Artificial Sequence: Synthetic peptide
IRFKKTKLIASIAMALC

SEQ ID NO: 69 - protein, 30 aa, synthetic construct
  REGION 1..30: note = Description of Artificial Sequence: Synthetic polypeptide
ALAGTIIAGASLTFQVLDKVLEELGKVSRK

SEQ ID NO: 70 - protein, 23 aa, synthetic construct
  REGION 1..23: note = Description of Artificial Sequence: Synthetic peptide
GLFEAIEGFIENGWEGMIDGWYG

SEQ ID NO: 71 - protein, 17 aa, synthetic construct
  REGION 1..17: note = Description of Artificial Sequence: Synthetic peptide
GYICRRARGDNPDDRCT

SEQ ID NO: 72 - protein, 22 aa, synthetic construct
  REGION 1..22: note = Description of Artificial Sequence: Synthetic peptide
GLFEAIAEFIEGGWEGLIEGCA

SEQ ID NO: 73 - protein, 24 aa, synthetic construct
  REGION 1..24: note = Description of Artificial Sequence: Synthetic peptide
GLFHAIAHFIHGGWHGLIHGWWYG

SEQ ID NO: 74 - protein, 30 aa, synthetic construct
  REGION 1..30: note = Description of Artificial Sequence: Synthetic polypeptide
RRRQRRKKRGGDIMGEWGNEIFGAIAGFLG

SEQ ID NO: 75 - protein, 22 aa, synthetic construct
  REGION 1..22: note = Description of Artificial Sequence: Synthetic peptide
GLFEAIADFIENGWEGMIDGGG

SEQ ID NO: 76 - protein, 31 aa, synthetic construct
  REGION 1..31: note = Description of Artificial Sequence: Synthetic polypeptide
ALAGTIIAGASLTFQVLDKVLEELGKVSRKK

SEQ ID NO: 77 - protein, 15 aa, synthetic construct
  REGION 1..15: note = Description of Artificial Sequence: Synthetic peptide
IRFKKTKLIASIAMA

SEQ ID NO: 78 - protein, 18 aa, synthetic construct
  REGION 1..18: note = Description of Artificial Sequence: Synthetic peptide
GLWHLLLHLWRRLLRLLR

SEQ ID NO: 79 - protein, 21 aa, synthetic construct
  REGION 1..21: note = Description of Artificial Sequence: Synthetic peptide
KKIMLLLMTLLLVSLPLAQEQ

SEQ ID NO: 80 - protein, 23 aa, synthetic construct
  REGION 1..23: note = Description of Artificial Sequence: Synthetic peptide
GLFEALLELLESLWELLLEAWYG

SEQ ID NO: 81 - protein, 18 aa, synthetic construct
  REGION 1..18: note = Description of Artificial Sequence: Synthetic peptide
RLLRLLLRLWRRLLRLLR

SEQ ID NO: 82 - protein, 26 aa, synthetic construct
  REGION 1..26: note = Description of Artificial Sequence: Synthetic peptide
LLELELLELELLLELELLELELLLEL

SEQ ID NO: 83 - protein, 28 aa, synthetic construct
  REGION 1..28: note = Description of Artificial Sequence: Synthetic peptide
GLFEALLELLESLWELLLEARRRRRRRR

SEQ ID NO: 84 - protein, 26 aa, synthetic construct
  REGION 1..26: note = Description of Artificial Sequence: Synthetic peptide
GLFEALLELLESLWELLLEARRRRRR

SEQ ID NO: 85 - protein, 28 aa, synthetic construct
  REGION 1..28: note = Description of Artificial Sequence: Synthetic peptide
GLFEALLELLESLWELLLEAKKKKKKKK

SEQ ID NO: 86 - protein, 26 aa, synthetic construct
  REGION 1..26: note = Description of Artificial Sequence: Synthetic peptide
GLFEALLELLESLWELLLEAKKKKKK

SEQ ID NO: 87 - protein, 22 aa, synthetic construct
  REGION 1..22: note = Description of Artificial Sequence: Synthetic peptide
GLFEALLELLESLWELLLEAKK

SEQ ID NO: 88 - protein, 24 aa, synthetic construct
  REGION 1..24: note = Description of Artificial Sequence: Synthetic peptide
GLFEALLELLESLWELLLEAKKKK

SEQ ID NO: 89 - protein, 22 aa, synthetic construct
  REGION 1..22: note = Description of Artificial Sequence: Synthetic peptide
GLFEALLELLESLWELLLEAEE

SEQ ID NO: 90 - protein, 24 aa, synthetic construct
  REGION 1..24: note = Description of Artificial Sequence: Synthetic peptide
GLFEALLELLESLWELLLEAEEEE

SEQ ID NO: 91 - protein, 26 aa, synthetic construct
  REGION 1..26: note = Description of Artificial Sequence: Synthetic peptide
GLFEALLELLESLWELLLEAEEEEEE

SEQ ID NO: 92 - protein, 18 aa, synthetic construct
  REGION 1..18: note = Description of Artificial Sequence: Synthetic peptide
GLFEALLELLESLWELLL

SEQ ID NO: 93 - protein, 36 aa, synthetic construct
  REGION 1..36: note = Description of Artificial Sequence: Synthetic polypeptide
PLSSIFSRIGDPRGARRYAKMKRRRRRVARRHRRRP

SEQ ID NO: 94 - protein, 13 aa, synthetic construct
  REGION 1..13: note = Description of Artificial Sequence: Synthetic peptide
GPFHYFQFLFPPV

SEQ ID NO: 95 - protein, 13 aa, synthetic construct
  REGION 1..13: note = Description of Artificial Sequence: Synthetic peptide
GSSSWWQRWWPPW

SEQ ID NO: 96 - protein, 9 aa, synthetic construct
  REGION 1..9: note = Description of Artificial Sequence: Synthetic peptide
RRRQRRKKR

SEQ ID NO: 97 - protein, 4 aa, synthetic construct
  REGION 1..4: note = Description of Artificial Sequence: Synthetic peptide
KKKK

SEQ ID NO: 98 - protein, 6 aa, synthetic construct
  REGION 1..6: note = Description of Artificial Sequence: Synthetic peptide
KKKKKK

SEQ ID NO: 99 - protein, 8 aa, synthetic construct
  REGION 1..8: note = Description of Artificial Sequence: Synthetic peptide
KKKKKKKK

SEQ ID NO: 100 - protein, 10 aa, synthetic construct
  REGION 1..10: note = Description of Artificial Sequence: Synthetic peptide
KKKKKKKKKK

SEQ ID NO: 101 - protein, 12 aa, synthetic construct
  REGION 1..12: note = Description of Artificial Sequence: Synthetic peptide
KKKKKKKKKKKK

SEQ ID NO: 102 - protein, 16 aa, synthetic construct
  REGION 1..16: note = Description of Artificial Sequence: Synthetic peptide
KKKKKKKKKKKKKKKK

SEQ ID NO: 103 - protein, 20 aa, synthetic construct
  REGION 1..20: note = Description of Artificial Sequence: Synthetic peptide
KKKKKKKKKKKKKKKKKKKK

SEQ ID NO: 104 - protein, 24 aa, synthetic construct
  REGION 1..24: note = Description of Artificial Sequence: Synthetic peptide
KKKKKKKKKKKKKKKKKKKKKKKK

SEQ ID NO: 105 - protein, 4 aa, synthetic construct
  REGION 1..4: note = Description of Artificial Sequence: Synthetic peptide
RRRR

SEQ ID NO: 106 - protein, 6 aa, synthetic construct
  REGION 1..6: note = Description of Artificial Sequence: Synthetic peptide
RRRRRR

SEQ ID NO: 107 - protein, 8 aa, synthetic construct
  REGION 1..8: note = Description of Artificial Sequence: Synthetic peptide
RRRRRRRR

SEQ ID NO: 108 - protein, 10 aa, synthetic construct
  REGION 1..10: note = Description of Artificial Sequence: Synthetic peptide
RRRRRRRRRR

SEQ ID NO: 109 - protein, 12 aa, synthetic construct
  REGION 1..12: note = Description of Artificial Sequence: Synthetic peptide
RRRRRRRRRRRR

SEQ ID NO: 110 - protein, 16 aa, synthetic construct
  REGION 1..16: note = Description of Artificial Sequence: Synthetic peptide
RRRRRRRRRRRRRRRR

SEQ ID NO: 111 - protein, 20 aa, synthetic construct
  REGION 1..20: note = Description of Artificial Sequence: Synthetic peptide
RRRRRRRRRRRRRRRRRRRR

SEQ ID NO: 112 - protein, 24 aa, synthetic construct
  REGION 1..24: note = Description of Artificial Sequence: Synthetic peptide
RRRRRRRRRRRRRRRRRRRRRRRR

SEQ ID NO: 113 - skipped

SEQ ID NO: 114 - protein, 25 aa, synthetic construct
  REGION 1..25: note = Description of Artificial Sequence: Synthetic peptide
KKKKKKKKWKGGGGACYGLPHLFCG

SEQ ID NO: 115 - protein, 13 aa, synthetic construct
  REGION 1..13: note = Description of Artificial Sequence: Synthetic peptide
YKAKKKKKKKKWK

SEQ ID NO: 116 - protein, 18 aa, synthetic construct
  REGION 1..18: note = Description of Artificial Sequence: Synthetic peptide
KTPKKAKKPKTPKKAKKP

SEQ ID NO: 117 - protein, 29 aa, synthetic construct
  REGION 1..29: note = Description of Artificial Sequence: Synthetic peptide
KKAKKPAATRKSSKNPKKPKTVKPKKVAK

SEQ ID NO: 118 - protein, 24 aa, synthetic construct
  REGION 1..24: note = Description of Artificial Sequence: Synthetic peptide
RGARRYSKMKRRRRRVARRHRRRP

SEQ ID NO: 119 - protein, 22 aa, synthetic construct
  REGION 1..22: note = Description of Artificial Sequence: Synthetic peptide
TRQARRNRRRRWRERQRGSGSG

SEQ ID NO: 120 - protein, 29 aa, synthetic construct
  REGION 1..29: note = Description of Artificial Sequence: Synthetic peptide
KRPRGRPKGSKKNWRRRKRRASRRSPRRR

SEQ ID NO: 121 - protein, 34 aa, synthetic construct
  REGION 1..34: note = Description of Artificial Sequence: Synthetic polypeptide
KRGRGRPRKQPPKEPSEVPTPKRPRGRPKGSKNK

SEQ ID NO: 122 - protein, 33 aa, synthetic construct
  REGION 1..33: note = Description of Artificial Sequence: Synthetic polypeptide
KEKYEKDIAAYRAKGKPAAKKGVVKAEKSKKKK

SEQ ID NO: 123 - protein, 15 aa, synthetic construct
  REGION 1..15: note = Description of Artificial Sequence: Synthetic peptide
YKAKKKKKKKKKKWK

SEQ ID NO: 124 - protein, 10 aa, synthetic construct
  REGION 1..10: note = Description of Artificial Sequence: Synthetic peptide
KKKKKKKGGC

SEQ ID NO: 125 - protein, 13 aa, synthetic construct
  REGION 1..13: note = Description of Artificial Sequence: Synthetic peptide
YRARRRRRRRRWR

SEQ ID NO: 126 - protein, 15 aa, synthetic construct
  REGION 1..15: note = Description of Artificial Sequence: Synthetic peptide
YRARRRRRRRRRRWR

SEQ ID NO: 127 - protein, 42 aa, synthetic construct
  REGION 1..42: note = Description of Artificial Sequence: Synthetic polypeptide
KGDPKKPRGKMSSYAFFVQTCREEHKKKHPDASVNFSEFSKK

SEQ ID NO: 128 - protein, 16 aa, synthetic construct
  REGION 1..16: note = Description of Artificial Sequence: Synthetic peptide
KKQLKKQLKKQLKQWK

SEQ ID NO: 129 - protein, 16 aa, synthetic construct
  REGION 1..16: note = Description of Artificial Sequence: Synthetic peptide
KKSPKKSPKKSPKKSK

SEQ ID NO: 130 - protein, 13 aa, synthetic construct
  REGION 1..13: note = Description of Artificial Sequence: Synthetic peptide
KLSKLEKKSKLEK

SEQ ID NO: 131 - protein, 20 aa, synthetic construct
  REGION 1..20: note = Description of Artificial Sequence: Synthetic peptide
KLSKLEKKLSKLEKKSKLEK

SEQ ID NO: 132 - protein, 15 aa, synthetic construct
  REGION 1..15: note = Description of Artificial Sequence: Synthetic peptide
KSLKKSLKKSLKKSK

SEQ ID NO: 133 - protein, 22 aa, synthetic construct
  REGION 1..22: note = Description of Artificial Sequence: Synthetic peptide
KIRRRGKNKVAARTCRQRRTDR

SEQ ID NO: 134 - protein, 22 aa, synthetic construct
  REGION 1..22: note = Description of Artificial Sequence: Synthetic peptide
KIRRRGKNKVAAQNCRKRKLET

SEQ ID NO: 135 - protein, 24 aa, synthetic construct
  REGION 1..24: note = Description of Artificial Sequence: Synthetic peptide
KRRIRREKNKMAAAKCRNRRRELT

SEQ ID NO: 136 - protein, 12 aa, synthetic construct
  REGION 1..12: note = Description of Artificial Sequence: Synthetic peptide
KDRSNLLERHTR

SEQ ID NO: 137 - protein, 16 aa, synthetic construct
  REGION 1..16: note = Description of Artificial Sequence: Synthetic peptide
KRPAATKKAGQAKKKL

SEQ ID NO: 138 - protein, 10 aa, synthetic construct
  REGION 1..10: note = Description of Artificial Sequence: Synthetic peptide
RRRRRREEEE

SEQ ID NO: 139 - protein, 12 aa, synthetic construct
  REGION 1..12: note = Description of Artificial Sequence: Synthetic peptide
RRRRRREEEEEE

SEQ ID NO: 140 - protein, 14 aa, synthetic construct
  REGION 1..14: note = Description of Artificial Sequence: Synthetic peptide
RRRRRREEEEEEEE

SEQ ID NO: 141 - protein, 12 aa, synthetic construct
  REGION 1..12: note = Description of Artificial Sequence: Synthetic peptide
RRRRRRRREEEE

SEQ ID NO: 142 - protein, 14 aa, synthetic construct
  REGION 1..14: note = Description of Artificial Sequence: Synthetic peptide
RRRRRRRREEEEEE

SEQ ID NO: 143 - protein, 16 aa, synthetic construct
  REGION 1..16: note = Description of Artificial Sequence: Synthetic peptide
RRRRRRRREEEEEEEE

SEQ ID NO: 144 - protein, 16 aa, synthetic construct
  REGION 1..16: note = Description of Artificial Sequence: Synthetic peptide
RRRRRRRRRRRREEEE

SEQ ID NO: 145 - protein, 18 aa, synthetic construct
  REGION 1..18: note = Description of Artificial Sequence: Synthetic peptide
RRRRRRRRRRRREEEEEE

SEQ ID NO: 146 - protein, 18 aa, synthetic construct
  REGION 1..18: note = Description of Artificial Sequence: Synthetic peptide
RRRRRRRRRRRREEEEEE

SEQ ID NO: 147 - protein, 8 aa, synthetic construct
  REGION 1..8: note = Description of Artificial Sequence: Synthetic peptide
KLSKLEKK

SEQ ID NO: 148 - protein, 5 aa, synthetic construct
  REGION 1..5: note = Description of Artificial Sequence: Synthetic peptide
SKLEK

SEQ ID NO: 149 - protein, 15 aa, synthetic construct
  REGION 1..15: note = Description of Artificial Sequence: Synthetic peptide
KLSKLEKKLSKLEKK

SEQ ID NO: 150 - protein, 15 aa, synthetic construct
  REGION 1..15: note = Description of Artificial Sequence: Synthetic peptide
PKKKRKVGGGRGDSP

SEQ ID NO: 151 - protein, 9 aa, synthetic construct
  REGION 1..9: note = Description of Artificial Sequence: Synthetic peptide
LPHKSMPCG

SEQ ID NO: 152 - protein, 12 aa, synthetic construct
  REGION 1..12: note = Description of Artificial Sequence: Synthetic peptide
GACLQHKSMPCG

SEQ ID NO: 153 - protein, 9 aa, synthetic construct
  REGION 1..9: note = Description of Artificial Sequence: Synthetic peptide
YGLPHLFCG

SEQ ID NO: 154 - protein, 9 aa, synthetic construct
  REGION 1..9: note = Description of Artificial Sequence: Synthetic peptide
SERSMNFCG

SEQ ID NO: 155 - protein, 15 aa, synthetic construct
  REGION 1..15: note = Description of Artificial Sequence: Synthetic peptide
DHYSLYEDLERGTDK

SEQ ID NO: 156 - protein, 16 aa, synthetic construct
  REGION 1..16: note = Description of Artificial Sequence: Synthetic peptide
ISLPRTSGAQRASTTR

SEQ ID NO: 157 - protein, 17 aa, synthetic construct
  REGION 1..17: note = Description of Artificial Sequence: Synthetic peptide
EKLQTKYGLPHKVEFCG

SEQ ID NO: 158 - protein, 12 aa, synthetic construct
  REGION 1..12: note = Description of Artificial Sequence: Synthetic peptide
TRISESQAKPGD

SEQ ID NO: 159 - protein, 6 aa, synthetic construct
  REGION 1..6: note = Description of Artificial Sequence: Synthetic peptide
LVFFDY

SEQ ID NO: 160 - protein, 21 aa, synthetic construct
  REGION 1..21: note = Description of Artificial Sequence: Synthetic peptide
WGGNGPTTFDCSGYTKYVFAK

SEQ ID NO: 161 - protein, 15 aa, synthetic construct
  REGION 1..15: note = Description of Artificial Sequence: Synthetic peptide
INIGTTGWGDHYSLY

SEQ ID NO: 162 - protein, 6 aa, synthetic construct
  REGION 1..6: note = Description of Artificial Sequence: Synthetic peptide
YDNIHG

SEQ ID NO: 163 - protein, 13 aa, synthetic construct
  REGION 1..13: note = Description of Artificial Sequence: Synthetic peptide
AGWGKFLVGFGRV

SEQ ID NO: 164 - protein, 7 aa, synthetic construct
  REGION 1..7: note = Description of Artificial Sequence: Synthetic peptide
SIGYPLP

SEQ ID NO: 165 - protein, 8 aa, synthetic construct
  REGION 1..8: note = Description of Artificial Sequence: Synthetic peptide
TTHWGFTL

SEQ ID NO: 166 - protein, 12 aa, synthetic construct
  REGION 1..12: note = Description of Artificial Sequence: Synthetic peptide
HLQIQPYPQISG

SEQ ID NO: 167 - protein, 9 aa, synthetic construct
  REGION 1..9: note = Description of Artificial Sequence: Synthetic peptide
KLNIVSVNG

SEQ ID NO: 168 - skipped

SEQ ID NO: 169 - protein, 11 aa, synthetic construct
  REGION 1..11: note = Description of Artificial Sequence: Synthetic peptide
DNRIRLQAKAA

SEQ ID NO: 170 - protein, 10 aa, synthetic construct
  REGION 1..10: note = Description of Artificial Sequence: Synthetic peptide
KIKMVISWKG

SEQ ID NO: 171 - protein, 12 aa, synthetic construct
  REGION 1..12: note = Description of Artificial Sequence: Synthetic peptide
LPWYSYLYAVSA

SEQ ID NO: 172 - protein, 12 aa, synthetic construct
  REGION 1..12: note = Description of Artificial Sequence: Synthetic peptide
WNLPWYYSVSPT

SEQ ID NO: 173 - skipped

SEQ ID NO: 174 - protein, 9 aa, synthetic construct
  REGION 1..9: note = Description of Artificial Sequence: Synthetic peptide
PWYYSVSPT

SEQ ID NO: 175 - protein, 14 aa, synthetic construct
  REGION 1..14: note = Description of Artificial Sequence: Synthetic peptide
SSWESYKSGGGTRL

SEQ ID NO: 176 - protein, 18 aa, synthetic construct
  REGION 1..18: note = Description of Artificial Sequence: Synthetic peptide
RDWSSQHPGRCNGETHLK

SEQ ID NO: 177 - protein, 7 aa, synthetic construct
  REGION 1..7: note = Description of Artificial Sequence: Synthetic peptide
SLPTLTL

SEQ ID NO: 178 - protein, 21 aa, synthetic construct
  REGION 1..21: note = Description of Artificial Sequence: Synthetic peptide
VICTGGDYSFALPVGQWPVMT

SEQ ID NO: 179 - protein, 23 aa, synthetic construct
  REGION 1..23: note = Description of Artificial Sequence: Synthetic peptide
DKPSYQFGGHNSVDFEEDTLPKV

SEQ ID NO: 180 - protein, 26 aa, synthetic construct
  REGION 1..26: note = Description of Artificial Sequence: Synthetic peptide
RARRRKRASATQLYQTCKASGTCPPD

SEQ ID NO: 181 - protein, 18 aa, synthetic construct
  REGION 1..18: note = Description of Artificial Sequence: Synthetic peptide
SGDYSFALPVGQWPWMTG

SEQ ID NO: 182 - protein, 17 aa, synthetic construct
  REGION 1..17: note = Description of Artificial Sequence: Synthetic peptide
CTGGDYSFALPVGQWPW

SEQ ID NO: 183 - protein, 15 aa, synthetic construct
  REGION 1..15: note = Description of Artificial Sequence: Synthetic peptide
FYYDYDFFFDYWGQG

SEQ ID NO: 184 - protein, 15 aa, synthetic construct
  REGION 1..15: note = Description of Artificial Sequence: Synthetic peptide
HLRRLRRRLLREAEG

SEQ ID NO: 185 - protein, 17 aa, synthetic construct
  REGION 1..17: note = Description of Artificial Sequence: Synthetic peptide
DYYCAAWDDSLNGYSVF

SEQ ID NO: 186 - protein, 15 aa, synthetic construct
  REGION 1..15: note = Description of Artificial Sequence: Synthetic peptide
YYCLQSMEDPYTFGG

SEQ ID NO: 187 - protein, 20 aa, synthetic construct
  REGION 1..20: note = Description of Artificial Sequence: Synthetic peptide
YYCARSDGNYGYYYALDYDY

SEQ ID NO: 188 - protein, 12 aa, synthetic construct
  REGION 1..12: note = Description of Artificial Sequence: Synthetic peptide
AARSPSYYRYDY

SEQ ID NO: 189 - protein, 9 aa, synthetic construct
  REGION 1..9: note = Description of Artificial Sequence: Synthetic peptide
GPYYAMDYD

SEQ ID NO: 190 - protein, 22 aa, synthetic construct
  REGION 1..22: note = Description of Artificial Sequence: Synthetic peptide
YYCQQRSSYPYTEGGAYPKAWK

SEQ ID NO: 191 - protein, 18 aa, synthetic construct
  REGION 1..18: note = Description of Artificial Sequence: Synthetic peptide
YYCQRYDSDWSFGQGTKL

SEQ ID NO: 192 - protein, 18 aa, synthetic construct
  REGION 1..18: note = Description of Artificial Sequence: Synthetic peptide
YYCARSGYYAMDYWGQGT

SEQ ID NO: 193 - protein, 13 aa, synthetic construct
  REGION 1..13: note = Description of Artificial Sequence: Synthetic peptide
RVRRGACRGDCLG

SEQ ID NO: 194 - protein, 13 aa, synthetic construct
  REGION 1..13: note = Description of Artificial Sequence: Synthetic peptide
RVRRGACRYDCLG

SEQ ID NO: 195 - protein, 23 aa, synthetic construct
  REGION 1..23: note = Description of Artificial Sequence: Synthetic peptide
YYCAKGTHWGFWSGYFDYWGQGT

SEQ ID NO: 196 - protein, 17 aa, synthetic construct
  REGION 1..17: note = Description of Artificial Sequence: Synthetic peptide
GRENYHGCTTHWGFTLC

SEQ ID NO: 197 - protein, 17 aa, synthetic construct
  REGION 1..17: note = Description of Artificial Sequence: Synthetic peptide
VQATQSNQHTPRGGGSK

SEQ ID NO: 198 - protein, 7 aa, synthetic construct
  REGION 1..7: note = Description of Artificial Sequence: Synthetic peptide
DPRAPGS

SEQ ID NO: 199 - protein, 15 aa, synthetic construct
  REGION 1..15: note = Description of Artificial Sequence: Synthetic peptide
YYCQQRSSYPYTFGG

SEQ ID NO: 200 - protein, 21 aa, synthetic construct
  REGION 1..21: note = Description of Artificial Sequence: Synthetic peptide
AARSPSYYRYDYGPYYAMDYD

SEQ ID NO: 201 - protein, 18 aa, synthetic construct
  REGION 1..18: note = Description of Artificial Sequence: Synthetic peptide
GPKLTGILISILSLFVES

SEQ ID NO: 202 - protein, 11 aa, synthetic construct
  REGION 1..11: note = Description of Artificial Sequence: Synthetic peptide
KYILRWRPKNS

SEQ ID NO: 203 - protein, 5 aa, synthetic construct
  REGION 1..5: note = Description of Artificial Sequence: Synthetic peptide
IKVAV

SEQ ID NO: 204 - protein, 20 aa, synthetic construct
  REGION 1..20: note = Description of Artificial Sequence: Synthetic peptide
WTPPRAQITGYRLTVGLTRR

SEQ ID NO: 205 - protein, 12 aa, synthetic construct
  REGION 1..12: note = Description of Artificial Sequence: Synthetic peptide
AASIKVAVSADR

SEQ ID NO: 206 - protein, 6 aa, synthetic construct
  REGION 1..6: note = Description of Artificial Sequence: Synthetic peptide
KLDAPT

SEQ ID NO: 207 - protein, 12 aa, synthetic construct
  REGION 1..12: note = Description of Artificial Sequence: Synthetic peptide
NRWHSIYITRFG

SEQ ID NO: 208 - protein, 5 aa, synthetic construct
  REGION 1..5: note = Description of Artificial Sequence: Synthetic peptide
PHSRN

SEQ ID NO: 209 - protein, 12 aa, synthetic construct
  REGION 1..12: note = Description of Artificial Sequence: Synthetic peptide
SSFHFDGSGYAM

SEQ ID NO: 210 - protein, 4 aa, synthetic construct
  REGION 1..4: note = Description of Artificial Sequence: Synthetic peptide
RGDS

SEQ ID NO: 211 - protein, 6 aa, synthetic construct
  REGION 1..6: note = Description of Artificial Sequence: Synthetic peptide
IAFQRN

SEQ ID NO: 212 - protein, 6 aa, synthetic construct
  REGION 1..6: note = Description of Artificial Sequence: Synthetic peptide
GRGDSP

SEQ ID NO: 213 - protein, 11 aa, synthetic construct
  REGION 1..11: note = Description of Artificial Sequence: Synthetic peptide
TWYKIAFQRRK

SEQ ID NO: 214 - protein, 7 aa, synthetic construct
  REGION 1..7: note = Description of Artificial Sequence: Synthetic peptide
EDGIHEL

SEQ ID NO: 215 - protein, 12 aa, synthetic construct
  REGION 1..12: note = Description of Artificial Sequence: Synthetic peptide
SLVRNRRVITIQ

SEQ ID NO: 216 - protein, 16 aa, synthetic construct
  REGION 1..16: note = Description of Artificial Sequence: Synthetic peptide
YRVRVTPKEKTGPMKE

SEQ ID NO: 217 - protein, 7 aa, synthetic construct
  REGION 1..7: note = Description of Artificial Sequence: Synthetic peptide
LQVQLSR

SEQ ID NO: 218 - protein, 9 aa, synthetic construct
  REGION 1..9: note = Description of Artificial Sequence: Synthetic peptide
SPPRRARVT

SEQ ID NO: 219 - protein, 12 aa, synthetic construct
  REGION 1..12: note = Description of Artificial Sequence: Synthetic peptide
RKRLQVQLSIRT

SEQ ID NO: 220 - protein, 9 aa, synthetic construct
  REGION 1..9: note = Description of Artificial Sequence: Synthetic peptide
ATETTITIS

SEQ ID NO: 221 - protein, 12 aa, synthetic construct
  REGION 1..12: note = Description of Artificial Sequence: Synthetic peptide
NAPFPKLSWTIQ

SEQ ID NO: 222 - protein, 31 aa, synthetic construct
  REGION 1..31: note = Description of Artificial Sequence: Synthetic polypeptide
VSPPRRARVTDATETTITISWRTKTETITGG

SEQ ID NO: 223 - protein, 12 aa, synthetic construct
  REGION 1..12: note = Description of Artificial Sequence: Synthetic peptide
WTIQTTVDRGLL

SEQ ID NO: 224 - protein, 11 aa, synthetic construct
  REGION 1..11: note = Description of Artificial Sequence: Synthetic peptide
KPDVRSYTITG

SEQ ID NO: 225 - protein, 13 aa, synthetic construct
  REGION 1..13: note = Description of Artificial Sequence: Synthetic peptide
DTINNGRDHMILI

SEQ ID NO: 226 - protein, 12 aa, synthetic construct
  REGION 1..12: note = Description of Artificial Sequence: Synthetic peptide
ANGQTPIQRYIK

SEQ ID NO: 227 - protein, 13 aa, synthetic construct
  REGION 1..13: note = Description of Artificial Sequence: Synthetic peptide
MILISIGKSQKRM

SEQ ID NO: 228 - protein, 30 aa, synthetic construct
  REGION 1..30: note = Description of Artificial Sequence: Synthetic polypeptide
PRARITGYIIKYEKPGSPPREVVPRPRPGV

SEQ ID NO: 229 - protein, 12 aa, synthetic construct
  REGION 1..12: note = Description of Artificial Sequence: Synthetic peptide
PPFLMLLKGSTR

SEQ ID NO: 230 - protein, 8 aa, synthetic construct
  REGION 1..8: note = Description of Artificial Sequence: Synthetic peptide
WQPPRARI

SEQ ID NO: 231 - protein, 13 aa, synthetic construct
  REGION 1..13: note = Description of Artificial Sequence: Synthetic peptide
NQRLASFSNAQQS

SEQ ID NO: 232 - protein, 19 aa, synthetic construct
  REGION 1..19: note = Description of Artificial Sequence: Synthetic peptide
WQPPRARITGYIIKYEKPG

SEQ ID NO: 233 - protein, 17 aa, synthetic construct
  REGION 1..17: note = Description of Artificial Sequence: Synthetic peptide
ISNVFVQRMSQSPEVLD

SEQ ID NO: 234 - protein, 19 aa, synthetic construct
  REGION 1..19: note = Description of Artificial Sequence: Synthetic peptide
YEKPGSPPREVVPRPRPGV

SEQ ID NO: 235 - protein, 13 aa, synthetic construct
  REGION 1..13: note = Description of Artificial Sequence: Synthetic peptide
KARSFNVNQLLQD

SEQ ID NO: 236 - protein, 15 aa, synthetic construct
  REGION 1..15: note = Description of Artificial Sequence: Synthetic peptide
KNNQKSEPLIGRKKT

SEQ ID NO: 237 - protein, 12 aa, synthetic construct
  REGION 1..12: note = Description of Artificial Sequence: Synthetic peptide
KNSFMALYLSKG

SEQ ID NO: 238 - protein, 8 aa, synthetic construct
  REGION 1..8: note = Description of Artificial Sequence: Synthetic peptide
EILDVPST

SEQ ID NO: 239 - protein, 19 aa, synthetic construct
  REGION 1..19: note = Description of Artificial Sequence: Synthetic peptide
KNSFMALYLSKGRLVFALG

SEQ ID NO: 240 - protein, 5 aa, synthetic construct
  REGION 1..5: note = Description of Artificial Sequence: Synthetic peptide
IDAPS

SEQ ID NO: 241 - protein, 20 aa, synthetic construct
  REGION 1..20: note = Description of Artificial Sequence: Synthetic peptide
RDSFVALYLSEGHVIFAGLG

SEQ ID NO: 242 - protein, 15 aa, synthetic construct
  REGION 1..15: note = Description of Artificial Sequence: Synthetic peptide
VVIDASTAIDAPSNL

SEQ ID NO: 243 - protein, 12 aa, synthetic construct
  REGION 1..12: note = Description of Artificial Sequence: Synthetic peptide
KPRLQFSLDIQT

SEQ ID NO: 244 - protein, 5 aa, synthetic construct
  REGION 1..5: note = Description of Artificial Sequence: Synthetic peptide
LDVPS

SEQ ID NO: 245 - protein, 12 aa, synthetic construct
  REGION 1..12: note = Description of Artificial Sequence: Synthetic peptide
DGQWHSVTVSIK

SEQ ID NO: 246 - protein, 4 aa, synthetic construct
  REGION 1..4: note = Description of Artificial Sequence: Synthetic peptide
REDV

SEQ ID NO: 247 - protein, 12 aa, synthetic construct
  REGION 1..12: note = Description of Artificial Sequence: Synthetic peptide
FVLYLGSKNAKK

SEQ ID NO: 248 - protein, 10 aa, synthetic construct
  REGION 1..10: note = Description of Artificial Sequence: Synthetic peptide
PHSRNRGDSP

SEQ ID NO: 249 - protein, 12 aa, synthetic construct
  REGION 1..12: note = Description of Artificial Sequence: Synthetic peptide
LAIKNDNLVYVY

SEQ ID NO: 250 - protein, 13 aa, synthetic construct
  REGION 1..13: note = Description of Artificial Sequence: Synthetic peptide
LWVTVRSQQRGLF

SEQ ID NO: 251 - protein, 12 aa, synthetic construct
  REGION 1..12: note = Description of Artificial Sequence: Synthetic peptide
AYFSIVKIERVG

SEQ ID NO: 252 - protein, 13 aa, synthetic construct
  REGION 1..13: note = Description of Artificial Sequence: Synthetic peptide
GTNNWWQSPSIQN

SEQ ID NO: 253 - protein, 12 aa, synthetic construct
  REGION 1..12: note = Description of Artificial Sequence: Synthetic peptide
DVISLYNFKHIY

SEQ ID NO: 254 - protein, 13 aa, synthetic construct
  REGION 1..13: note = Description of Artificial Sequence: Synthetic peptide
WVTVTLDLRQVFQ

SEQ ID NO: 255 - protein, 12 aa, synthetic construct
  REGION 1..12: note = Description of Artificial Sequence: Synthetic peptide
FFDGSSYAVVRD

SEQ ID NO: 256 - protein, 13 aa, synthetic construct
  REGION 1..13: note = Description of Artificial Sequence: Synthetic peptide
RQVFQVAYIIIKA

SEQ ID NO: 257 - protein, 14 aa, synthetic construct
  REGION 1..14: note = Description of Artificial Sequence: Synthetic peptide
LHVFYDFGFGFSNG

SEQ ID NO: 258 - protein, 14 aa, synthetic construct
  REGION 1..14: note = Description of Artificial Sequence: Synthetic peptide
LTRYKITPRRGPPT

SEQ ID NO: 259 - protein, 20 aa, synthetic construct
  REGION 1..20: note = Description of Artificial Sequence: Synthetic peptide
LKKAQINDAKYREISIIYHN

SEQ ID NO: 260 - protein, 12 aa, synthetic construct
  REGION 1..12: note = Description of Artificial Sequence: Synthetic peptide
LLEFTSARYIRL

SEQ ID NO: 261 - protein, 12 aa, synthetic construct
  REGION 1..12: note = Description of Artificial Sequence: Synthetic peptide
RAYFNGQSFIAS

SEQ ID NO: 262 - protein, 12 aa, synthetic construct
  REGION 1..12: note = Description of Artificial Sequence: Synthetic peptide
YIRLRLQRIRTL

SEQ ID NO: 263 - protein, 12 aa, synthetic construct
  REGION 1..12: note = Description of Artificial Sequence: Synthetic peptide
SRLRGKNPTKGK

SEQ ID NO: 264 - protein, 12 aa, synthetic construct
  REGION 1..12: note = Description of Artificial Sequence: Synthetic peptide
RRYYYSIKDISV

SEQ ID NO: 265 - protein, 12 aa, synthetic construct
  REGION 1..12: note = Description of Artificial Sequence: Synthetic peptide
LHKKGKNSSKPK

SEQ ID NO: 266 - protein, 12 aa, synthetic construct
  REGION 1..12: note = Description of Artificial Sequence: Synthetic peptide
SINNTAVNQRLT

SEQ ID NO: 267 - protein, 12 aa, synthetic construct
  REGION 1..12: note = Description of Artificial Sequence: Synthetic peptide
RLKTRSSHGMIF

SEQ ID NO: 268 - protein, 12 aa, synthetic construct
  REGION 1..12: note = Description of Artificial Sequence: Synthetic peptide
GGFLKYTVSYDI

SEQ ID NO: 269 - protein, 12 aa, synthetic construct
  REGION 1..12: note = Description of Artificial Sequence: Synthetic peptide
GEKSQFSIRLKT

SEQ ID NO: 270 - protein, 12 aa, synthetic construct
  REGION 1..12: note = Description of Artificial Sequence: Synthetic peptide
RDQLMTVLANVT

SEQ ID NO: 271 - protein, 12 aa, synthetic construct
  REGION 1..12: note = Description of Artificial Sequence: Synthetic peptide
TLFLAHGRLVFM

SEQ ID NO: 272 - protein, 12 aa, synthetic construct
  REGION 1..12: note = Description of Artificial Sequence: Synthetic peptide
ANVTHLLIRANY

SEQ ID NO: 273 - protein, 12 aa, synthetic construct
  REGION 1..12: note = Description of Artificial Sequence: Synthetic peptide
LVFMFNVGHKKL

SEQ ID NO: 274 - protein, 13 aa, synthetic construct
  REGION 1..13: note = Description of Artificial Sequence: Synthetic peptide
AGTFALRGDNPQG

SEQ ID NO: 275 - protein, 20 aa, synthetic construct
  REGION 1..20: note = Description of Artificial Sequence: Synthetic peptide
TLFLAHGRLVFMFNVGHKKL

SEQ ID NO: 276 - protein, 12 aa, synthetic construct
  REGION 1..12: note = Description of Artificial Sequence: Synthetic peptide
VLIKGGRARKHV

SEQ ID NO: 277 - protein, 19 aa, synthetic construct
  REGION 1..19: note = Description of Artificial Sequence: Synthetic peptide
DFMTLFLAHGRLVFMGNVG

SEQ ID NO: 278 - protein, 11 aa, synthetic construct
  REGION 1..11: note = Description of Artificial Sequence: Synthetic peptide
LSNIDYLIKAS

SEQ ID NO: 279 - protein, 12 aa, synthetic construct
  REGION 1..12: note = Description of Artificial Sequence: Synthetic peptide
HKKLKIRSQEKY

SEQ ID NO: 280 - protein, 12 aa, synthetic construct
  REGION 1..12: note = Description of Artificial Sequence: Synthetic peptide
LQQSRIANISME

SEQ ID NO: 281 - protein, 12 aa, synthetic construct
  REGION 1..12: note = Description of Artificial Sequence: Synthetic peptide
GAAWKIKGPIYL

SEQ ID NO: 282 - protein, 12 aa, synthetic construct
  REGION 1..12: note = Description of Artificial Sequence: Synthetic peptide
NLLLLLVKANLK

SEQ ID NO: 283 - protein, 12 aa, synthetic construct
  REGION 1..12: note = Description of Artificial Sequence: Synthetic peptide
VIRDSNVVQLDV

SEQ ID NO: 284 - protein, 12 aa, synthetic construct
  REGION 1..12: note = Description of Artificial Sequence: Synthetic peptide
HRDELLLWARKI

SEQ ID NO: 285 - protein, 12 aa, synthetic construct
  REGION 1..12: note = Description of Artificial Sequence: Synthetic peptide
GLIYYVAHQNQM

SEQ ID NO: 286 - protein, 12 aa, synthetic construct
  REGION 1..12: note = Description of Artificial Sequence: Synthetic peptide
KRRARDLVHRAE

SEQ ID NO: 287 - protein, 19 aa, synthetic construct
  REGION 1..19: note = Description of Artificial Sequence: Synthetic peptide
DYATLQLQEGRLHFMFDLG

SEQ ID NO: 288 - protein, 12 aa, synthetic construct
  REGION 1..12: note = Description of Artificial Sequence: Synthetic peptide
SQFQESVDNITK

SEQ ID NO: 289 - protein, 12 aa, synthetic construct
  REGION 1..12: note = Description of Artificial Sequence: Synthetic peptide
KKGSYNNIVVHV

SEQ ID NO: 290 - protein, 12 aa, synthetic construct
  REGION 1..12: note = Description of Artificial Sequence: Synthetic peptide
PGGMREKGRKAR

SEQ ID NO: 291 - protein, 12 aa, synthetic construct
  REGION 1..12: note = Description of Artificial Sequence: Synthetic peptide
ADNLLFYLGSAK

SEQ ID NO: 292 - protein, 12 aa, synthetic construct
  REGION 1..12: note = Description of Artificial Sequence: Synthetic peptide
MEMQANLLLDRL

SEQ ID NO: 293 - protein, 12 aa, synthetic construct
  REGION 1..12: note = Description of Artificial Sequence: Synthetic peptide
GSAKFIDFLAIE

SEQ ID NO: 294 - protein, 11 aa, synthetic construct
  REGION 1..11: note = Description of Artificial Sequence: Synthetic peptide
LSEIKLLISAR

SEQ ID NO: 295 - protein, 12 aa, synthetic construct
  REGION 1..12: note = Description of Artificial Sequence: Synthetic peptide
KVSFLWWVGSGV

SEQ ID NO: 296 - protein, 15 aa, synthetic construct
  REGION 1..15: note = Description of Artificial Sequence: Synthetic peptide
RDFTKATNIRLRFLR

SEQ ID NO: 297 - protein, 12 aa, synthetic construct
  REGION 1..12: note = Description of Artificial Sequence: Synthetic peptide
SYWYRIEASRTG

SEQ ID NO: 298 - protein, 12 aa, synthetic construct
  REGION 1..12: note = Description of Artificial Sequence: Synthetic peptide
ISTVMFKFRTFS

SEQ ID NO: 299 - protein, 12 aa, synthetic construct
  REGION 1..12: note = Description of Artificial Sequence: Synthetic peptide
YFDGTGFAKAVG

SEQ ID NO: 300 - protein, 13 aa, synthetic construct
  REGION 1..13: note = Description of Artificial Sequence: Synthetic peptide
KQANISIVDIDSN

SEQ ID NO: 301 - protein, 12 aa, synthetic construct
  REGION 1..12: note = Description of Artificial Sequence: Synthetic peptide
NGQWHKVTAKKI

SEQ ID NO: 302 - protein, 12 aa, synthetic construct
  REGION 1..12: note = Description of Artificial Sequence: Synthetic peptide
FSTRNESGIILL

SEQ ID NO: 303 - protein, 12 aa, synthetic construct
  REGION 1..12: note = Description of Artificial Sequence: Synthetic peptide
AKKIKNRLELVV

SEQ ID NO: 304 - protein, 12 aa, synthetic construct
  REGION 1..12: note = Description of Artificial Sequence: Synthetic peptide
RRQTTQAYYAIF

SEQ ID NO: 305 - protein, 14 aa, synthetic construct
  REGION 1..14: note = Description of Artificial Sequence: Synthetic peptide
GFPGGLNQFGLTTN

SEQ ID NO: 306 - protein, 12 aa, synthetic construct
  REGION 1..12: note = Description of Artificial Sequence: Synthetic peptide
YAIFLNKGRLEV

SEQ ID NO: 307 - protein, 13 aa, synthetic construct
  REGION 1..13: note = Description of Artificial Sequence: Synthetic peptide
NQFGLTTNIRFRG

SEQ ID NO: 308 - protein, 12 aa, synthetic construct
  REGION 1..12: note = Description of Artificial Sequence: Synthetic peptide
KNRLTIELEVRT

SEQ ID NO: 309 - protein, 13 aa, synthetic construct
  REGION 1..13: note = Description of Artificial Sequence: Synthetic peptide
IRSLKLTKGTGKP

SEQ ID NO: 310 - protein, 12 aa, synthetic construct
  REGION 1..12: note = Description of Artificial Sequence: Synthetic peptide
GLLFYMARINHA

SEQ ID NO: 311 - protein, 13 aa, synthetic construct
  REGION 1..13: note = Description of Artificial Sequence: Synthetic peptide
AKALELRGVQPVS

SEQ ID NO: 312 - protein, 12 aa, synthetic construct
  REGION 1..12: note = Description of Artificial Sequence: Synthetic peptide
VQLRNGFPYFSY

SEQ ID NO: 313 - protein, 13 aa, synthetic construct
  REGION 1..13: note = Description of Artificial Sequence: Synthetic peptide
GQLFHVAYILIKF

SEQ ID NO: 314 - protein, 12 aa, synthetic construct
  REGION 1..12: note = Description of Artificial Sequence: Synthetic peptide
HKIKIVRVKQEG

SEQ ID NO: 315 - protein, 12 aa, synthetic construct
  REGION 1..12: note = Description of Artificial Sequence: Synthetic peptide
NVLSLYNFKTTF

SEQ ID NO: 316 - protein, 19 aa, synthetic construct
  REGION 1..19: note = Description of Artificial Sequence: Synthetic peptide
DFGTVQLRNGFPFFSYDLG

SEQ ID NO: 317 - protein, 13 aa, synthetic construct
  REGION 1..13: note = Description of Artificial Sequence: Synthetic peptide
SQRIYQFAKLNYT

SEQ ID NO: 318 - protein, 12 aa, synthetic construct
  REGION 1..12: note = Description of Artificial Sequence: Synthetic peptide
NIRLRFLRTNTL

SEQ ID NO: 319 - protein, 13 aa, synthetic construct
  REGION 1..13: note = Description of Artificial Sequence: Synthetic peptide
EVNVTLDLGQVFH

SEQ ID NO: 320 - protein, 12 aa, synthetic construct
  REGION 1..12: note = Description of Artificial Sequence: Synthetic peptide
GKNTGDHFVLYM

SEQ ID NO: 321 - protein, 13 aa, synthetic construct
  REGION 1..13: note = Description of Artificial Sequence: Synthetic peptide
GQVFHVAYVLIKF

SEQ ID NO: 322 - protein, 13 aa, synthetic construct
  REGION 1..13: note = Description of Artificial Sequence: Synthetic peptide
VVSLYNFEQTFML

SEQ ID NO: 323 - protein, 21 aa, synthetic construct
  REGION 1..21: note = Description of Artificial Sequence: Synthetic peptide
HQQDLGTAGSCLRKFSTMFLF

SEQ ID NO: 324 - protein, 12 aa, synthetic construct
  REGION 1..12: note = Description of Artificial Sequence: Synthetic peptide
RFDQELRLVSYN

SEQ ID NO: 325 - protein, 24 aa, synthetic construct
  REGION 1..24: note = Description of Artificial Sequence: Synthetic peptide
HQQDLGTAGSCLRKFSTMFLFCNI

SEQ ID NO: 326 - protein, 13 aa, synthetic construct
  REGION 1..13: note = Description of Artificial Sequence: Synthetic peptide
RLVSYSGVLFFLK

SEQ ID NO: 327 - protein, 9 aa, synthetic construct
  REGION 1..9: note = Description of Artificial Sequence: Synthetic peptide
VAEIDGIEL

SEQ ID NO: 328 - protein, 12 aa, synthetic construct
  REGION 1..12: note = Description of Artificial Sequence: Synthetic peptide
NWRHISYITRFG

SEQ ID NO: 329 - protein, 6 aa, synthetic construct
  REGION 1..6: note = Description of Artificial Sequence: Synthetic peptide
GIIFFL

SEQ ID NO: 330 - protein, 12 aa, synthetic construct
  REGION 1..12: note = Description of Artificial Sequence: Synthetic peptide
KRLQVQLRSIRT

SEQ ID NO: 331 - protein, 12 aa, synthetic construct
  REGION 1..12: note = Description of Artificial Sequence: Synthetic peptide
ASKAIQVFLLGG

SEQ ID NO: 332 - protein, 12 aa, synthetic construct
  REGION 1..12: note = Description of Artificial Sequence: Synthetic peptide
TWYKIAFQRNRK

SEQ ID NO: 333 - protein, 12 aa, synthetic construct
  REGION 1..12: note = Description of Artificial Sequence: Synthetic peptide
VLVRVERATVFS

SEQ ID NO: 334 - protein, 12 aa, synthetic construct
  REGION 1..12: note = Description of Artificial Sequence: Synthetic peptide
QVFQVAYIIIKA

SEQ ID NO: 335 - protein, 12 aa, synthetic construct
  REGION 1..12: note = Description of Artificial Sequence: Synthetic peptide
TVFSVDQDNMLE

SEQ ID NO: 336 - protein, 13 aa, synthetic construct
  REGION 1..13: note = Description of Artificial Sequence: Synthetic peptide
GEFYFDLRLKGDK

SEQ ID NO: 337 - protein, 12 aa, synthetic construct
  REGION 1..12: note = Description of Artificial Sequence: Synthetic peptide
RLRGPQRVFDLH

SEQ ID NO: 338 - protein, 9 aa, synthetic construct
  REGION 1..9: note = Description of Artificial Sequence: Synthetic peptide
GTPGPQGIA

SEQ ID NO: 339 - protein, 11 aa, synthetic construct
  REGION 1..11: note = Description of Artificial Sequence: Synthetic peptide
FDLHQNMGSVN

SEQ ID NO: 340 - protein, 6 aa, synthetic construct
  REGION 1..6: note = Description of Artificial Sequence: Synthetic peptide
GQRDVV

SEQ ID NO: 341 - protein, 10 aa, synthetic construct
  REGION 1..10: note = Description of Artificial Sequence: Synthetic peptide
LRAHAVDVNG

SEQ ID NO: 342 - protein, 12 aa, synthetic construct
  REGION 1..12: note = Description of Artificial Sequence: Synthetic peptide
TAGSCLRKFSTM

SEQ ID NO: 343 - protein, 10 aa, synthetic construct
  REGION 1..10: note = Description of Artificial Sequence: Synthetic peptide
LFSHAVSSNG

SEQ ID NO: 344 - protein, 6 aa, synthetic construct
  REGION 1..6: note = Description of Artificial Sequence: Synthetic peptide
KGHRGF

SEQ ID NO: 345 - protein, 15 aa, synthetic construct
  REGION 1..15: note = Description of Artificial Sequence: Synthetic peptide
TAGSCLRKFSTMFLF

SEQ ID NO: 346 - protein, 18 aa, synthetic construct
  REGION 1..18: note = Description of Artificial Sequence: Synthetic peptide
TAGSCLRKFSTMFLFCNI

SEQ ID NO: 347 - protein, 15 aa, synthetic construct
  REGION 1..15: note = Description of Artificial Sequence: Synthetic peptide
DLGTAGSCLRKFSTM

SEQ ID NO: 348 - protein, 18 aa, synthetic construct
  REGION 1..18: note = Description of Artificial Sequence: Synthetic peptide
HQQDLGTAGSCLRKFSTM

SEQ ID NO: 349 - protein, 10 aa, synthetic construct
  REGION 1..10: note = Description of Artificial Sequence: Synthetic peptide
RNIAEIIKDI

SEQ ID NO: 350 - protein, 11 aa, synthetic construct
  REGION 1..11: note = Description of Artificial Sequence: Synthetic peptide
SIGFRGDGQTC

SEQ ID NO: 351 - protein, 10 aa, synthetic construct
  REGION 1..10: note = Description of Artificial Sequence: Synthetic peptide
LNRQELFPFG

SEQ ID NO: 352 - protein, 17 aa, synthetic construct
  REGION 1..17: note = Description of Artificial Sequence: Synthetic peptide
RIQNLLKITNLRIKFVK

SEQ ID NO: 353 - protein, 16 aa, synthetic construct
  REGION 1..16: note = Description of Artificial Sequence: Synthetic peptide
KKQRFRHRNRKGYRSQ

SEQ ID NO: 354 - protein, 12 aa, synthetic construct
  REGION 1..12: note = Description of Artificial Sequence: Synthetic peptide
SINNTAVMQRLT

SEQ ID NO: 355 - protein, 9 aa, synthetic construct
  REGION 1..9: note = Description of Artificial Sequence: Synthetic peptide
FRHRNRKGY

SEQ ID NO: 356 - protein, 17 aa, synthetic construct
  REGION 1..17: note = Description of Artificial Sequence: Synthetic peptide
RYRVRVTPKEKTGPMKE

SEQ ID NO: 357 - protein, 13 aa, synthetic construct
  REGION 1..13: note = Description of Artificial Sequence: Synthetic peptide
SETTVKYIFRLHE

SEQ ID NO: 358 - protein, 21 aa, synthetic construct
  REGION 1..21: note = Description of Artificial Sequence: Synthetic peptide
GHRGPTGRPGKRGKQGQKGDS

SEQ ID NO: 359 - protein, 12 aa, synthetic construct
  REGION 1..12: note = Description of Artificial Sequence: Synthetic peptide
KAFDITYVRLKF

SEQ ID NO: 360 - protein, 15 aa, synthetic construct
  REGION 1..15: note = Description of Artificial Sequence: Synthetic peptide
GDLGRPGRKGRPGPP

SEQ ID NO: 361 - protein, 5 aa, synthetic construct
  REGION 1..5: note = Description of Artificial Sequence: Synthetic peptide
YIGSR

SEQ ID NO: 362 - protein, 14 aa, synthetic construct
  REGION 1..14: note = Description of Artificial Sequence: Synthetic peptide
RGEFYFDLRLKGDK

SEQ ID NO: 363 - protein, 12 aa, synthetic construct
  REGION 1..12: note = Description of Artificial Sequence: Synthetic peptide
LAGSCLARFSTM

SEQ ID NO: 364 - protein, 12 aa, synthetic construct
  REGION 1..12: note = Description of Artificial Sequence: Synthetic peptide
LALFLSNGHFVA

SEQ ID NO: 365 - protein, 12 aa, synthetic construct
  REGION 1..12: note = Description of Artificial Sequence: Synthetic peptide
ISRCQVCMKKRH

SEQ ID NO: 366 - protein, 12 aa, synthetic construct
  REGION 1..12: note = Description of Artificial Sequence: Synthetic peptide
PGRWHKVSVRWE

SEQ ID NO: 367 - protein, 15 aa, synthetic construct
  REGION 1..15: note = Description of Artificial Sequence: Synthetic peptide
TDIPPCPHGWISLWK

SEQ ID NO: 368 - protein, 12 aa, synthetic construct
  REGION 1..12: note = Description of Artificial Sequence: Synthetic peptide
VRWGMQQIQLVV

SEQ ID NO: 369 - protein, 15 aa, synthetic construct
  REGION 1..15: note = Description of Artificial Sequence: Synthetic peptide
TAIPSCPEGTVPLYS

SEQ ID NO: 370 - protein, 12 aa, synthetic construct
  REGION 1..12: note = Description of Artificial Sequence: Synthetic peptide
KMPYVSLELEMR

SEQ ID NO: 371 - protein, 13 aa, synthetic construct
  REGION 1..13: note = Description of Artificial Sequence: Synthetic peptide
GPAGKDGEAGAQG

SEQ ID NO: 372 - protein, 12 aa, synthetic construct
  REGION 1..12: note = Description of Artificial Sequence: Synthetic peptide
VLLQANDGAGEF

SEQ ID NO: 373 - protein, 6 aa, synthetic construct
  REGION 1..6: note = Description of Artificial Sequence: Synthetic peptide
GLPGER

SEQ ID NO: 374 - protein, 12 aa, synthetic construct
  REGION 1..12: note = Description of Artificial Sequence: Synthetic peptide
DGRWHRVAVIMG

SEQ ID NO: 375 - protein, 12 aa, synthetic construct
  REGION 1..12: note = Description of Artificial Sequence: Synthetic peptide
LAGSCLPVFSTL

SEQ ID NO: 376 - protein, 12 aa, synthetic construct
  REGION 1..12: note = Description of Artificial Sequence: Synthetic peptide
APVNVTASVQIQ

SEQ ID NO: 377 - protein, 12 aa, synthetic construct
  REGION 1..12: note = Description of Artificial Sequence: Synthetic peptide
TAGSCLRRFSTM

SEQ ID NO: 378 - protein, 12 aa, synthetic construct
  REGION 1..12: note = Description of Artificial Sequence: Synthetic peptide
KQGKALTQRHAK

SEQ ID NO: 379 - protein, 9 aa, synthetic construct
  REGION 1..9: note = Description of Artificial Sequence: Synthetic peptide
TAGSCLRKF

SEQ ID NO: 380 - protein, 7 aa, synthetic construct
  REGION 1..7: note = Description of Artificial Sequence: Synthetic peptide
RYVVLPR

SEQ ID NO: 381 - protein, 6 aa, synthetic construct
  REGION 1..6: note = Description of Artificial Sequence: Synthetic peptide
TAGSCL

SEQ ID NO: 382 - protein, 14 aa, synthetic construct
  REGION 1..14: note = Description of Artificial Sequence: Synthetic peptide
SPYTFIDSLVLMPY

SEQ ID NO: 383 - skipped

SEQ ID NO: 384 - protein, 5 aa, synthetic construct
  REGION 1..5: note = Description of Artificial Sequence: Synthetic peptide
PDSGR

SEQ ID NO: 385 - protein, 12 aa, synthetic construct
  REGION 1..12: note = Description of Artificial Sequence: Synthetic peptide
QQNLGSVNVSTG

SEQ ID NO: 386 - protein, 12 aa, synthetic construct
  REGION 1..12: note = Description of Artificial Sequence: Synthetic peptide
SRATAQKVSRRS

SEQ ID NO: 387 - protein, 8 aa, synthetic construct
  REGION 1..8: note = Description of Artificial Sequence: Synthetic peptide
DPGYIGSR

SEQ ID NO: 388 - protein, 12 aa, synthetic construct
  REGION 1..12: note = Description of Artificial Sequence: Synthetic peptide
GSLSSHLEFVGI

SEQ ID NO: 389 - protein, 12 aa, synthetic construct
  REGION 1..12: note = Description of Artificial Sequence: Synthetic peptide
VILQQSAADIAR

SEQ ID NO: 390 - protein, 12 aa, synthetic construct
  REGION 1..12: note = Description of Artificial Sequence: Synthetic peptide
RNRLHLSMLVRP

SEQ ID NO: 391 - protein, 12 aa, synthetic construct
  REGION 1..12: note = Description of Artificial Sequence: Synthetic peptide
KDISEKVAVYST

SEQ ID NO: 392 - protein, 13 aa, synthetic construct
  REGION 1..13: note = Description of Artificial Sequence: Synthetic peptide
APMSGRSPSLVLK

SEQ ID NO: 393 - protein, 6 aa, synthetic construct
  REGION 1..6: note = Description of Artificial Sequence: Synthetic peptide
LGTIPG

SEQ ID NO: 394 - protein, 12 aa, synthetic construct
  REGION 1..12: note = Description of Artificial Sequence: Synthetic peptide
AFGVLALWGTRV

SEQ ID NO: 395 - protein, 13 aa, synthetic construct
  REGION 1..13: note = Description of Artificial Sequence: Synthetic peptide
TDIRVTLNRLNTF

SEQ ID NO: 396 - protein, 12 aa, synthetic construct
  REGION 1..12: note = Description of Artificial Sequence: Synthetic peptide
IENVVTTFAPNR

SEQ ID NO: 397 - protein, 12 aa, synthetic construct
  REGION 1..12: note = Description of Artificial Sequence: Synthetic peptide
AFSTLEGRPSAY

SEQ ID NO: 398 - protein, 12 aa, synthetic construct
  REGION 1..12: note = Description of Artificial Sequence: Synthetic peptide
LEAEFHFTHLIM

SEQ ID NO: 399 - protein, 12 aa, synthetic construct
  REGION 1..12: note = Description of Artificial Sequence: Synthetic peptide
TSAEAYNLLLRT

SEQ ID NO: 400 - protein, 12 aa, synthetic construct
  REGION 1..12: note = Description of Artificial Sequence: Synthetic peptide
HLIMTFKTFRPA

SEQ ID NO: 401 - protein, 12 aa, synthetic construct
  REGION 1..12: note = Description of Artificial Sequence: Synthetic peptide
LNRRYEQARNIS

SEQ ID NO: 402 - protein, 12 aa, synthetic construct
  REGION 1..12: note = Description of Artificial Sequence: Synthetic peptide
KTWGVYRYFAYD

SEQ ID NO: 403 - protein, 12 aa, synthetic construct
  REGION 1..12: note = Description of Artificial Sequence: Synthetic peptide
SLLSQLNNLLDQ

SEQ ID NO: 404 - protein, 12 aa, synthetic construct
  REGION 1..12: note = Description of Artificial Sequence: Synthetic peptide
TNLRIKFVKLHT

SEQ ID NO: 405 - protein, 10 aa, synthetic construct
  REGION 1..10: note = Description of Artificial Sequence: Synthetic peptide
RDIAEIIKDI

SEQ ID NO: 406 - protein, 8 aa, synthetic construct
  REGION 1..8: note = Description of Artificial Sequence: Synthetic peptide
KRLVTGQR

SEQ ID NO: 407 - protein, 6 aa, synthetic construct
  REGION 1..6: note = Description of Artificial Sequence: Synthetic peptide
SHAVSS

SEQ ID NO: 408 - protein, 12 aa, synthetic construct
  REGION 1..12: note = Description of Artificial Sequence: Synthetic peptide
GPGVVVVERQYI

SEQ ID NO: 409 - protein, 6 aa, synthetic construct
  REGION 1..6: note = Description of Artificial Sequence: Synthetic peptide
ADTPPV

SEQ ID NO: 410 - protein, 13 aa, synthetic construct
  REGION 1..13: note = Description of Artificial Sequence: Synthetic peptide
NEPKVLKSYYYAI

SEQ ID NO: 411 - protein, 10 aa, synthetic construct
  REGION 1..10: note = Description of Artificial Sequence: Synthetic peptide
LRAHAVDING

SEQ ID NO: 412 - protein, 12 aa, synthetic construct
  REGION 1..12: note = Description of Artificial Sequence: Synthetic peptide
YYAISDFAVGGR

SEQ ID NO: 413 - protein, 12 aa, synthetic construct
  REGION 1..12: note = Description of Artificial Sequence: Synthetic peptide
DSITKYFQMSLE

SEQ ID NO: 414 - protein, 13 aa, synthetic construct
  REGION 1..13: note = Description of Artificial Sequence: Synthetic peptide
LPFFNDRPWRRAT

SEQ ID NO: 415 - protein, 10 aa, synthetic construct
  REGION 1..10: note = Description of Artificial Sequence: Synthetic peptide
YTALIIATDN

SEQ ID NO: 416 - protein, 14 aa, synthetic construct
  REGION 1..14: note = Description of Artificial Sequence: Synthetic peptide
FDPELYRSTGHGGH

SEQ ID NO: 417 - protein, 10 aa, synthetic construct
  REGION 1..10: note = Description of Artificial Sequence: Synthetic peptide
VITVKDINDN

SEQ ID NO: 418 - protein, 12 aa, synthetic construct
  REGION 1..12: note = Description of Artificial Sequence: Synthetic peptide
TNAVGYSVYDIS

SEQ ID NO: 419 - protein, 10 aa, synthetic construct
  REGION 1..10: note = Description of Artificial Sequence: Synthetic peptide
GLDRESYPYY

SEQ ID NO: 420 - protein, 13 aa, synthetic construct
  REGION 1..13: note = Description of Artificial Sequence: Synthetic peptide
APVKFLGNQVLSY

SEQ ID NO: 421 - protein, 10 aa, synthetic construct
  REGION 1..10: note = Description of Artificial Sequence: Synthetic peptide
MKVSATDADD

SEQ ID NO: 422 - protein, 12 aa, synthetic construct
  REGION 1..12: note = Description of Artificial Sequence: Synthetic peptide
SFSFRVDRRDTR

SEQ ID NO: 423 - protein, 12 aa, synthetic construct
  REGION 1..12: note = Description of Artificial Sequence: Synthetic peptide
PQVTRGDVFTMP

SEQ ID NO: 424 - protein, 17 aa, synthetic construct
  REGION 1..17: note = Description of Artificial Sequence: Synthetic peptide
TWSKVGGHLRPGIVQSG

SEQ ID NO: 425 - protein, 12 aa, synthetic construct
  REGION 1..12: note = Description of Artificial Sequence: Synthetic peptide
KEAEREVTDLLR

SEQ ID NO: 426 - protein, 4 aa, synthetic construct
  REGION 1..4: note = Description of Artificial Sequence: Synthetic peptide
RGDV

SEQ ID NO: 427 - protein, 12 aa, synthetic construct
  REGION 1..12: note = Description of Artificial Sequence: Synthetic peptide
AAEPLKNIGILF

SEQ ID NO: 428 - protein, 11 aa, synthetic construct
  REGION 1..11: note = Description of Artificial Sequence: Synthetic peptide
FALWDAIIGEL

SEQ ID NO: 429 - protein, 6 aa, synthetic construct
  REGION 1..6: note = Description of Artificial Sequence: Synthetic peptide
VGVAPG

SEQ ID NO: 430 - protein, 12 aa, synthetic construct
  REGION 1..12: note = Description of Artificial Sequence: Synthetic peptide
LWPLLAVLAAVA

SEQ ID NO: 431 - protein, 5 aa, synthetic construct
  REGION 1..5: note = Description of Artificial Sequence: Synthetic peptide
PGVGV

SEQ ID NO: 432 - protein, 8 aa, synthetic construct
  REGION 1..8: note = Description of Artificial Sequence: Synthetic peptide
VFDNFVLK

SEQ ID NO: 433 - protein, 12 aa, synthetic construct
  REGION 1..12: note = Description of Artificial Sequence: Synthetic peptide
TSIKIRGTYSER

SEQ ID NO: 434 - protein, 10 aa, synthetic construct
  REGION 1..10: note = Description of Artificial Sequence: Synthetic peptide
TTSWSQCSKS

SEQ ID NO: 435 - protein, 6 aa, synthetic construct
  REGION 1..6: note = Description of Artificial Sequence: Synthetic peptide
DPETGV

SEQ ID NO: 436 - protein, 4 aa, synthetic construct
  REGION 1..4: note = Description of Artificial Sequence: Synthetic peptide
KRSR

SEQ ID NO: 437 - protein, 10 aa, synthetic construct
  REGION 1..10: note = Description of Artificial Sequence: Synthetic peptide
QGADTPPVGV

SEQ ID NO: 438 - protein, 7 aa, synthetic construct
  REGION 1..7: note = Description of Artificial Sequence: Synthetic peptide
SVVYGLR

SEQ ID NO: 439 - protein, 10 aa, synthetic construct
  REGION 1..10: note = Description of Artificial Sequence: Synthetic peptide
PLDREAIAKY

SEQ ID NO: 440 - protein, 10 aa, synthetic construct
  REGION 1..10: note = Description of Artificial Sequence: Synthetic peptide
DGRGDSVAYG

SEQ ID NO: 441 - protein, 5 aa, synthetic construct
  REGION 1..5: note = Description of Artificial Sequence: Synthetic peptide
HAVDI

SEQ ID NO: 442 - protein, 10 aa, synthetic construct
  REGION 1..10: note = Description of Artificial Sequence: Synthetic peptide
LALERKDHSG

SEQ ID NO: 443 - protein, 5 aa, synthetic construct
  REGION 1..5: note = Description of Artificial Sequence: Synthetic peptide
DQNDN

SEQ ID NO: 444 - protein, 19 aa, synthetic construct
  REGION 1..19: note = Description of Artificial Sequence: Synthetic peptide
YSMKKTTMKIIPFNRLTIG

SEQ ID NO: 445 - protein, 10 aa, synthetic construct
  REGION 1..10: note = Description of Artificial Sequence: Synthetic peptide
QDPELPDKNM

SEQ ID NO: 446 - protein, 4 aa, synthetic construct
  REGION 1..4: note = Description of Artificial Sequence: Synthetic peptide
RGDF

SEQ ID NO: 447 - protein, 10 aa, synthetic construct
  REGION 1..10: note = Description of Artificial Sequence: Synthetic peptide
LVVQAADLQG

SEQ ID NO: 448 - protein, 13 aa, synthetic construct
  REGION 1..13: note = Description of Artificial Sequence: Synthetic peptide
GVYYQGGTYSKAS

SEQ ID NO: 449 - protein, 10 aa, synthetic construct
  REGION 1..10: note = Description of Artificial Sequence: Synthetic peptide
NDDGGQFVVT

SEQ ID NO: 450 - protein, 12 aa, synthetic construct
  REGION 1..12: note = Description of Artificial Sequence: Synthetic peptide
TAGSCLRKFSCL

SEQ ID NO: 451 - protein, 9 aa, synthetic construct
  REGION 1..9: note = Description of Artificial Sequence: Synthetic peptide
YILHVAVTN

SEQ ID NO: 452 - protein, 19 aa, synthetic construct
  REGION 1..19: note = Description of Artificial Sequence: Synthetic peptide
CNYYSNSYSFWLASLNPER

SEQ ID NO: 453 - protein, 10 aa, synthetic construct
  REGION 1..10: note = Description of Artificial Sequence: Synthetic peptide
TYRIWRDTAN

SEQ ID NO: 454 - protein, 12 aa, synthetic construct
  REGION 1..12: note = Description of Artificial Sequence: Synthetic peptide
TGLSCLQRFTTM

SEQ ID NO: 455 - protein, 26 aa, synthetic construct
  REGION 1..26: note = Description of Artificial Sequence: Synthetic peptide
GFTCECSIGFRGDGQTCYGIVFWSEV

SEQ ID NO: 456 - protein, 12 aa, synthetic construct
  REGION 1..12: note = Description of Artificial Sequence: Synthetic peptide
HHLGGAKQAGDV

SEQ ID NO: 457 - protein, 21 aa, synthetic construct
  REGION 1..21: note = Description of Artificial Sequence: Synthetic peptide
SCLPGFSGDGRACRDVDECGH

SEQ ID NO: 458 - protein, 30 aa, synthetic construct
  REGION 1..30: note = Description of Artificial Sequence: Synthetic polypeptide
MAPRPSLAKKQRFRHRNRKGYRSQRGHSRG

SEQ ID NO: 459 - protein, 16 aa, synthetic construct
  REGION 1..16: note = Description of Artificial Sequence: Synthetic peptide
KKQKFRHRNRKGYRSQ

SEQ ID NO: 460 - protein, 15 aa, synthetic construct
  REGION 1..15: note = Description of Artificial Sequence: Synthetic peptide
KKQKFKHRNRKGYRS

SEQ ID NO: 461 - protein, 16 aa, synthetic construct
  REGION 1..16: note = Description of Artificial Sequence: Synthetic peptide
KKQKFRRRNRKGYRSH

SEQ ID NO: 462 - protein, 15 aa, synthetic construct
  REGION 1..15: note = Description of Artificial Sequence: Synthetic peptide
TAIPPCPHGWISLWK

SEQ ID NO: 463 - protein, 15 aa, synthetic construct
  REGION 1..15: note = Description of Artificial Sequence: Synthetic peptide
KKQKSRHRSRKRYRS

SEQ ID NO: 464 - protein, 15 aa, synthetic construct
  REGION 1..15: note = Description of Artificial Sequence: Synthetic peptide
KKQKSRRRSRKGYRS

SEQ ID NO: 465 - protein, 7 aa, synthetic construct
  REGION 1..7: note = Description of Artificial Sequence: Synthetic peptide
ISRCTVC

SEQ ID NO: 466 - protein, 12 aa, synthetic construct
  REGION 1..12: note = Description of Artificial Sequence: Synthetic peptide
ISRCQVCMKRRH

SEQ ID NO: 467 - protein, 7 aa, synthetic construct
  REGION 1..7: note = Description of Artificial Sequence: Synthetic peptide
VSRCTVC

SEQ ID NO: 468 - protein, 15 aa, synthetic construct
  REGION 1..15: note = Description of Artificial Sequence: Synthetic peptide
TDIPPCPQGWISLWK

SEQ ID NO: 469 - protein, 21 aa, synthetic construct
  REGION 1..21: note = Description of Artificial Sequence: Synthetic peptide
TVKAGELEKIISRCQVMKKRH

SEQ ID NO: 470 - protein, 15 aa, synthetic construct
  REGION 1..15: note = Description of Artificial Sequence: Synthetic peptide
TDIPSCPHGWISLWK

SEQ ID NO: 471 - protein, 15 aa, synthetic construct
  REGION 1..15: note = Description of Artificial Sequence: Synthetic peptide
TDIPPCPAGWISLWK

SEQ ID NO: 472 - protein, 15 aa, synthetic construct
  REGION 1..15: note = Description of Artificial Sequence: Synthetic peptide
TEIPPCPQGWISLWK

SEQ ID NO: 473 - protein, 15 aa, synthetic construct
  REGION 1..15: note = Description of Artificial Sequence: Synthetic peptide
TDVPPCPQGWISLWK

SEQ ID NO: 474 - protein, 13 aa, synthetic construct
  REGION 1..13: note = Description of Artificial Sequence: Synthetic peptide
RLVSYNGILFFLK

SEQ ID NO: 475 - protein, 13 aa, synthetic construct
  REGION 1..13: note = Description of Artificial Sequence: Synthetic peptide
RLVSYSGVIFFLK

SEQ ID NO: 476 - protein, 12 aa, synthetic construct
  REGION 1..12: note = Description of Artificial Sequence: Synthetic peptide
RLVSYNGILFFL

SEQ ID NO: 477 - protein, 13 aa, synthetic construct
  REGION 1..13: note = Description of Artificial Sequence: Synthetic peptide
RLVSYSGIIFFLK

SEQ ID NO: 478 - protein, 20 aa, synthetic construct
  REGION 1..20: note = Description of Artificial Sequence: Synthetic peptide
RFEQELRLVSYSGVLFFLKQ

SEQ ID NO: 479 - protein, 13 aa, synthetic construct
  REGION 1..13: note = Description of Artificial Sequence: Synthetic peptide
RLVSYNGIIFFLK

SEQ ID NO: 480 - protein, 30 aa, synthetic construct
  REGION 1..30: note = Description of Artificial Sequence: Synthetic polypeptide
DPAFKIEDPYSPRIQNLLKITNLRIKFVKL

SEQ ID NO: 481 - protein, 20 aa, synthetic construct
  REGION 1..20: note = Description of Artificial Sequence: Synthetic peptide
TKRFEQELRLVSYSGVLFFL

SEQ ID NO: 482 - protein, 10 aa, synthetic construct
  REGION 1..10: note = Description of Artificial Sequence: Synthetic peptide
GGRLKYSVAF

SEQ ID NO: 483 - protein, 12 aa, synthetic construct
  REGION 1..12: note = Description of Artificial Sequence: Synthetic peptide
GGFLRYTVSYDI

SEQ ID NO: 484 - protein, 12 aa, synthetic construct
  REGION 1..12: note = Description of Artificial Sequence: Synthetic peptide
GGFLKYTVSYDV

SEQ ID NO: 485 - protein, 22 aa, synthetic construct
  REGION 1..22: note = Description of Artificial Sequence: Synthetic peptide
LGNKLTAFGGFLKYTVSYDIPV

SEQ ID NO: 486 - protein, 12 aa, synthetic construct
  REGION 1..12: note = Description of Artificial Sequence: Synthetic peptide
GGYLKYTVSYDI

SEQ ID NO: 487 - protein, 13 aa, synthetic construct
  REGION 1..13: note = Description of Artificial Sequence: Synthetic peptide
GEIFFDMRLKGDK

SEQ ID NO: 488 - protein, 13 aa, synthetic construct
  REGION 1..13: note = Description of Artificial Sequence: Synthetic peptide
GEIYFDLRLKGDK

SEQ ID NO: 489 - protein, 13 aa, synthetic construct
  REGION 1..13: note = Description of Artificial Sequence: Synthetic peptide
GEIYLDMRLKGDK

SEQ ID NO: 490 - protein, 30 aa, synthetic construct
  REGION 1..30: note = Description of Artificial Sequence: Synthetic polypeptide
IGQPGAKGEPGEFYFDLRLKGDKGDPGFPG

SEQ ID NO: 491 - protein, 13 aa, synthetic construct
  REGION 1..13: note = Description of Artificial Sequence: Synthetic peptide
GEVFFDMRLKGDK

SEQ ID NO: 492 - protein, 12 aa, synthetic construct
  REGION 1..12: note = Description of Artificial Sequence: Synthetic peptide
LAGSCLPIFSTL

SEQ ID NO: 493 - protein, 30 aa, synthetic construct
  REGION 1..30: note = Description of Artificial Sequence: Synthetic polypeptide
AHNQDLGLAGSCLARFSTMPFLYCNPGDIC

SEQ ID NO: 494 - protein, 30 aa, synthetic construct
  REGION 1..30: note = Description of Artificial Sequence: Synthetic polypeptide
QEKAHNQDLGLAGSCLPVFSTLPFAYCNIH

SEQ ID NO: 495 - protein, 12 aa, synthetic construct
  REGION 1..12: note = Description of Artificial Sequence: Synthetic peptide
LAGSCLPVFSTM

SEQ ID NO: 496 - protein, 30 aa, synthetic construct
  REGION 1..30: note = Description of Artificial Sequence: Synthetic polypeptide
GNKRAHGQDLGTAGSCLRRFSTMPFMFCNI

SEQ ID NO: 497 - protein, 21 aa, synthetic construct
  REGION 1..21: note = Description of Artificial Sequence: Synthetic peptide
RAHGQDLGTAGSCLRRFSTMP

SEQ ID NO: 498 - protein, 12 aa, synthetic construct
  REGION 1..12: note = Description of Artificial Sequence: Synthetic peptide
RKRLQVQLNIRT

SEQ ID NO: 499 - protein, 30 aa, synthetic construct
  REGION 1..30: note = Description of Artificial Sequence: Synthetic polypeptide
HLVLPLQQSDVRKRLQVQLSIRTFASSGLI

SEQ ID NO: 500 - protein, 12 aa, synthetic construct
  REGION 1..12: note = Description of Artificial Sequence: Synthetic peptide
RKRLSVQLRIRT

SEQ ID NO: 501 - protein, 15 aa, synthetic construct
  REGION 1..15: note = Description of Artificial Sequence: Synthetic peptide
DLGTAGSCLRRFSTM

SEQ ID NO: 502 - protein, 10 aa, synthetic construct
  REGION 1..10: note = Description of Artificial Sequence: Synthetic peptide
RNIAEIIKDI

SEQ ID NO: 503 - protein, 24 aa, synthetic construct
  REGION 1..24: note = Description of Artificial Sequence: Synthetic peptide
TAGSCLRKFSTMRRRRRRRRRRRR

SEQ ID NO: 504 - protein, 17 aa, synthetic construct
  REGION 1..17: note = Description of Artificial Sequence: Synthetic peptide
FTLTGLLGTLVTMGLLT

SEQ ID NO: 505 - protein, 7 aa, synthetic construct
  REGION 1..7: note = Description of Artificial Sequence: Synthetic peptide
APYKAWK

SEQ ID NO: 506 - protein, 23 aa, synthetic construct
  REGION 1..23: note = Description of Artificial Sequence: Synthetic peptide
STSKTNRGDDSNWSKRVTNNKPS

SEQ ID NO: 507 - protein, 23 aa, synthetic construct
  REGION 1..23: note = Description of Artificial Sequence: Synthetic peptide
STSKRKRGDDSNWSKRVTKKKPS

SEQ ID NO: 508 - protein, 23 aa, synthetic construct
  REGION 1..23: note = Description of Artificial Sequence: Synthetic peptide
STSKRKRGDDSNWSKRVSKKKPS

SEQ ID NO: 509 - protein, 23 aa, synthetic construct
  REGION 1..23: note = Description of Artificial Sequence: Synthetic peptide
STSKRKRGDDANWSKRVTKKKPS

SEQ ID NO: 510 - protein, 26 aa, synthetic construct
  REGION 1..26: note = Description of Artificial Sequence: Synthetic peptide
PLAGSKRKRADEVAWSKRGTKKKPER

SEQ ID NO: 511 - protein, 39 aa, synthetic construct
  REGION 1..39: note = Description of Artificial Sequence: Synthetic polypeptide
PLAGSKRKRADEVAWSKRGTKKKPERTSAARAGPSRRIR

SEQ ID NO: 512 - protein, 24 aa, synthetic construct
  REGION 1..24: note = Description of Artificial Sequence: Synthetic peptide
STSKRKRGDDANWSKRTTKKKPSS

SEQ ID NO: 513 - protein, 39 aa, synthetic construct
  REGION 1..39: note = Description of Artificial Sequence: Synthetic polypeptide
STSKRKRGDDANWSKRTTKKKPSSAGLKRAGSKADRPSL

SEQ ID NO: 514 - protein, 26 aa, synthetic construct
  REGION 1..26: note = Description of Artificial Sequence: Synthetic peptide
PTTAGKRKRSDDAAWSKRARPKAGRT

SEQ ID NO: 515 - protein, 39 aa, synthetic construct
  REGION 1..39: note = Description of Artificial Sequence: Synthetic polypeptide
PTTAGKRKRSDDAAWSKRARPKAGRTSAARPGTSVRRIR

SEQ ID NO: 516 - protein, 26 aa, synthetic construct
  REGION 1..26: note = Description of Artificial Sequence: Synthetic peptide
SSSLGKRKRSDEGAWSKGKSKKKAMR

SEQ ID NO: 517 - protein, 39 aa, synthetic construct
  REGION 1..39: note = Description of Artificial Sequence: Synthetic polypeptide
SSSLGKRKRSDEGAWSKGKSKKKAMRGSSSRRPGPVRGP

SEQ ID NO: 518 - protein, 25 aa, synthetic construct
  REGION 1..25: note = Description of Artificial Sequence: Synthetic peptide
PTTAGKRKRTDDAAWSKRARPKAGR

SEQ ID NO: 519 - protein, 39 aa, synthetic construct
  REGION 1..39: note = Description of Artificial Sequence: Synthetic polypeptide
PTTAGKRKRTDDAAWSKRARPKAGRTSAARPGTAVRRVR

SEQ ID NO: 520 - protein, 30 aa, synthetic construct
  REGION 1..30: note = Description of Artificial Sequence: Synthetic polypeptide
PATAGKRKRSDDAAWSKRARPKAGRTSAAR

SEQ ID NO: 521 - protein, 39 aa, synthetic construct
  REGION 1..39: note = Description of Artificial Sequence: Synthetic polypeptide
PATAGKRKRSDDAAWSKRARPKAGRTSAARPGTSVRRIR

SEQ ID NO: 522 - protein, 25 aa, synthetic construct
  REGION 1..25: note = Description of Artificial Sequence: Synthetic peptide
SSSLGKRKRSNGGDWSKRSAKKKPA

SEQ ID NO: 523 - protein, 39 aa, synthetic construct
  REGION 1..39: note = Description of Artificial Sequence: Synthetic polypeptide
SSSLGKRKRSNGGDWSKRSAKKKPAGTPSRRAGPGRGPR

SEQ ID NO: 524 - protein, 26 aa, synthetic construct
  REGION 1..26: note = Description of Artificial Sequence: Synthetic peptide
SSSLGKRKRSDEGAWSKGKSKKKAMR

SEQ ID NO: 525 - protein, 39 aa, synthetic construct; identical to SEQ ID NO: 517
  REGION 1..39: note = Description of Artificial Sequence: Synthetic polypeptide

SEQ ID NO: 526 - protein, 24 aa, synthetic construct
  REGION 1..24: note = Description of Artificial Sequence: Synthetic peptide
STSKRKRGDDANWNKRPTKKKPSS

SEQ ID NO: 527 - protein, 39 aa, synthetic construct
  REGION 1..39: note = Description of Artificial Sequence: Synthetic polypeptide
STSKRKRGDDANWNKRPTKKKPSSAGLKKAGSKAERPSL

SEQ ID NO: 528 - protein, 24 aa, synthetic construct
  REGION 1..24: note = Description of Artificial Sequence: Synthetic peptide
SGALKRKRSDEVAWSRRRPVKKPV

SEQ ID NO: 529 - protein, 39 aa, synthetic construct
  REGION 1..39: note = Description of Artificial Sequence: Synthetic polypeptide
SGALKRKRSDEVAWSRRRPVKKPVRRAPPPRAGPSVRRG

SEQ ID NO: 530 - protein, 25 aa, synthetic construct
  REGION 1..25: note = Description of Artificial Sequence: Synthetic peptide
SGALKRKRSDEVAWSRRKPAKKPAR

SEQ ID NO: 531 - protein, 39 aa, synthetic construct
  REGION 1..39: note = Description of Artificial Sequence: Synthetic polypeptide
SGALKRKRSDEVAWSRRKPAKKPARQPPPPRAGPSVRRG

SEQ ID NO: 532 - protein, 25 aa, synthetic construct
  REGION 1..25: note = Description of Artificial Sequence: Synthetic peptide
AGALKRKRSDEVAWSRRKPAKKPAR

SEQ ID NO: 533 - protein, 39 aa, synthetic construct
  REGION 1..39: note = Description of Artificial Sequence: Synthetic polypeptide
AGALKRKRSDEVAWSRRKPAKKPARAPPPRAGPSVRRGL

SEQ ID NO: 534 - protein, 243 aa, synthetic construct
  REGION 1..243: note = Description of Artificial Sequence: Synthetic polypeptide
STSKRKRGDDSNWSKRVTKKKPSSAGLKRAGSKADRPSLQIQTLQHAGTTMITVPSGGVCDLINTYARGSDEGNRHTSETLTYKIAIDYHFVADAAACRYSNTGTGVMWLVYDTTPGGQAPTPQTIFSYPDTLKAWPATWKVSRELCHRFVVKRRWLFNMETDGRIGSDIPPSNASWKPCKRNIYFHKFTSGLGVRTQWKNVTDGGVGAIQRGALYMVIAPGNGLTFTAHGQTRLYFKSVGNQ

SEQ ID NO: 535 - protein, 100 aa, synthetic construct
  REGION 1..100: note = Description of Artificial Sequence: Synthetic polypeptide
DPQNALYYQPRVPTAAPTSGGVPWSRVGEVAILSFVALICFYLLYLWVLRDLILVLKARQGRSTEELIFGGQAVDRSNPIPNIPAPPSQGNPGPFVPGTG

SEQ ID NO: 536 - protein, 292 aa, synthetic construct
  REGION 1..292: note = Description of Artificial Sequence: Synthetic polypeptide
GSQLVPPPSAFNYIESQRDEFQLSHDLTEIVLQFPSTASQITARLSRSCMKIDHCVIEYRQQVPINASGTVIVEIHDKRMTDNESLQASWTFPIRCNIDLHYFSSSFFSLKDPIPWKLYYRVSDSNVHQMTHFAKFKGKLKLSSAKHSVDIPFRAPTVKILAKQFSEKDIDFWHVGYGKWERRLVKSASSSRFGLRGPIEINPGESWATKSAIVTPNRNADLDIEEELLPYRELNRLGTNILDPGESASIVGIQRSQSNITMSMSQLNELVRSTVHECIKTSCIPSTPKSLS

SEQ ID NO: 537 - protein, 22 aa, synthetic construct
  REGION 1..22: note = Description of Artificial Sequence: Synthetic peptide
RTGVKRSYGAARGDDRRRPNVV

SEQ ID NO: 538 - protein, 19 aa, synthetic construct
  REGION 1..19: note = Description of Artificial Sequence: Synthetic peptide
SYVKTVPNRTRTYIKLRVR

SEQ ID NO: 539 - protein, 255 aa, synthetic construct
  REGION 1..255: note = Description of Artificial Sequence: Synthetic polypeptide
MYSTSNRRGRSQTQRGSHVRRTGVKRSYGAARGDDRRRPNVVSKTQVEPRMTIQRVQENQFGPEFVLSQNSALSTFVTYPSYVKTVPNRTRTYIKLKRVRFKGTLKIERGQGDTIMDGPSSNIEGVFSMVIVVDRKPHVSQSGRLHTFDELFGARIHCHGNLSVVPALKDRYYIRHVTKRVVSLEKDTLLIDLHGTTQLSNKRYNCWASFSDLERDCNGVYGNITKNALLVYYCWLSDAQSKASTYVSFELDYLG

SEQ ID NO: 540 - protein, 30 aa, synthetic construct
  REGION 1..30: note = Description of Artificial Sequence: Synthetic polypeptide
RRRRRRRRRRRRVDYGKWERKPIRCASMSR

SEQ ID NO: 541 - protein, 24 aa, synthetic construct
  REGION 1..24: note = Description of Artificial Sequence: Synthetic peptide
RRRRRRRRRRRRGKWERKPIRCAS

SEQ ID NO: 542 - protein, 28 aa, synthetic construct
  REGION 1..28: note = Description of Artificial Sequence: Synthetic peptide
KKKKKKKKKKKKKKKKGKWERKPIRCAS

SEQ ID NO: 543 - protein, 40 aa, synthetic construct
  REGION 1..40: note = Description of Artificial Sequence: Synthetic polypeptide
RRRRRRRRRRRRVDFSHVDYGKWERKPIRCASMSRLGLRG

SEQ ID NO: 544 - protein, 38 aa, synthetic construct
  REGION 1..38: note = Description of Artificial Sequence: Synthetic polypeptide
GVKRSYGAARGDDRRRPNVVAPYKAWRRRRRRRRRRRR

SEQ ID NO: 545 - protein, 38 aa, synthetic construct
  REGION 1..38: note = Description of Artificial Sequence: Synthetic polypeptide
KSVPNRTRTYIKLKRLRFKGAPYKAWRRRRRRRRRRRR

SEQ ID NO: 546 - protein, 34 aa, synthetic construct
  REGION 1..34: note = Description of Artificial Sequence: Synthetic polypeptide
RTGVKRSYGAARGDDRRRPNVVRRRRRRRRRRRR

SEQ ID NO: 547 - protein, 32 aa, synthetic construct
  REGION 1..32: note = Description of Artificial Sequence: Synthetic polypeptide
SYVKTVPNRTRTYIKGGGGGRRRRRRRRRRRR

SEQ ID NO: 548 - protein, 50 aa, synthetic construct
  REGION 1..50: note = Description of Artificial Sequence: Synthetic polypeptide
VDIPFRAPTIKILSKQFTEDDIDFWHVGYGKWERKLVRPASLSGRRGLRR

SEQ ID NO: 549 - protein, 29 aa, synthetic construct
  REGION 1..29: note = Description of Artificial Sequence: Synthetic peptide
IDFWHVGYGKWERKLVRPASLSGRRGLRR

SEQ ID NO: 550 - protein, 31 aa, synthetic construct
  REGION 1..31: note = Description of Artificial Sequence: Synthetic polypeptide
IDFWSVEKGETRRRLLNPTPHAHSPRPIAHR

SEQ ID NO: 551 - protein, 28 aa, synthetic construct
  REGION 1..28: note = Description of Artificial Sequence: Synthetic peptide
IDFSHVGYGKWERKMIRSASISRLGLHN

SEQ ID NO: 552 - protein, 28 aa, synthetic construct
  REGION 1..28: note = Description of Artificial Sequence: Synthetic peptide
VDFSHVGYGKWERKLIRSASTVKYGLPS

SEQ ID NO: 553 - protein, 28 aa, synthetic construct
  REGION 1..28: note = Description of Artificial Sequence: Synthetic peptide
IDFSHVDYGKVERKLVKCESSSRLGLHS

SEQ ID NO: 554 - protein, 29 aa, synthetic construct
  REGION 1..29: note = Description of Artificial Sequence: Synthetic peptide
IDFWSVGRKAQQRKLVQGPSLIGSRSMRY

SEQ ID NO: 555 - protein, 31 aa, synthetic construct
  REGION 1..31: note = Description of Artificial Sequence: Synthetic polypeptide
IDFWSVGSKPQTRRLVDGSRLIGHSSRSLRV

SEQ ID NO: 556 - protein, 31 aa, synthetic construct
  REGION 1..31: note = Description of Artificial Sequence: Synthetic polypeptide
IDFWSVERGETRRRLLNPTPSAGSNRALSKR

SEQ ID NO: 557 - protein, 31 aa, synthetic construct
  REGION 1..31: note = Description of Artificial Sequence: Synthetic polypeptide
VDFWSVGKPKPIRRLIQNDPGTDYDTGPKYR

SEQ ID NO: 558 - protein, 31 aa, synthetic construct
  REGION 1..31: note = Description of Artificial Sequence: Synthetic polypeptide
VDFWSVEKPKPIRRLLNPGPNQGPYPNTGHR

SEQ ID NO: 559 - protein, 28 aa, synthetic construct
  REGION 1..28: note = Description of Artificial Sequence: Synthetic peptide
VDFSHVDYGKWERKLIRSASTSRYGLRS

SEQ ID NO: 560 - protein, 28 aa, synthetic construct
  REGION 1..28: note = Description of Artificial Sequence: Synthetic peptide
VDFSHVDYGKWERKTLRSRSLSRIGLTG

SEQ ID NO: 561 - protein, 28 aa, synthetic construct
  REGION 1..28: note = Description of Artificial Sequence: Synthetic peptide
IDFWHVGYGKWERRLVKSASSSRFGIRG

SEQ ID NO: 562 - protein, 28 aa, synthetic construct
  REGION 1..28: note = Description of Artificial Sequence: Synthetic peptide
VDFFHVDYGRWERKHIRCASMSRVGLRG

SEQ ID NO: 563 - protein, 28 aa, synthetic construct
  REGION 1..28: note = Description of Artificial Sequence: Synthetic peptide
GTFQHVDYGKWERKPIRCQSMSRVGYRR

SEQ ID NO: 564 - protein, 17 aa, synthetic construct
  REGION 1..17: note = Description of Artificial Sequence: Synthetic peptide
VGYGKWERKLVRPASLS

SEQ ID NO: 565 - protein, 17 aa, synthetic construct
  REGION 1..17: note = Description of Artificial Sequence: Synthetic peptide
VEKGETRRRLLNPTPHA

SEQ ID NO: 566 - protein, 17 aa, synthetic construct
  REGION 1..17: note = Description of Artificial Sequence: Synthetic peptide
VGYGKWERKLIRSASTV

SEQ ID NO: 567 - protein, 17 aa, synthetic construct
  REGION 1..17: note = Description of Artificial Sequence: Synthetic peptide
VEKPKPIRRLLNPGPNQ

SEQ ID NO: 568 - protein, 17 aa, synthetic construct
  REGION 1..17: note = Description of Artificial Sequence: Synthetic peptide
VDYGKWERKLIRSASTS

SEQ ID NO: 569 - protein, 17 aa, synthetic construct
  REGION 1..17: note = Description of Artificial Sequence: Synthetic peptide
VDYGKWERKTLRSRSLS

SEQ ID NO: 570 - protein, 17 aa, synthetic construct
  REGION 1..17: note = Description of Artificial Sequence: Synthetic peptide
VGYGKWERRLVKSASSS

SEQ ID NO: 571 - protein, 17 aa, synthetic construct
  REGION 1..17: note = Description of Artificial Sequence: Synthetic peptide
VDYGRWERKHIRCASMS

SEQ ID NO: 572 - protein, 17 aa, synthetic construct
  REGION 1..17: note = Description of Artificial Sequence: Synthetic peptide
VERPKPIRRLLTPTPGC

SEQ ID NO: 573 - protein, 47 aa, synthetic construct
  REGION 1..47: note = Description of Artificial Sequence: Synthetic polypeptide
PFRAPTIKILSKQFTEDDIDFWHVGYGKWERKLVRPASLSGRRGLRR

SEQ ID NO: 574 - protein, 44 aa, synthetic construct
  REGION 1..44: note = Description of Artificial Sequence: Synthetic polypeptide
PFRAPTVKILSKQFTDKDIDFSHVGYGKWERKMIRSASISRLGL

SEQ ID NO: 575 - protein, 46 aa, synthetic construct
  REGION 1..46: note = Description of Artificial Sequence: Synthetic polypeptide
DIAFRAPTVKILSKQFTDRDVDFSHVGYGKWERKLIRSASTVKYGL

SEQ ID NO: 576 - protein, 49 aa, synthetic construct
  REGION 1..49: note = Description of Artificial Sequence: Synthetic polypeptide
DIRFKPPTINILSKDYTADCVDFWSVEKPKPIRRLLNPGPNQGPYPNTG

SEQ ID NO: 577 - protein, 46 aa, synthetic construct
  REGION 1..46: note = Description of Artificial Sequence: Synthetic polypeptide
DIPFRAPTVKIHSKQFSHRDVDFSHVDYGKWERKTLRSRSLSRIGL

SEQ ID NO: 578 - protein, 46 aa, synthetic construct
  REGION 1..46: note = Description of Artificial Sequence: Synthetic polypeptide
DIPFRAPTVKILAKQFSEKDIDFWHVGYGKWERRLVKSASSSRFGI

SEQ ID NO: 579 - protein, 46 aa, synthetic construct
  REGION 1..46: note = Description of Artificial Sequence: Synthetic polypeptide
DIPFRAPTVKILSKQFTDKDVDFFHVDYGRWERKHIRCASMSRVGL

SEQ ID NO: 580 - protein, 43 aa, synthetic construct
  REGION 1..43: note = Description of Artificial Sequence: Synthetic polypeptide
DIKYKPPTIKILSKDYTADCVDFWSVERPKPIRRLLTPTPGCG

SEQ ID NO: 581 - protein, 47 aa, synthetic construct
  REGION 1..47: note = Description of Artificial Sequence: Synthetic polypeptide
ARTKQTARKSTGGKAPRKQLATKAARKSAPATGGVKKPHRYRPGTVA

SEQ ID NO: 582 - protein, 34 aa, synthetic construct
  REGION 1..34: note = Description of Artificial Sequence: Synthetic polypeptide
SGRGKGGKGLGKGGAKRHRKVLRDNIQGITKPAI

SEQ ID NO: 583 - protein, 10 aa, synthetic construct
  REGION 1..10: note = Description of Artificial Sequence: Synthetic peptide
GRKKRRQRRR

SEQ ID NO: 584 - DNA, 49 bp, synthetic construct
  misc_feature 1..49: note = Description of Artificial Sequence: Synthetic oligonucleotide
  source 1..49: mol_type = other DNA
ttgaggttgctagtgaaggctagctacaacgaacagttgtgtcagaagc

SEQ ID NO: 585 - DNA, 45 bp, synthetic construct
  misc_feature 1..45: note = Description of Artificial Sequence: Synthetic primer
  source 1..45: mol_type = other DNA
agtaatacgactcactatagggagaacatttgcttctgacacaac

SEQ ID NO: 586 - DNA, 45 bp, synthetic construct
  misc_feature 1..45: note = Description of Artificial Sequence: Synthetic primer
  source 1..45: mol_type = other DNA
agtaatacgactcactataaggagaacatttgcttctgacacaac

SEQ ID NO: 587 - DNA, 45 bp, synthetic construct
  misc_feature 1..45: note = Description of Artificial Sequence: Synthetic primer
  source 1..45: mol_type = other DNA
agtaatacgactcactatagtgagaacatttgcttctgacacaac

SEQ ID NO: 588 - DNA, 45 bp, synthetic construct
  misc_feature 1..45: note = Description of Artificial Sequence: Synthetic primer
  source 1..45: mol_type = other DNA
agtaatacgactcactatagcgagaacatttgcttctgacacaac

SEQ ID NO: 589 - DNA, 45 bp, synthetic construct
  misc_feature 1..45: note = Description of Artificial Sequence: Synthetic primer
  source 1..45: mol_type = other DNA
agtaatacgactcactatagagagaacatttgcttctgacacaac

SEQ ID NO: 590 - DNA, 29 bp, synthetic construct
  misc_feature 1..29: note = Description of Artificial Sequence: Synthetic primer
  source 1..29: mol_type = other DNA
gccctctagatcaaccactttggccctct

SEQ ID NO: 591 - DNA, 23 bp, unidentified
  misc_feature 1..23: note = Description of Unknown: T7 phage sequence
  source 1..23: mol_type = other DNA
taatacgactcactatagggaga

SEQ ID NO: 592 - DNA, 23 bp, unidentified
  misc_feature 1..23: note = Description of Unknown: T3 phage sequence
  source 1..23: mol_type = other DNA
aattaaccctcactaaagggaga

SEQ ID NO: 593 - DNA, 23 bp, unidentified
  misc_feature 1..23: note = Description of Unknown: SP6 phage sequence
  source 1..23: mol_type = other DNA
atttaggtgacactatagaagng

SEQ ID NO: 594 - DNA, 23 bp, unidentified
  misc_feature 1..23: note = Description of Unknown: K11 phage sequence
  source 1..23: mol_type = other DNA
aattagggcacactatagggaac

SEQ ID NO: 595 - DNA, 10 bp, synthetic construct
  misc_feature 1..10: note = Description of Artificial Sequence: Synthetic oligonucleotide
  source 1..10: mol_type = other DNA
tatagggaga

SEQ ID NO: 596 - DNA, 10 bp, synthetic construct
  misc_feature 1..10: note = Description of Artificial Sequence: Synthetic oligonucleotide
  source 1..10: mol_type = other DNA
tataaggaga

SEQ ID NO: 597 - DNA, 10 bp, synthetic construct
  misc_feature 1..10: note = Description of Artificial Sequence: Synthetic oligonucleotide
  source 1..10: mol_type = other DNA
tatagtgaga

(19) United States
(12) Patent Application Publication
(10) Pub. No.: US 2025/0333729 A1
(43) Pub. Date: Oct. 30, 2025

(54) TRINUCLEOTIDE CAP ANALOGS, PREPARATION AND USES THEREOF

(71) Applicant: LIFE TECHNOLOGIES CORPORATION, Carlsbad, CA (US)

(72) Inventors: Anilkumar KORE, Austin, TX (US); Senthilvelan ANNAMALAI, Austin, TX (US); Shanmugasundaram MUTHIAN, Austin, TX (US); Robert POTTER, San Marcos, CA (US); Tyson VONDERFECT, Carlsbad, CA (US)

(21) Appl. No.: 19/261,919

(22) Filed: Jul. 7, 2025

Related U.S. Application Data
(62) Division of application No. 17/364,956, filed on Jul. 1, 2021, now Pat. No. 12,385,039.
(60) Provisional application No. 63/047,465, filed on Jul. 2, 2020.
(60) Provisional application No. 63/197,607, filed on Jun. 7, 2021.

(51) Int. Cl. C12N 15/11; A61K 31/7088; A61K 38/45; A61K 47/54; C07H 21/00; C12N 9/12

(52) U.S. Cl. CPC C12N 15/11; A61K 31/7088; A61K 38/45; A61K 47/549; C07H 21/00; C12N 9/1247; C12Y 207/07006; C12N 2310/3231; C12N 2310/3519; C12N 2320/32

(57) ABSTRACT

This specification generally relates to trinucleotide RNA cap analogs, methods of use thereof, and kits comprising same. In particular, the trinucleotide cap analogs provided herein permit ready detection and/or isolation of capped RNA transcripts in vitro and translation of capped mRNAs in vivo.

### CROSS-REFERENCE TO RELATED APPLICATIONS

This application is a divisional of U.S. patent application Ser. No. 17/364,956 filed Jul. 1, 2021, which claims the benefit of priority to U.S. Provisional Patent Application No. 63/197,607 filed Jun. 7, 2021 and U.S. Provisional Patent Application No. 63/047,465 filed Jul. 2, 2020, which disclosures are herein incorporated by reference in their entirety.

### FIELD

This specification generally relates to trinucleotide RNA cap analogs, methods of use thereof, and kits comprising same. In particular, the trinucleotide cap analogs provided herein permit ready detection and/or isolation of capped RNA transcripts in vitro and translation of capped mRNAs in vivo.

### SEQUENCE LISTING

The instant application contains a Sequence Listing which has been submitted electronically in XML format and is hereby incorporated by reference in its entirety. Said XML sequence listing is named TP108951USDIV1, was created on Jun. 23, 2025 and is 766,778 bytes in size.

### BACKGROUND

Eukaryotic mRNAs bear a “cap” structure at their 5′-termini that is well known to play an important role in translation. Naturally occurring cap structures consist of a 7-methyl guanosine that is linked via a triphosphate bridge to the 5′-end of the first transcribed nucleotide, resulting in m⁷G(5′)ppp(5′)N, where N is any nucleotide. The mRNA cap plays an important role in gene expression. It protects the mRNAs from degradation by exonucleases, enables transport of RNAs from the nucleus to the cytoplasm, and participates in assembly of the translation initiation complex. A dinucleotide in the form of m⁷G(5′)ppp(5′)G (mCAP) has been used as the primer in transcription with T7 or SP6 RNA polymerase in vitro to obtain RNAs having a cap structure at their 5′-termini. In vivo, the cap is added enzymatically. However, over the past 20 years or so, numerous studies have required the synthesis of proteins in an in vitro translation extract supplemented with in vitro synthesized mRNA. The prevailing method for the in vitro synthesis of capped mRNA employs mCAP as an initiator of transcription. A disadvantage of using mCAP, a pseudosymmetrical dinucleotide, has always been the propensity of the 3′-OH of either the G or m⁷G (m⁷Guo) moiety to serve as the initiating nucleophile for transcriptional elongation resulting in ˜50% of capped RNA that is translatable. This disadvantage was addressed by provision of modified cap analogs having the 3′-OH group of the m⁷G portion of the cap blocked to prevent transcription from that position (e.g., ARCA).

While caps may also be added to RNA molecules by the enzyme guanylyl transferase in the cell, caps are initially added to RNA during in vitro transcription where the cap is used as a primer for RNA polymerase. The 5′ terminal nucleoside is normally a guanine, and is in the reverse orientation to all the other nucleotides, i.e., 5′Gppp5′GpNpNp . . . and, in most instances, the cap contains two nucleotides, connected by a 5′-5′ triphosphate linkage.

Transcription of RNA usually starts with a nucleoside triphosphate (usually a purine, A or G). When transcription occurs in vitro, it typically includes a phage RNA polymerase such as T7, T3 or SP6, a DNA template containing a phage polymerase promoter, nucleotides (ATP, GTP, CTP and UTP) and a buffer containing magnesium salt. The 5′ cap structure enhances the translation of mRNA by helping to bind the eukaryotic ribosome and assuring recognition of the proper AUG initiator codon. This function may vary with the translation system and with the specific mRNA being synthesized.

During translation the cap is bound by translational initiation factor eIF4E and the cap-binding complex (CBC) recruits additional initiation factors. Decapping is catalyzed by proteins dcp1 and dcp2 which compete with eIF4E to bind to the cap. Translation results in amino acids as encoded by the mRNA to join together to form a peptide and occurs as three processes: initiation, elongation, and termination. Initiation in eukaryotes involves attachment of a ribosome which scans the mRNA for the first methionine codon. Elongation proceeds with the successive addition of amino acids until a stop codon is reached, terminating translation.

Capped RNA encoding specific genes can be transfected into eukaryotic cells or microinjected into cells or embryos to study the effect of translated product in the cell or embryo. If uncapped RNA is used, the RNA in these experiments is rapidly degraded and the yield of translated protein is much reduced.

Capped RNA can also be used to treat disease. Isolated dendritic cells from a patient can be transfected with capped RNA encoding immunogen. The dendritic cells translate the capped RNA into a protein that induces an immune response against this protein. In a small human study, immunotherapy with dendritic cells loaded with CEA capped RNA was shown to be safe and feasible for pancreatic patients (Morse et al., Int. J. Gastrointest. Cancer, 32, 1-6, (2002)). It was also noted that introducing a single capped RNA species into immature dendritic cells induced a specific T-cell response (Heiser et al., J. Clin. Invest., 109, 409-417 (2002)).

However, capped RNA known in the art still has limitations with respect to their intracellular stability as well as their efficiency of in vitro transcription, for example with substrates such as T7-RNA-polymerase. Thus, there is still a need for mRNA cap analogs, such as locked capped RNA that can result in high levels of capping efficiency, improved translation efficiencies, and improved intracellular molecular stability of 5′ capped mRNAs.

### SUMMARY

The present disclosure relates to new modified trinucleotide cap analogs of Formula (I) as defined herein:

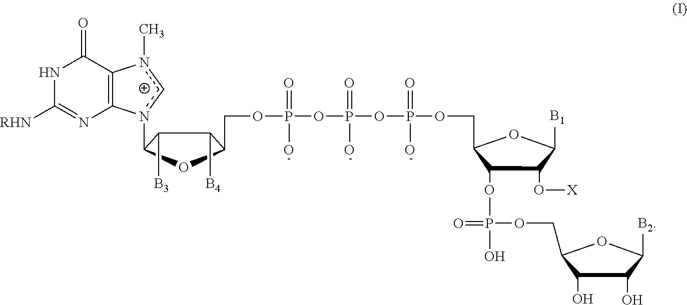

The trinucleotide cap analogs disclosed herein can result in high levels of capping efficiency and improved translation efficiencies. In at least one aspect, the trinucleotide cap analogs disclosed herein are improved substrates for T7-RNA polymerase and lead to a better transcription yield.

The trinucleotide cap analogs disclosed herein can result in improved intracellular molecular stability of 5′ capped mRNAs. In at least one aspect, the trinucleotide cap analogs disclosed herein increase the intracellular stability of mRNA in vaccines.

In at least one aspect, the trinucleotide cap analogs disclosed herein can also serve as reporter moieties. In at least one aspect, the trinucleotide cap analogs disclosed herein improve transfection into specific cell lines.

The present disclosure also relates to compositions comprising the cap analogs, compositions comprising RNA having the cap analogs described herein covalently bonded thereto, methods for using mRNA species containing such analogs, as well as kits containing the novel cap analogs.

In a first aspect, this disclosure is directed to a trinucleotide cap analog of Formula (I):

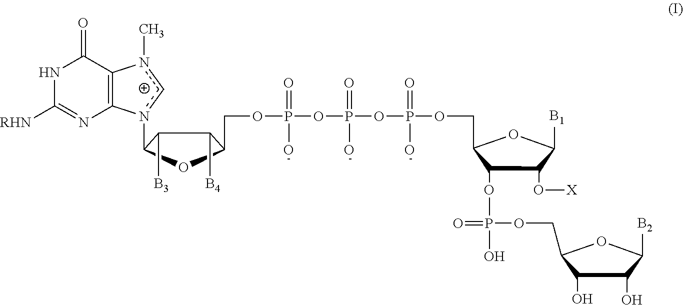

wherein
B₃ is chosen from —OH, halogen, dyes, —OR¹,
   wherein R¹ is chosen from propargyl, tert-butyldimethylsilyl, and a methylene bridge with the 4′C;
B₄ is chosen from —OH, dyes, and —OR², wherein R² is chosen from propargyl and tert-butyldimethylsilyl;
   or R¹ joins with R² such that B₃ and B₄ form -2′,3′-O-isopropylidine; on the condition that B₃ and B₄ cannot both be —OH
X is chosen from —H and —CH₃;
B₁ and B₂ are each independently chosen from adenine, guanine, cytosine, and uracil;
R is chosen from H, a linker-bound cell-penetrating peptide, a linker-bound cell-penetrating peptide covalently linked to a dye, and a linker-bound dye.

In a second aspect, this disclosure is directed to a composition comprising a trinucleotide cap analog of Formula (I), or any of the embodiments thereof described herein.

In a third aspect, this disclosure is directed to a composition comprising RNA having a trinucleotide cap analog of Formula (I), or any of the embodiments thereof described herein.

In a fourth aspect, this disclosure is directed to a kit comprising a trinucleotide cap analog of Formula (I) or any of the embodiments thereof described herein; nucleotide triphosphate molecules; and an RNA polymerase.

In a fifth aspect, this disclosure is directed to a method of producing trinucleotide capped RNA comprising contacting a nucleic acid substrate with an RNA polymerase and a trinucleotide cap analog of Formula (I), or any of the embodiments thereof described herein, in the presence of nucleotide triphosphates under conditions and for a time sufficient to produce a trinucleotide capped RNA.

In a sixth aspect, this disclosure is directed to a method comprising contacting a cell with the trinucleotide cap analog of Formula (I), or any of the embodiments thereof described herein.

In a seventh aspect, this disclosure is directed to a method of increasing intracellular stability of an RNA, comprising incorporating a trinucleotide cap analog according to Formula (I), or any of the embodiments thereof described herein, into the RNA.

In an eighth aspect, this disclosure is directed to a method for introducing an RNA into a cell, comprising contacting the cell with a composition according to the present disclosure comprising a trinucleotide cap analog according to Formula (I), or any of the embodiments thereof described herein. In some examples, the cell is a dendritic cell, a tumor cell, a stem cell (iPSC, HSC, adult stem cell) or the like.

In a ninth aspect, this disclosure is directed to a method for RNA translation inhibition in a cell comprising contacting the cell with a composition according to the present disclosure comprising a trinucleotide cap analog according to Formula (I), or any of the embodiments thereof described herein.

Also provided herein are transcriptional initiation complexes comprising: (a) a nucleic acid molecule comprising a promoter region, the promoter region comprising a transcriptional initiation site, the transcriptional initiation site comprising a template strand, and (b) a capped primer comprising two or more (e.g., from about two to about twelve, from about two to about ten, from about two to about nine, from about two to about eight, from about two to about six, from about three to about eight, etc.) bases hybridized to the transcriptional initiation site comprising a template strand at least at positions −1 and +1, +1 and +2, or +2 and +3. In some instances, at least one (e.g., one, two, three, four, etc.) nucleotide at one or both adjacent positions (5′ and/or 3′) of the non-template strand of the initiation site is a transcriptional initiation blocking nucleotide. In some instances, the one or more transcriptional initiation blocking nucleotides are selected from the group consisting of (A) thymidine, (B) cytosine, (C) adenosine, and (D) a chemically modified nucleotide. Further, the initiation complex may comprise a template strand that is hybridized (e.g., partially hybridized) to a complementary non-template strand. Additionally, the template and/or non-template strand may contain a chemically modified nucleotide (e.g., deoxythymidine residue, 2′-deoxycytidine, etc.) at positions −1 and/or +1.

Positions −1, +1, and +2 of non-template strand of the transcriptional start site of promoters and transcriptional initiation complexes set out herein may comprise a nucleotide sequence selected from the group consisting of: A G T, A A T, A G C, A A C, A G A, A A A, G A T, G A C, G A A, G G T, G G C, G G A, A T T, A T C, and A T A.

Also provided herein are transcriptional initiation complexes comprising: (a) a nucleic acid molecule comprising a promoter region, the promoter region comprising a transcriptional initiation site, the transcriptional initiation site comprising a template strand, and (b) a non-naturally occurring capped primer comprising three or more bases hybridized to the DNA template at least at nucleotide positions −1 and +1, +1 and +2, or +2 and +3. Further, initiation complexes set out herein may comprise a non-naturally occurring capped primer is a capped primer set out herein.

Further provided herein are nucleic acid molecules comprising a promoter, wherein the promoter comprises the following non-template strand nucleotide sequence: TATY₁ Y₂Z, wherein Y₁ is at the −1 position, Y₂ is at the +1 position, and Z is at position +2, and wherein Z is a transcriptional initiation blocking nucleotide. Further, Z may be adenosine, cytosine, thymidine, or a chemically modified nucleotide. Such nucleic acid molecules may comprise a nucleotide sequence selected from the group consisting of (a) 5′-T A T A G T-3′, (b) 5′-T A T A G C-3′, and (c) 5′-T A T A A C-3′.

Also, provided herein are methods for producing mRNA molecules. Such methods may comprise contacting a DNA template with a capped primer and an RNA polymerase under condition that allow for the production of the mRNA molecules by a transcription reaction, wherein the DNA template comprises: (a) a nucleic acid molecule comprising a promoter region, the promoter region comprising a transcriptional initiation site, the transcriptional initiation site comprising a template strand, and (b) a capped primer comprising two or more bases hybridized to the transcriptional initiation site comprising a template strand at least at positions −1 and +1, +1 and +2, or +2 and +3, and wherein at least the nucleotide at the 5′ adjacent position of the template strand of the initiation site is a transcriptional initiation blocking nucleotide. Further, RNA polymerases used in such methods include bacteriophage, bacterial, and eukaryotic (e.g., mammalian) RNA polymerases. In some instances, a bacteriophage RNA polymerase such as a T7 bacteriophage, a T3 bacteriophage, an SP6 bacteriophage, or a K11 bacteriophage RNA polymerase or variant thereof may be used in methods set out herein.

Further, mRNA molecules produced by methods set out herein may comprise a nucleotide sequence encoding one or more protein. Also, mRNA molecules produced by methods set out herein may be produced by in vitro or in vivo transcription reaction. Additionally, mRNA molecules produced by methods set out herein may be translated to produce proteins, for example by a coupled transcription/translation system.

In many instances, at least 70% (e.g., from about 70% to about 100%, from about 75% to about 100%, from about 80% to about 100%, from about 85% to about 100%, from about 90% to about 100%, from about 70% to about 98%, from about 80% to about 98%, etc.) of the mRNA molecules produced by methods set out herein will be capped.

In many instances, the yield of mRNA molecules (e.g., capped mRNA molecules) produced by methods set out herein will be greater than 3 mg/ml of reaction mixture.

### BRIEF DESCRIPTION OF THE DRAWINGS

The skilled artisan will understand that the drawings, described below, are for illustration purposes only. The drawings are not intended to limit the scope of the present teachings in any way.

FIG. 1 illustrates an exemplary synthetic scheme for a trinucleotide cap analog described herein. In this illustration, the synthetic scheme combines imidazolide salt of 7 methyl (LNA) Guanosine 5′ diphosphate (Imm7(LNA)GDP) (A) with (B) to form an exemplary trinucleotide analog of the present disclosure (C).

FIG. 2 illustrates an exemplary synthetic scheme to make imidazolide salt of 7 methyl (LNA) Guanosine 5′ diphosphate (Imm7(LNA)GDP), which is part (A) of FIG. 1.

FIG. 3 illustrates an exemplary synthetic scheme to make the dinucleotide 5′ phosphate dinucleotide such as pApG, which is part (B) of FIG. 1.

FIG. 4 illustrates an exemplary synthetic scheme for the trinucleotide cap analogs containing 3′-O-propargyl described herein. B₁=G, A, U, or C; B₂=G, A, U, or C

FIG. 5 illustrates an exemplary synthetic scheme to make imidazolide salt of 7 methyl 3′-O-propargyl Guanosine 5′-diphosphate (Imm^{7,3′-O-propagyl}GDP), which is used in the synthetic scheme for the trinucleotide cap analogs containing 3′-O-propargyl of FIG. 4.

FIG. 6 illustrates an exemplary synthetic scheme to make dinucleotides to be used in synthesizing trinucleotide caps, including the trinucleotide cap analogs containing 3′-O-propargyl of FIG. 4. B₁=G, A, U, or C; B₂=G, A, U, or C

FIG. 7 illustrates an exemplary synthetic scheme to make trinucleotide cap analogs containing a dye as described herein. B₁=G, A, U, or C; B₂=G, A, U, or C

FIGS. 8A and 8B illustrate an exemplary synthetic scheme to make Imm⁷GDP Containing Dye, which is used in the synthetic scheme for the trinucleotide cap analogs containing a dye of FIG. 7.

FIG. 9 illustrates an exemplary synthetic scheme to make trinucleotide cap analogs containing isopropylidene moieties as described herein. B₁=G, A, U, or C; B₂=G, A, U, or C

FIG. 10 illustrates an exemplary chemical structure of a trinucleotide cap analog containing linker-bound cell-penetrating peptide as disclosed herein.

FIG. 11 is a bar graph showing the mRNA yield of IVT reactions performed in the presence of different mRNA CAP analogs, as indicated and as described in Example 3.

FIG. 12 is a bar graph showing the mRNA capping efficiency of mRNAs capped with ARCA, GAG cap, or LNA-modified GAG cap.

FIG. 13 is a bar graph showing the % cells that are GFP positive, when transfected with crude or HPLC purified mRNA's having no cap, ARCA, GAG cap, or LNA-modified GAG cap as described in Example 4.

FIG. 14 is a bar graph showing the median fluorescence intensity (MFI), of cells transfected with “crude” or “HPLC purified” mRNA preparations capped with no cap, ARCA cap analog, GAG cap analog, or LNA-modified GAG cap analog, as described in Example 4.

FIG. 15 shows the general structure of an exemplary 7-methylguanosine trinucleotide cap structure, including Cap 0 (R₁=H) and Cap 1 (R₁=CH₃) types. B₁ and B₂ are bases that may be the same or different. R₂ represents one or more additional nucleosides linked by an intervening phosphate.

FIG. 16 is a schematic representation of non-template strands of four exemplary wildtype bacteriophage promoters. These promoters are each 23 base pairs in length. Further, each promoter contains an AT rich region, a polymerase specificity loop region, an unwinding region (positions −4 to −1), and an initiation region (positions +1 to +6). The “N” at position +5 of the SP6 promoter may be any nucleotide but, in many instances, will be a G or an A. From top to bottom SEQ ID NO: 591, SEQ ID NO: 592, SEQ ID NO: 593, and SEQ ID NO: 594. “UTR” refers to DNA that forms untranslated regions of transcribe mRNA molecules.

FIG. 17 is a schematic representation of three different mRNA caps binding to a transcriptional initiation site. CAP1 is a dinucleotide cap in which both bases are guanine. CAP2 is a trinucleotide cap in which two bases are guanine with an intervening adenine base. CAP3 is a trinucleotide cap in which all three bases are guanine. The first base in all three instances (guanine) is shown above another base because this first base does not directly interact with the promoter. The lower portion of this figure shows the non-template (5′ to 3′) and template strands (3′ to 5′) of a portion of the promoter where four bases of an unwinding region (shown underlined) meet a transcriptional initiation site. CAP1, CAP2 and CAP3 are positioned in the figure in the location to which they are complementary to the template strand of the initiation region (+1, −1 to +1, and +1 to +2, respectively, for each cap). FIG. 17 discloses SEQ ID NO: 595.

FIG. 18 is a schematic representation similar to that of FIG. 17 with the following differences. CAP4 is a dinucleotide cap in which the first bases is guanine and the second base is adenine. CAP5 is a trinucleotide cap in which the first base is guanine the following two bases are adenine. CAP2 is the same as set out in FIG. 17. Further, the base at position +1 of the non-template strand is adenine instead of guanine. FIG. 18 discloses SEQ ID NO: 596.

FIG. 19 is also a schematic representation that is similar to that of FIG. 17. This figure shows CAP2 associated with the −1 to +1 positions of an initiation region (see dashed line box). Further, the base pair at position +2 of the of the non-template strand is thymine (see downward arrow) with adenine being present in the template strand (not shown). FIG. 19 discloses SEQ ID NO: 597.

FIG. 20 shows RNA yield generated with different promoter sequences and position −1 initiation for HB-WT, HB-MOD2, HB-MOD4, and HB-MOD29. An AGG cap and a +1 initiation site were used for WT-MOD. The template for HB-WT was designed for +1 initiation but no cap was in the reaction mixture. The data used to generate this figure is set out Table 5. HB-GFP refers to a template composed of human beta globin 5′ and 3′ untranslated regions (UTRs).

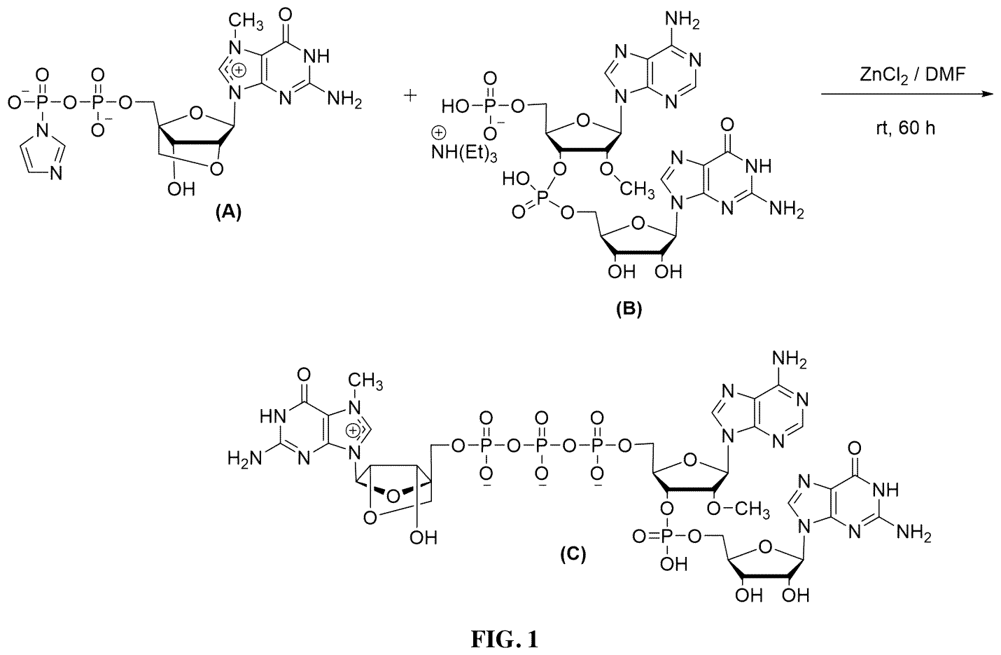

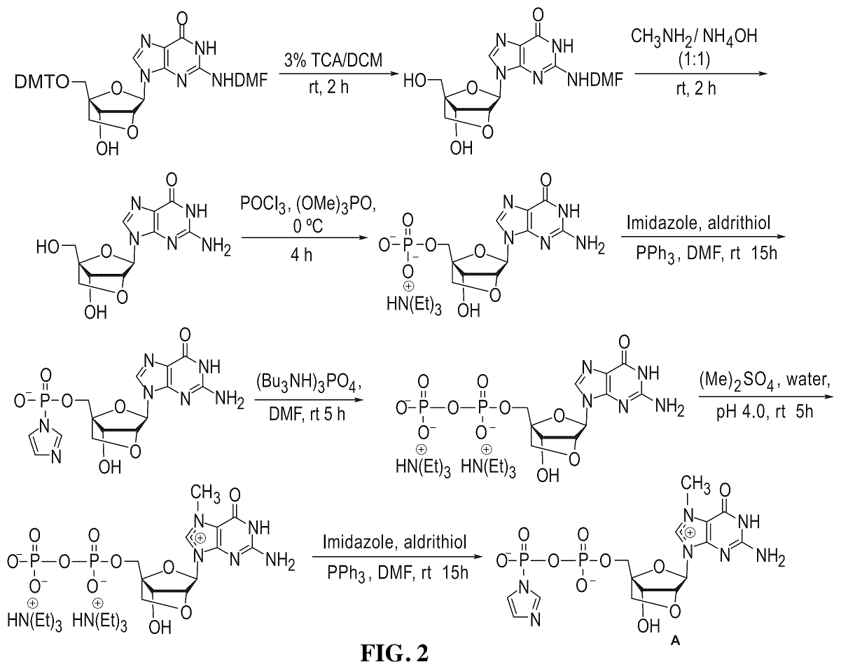

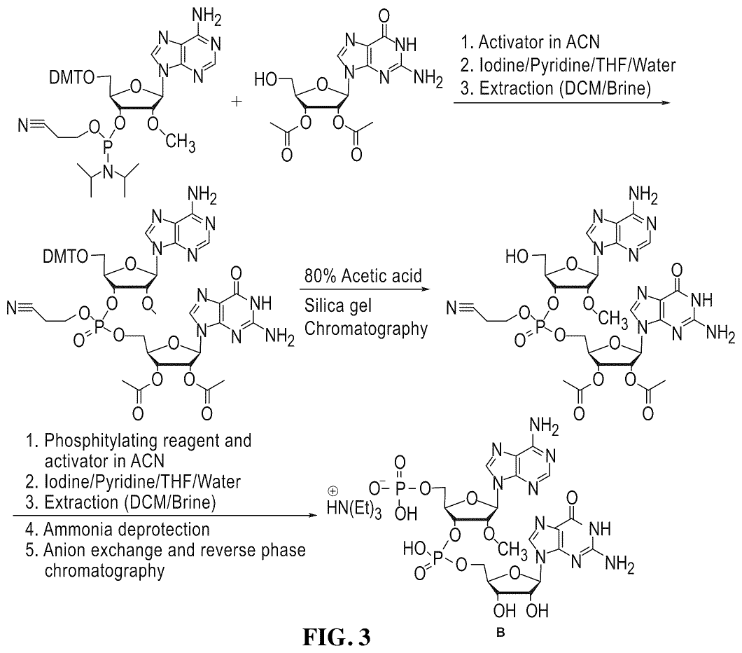

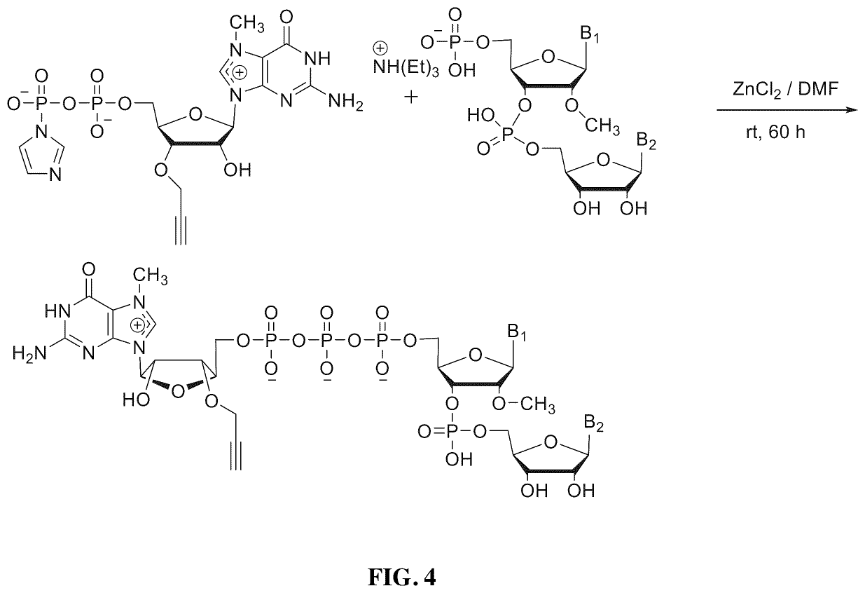

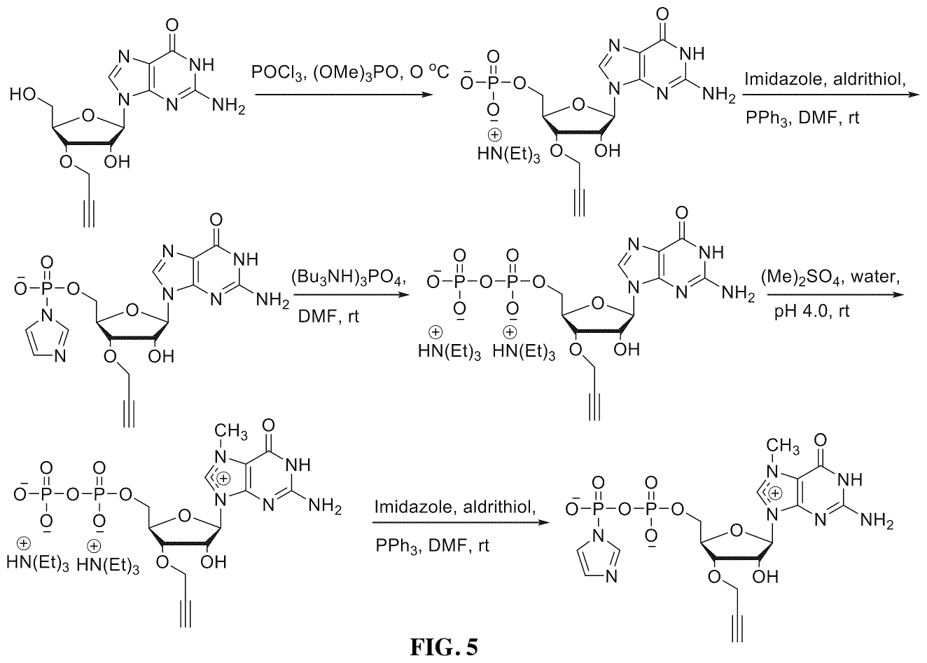

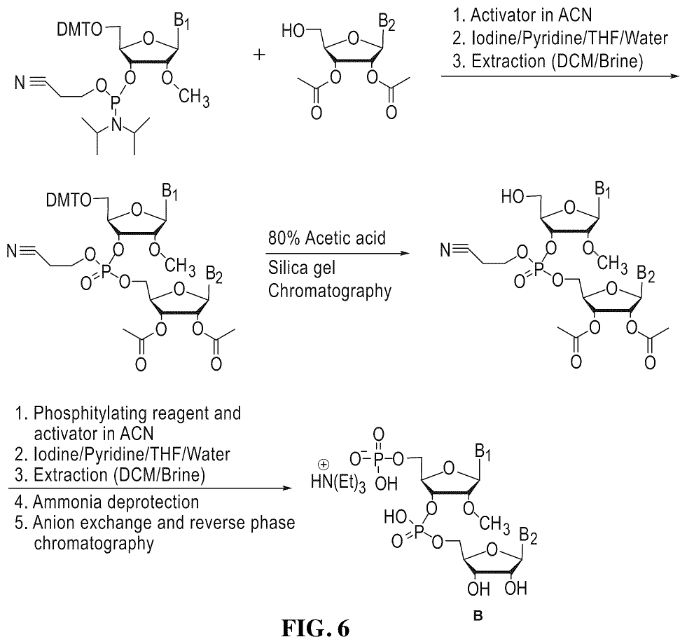

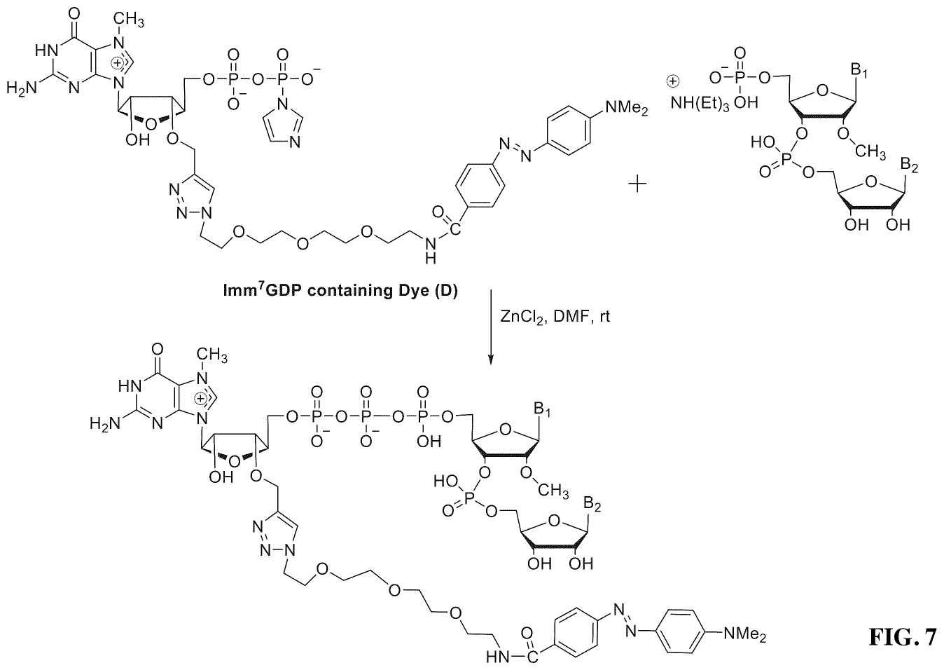

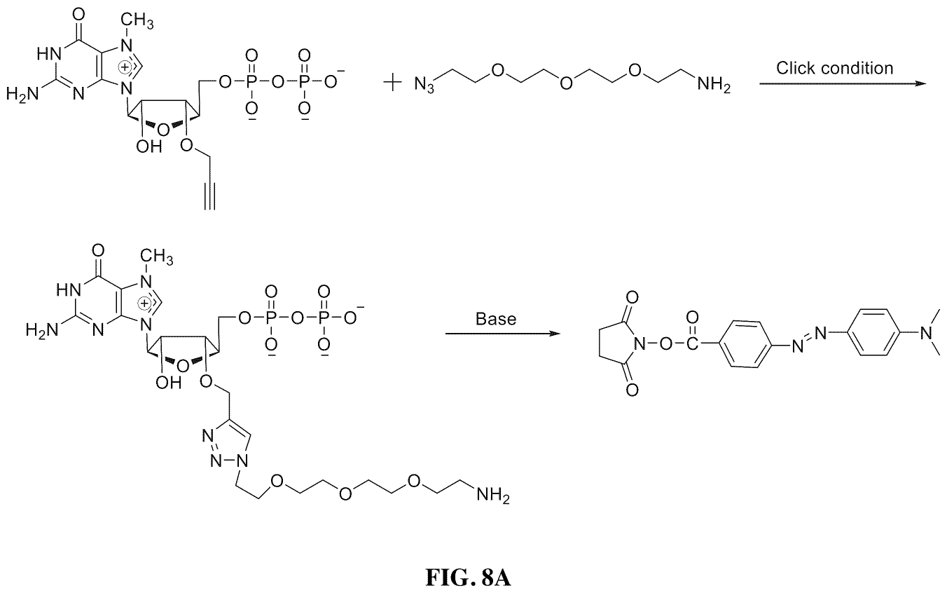

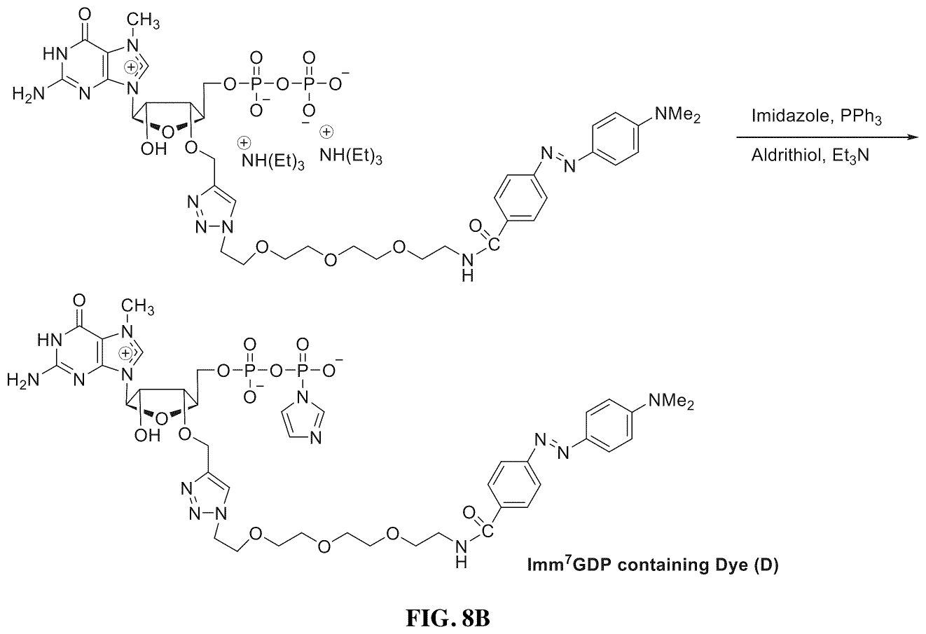

### DESCRIPTION OF THE SEQUENCES

Table 1 provides a listing of sequences used herein.

TABLE 1

| SEQ ID | | |
|---|---|---|
| No. | Sequence | Description |
| 1 | GYSTPPKKKRKVEDP | cell-penetrating peptide |
| 2 | GYSTPPKTRRRP | cell-penetrating peptide |
| 3 | GYSTPGRKKR | cell-penetrating peptide |
| 4 | GYSTPRRNRRRRW | cell-penetrating peptide |
| 5 | PDEVKRKKKPPTSYG | cell-penetrating peptide |
| 6 | PRRRTKPPTSYG | cell-penetrating peptide |
| 7 | RKKRGPTSYG | cell-penetrating peptide |
| 8 | WRRRRNRRPTSYG | cell-penetrating peptide |
| 9 | GYGPPKKKRKVEAPYKA | cell-penetrating peptide |
| 10 | PAAKRVKLD | cell-penetrating peptide |
| 11 | RQRRNELKRSP | cell-penetrating peptide |
| 12 | KRPAATKKAGQAKKKK | cell-penetrating peptide |
| 13 | VRKKRKTEEESPLKDKDAKKSKQE | cell-penetrating peptide |
| 14 | RLRRDAGGRGGVYEHLGGAPRRRK | cell-penetrating peptide |
| 15 | KRKGDEVDGVDECAKKSKK | cell-penetrating peptide |
| 16 | NQSSNFGPMKGGNFGGRSSGPYGGGGQYFAKPRNQG | cell-penetrating peptide |
| | GY | |
| 17 | GGKRTADGSEFESPKKARKVEAYPKAW | cell-penetrating peptide |
| 18 | GGKRTADGSEFESPKKKRAVEAYPKAW | cell-penetrating peptide |
| 19 | GGKRTADGSEFESPKKKAKVEAYPKAW | cell-penetrating peptide |
| 20 | GGKRTADGSEFESPKKKRKVEAPYKAWK | cell-penetrating peptide |
| 21 | GGKRTADGSEFESPKKKRKVEYKAWK | cell-penetrating peptide |
| 22 | GYGPAAKRVKLDEAYPKAWK | cell-penetrating peptide |
| 23 | GGKRTADGSEFEPAAKRVKLDEAYPKAWK | cell-penetrating peptide |
| 24 | GTGPKKKRKVGGGGYGPKKKRLVG | cell-penetrating peptide |
| 25 | KRPAATKKAGQAKKKKLEAYPKAWK | cell-penetrating peptide |
| 26 | ATKGTKRSYEQMETGE | cell-penetrating peptide |
| 27 | GKWERKPIRCAS | cell-penetrating peptide |
| 28 | GYGKRTADSQHSTPPKKKRKVEAPYKAWK | cell-penetrating peptide |
| 29 | KRTADSQHSTPPKKKRKVEAPYKAWK | cell-penetrating peptide |
| 30 | GYGPPKKKRKVEAPYKAWKWAKYPAMRRAHHRRRR | cell-penetrating peptide |
| | ASHRRRTTTGT | |
| 31 | GYGPPKKKRKVEAPYKAWKRGARRYSKMKRRRRRV | cell-penetrating peptide |
| | ARRHRRRP | |
| 32 | FWGYGYGPPKKKRKVEAPYKAWK | cell-penetrating peptide |
| 33 | GKPSSDDEATADSQHSTPPKKKERKVED | cell-penetrating peptide |
| | | cell-penetrating peptide |
| 34 | GKPTADDQHSTPPKKKRKVED | cell-penetrating peptide |
| 35 | GGKRTADGSEFESPKKARKVEAYPKAK | cell-penetrating peptide |
| 36 | EKIRLRPGRKKRYRLKHL | cell-penetrating peptide |
| 37 | PEGTRQARRNRRRRWRKR | cell-penetrating peptide |
| 38 | PEGTRQPRRNRRRRWRKR | cell-penetrating peptide |
| 39 | GVKRSYGAARGDDRRRPNVVAPYKAW | cell-penetrating peptide |
| 40 | KSVPNRTRTYIKLKRLRFKGAPYKAW | cell-penetrating peptide |
| 41 | EMRRRREEEGLQLRKQKREEQLFKRRN | cell-penetrating peptide |
| 42 | FEAALAEALAEALA | cell-penetrating peptide |
| 43 | Ac-LARLLPRLLARL-NHCH₃ | cell-penetrating peptide |
| 44 | GLLEELLELLEELWEELLEG | cell-penetrating peptide |
| 45 | GWEGLIEGIEGGWEGLIEG | cell-penetrating peptide |
| 46 | GLFEALAEFIEGGWEGLIEG | cell-penetrating peptide |
| 47 | GLFEALLELLESLWELLLEA | cell-penetrating peptide |
| 48 | GGYCLEKWMIVASELKCFGNTA | cell-penetrating peptide |
| 49 | GGYCLTRWMLIEAELKCFGNTAV | cell-penetrating peptide |
| 50 | WEAALAEALAEALAEHLAEALAEALEALAA | cell-penetrating peptide |
| 51 | GLFGAIAGFIENGWEGMIDGWYG | cell-penetrating peptide |
| 52 | GIGAVLKVLTTGLPALISWIKRKRQQ | cell-penetrating peptide |
| 53 | GRKKRRQRRRPPQ | cell-penetrating peptide |
| 54 | RQIKIWFQNRRMKWKK | cell-penetrating peptide |
| 55 | GWTLNSAGYLLGKINLKALAALAKKIL | cell-penetrating peptide |
| 56 | WEAKLAKALAKALAKHLAKALAKALKACEA | cell-penetrating peptide |
| 57 | GLFKALLKLLKSLWKLLLKA | cell-penetrating peptide |
| 58 | GLFRALLRLLRSLWRLLLRA | cell-penetrating peptide |
| 59 | GLFEALLELLESLYELLLEA | cell-penetrating peptide |
| 60 | GLFEALEELWEA | cell-penetrating peptide |
| 61 | GLFLLEEWLE | cell-penetrating peptide |
| 62 | GLFLLEEWLEK | cell-penetrating peptide |
| 63 | GLFEALLELLESLWELLLEAK | cell-penetrating peptide |
| 64 | Suc-GLFKLLEEWLE | cell-penetrating peptide |
| 65 | Suc-GLFKLLEEWLEK | cell-penetrating peptide |
| 66 | GLFEAIAEFIEGGWEGLIEG | cell-penetrating peptide |
| 67 | GLFKAIAKFIKGGWKGLIKG | cell-penetrating peptide |
| 68 | IRFKKTKLIASIAMALC | cell-penetrating peptide |
| 69 | ALAGTHAGASLTFQVLDKV1EELGKVSRK | cell-penetrating peptide |
| 70 | GLFEAIEGFIENGWEGMIDGWYG | cell-penetrating peptide |
| 71 | GYICRRARGDNPDDRCT | cell-penetrating peptide |
| 72 | GLFEAIAEFIEGGWEGLIEGCA | cell-penetrating peptide |
| 73 | GLFHAIAHFIHGGWHGLIHGWWYG | cell-penetrating peptide |
| 74 | RRRQRRKKRGGDIMGEWGNEIFGAIAGFLG | cell-penetrating peptide |
| 75 | GLFEAIADFIENGWEGMIDGGG | cell-penetrating peptide |
| 76 | ALAGTIIAGASLTFQVLDKVIEELGKVSRKK | cell-penetrating peptide |
| 77 | IRFKKTKLIASIAMA | cell-penetrating peptide |
| 78 | GLWHLLLHLWRRLLRLLR | cell-penetrating peptide |
| 79 | KKIMLLLMTLLLVSLPLAQEQ | cell-penetrating peptide |
| 80 | GLFEALLELLESLWELLLEAWYG | cell-penetrating peptide |
| 81 | RLLRLLLRLWRRLLRLLR | cell-penetrating peptide |
| 82 | LLELELLELELLLELELLELELLLEL | cell-penetrating peptide |
| 83 | GLFEALLELLESLWELLLEARRRRRRRR | cell-penetrating peptide |
| 84 | GLFEALLELLESLWELLLEARRRRRR | cell-penetrating peptide |
| 85 | GLFEALLELLESLWELLLEAKKKKKKKK | cell-penetrating peptide |
| 86 | GLFEALLELLESLWELLLEAKKKKKK | cell-penetrating peptide |
| 87 | GLFEALLELLESLWELLLEAKK | cell-penetrating peptide |
| 88 | GLFEALLELLESLWELLLEAKKKK | cell-penetrating peptide |
| 89 | GLFEALLELLESLWELLLEAEE | cell-penetrating peptide |
| 90 | GLFEALLELLESLWELLLEAEEEE | cell-penetrating peptide |
| 91 | GLFEALLELLESLWELLLEAEEEEEE | cell-penetrating peptide |
| 92 | GLFEALLELLESLWELLL | cell-penetrating peptide |
| 93 | PLSSIFSRIGDPRGARRYAKMKRRRRRVARRHRRRP | cell-penetrating peptide |
| 94 | GPFHYFQFLFPPV | cell-penetrating peptide |
| 95 | GSSSWWQRWWPPW | cell-penetrating peptide |
| 96 | RRRQRRKKR | cell-penetrating peptide |
| 97 | KKKK | cell-penetrating peptide |
| 98 | KKKKKK | cell-penetrating peptide |
| 99 | KKKKKKKK | cell-penetrating peptide |
| 100 | KKKKKKKKKK | cell-penetrating peptide |
| 101 | KKKKKKKKKKKK | cell-penetrating peptide |
| 102 | KKKKKKKKKKKKKKKK | cell-penetrating peptide |
| 103 | KKKKKKKKKKKKKKKKKKKK | cell-penetrating peptide |
| 104 | KKKKKKKKKKKKKKKKKKKKKKKK | cell-penetrating peptide |
| 105 | RRRR | cell-penetrating peptide |
| 106 | RRRRRR | cell-penetrating peptide |
| 107 | RRRRRRRR | cell-penetrating peptide |
| 108 | RRRRRRRRRR | cell-penetrating peptide |
| 109 | RRRRRRRRRRRR | cell-penetrating peptide |
| 110 | RRRRRRRRRRRRRRRR | cell-penetrating peptide |
| 111 | RRRRRRRRRRRRRRRRRRRR | cell-penetrating peptide |
| 112 | RRRRRRRRRRRRRRRRRRRRRRRR | cell-penetrating peptide |
| 113 | YKA | cell-penetrating peptide |
| 114 | KKKKKKKKWKGGGGACYGLPHLFCG | cell-penetrating peptide |
| 115 | YKAKKKKKKKKWK | cell-penetrating peptide |
| 116 | KTPKKAKKPKTPKKAKKP | cell-penetrating peptide |
| 117 | KKAKKPAATRKSSKNPKKPKTVKPKKVAK | cell-penetrating peptide |
| 118 | RGARRYSKMKRRRRRVARRHRRRP | cell-penetrating peptide |
| 119 | TRQARRNRRRRWRERQRGSGSG | cell-penetrating peptide |
| 120 | KRPRGRPKGSKKNWRRRKRRASRRSPRRR | cell-penetrating peptide |
| 121 | KRGRGRPRKQPPKEPSEVPTPKRPRGRPKGSKNK | cell-penetrating peptide |
| 122 | KEKYEKDIAAYRAKGKPAAKKGVVKAEKSKKKK | cell-penetrating peptide |
| 123 | YKAKKKKKKKKKKWK | cell-penetrating peptide |
| 124 | KKKKKKKGGC | cell-penetrating peptide |
| 125 | YRARRRRRRRRWR | cell-penetrating peptide |
| 126 | YRARRRRRRRRRRWR | cell-penetrating peptide |
| 127 | KGDPKKPRGKMSSYAFFVQTCREEHKKKHPDASVNFS | cell-penetrating peptide |
| | EFSKK | |
| 128 | KKQLKKQLKKQLKQWK | cell-penetrating peptide |
| 129 | KKSPKKSPKKSPKKSK | cell-penetrating peptide |
| 130 | KLSKLEKKSKLEK | cell-penetrating peptide |
| 131 | KLSKLEKKLSKLEKKSKLEK | cell-penetrating peptide |
| 132 | KSLKKSLKKSLKKSK | cell-penetrating peptide |
| 133 | KIRRRGKNKVAARTCRQRRTDR | cell-penetrating peptide |
| 134 | KIRRRGKNKVAAQNCRKRKLET | cell-penetrating peptide |
| 135 | KRRIRREKNKMAAAKCRNRRRELT | cell-penetrating peptide |
| 136 | KDRSNLLERHTR | cell-penetrating peptide |
| 137 | KRPAATKKAGQAKKKL | cell-penetrating peptide |
| 138 | RRRRRREEEE | cell-penetrating peptide |
| 139 | RRRRRREEEEEE | cell-penetrating peptide |
| 140 | RRRRRREEEEEEEE | cell-penetrating peptide |
| 141 | RRRRRRRREEEE | cell-penetrating peptide |
| 142 | RRRRRRRREEEEEE | cell-penetrating peptide |
| 143 | RRRRRRRREEEEEEEE | cell-penetrating peptide |
| 144 | RRRRRRRRRRRREEEE | cell-penetrating peptide |
| 145 | RRRRRRRRRRRREEEEEE | cell-penetrating peptide |
| 146 | RRRRRRRRRRRREEEEEE | cell-penetrating peptide |
| 147 | KLSKLEKK | cell-penetrating peptide |
| 148 | SKLEK | cell-penetrating peptide |
| 149 | KLSKLEKKLSKLEKK | cell-penetrating peptide |
| 150 | PKKKRKVGGGRGDSP | cell-penetrating peptide |
| 151 | LPHKSMPCG | cell-penetrating peptide |
| 152 | GACLQHKSMPCG | cell-penetrating peptide |
| 153 | YGLPHLFCG | cell-penetrating peptide |
| 154 | SERSMNFCG | cell-penetrating peptide |
| 155 | DHYSLYEDLERGTDK | cell-penetrating peptide |
| 156 | ISLPRTSGAQRASTTR | cell-penetrating peptide |
| 157 | EKLQTKYGLPHKVEFCG | cell-penetrating peptide |
| 158 | TRISESQAKPGD | cell-penetrating peptide |
| 159 | LVFFDY | cell-penetrating peptide |
| 160 | WGGNGPTTFDCSGYTKYVFAK | cell-penetrating peptide |
| 161 | INIGTTGWGDHYSLY | cell-penetrating peptide |
| 162 | YDNIHG | cell-penetrating peptide |
| 163 | AGWGKFLVGFGRV | cell-penetrating peptide |
| 164 | SIGYPLP | cell-penetrating peptide |
| 165 | TTHWGFTL | cell-penetrating peptide |
| 166 | HLQIQPYPQISG | cell-penetrating peptide |
| 167 | KLNIVSVNG | cell-penetrating peptide |
| 168 | RGH | cell-penetrating peptide |
| 169 | DNRIRLQAKAA | cell-penetrating peptide |
| 170 | KIKMVISWKG | cell-penetrating peptide |
| 171 | LPWYSYLYAVSA | cell-penetrating peptide |
| 172 | WNLPWYYSVSPT | cell-penetrating peptide |
| 173 | WNL | cell-penetrating peptide |
| 174 | PWYYSVSPT | cell-penetrating peptide |
| 175 | SSWESYKSGGGTRL | cell-penetrating peptide |
| 176 | RDWSSQHPGRCNGETHLK | cell-penetrating peptide |
| 177 | SLPTLTL | cell-penetrating peptide |
| 178 | VICTGGDYSFALPVGQWPVMT | cell-penetrating peptide |
| 179 | DKPSYQFGGHNSVDFEEDTLPKV | cell-penetrating peptide |
| 180 | RARRRKRASATQLYQTCKASGTCPPD | cell-penetrating peptide |
| 181 | SGDYSFALPVGQWPWMTG | cell-penetrating peptide |
| 182 | CTGGDYSFALPVGQWPW | cell-penetrating peptide |
| 183 | FYYDYDFFFDYWGQG | cell-penetrating peptide |
| 184 | HLRRLRRRLLREAEG | cell-penetrating peptide |
| 185 | DYYCAAWDDSLNGYSVF | cell-penetrating peptide |
| 186 | YYCLQSMEDPYTFGG | cell-penetrating peptide |
| 187 | YYCARSDGNYGYYYALDYDY | cell-penetrating peptide |
| 188 | AARSPSYYRYDY | cell-penetrating peptide |
| 189 | GPYYAMDYD | cell-penetrating peptide |
| 190 | YYCQQRSSYPYTEGGAYPKAWK | cell-penetrating peptide |
| 191 | YYCQRYDSDWSFGQGTKL | cell-penetrating peptide |
| 192 | YYCARSGYYAMDYWGQGT | cell-penetrating peptide |
| 193 | RVRRGACRGDCLG | cell-penetrating peptide |
| 194 | RVRRGACRYDCLG | cell-penetrating peptide |
| 195 | YYCAKGTHWGFWSGYFDYWGQGT | cell-penetrating peptide |
| 196 | GRENYHGCTTHWGFTLC | cell-penetrating peptide |
| 197 | VQATQSNQHTPRGGGSK | cell-penetrating peptide |
| 198 | DPRAPGS | cell-penetrating peptide |
| 199 | YYCQQRSSYPYTFGG | cell-penetrating peptide |
| 200 | AARSPSYYRYDYGPYYAMDYD | cell-penetrating peptide |
| 201 | GPKLTGILISILSLFVES | cell-penetrating peptide |
| 202 | KYILRWRPKNS | cell-penetrating peptide |
| 203 | IKVAV | cell-penetrating peptide |
| 204 | WTPPRAQITGYRLTVGLTRR | cell-penetrating peptide |
| 205 | AASIKVAVSADR | cell-penetrating peptide |
| 206 | KLDAPT | cell-penetrating peptide |
| 207 | NRWHSIYITRFG | cell-penetrating peptide |
| 208 | PHSRN | cell-penetrating peptide |
| 209 | SSFHFDGSGYAM | cell-penetrating peptide |
| 210 | RGDS | cell-penetrating peptide |
| 211 | IAFQRN | cell-penetrating peptide |
| 212 | GRGDSP | cell-penetrating peptide |
| 213 | TWYKIAFQRRK | cell-penetrating peptide |
| 214 | EDGIHEL | cell-penetrating peptide |
| 215 | SLVRNRRVITIQ | cell-penetrating peptide |
| 216 | YRVRVTPKEKTGPMKE | cell-penetrating peptide |
| 217 | LQVQLSR | cell-penetrating peptide |
| 218 | SPPRRARVT | cell-penetrating peptide |
| 219 | RKRLQVQLSIRT | cell-penetrating peptide |
| 220 | ATETTITIS | cell-penetrating peptide |
| 221 | NAPFPKLSWTIQ | cell-penetrating peptide |
| 222 | VSPPRRARVTDATETTITISWRTKTETITGG | cell-penetrating peptide |
| 223 | WTIQTTVDRGLL | cell-penetrating peptide |
| 224 | KPDVRSYTITG | cell-penetrating peptide |
| 225 | DTINNGRDHMILI | cell-penetrating peptide |
| 226 | ANGQTPIQRYIK | cell-penetrating peptide |
| 227 | MILISIGKSQKRM | cell-penetrating peptide |
| 228 | PRARITGYIIKYEKPGSPPREVVPRPRPGV | cell-penetrating peptide |
| 229 | PPFLMLLKGSTR | cell-penetrating peptide |
| 230 | WQPPRARI | cell-penetrating peptide |
| 231 | NQRLASFSNAQQS | cell-penetrating peptide |
| 232 | WQPPRARITGYIIKYEKPG | cell-penetrating peptide |
| 233 | ISNVFVQRMSQSPEVLD | cell-penetrating peptide |
| 234 | YEKPGSPPREVVPRPRPGV | cell-penetrating peptide |
| 235 | KARSFNVNQLLQD | cell-penetrating peptide |
| 236 | KNNQKSEPLIGRKKT | cell-penetrating peptide |
| 237 | KNSFMALYLSKG | cell-penetrating peptide |
| 238 | EILDVPST | cell-penetrating peptide |
| 239 | KNSFMALYLSKGRLVFALG | cell-penetrating peptide |
| 240 | IDAPS | cell-penetrating peptide |
| 241 | RDSFVALYLSEGHVIFAGLG | cell-penetrating peptide |
| 242 | VVIDASTAIDAPSNL | cell-penetrating peptide |
| 243 | KPRLQFSLDIQT | cell-penetrating peptide |
| 244 | LDVPS | cell-penetrating peptide |
| 245 | DGQWHSVTVSIK | cell-penetrating peptide |
| 246 | REDV | cell-penetrating peptide |
| 247 | FVLYLGSKNAKK | cell-penetrating peptide |
| 248 | PHSRNRGDSP | cell-penetrating peptide |
| 249 | LAIKNDNLVYVY | cell-penetrating peptide |
| 250 | LWVTVRSQQRGLF | cell-penetrating peptide |
| 251 | AYFSIVKIERVG | cell-penetrating peptide |
| 252 | GTNNWWQSPSIQN | cell-penetrating peptide |
| 253 | DVISLYNFKHIY | cell-penetrating peptide |
| 254 | WVTVTLDLRQVFQ | cell-penetrating peptide |
| 255 | FFDGSSYAVVRD | cell-penetrating peptide |
| 256 | RQVFQVAYIIIKA | cell-penetrating peptide |
| 257 | LHVFYDFGFGFSNG | cell-penetrating peptide |
| 258 | LTRYKITPRRGPPT | cell-penetrating peptide |
| 259 | LKKAQINDAKYREISIIYHN | cell-penetrating peptide |
| 260 | LLEFTSARYIRL | cell-penetrating peptide |
| 261 | RAYFNGQSFIAS | cell-penetrating peptide |
| 262 | YIRLRLQRIRTL | cell-penetrating peptide |
| 263 | SRLRGKNPTKGK | cell-penetrating peptide |
| 264 | RRYYYSIKDISV | cell-penetrating peptide |
| 265 | LHKKGKNSSKPK | cell-penetrating peptide |
| 266 | SINNTAVNQRLT | cell-penetrating peptide |
| 267 | RLKTRSSHGMIF | cell-penetrating peptide |
| 268 | GGFLKYTVSYDI | cell-penetrating peptide |
| 269 | GEKSQFSIRLKT | cell-penetrating peptide |
| 270 | RDQLMTVLANVT | cell-penetrating peptide |
| 271 | TLFLAHGRLVFM | cell-penetrating peptide |
| 272 | ANVTHLLIRANY | cell-penetrating peptide |
| 273 | LVFMFNVGHKKL | cell-penetrating peptide |
| 274 | AGTFALRGDNPQG | cell-penetrating peptide |
| 275 | TLFLAHGRLVFMFNVGHKKL | cell-penetrating peptide |
| 276 | VLIKGGRARKHV | cell-penetrating peptide |
| 277 | DFMTLFLAHGRLVFMGNVG | cell-penetrating peptide |
| 278 | LSNIDYLIKAS | cell-penetrating peptide |
| 279 | HKKLKIRSQEKY | cell-penetrating peptide |
| 280 | LQQSRIANISME | cell-penetrating peptide |
| 281 | GAAWKIKGPIYL | cell-penetrating peptide |
| 282 | NLLLLLVKANLK | cell-penetrating peptide |
| 283 | VIRDSNVVQLDV | cell-penetrating peptide |
| 284 | HRDELLLWARKI | cell-penetrating peptide |
| 285 | GLIYYVAHQNQM | cell-penetrating peptide |
| 286 | KRRARDLVHRAE | cell-penetrating peptide |
| 287 | DYATLQLQEGRLHFMFDLG | cell-penetrating peptide |
| 288 | SQFQESVDNITK | cell-penetrating peptide |
| 289 | KKGSYNNIVVHV | cell-penetrating peptide |
| 290 | PGGMREKGRKAR | cell-penetrating peptide |
| 291 | ADNLLFYLGSAK | cell-penetrating peptide |
| 292 | MEMQANLLLDRL | cell-penetrating peptide |
| 293 | GSAKFIDFLAIE | cell-penetrating peptide |
| 294 | LSEIKLLISAR | cell-penetrating peptide |
| 295 | KVSFLWWVGSGV | cell-penetrating peptide |
| 296 | RDFTKATNIRLRFLR | cell-penetrating peptide |
| 297 | SYWYRIEASRTG | cell-penetrating peptide |
| 298 | ISTVMFKFRTFS | cell-penetrating peptide |
| 299 | YFDGTGFAKAVG | cell-penetrating peptide |
| 300 | KQANISIVDIDSN | cell-penetrating peptide |
| 301 | NGQWHKVTAKKI | cell-penetrating peptide |
| 302 | FSTRNESGIILL | cell-penetrating peptide |
| 303 | AKKIKNRLELVV | cell-penetrating peptide |
| 304 | RRQTTQAYYAIF | cell-penetrating peptide |
| 305 | GFPGGLNQFGLTTN | cell-penetrating peptide |
| 306 | YAIFLNKGRLEV | cell-penetrating peptide |
| 307 | NQFGLTTNIRFRG | cell-penetrating peptide |
| 308 | KNRLTIELEVRT | cell-penetrating peptide |
| 309 | IRSLKLTKGTGKP | cell-penetrating peptide |
| 310 | GLLFYMARINHA | cell-penetrating peptide |
| 311 | AKALELRGVQPVS | cell-penetrating peptide |
| 312 | VQLRNGFPYFSY | cell-penetrating peptide |
| 313 | GQLFHVAYILIKF | cell-penetrating peptide |
| 314 | HKIKIVRVKQEG | cell-penetrating peptide |
| 315 | NVLSLYNFKTTF | cell-penetrating peptide |
| 316 | DFGTVQLRNGFPFFSYDLG | cell-penetrating peptide |
| 317 | SQRIYQFAKLNYT | cell-penetrating peptide |
| 318 | NIRLRFLRTNTL | cell-penetrating peptide |
| 319 | EVNVTLDLGQVFH | cell-penetrating peptide |
| 320 | GKNTGDHFVLYM | cell-penetrating peptide |
| 321 | GQVFHVAYVLIKF | cell-penetrating peptide |
| 322 | VVSLYNFEQTFML | cell-penetrating peptide |
| 323 | HQQDLGTAGSCLRKFSTMFLF | cell-penetrating peptide |
| 324 | RFDQELRLVSYN | cell-penetrating peptide |
| 325 | HQQDLGTAGSCLRKFSTMFLFCNI | cell-penetrating peptide |
| 326 | RLVSYSGVLFFLK | cell-penetrating peptide |
| 327 | VAEIDGIEL | cell-penetrating peptide |
| 328 | NWRHISYITRFG | cell-penetrating peptide |
| 329 | GIIFFL | cell-penetrating peptide |
| 330 | KRLQVQLRSIRT | cell-penetrating peptide |
| 331 | ASKAIQVFLLGG | cell-penetrating peptide |
| 332 | TWYKIAFQRNRK | cell-penetrating peptide |
| 333 | VLVRVERATVFS | cell-penetrating peptide |
| 334 | QVFQVAYIIIKA | cell-penetrating peptide |
| 335 | TVFSVDQDNMLE | cell-penetrating peptide |
| 336 | GEFYFDLRLKGDK | cell-penetrating peptide |
| 337 | RLRGPQRVFDLH | cell-penetrating peptide |
| 338 | GTPGPQGIA | cell-penetrating peptide |
| 339 | FDLHQNMGSVN | cell-penetrating peptide |
| 340 | GQRDVV | cell-penetrating peptide |
| 341 | LRAHAVDVNG | cell-penetrating peptide |
| 342 | TAGSCLRKFSTM | cell-penetrating peptide |
| 343 | LFSHAVSSNG | cell-penetrating peptide |
| 344 | KGHRGF | cell-penetrating peptide |
| 345 | TAGSCLRKFSTMFLF | cell-penetrating peptide |
| 346 | TAGSCLRKFSTMFLFCNI | cell-penetrating peptide |
| 347 | DLGTAGSCLRKFSTM | cell-penetrating peptide |
| 348 | HQQDLGTAGSCLRKFSTM | cell-penetrating peptide |
| 349 | RNIAEIIKDI | cell-penetrating peptide |
| 350 | SIGFRGDGQTC | cell-penetrating peptide |
| 351 | LNRQELFPFG | cell-penetrating peptide |
| 352 | RIQNLLKITNLRIKFVK | cell-penetrating peptide |
| 353 | KKQRFRHRNRKGYRSQ | cell-penetrating peptide |
| 354 | SINNTAVMQRLT | cell-penetrating peptide |
| 355 | FRHRNRKGY | cell-penetrating peptide |
| 356 | RYRVRVTPKEKTGPMKE | cell-penetrating peptide |
| 357 | SETTVKYIFRLHE | cell-penetrating peptide |
| 358 | GHRGPTGRPGKRGKQGQKGDS | cell-penetrating peptide |
| 359 | KAFDITYVRLKF | cell-penetrating peptide |
| 360 | GDLGRPGRKGRPGPP | cell-penetrating peptide |
| 361 | YIGSR | cell-penetrating peptide |
| 362 | RGEFYFDLRLKGDK | cell-penetrating peptide |
| 363 | LAGSCLARFSTM | cell-penetrating peptide |
| 364 | LALFLSNGHFVA | cell-penetrating peptide |
| 365 | ISRCQVCMKKRH | cell-penetrating peptide |
| 366 | PGRWHKVSVRWE | cell-penetrating peptide |
| 367 | TDIPPCPHGWISLWK | cell-penetrating peptide |
| 368 | VRWGMQQIQLVV | cell-penetrating peptide |
| 369 | TAIPSCPEGTVPLYS | cell-penetrating peptide |
| 370 | KMPYVSLELEMR | cell-penetrating peptide |
| 371 | GPAGKDGEAGAQG | cell-penetrating peptide |
| 372 | VLLQANDGAGEF | cell-penetrating peptide |
| 373 | GLPGER | cell-penetrating peptide |
| 374 | DGRWHRVAVIMG | cell-penetrating peptide |
| 375 | LAGSCLPVFSTL | cell-penetrating peptide |
| 376 | APVNVTASVQIQ | cell-penetrating peptide |
| 377 | TAGSCLRRFSTM | cell-penetrating peptide |
| 378 | KQGKALTQRHAK | cell-penetrating peptide |
| 379 | TAGSCLRKF | cell-penetrating peptide |
| 380 | RYVVLPR | cell-penetrating peptide |
| 381 | TAGSCL | cell-penetrating peptide |
| 382 | SPYTFIDSLVLMPY | cell-penetrating peptide |
| 383 | TAG | cell-penetrating peptide |
| 384 | PDSGR | cell-penetrating peptide |
| 385 | QQNLGSVNVSTG | cell-penetrating peptide |
| 386 | SRATAQKVSRRS | cell-penetrating peptide |
| 387 | DPGYIGSR | cell-penetrating peptide |
| 388 | GSLSSHLEFVGI | cell-penetrating peptide |
| 389 | VILQQSAADIAR | cell-penetrating peptide |
| 390 | RNRLHLSMLVRP | cell-penetrating peptide |
| 391 | KDISEKVAVYST | cell-penetrating peptide |
| 392 | APMSGRSPSLVLK | cell-penetrating peptide |
| 393 | LGTIPG | cell-penetrating peptide |
| 394 | AFGVLALWGTRV | cell-penetrating peptide |
| 395 | TDIRVTLNRLNTF | cell-penetrating peptide |
| 396 | IENVVTTFAPNR | cell-penetrating peptide |
| 397 | AFSTLEGRPSAY | cell-penetrating peptide |
| 398 | LEAEFHFTHLIM | cell-penetrating peptide |
| 399 | TSAEAYNLLLRT | cell-penetrating peptide |
| 400 | HLIMTFKTFRPA | cell-penetrating peptide |
| 401 | LNRRYEQARNIS | cell-penetrating peptide |
| 402 | KTWGVYRYFAYD | cell-penetrating peptide |
| 403 | SLLSQLNNLLDQ | cell-penetrating peptide |
| 404 | TNLRIKFVKLHT | cell-penetrating peptide |
| 405 | RDIAEIIKDI | cell-penetrating peptide |
| 406 | KRLVTGQR | cell-penetrating peptide |
| 407 | SHAVSS | cell-penetrating peptide |
| 408 | GPGVVVVERQYI | cell-penetrating peptide |
| 409 | ADTPPV | cell-penetrating peptide |
| 410 | NEPKVLKSYYYAI | cell-penetrating peptide |
| 411 | LRAHAVDING | cell-penetrating peptide |
| 412 | YYAISDFAVGGR | cell-penetrating peptide |
| 413 | DSITKYFQMSLE | cell-penetrating peptide |
| 414 | LPFFNDRPWRRAT | cell-penetrating peptide |
| 415 | YTALIIATDN | cell-penetrating peptide |
| 416 | FDPELYRSTGHGGH | cell-penetrating peptide |
| 417 | VITVKDINDN | cell-penetrating peptide |
| 418 | TNAVGYSVYDIS | cell-penetrating peptide |
| 419 | GLDRESYPYY | cell-penetrating peptide |
| 420 | APVKFLGNQVLSY | cell-penetrating peptide |
| 421 | MKVSATDADD | cell-penetrating peptide |
| 422 | SFSFRVDRRDTR | cell-penetrating peptide |
| 423 | PQVTRGDVFTMP | cell-penetrating peptide |
| 424 | TWSKVGGHLRPGIVQSG | cell-penetrating peptide |
| 425 | KEAEREVTDLLR | cell-penetrating peptide |
| 426 | RGDV | cell-penetrating peptide |
| 427 | AAEPLKNIGILF | cell-penetrating peptide |
| 428 | FALWDAIIGEL | cell-penetrating peptide |
| 429 | VGVAPG | cell-penetrating peptide |
| 430 | LWPLLAVLAAVA | cell-penetrating peptide |
| 431 | PGVGV | cell-penetrating peptide |
| 432 | VFDNFVLK | cell-penetrating peptide |
| 433 | TSIKIRGTYSER | cell-penetrating peptide |
| 434 | TTSWSQCSKS | cell-penetrating peptide |
| 435 | DPETGV | cell-penetrating peptide |
| 436 | KRSR | cell-penetrating peptide |
| 437 | QGADTPPVGV | cell-penetrating peptide |
| 438 | SVVYGLR | cell-penetrating peptide |
| 439 | PLDREAIAKY | cell-penetrating peptide |
| 440 | DGRGDSVAYG | cell-penetrating peptide |
| 441 | HAVDI | cell-penetrating peptide |
| 442 | LALERKDHSG | cell-penetrating peptide |
| 443 | DQNDN | cell-penetrating peptide |
| 444 | YSMKKTTMKIIPFNRLTIG | cell-penetrating peptide |
| 445 | QDPELPDKNM | cell-penetrating peptide |
| 446 | RGDF | cell-penetrating peptide |
| 447 | LVVQAADLQG | cell-penetrating peptide |
| 448 | GVYYQGGTYSKAS | cell-penetrating peptide |
| 449 | NDDGGQFVVT | cell-penetrating peptide |
| 450 | TAGSCLRKFSCL | cell-penetrating peptide |
| 451 | YILHVAVTN | cell-penetrating peptide |
| 452 | CNYYSNSYSFWLASLNPER | cell-penetrating peptide |
| 453 | TYRIWRDTAN | cell-penetrating peptide |
| 454 | TGLSCLQRFTTM | cell-penetrating peptide |
| 455 | GFTCECSIGFRGDGQTCYGIVFWSEV | cell-penetrating peptide |
| 456 | HHLGGAKQAGDV | cell-penetrating peptide |
| 457 | SCLPGFSGDGRACRDVDECGH | cell-penetrating peptide |
| 458 | MAPRPSLAKKQRFRHRNRKGYRSQRGHSRG | cell-penetrating peptide |
| 459 | KKQKFRHRNRKGYRSQ | cell-penetrating peptide |
| 460 | KKQKFKHRNRKGYRS | cell-penetrating peptide |
| 461 | KKQKFRRRNRKGYRSH | cell-penetrating peptide |
| 462 | TAIPPCPHGWISLWK | cell-penetrating peptide |
| 463 | KKQKSRHRSRKRYRS | cell-penetrating peptide |
| 464 | KKQKSRRRSRKGYRS | cell-penetrating peptide |
| 465 | ISRCTVC | cell-penetrating peptide |
| 466 | ISRCQVCMKRRH | cell-penetrating peptide |
| 467 | VSRCTVC | cell-penetrating peptide |
| 468 | TDIPPCPQGWISLWK | cell-penetrating peptide |
| 469 | TVKAGELEKIISRCQVMKKRH | cell-penetrating peptide |
| 470 | TDIPSCPHGWISLWK | cell-penetrating peptide |
| 471 | TDIPPCPAGWISLWK | cell-penetrating peptide |
| 472 | TEIPPCPQGWISLWK | cell-penetrating peptide |
| 473 | TDVPPCPQGWISLWK | cell-penetrating peptide |
| 474 | RLVSYNGILFFLK | cell-penetrating peptide |
| 475 | RLVSYSGVIFFLK | cell-penetrating peptide |
| 476 | RLVSYNGILFFL | cell-penetrating peptide |
| 477 | RLVSYSGIIFFLK | cell-penetrating peptide |
| 478 | RFEQELRLVSYSGVLFFLKQ | cell-penetrating peptide |
| 479 | RLVSYNGIIFFLK | cell-penetrating peptide |
| 480 | DPAFKIEDPYSPRIQNLLKITNLRIKFVKL | cell-penetrating peptide |
| 481 | TKRFEQELRLVSYSGVLFFL | cell-penetrating peptide |
| 482 | GGRLKYSVAF | cell-penetrating peptide |
| 483 | GGFLRYTVSYDI | cell-penetrating peptide |
| 484 | GGFLKYTVSYDV | cell-penetrating peptide |
| 485 | LGNKLTAFGGFLKYTVSYDIPV | cell-penetrating peptide |
| 486 | GGYLKYTVSYDI | cell-penetrating peptide |
| 487 | GEIFFDMRLKGDK | cell-penetrating peptide |
| 488 | GEIYFDLRLKGDK | cell-penetrating peptide |
| 489 | GEIYLDMRLKGDK | cell-penetrating peptide |
| 490 | IGQPGAKGEPGEFYFDLRLKGDKGDPGFPG | cell-penetrating peptide |
| 491 | GEVFFDMRLKGDK | cell-penetrating peptide |
| 492 | LAGSCLPIFSTL | cell-penetrating peptide |
| 493 | AHNQDLGLAGSCLARFSTMPFLYCNPGDIC | cell-penetrating peptide |
| 494 | QEKAHNQDLGLAGSCLPVFSTLPFAYCNIH | cell-penetrating peptide |
| 495 | LAGSCLPVFSTM | cell-penetrating peptide |
| 496 | GNKRAHGQDLGTAGSCLRRFSTMPFMFCNI | cell-penetrating peptide |
| 497 | RAHGQDLGTAGSCLRRFSTMP | cell-penetrating peptide |
| 498 | RKRLQVQLNIRT | cell-penetrating peptide |
| 499 | HLVLPLQQSDVRKRLQVQLSIRTFASSGLI | cell-penetrating peptide |
| 500 | RKRLSVQLRIRT | cell-penetrating peptide |
| 501 | DLGTAGSCLRRFSTM | cell-penetrating peptide |
| 502 | RNIAEIIKDI | cell-penetrating peptide |
| 503 | TAGSCLRKFSTMRRRRRRRRRRRR | cell-penetrating peptide |
| 504 | FTLTGLLGTLVTMGLLT | cell-penetrating peptide |
| 505 | APYKAWK | cell-penetrating peptide |
| 506 | STSKTNRGDDSNWSKRVTNNKPS | cell-penetrating peptide |
| 507 | STSKRKRGDDSNWSKRVTKKKPS | cell-penetrating peptide |
| 508 | STSKRKRGDDSNWSKRVSKKKPS | cell-penetrating peptide |
| 509 | STSKRKRGDDANWSKRVTKKKPS | cell-penetrating peptide |
| 510 | PLAGSKRKRADEVAWSKRGTKKKPER | cell-penetrating peptide |
| 511 | PLAGSKRKRADEVAWSKRGTKKKPERTSAARAGPSR | cell-penetrating peptide |
| | RIR | |
| 512 | STSKRKRGDDANWSKRTTKKKPSS | cell-penetrating peptide |
| 513 | STSKRKRGDDANWSKRTTKKKPSSAGLKRAGSKADR | cell-penetrating peptide |
| | PSL | |
| 514 | PTTAGKRKRSDDAAWSKRARPKAGRT | cell-penetrating peptide |
| 515 | PTTAGKRKRSDDAAWSKRARPKAGRTSAARPGTSVR | cell-penetrating peptide |
| | RIR | |
| 516 | SSSLGKRKRSDEGAWSKGKSKKKAMR | cell-penetrating peptide |
| 517 | SSSLGKRKRSDEGAWSKGKSKKKAMRGSSSRRP | cell-penetrating peptide |
| | GPVRGP | |
| 518 | PTTAGKRKRTDDAAWSKRARPKAGR | cell-penetrating peptide |
| 519 | PTTAGKRKRTDDAAWSKRARPKAGRTSAARPGT | cell-penetrating peptide |
| | AVRRVR | |
| 520 | PATAGKRKRSDDAAWSKRARPKAGRTSAAR | cell-penetrating peptide |
| 521 | PATAGKRKRSDDAAWSKRARPKAGRTSAARPGT | cell-penetrating peptide |
| | SVRRIR | |
| 522 | SSSLGKRKRSNGGDWSKRSAKKKPA | cell-penetrating peptide |
| 523 | SSSLGKRKRSNGGDWSKRSAKKKPAGTPSRRAGP | cell-penetrating peptide |
| | GRGPR | |
| 524 | SSSLGKRKRSDEGAWSKGKSKKKAMR | cell-penetrating peptide |
| 525 | SSSLGKRKRSDEGAWSKGKSKKKAMRGSSSRRP | cell-penetrating peptide |
| | GPVRGP | |
| 526 | STSKRKRGDDANWNKRPTKKKPSS | cell-penetrating peptide |
| 527 | STSKRKRGDDANWNKRPTKKKPSSAGLKKAGSK | cell-penetrating peptide |
| | AERPSL | |
| 528 | SGALKRKRSDEVAWSRRRPVKKPV | cell-penetrating peptide |
| 529 | SGALKRKRSDEVAWSRRRPVKKPVRRAPPPRAGP | cell-penetrating peptide |
| | SVRRG | |
| 530 | SGALKRKRSDEVAWSRRKPAKKPAR | cell-penetrating peptide |
| 531 | SGALKRKRSDEVAWSRRKPAKKPARQPPPPRAGP | cell-penetrating peptide |
| | SVRRG | |
| 532 | AGALKRKRSDEVAWSRRKPAKKPAR | cell-penetrating peptide |
| 533 | AGALKRKRSDEVAWSRRKPAKKPARAPPPRAGP | cell-penetrating peptide |
| | SVRRGL | |
| 534 | STSKRKRGDDSNWSKRVTKKKPSSAGLKRAGSK | cell-penetrating peptide |
| | ADRPSLQIQTLQHAGTTMITVPSGGVCDLINTYAR | |
| | GSDEGNRHTSETLTYKIAIDYHFVADAAACRYSN | |
| | TGTGVMWLVYDTTPGGQAPTPQTIFSYPDTLKA | |
| | WPATWKVSRELCHRFVVKRRWLFNMETDGRIGS | |
| | DIPPSNASWKPCKRNIYFHKFTSGLGVRTQWKNV | |
| | TDGGVGAIQRGALYMVIAPGNGLTFTAHGQTRLY | |
| | FKSVGNQ | |
| 535 | DPQNALYYQPRVPTAAPTSGGVPWSRVGEVAILS | cell-penetrating peptide |
| | FVALICFYLLYLWVLRDLILVLKARQGRSTEELIF | |
| | GGQAVDRSNPIPNIPAPPSQGNPGPFVPGTG | |
| 536 | GSQLVPPPSAFNYIESQRDEFQLSHDLTEIVLQFPS | cell-penetrating peptide |
| | TASQITARLSRSCMKIDHCVIEYRQQVPINASGTVI | |
| | VEIHDKRMTDNESLQASWTFPIRCNIDLHYFSSSF | |
| | FSLKDPIPWKLYYRVSDSNVHQMTHFAKFKGKL | |
| | KLSSAKHSVDIPFRAPTVKILAKQFSEKDIDFWHV | |
| | GYGKWERRLVKSASSSRFGLRGPIEINPGESWAT | |
| | KSAIVTPNRNADLDIEEELLPYRELNRLGTNILDPG | |
| | ESASIVGIQRSQSNITMSMSQLNELVRSTVHECIKT | |
| | SCIPSTPKSLS | |
| 537 | RTGVKRSYGAARGDDRRRPNVV | cell-penetrating peptide |
| 538 | SYVKTVPNRTRTYIKLRVR | cell-penetrating peptide |
| 539 | MYSTSNRRGRSQTQRGSHVRRTGVKRSYGAARG | cell-penetrating peptide |
| | DDRRRPNVVSKTQVEPRMTIQRVQENQFGPEFVL | |
| | SQNSALSTFVTYPSYVKTVPNRTRTYIKLKRVRFK | |
| | GTLKIERGQGDTIMDGPSSNIEGVFSMVIVVDRKP | |
| | HVSQSGRLHTFDELFGARIHCHGNLSVVPALKDR | |
| | YYIRHVTKRVVSLEKDTLLIDLHGTTQLSNKRYN | |
| | CWASFSDLERDCNGVYGNITKNALLVYYCWLSD | |
| | AQSKASTYVSFELDYLG | |
| 540 | RRRRRRRRRRRRVDYGKWERKPIRCASMSR | cell-penetrating peptide |
| 541 | RRRRRRRRRRRRGKWERKPIRCAS | cell-penetrating peptide |
| 542 | KKKKKKKKKKKKKKKKGKWERKPIRCAS | cell-penetrating peptide |
| 543 | RRRRRRRRRRRRVDFSHVDYGKWERKPIRCASM | cell-penetrating peptide |
| | SRLGLRG | |
| 544 | GVKRSYGAARGDDRRRPNVVAPYKAWRRRRRR | cell-penetrating peptide |
| | RRRRRR | |
| 545 | KSVPNRTRTYIKLKRLRFKGAPYKAWRRRRRRRR | cell-penetrating peptide |
| | RRRR | |
| 546 | RTGVKRSYGAARGDDRRRPNVVRRRRRRRRRRR | cell-penetrating peptide |
| | R | |
| 547 | SYVKTVPNRTRTYIKGGGGGRRRRRRRRRRRR | cell-penetrating peptide |
| 548 | VDIPFRAPTIKILSKQFTEDDIDFWHVGYGKWERK | cell-penetrating peptide |
| | LVRPASLSGRRGLRR | |
| 549 | IDFWHVGYGKWERKLVRPASLSGRRGLRR | cell-penetrating peptide |
| 550 | IDFWSVEKGETRRRLLNPTPHAHSPRPIAHR | cell-penetrating peptide |
| 551 | IDFSHVGYGKWERKMIRSASISRLGLHN | cell-penetrating peptide |
| 552 | VDFSHVGYGKWERKLIRSASTVKYGLPS | cell-penetrating peptide |
| 553 | IDFSHVDYGKVERKLVKCESSSRLGLHS | cell-penetrating peptide |
| 554 | IDFWSVGRKAQQRKLVQGPSLIGSRSMRY | cell-penetrating peptide |
| 555 | IDFWSVGSKPQTRRLVDGSRLIGHSSRSLRV | cell-penetrating peptide |
| 556 | IDFWSVERGETRRRLLNPTPSAGSNRALSKR | cell-penetrating peptide |
| 557 | VDFWSVGKPKPIRRLIQNDPGTDYDTGPKYR | cell-penetrating peptide |
| 558 | VDFWSVEKPKPIRRLLNPGPNQGPYPNTGHR | cell-penetrating peptide |
| 559 | VDFSHVDYGKWERKLIRSASTSRYGLRS | cell-penetrating peptide |
| 560 | VDFSHVDYGKWERKTLRSRSLSRIGLTG | cell-penetrating peptide |
| 561 | IDFWHVGYGKWERRLVKSASSSRFGIRG | cell-penetrating peptide |
| 562 | VDFFHVDYGRWERKHIRCASMSRVGLRG | cell-penetrating peptide |
| 563 | GTFQHVDYGKWERKPIRCQSMSRVGYRR | cell-penetrating peptide |
| 564 | VGYGKWERKLVRPASLS | cell-penetrating peptide |
| 565 | VEKGETRRRLLNPTPHA | cell-penetrating peptide |
| 566 | VGYGKWERKLIRSASTV | cell-penetrating peptide |
| 567 | VEKPKPIRRLLNPGPNQ | cell-penetrating peptide |
| 568 | VDYGKWERKLIRSASTS | cell-penetrating peptide |
| 569 | VDYGKWERKTLRSRSLS | cell-penetrating peptide |
| 570 | VGYGKWERRLVKSASSS | cell-penetrating peptide |
| 571 | VDYGRWERKHIRCASMS | cell-penetrating peptide |
| 572 | VERPKPIRRLLTPTPGC | cell-penetrating peptide |
| 573 | PFRAPTIKILSKQFTEDDIDFWHVGYGKWERKLVRPAS | cell-penetrating peptide |
| | LSGRRGLRR | |
| 574 | PFRAPTVKILSKQFTDKDIDFSHVGYGKWERKMIRSAS | cell-penetrating peptide |
| | ISRLGL | |
| 575 | DIAFRAPTVKILSKQFTDRDVDFSHVGYGKWERKLIRS | cell-penetrating peptide |
| | ASTVKYGL | |
| 576 | DIRFKPPTINILSKDYTADCVDFWSVEKPKPIRRLLNPG | cell-penetrating peptide |
| | PNQGPYPNTG | |
| 577 | DIPFRAPTVKIHSKQFSHRDVDFSHVDYGKWERKTLRS | cell-penetrating peptide |
| | RSLSRIGL | |
| 578 | DIPFRAPTVKILAKQFSEKDIDFWHVGYGKWERRLVK | cell-penetrating peptide |
| | SASSSRFGI | |
| 579 | DIPFRAPTVKILSKQFTDKDVDFFHVDYGRWERKHIRC | cell-penetrating peptide |
| | ASMSRVGL | |
| 580 | DIKYKPPTIKILSKDYTADCVDFWSVERPKPIRRLLTPT | cell-penetrating peptide |
| | PGCG | |
| 581 | ARTKQTARKSTGGKAPRKQLATKAARKSAPATGGVK | cell-penetrating peptide |
| | KPHRYRPGTVA | |
| 582 | SGRGKGGKGLGKGGAKRHRKVLRDNIQGITKPAI | cell-penetrating peptide |
| 583 | GRKKRRQRRR | cell-penetrating peptide |

### DETAILED DESCRIPTION

The present disclosure relates, in part, to trinucleotide cap analogs, compositions comprising trinucleotide cap analogs, and methods of use thereof, for example, for use in transcription, for use in intracellular stability, for use in detection, and isolation of capped RNA, and for use of the resultant isolated RNA in translation both in vitro and in vivo. Trinucleotide cap analogs as disclosed herein can have the advantage of being improved substrates for T7-RNA or other RNA polymerases, and can lead to a better in vitro transcription yield, improved intracellular molecular stability of 5′ capped mRNAs, improved translational efficiency as compared to other anti-reverse cap analog (ARCA) substrates, and improved transfection into specific cell lines.

In addition to the caps themselves, the present disclosure relates to compositions and methods for producing capped mRNA molecules. Such compositions and methods include those where caps are designed to match initiation site nucleotide sequences and formulations (e.g., in vitro transcription formulations) are designed to facilitate efficient mRNA (e.g., capped mRNA) production. Such efficient mRNA production includes compositions and methods for the production of mRNA molecules where a high percentage of the mRNA molecules are capped mRNA (e.g., from about 75% to about 99%, from about 80% to about 99%, from about 85% to about 99%, from about 90% to about 99%, from about 95% to about 99%, from about 80% to about 96%, from about 85% to about 96%, from about 90% to about 96%, etc. of the total number of mRNA molecules produced) and where mRNA is produced in high yield (i.e., 3 milligrams of RNA per 1 milliliter of reaction mixture).

RNA yield (e.g., mRNA yield) may be determined by comparison of the amount of RNA produced to the amount of one or more components of the reaction mixture used to produce the RNA. One formula that may be used is the amount of RNA produced for a fixed amount of a single reaction mixture component for a specific volume of reaction mixture. By way of example, a 20 μl reaction mixture with a CTP concentration of 7.5 mM is used for in vitro transcription, then RNA yields of over 80 μg. A second example is where a 20 μl reaction mixture with a cap concentration of 10 mM is used, with an RNA yield of 120 μg.

Composition and methods set out herein allow for the production of RNA in amount greater than 40 μg/20 μl (e.g., from about 40 μg to about 200 μg, from about 40 μg to about 160 μg, from about 40 μg to about 120 μg, from about 80 μg to about 200 μg, from about 80 μg to about 200 μg, from about 80 μg to about 180 μg, from about 80 μg to about 160 μg, from about 80 μg to about 120 μg, from about 100 μg to about 150 μg, etc.).

RNA yield may also be expressed as the amount of RNA produced as a function of reaction mixture volume. For example, 100 μg of RNA produced in 20 μl is 5 mg of RNA produced in 1 milliliter of reaction mixture. When corrected for volume, composition and methods set out herein allow for the production of RNA in amount greater than or equal to 2 mg/ml (e.g., from about 2 mg/ml to about 20 mg/ml, from about 2 mg/ml to about 16 mg/ml, from about 2 mg/ml to about 10 mg/ml, from about 2 mg/ml to about 7, mg/ml, from about 4 mg/ml to about 20 mg/ml, from about 4 mg/ml to about 20 mg/ml, from about 5 mg/ml to about 20 mg/ml, from about 6 mg/ml to about 20 mg/ml, from about 4 mg/ml to about 20 mg/ml, from about 7 mg/ml to about 20 mg/ml, from about 4 mg/ml to about 16 mg/ml, from about 4 mg/ml to about 18 mg/ml, from about 4 mg/ml to about 14 mg/ml, from about 6 mg/ml to about 16 mg/ml, from about 7 mg/ml to about 19 mg/ml, etc.).

In one aspect is a trinucleotide cap analog of Formula (I):

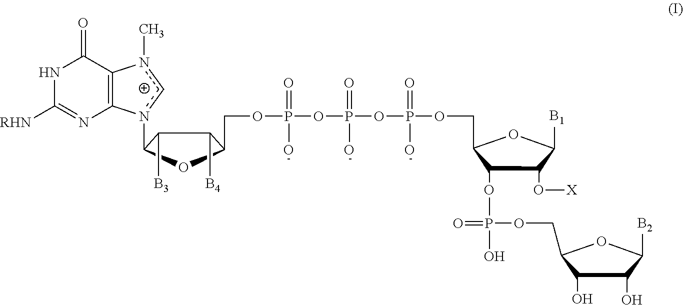

wherein
B₃ is chosen from —OH, halogen, dyes, —OR¹,
   wherein R¹ is chosen from propargyl, tert-butyldimethylsilyl, and a methylene bridge with the 4′C;
B₄ is chosen from —OH, dyes, and —OR², wherein R² is chosen from propargyl and tert-butyldimethylsilyl;
   or R¹ joins with R² such that B₃ and B₄ form -2′,3′-O-isopropylidine; on the condition that B₃ and B₄ cannot both be —OH
X is chosen from —H and —CH₃;
B₁ and B₂ are each independently chosen from adenine, guanine, cytosine, and uracil;
R is chosen from H, a linker-bound cell-penetrating peptide, a linker-bound cell-penetrating peptide covalently linked to a dye, and a linker-bound dye.

In some embodiments, R is chosen from a linker-bound cell-penetrating peptide chosen from any of SEQ ID NO:1-583, wherein the linker bound to the cell penetrating peptides can be chosen from those commercially available, such as biotin, 3′ maleimidobenzoic acid N-hydroxysuccinimide ester, or

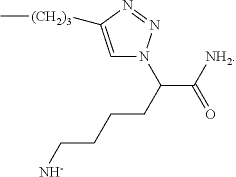

In some embodiments, R is

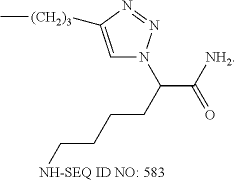

In some embodiments, R is chosen from a linker-bound cell-penetrating peptide covalently linked to a dye, wherein the cell penetrating peptide is chosen from any of SEQ ID NO:1-583. In some embodiments, R is a linker-bound dye.

In some embodiments, each dye is independently chosen from azobenzene dyes, naphthalene containing dyes, cyanine dyes, rhodamine dyes, coumarin, and pyrene dyes. In some embodiments, each dye is independently chosen from:

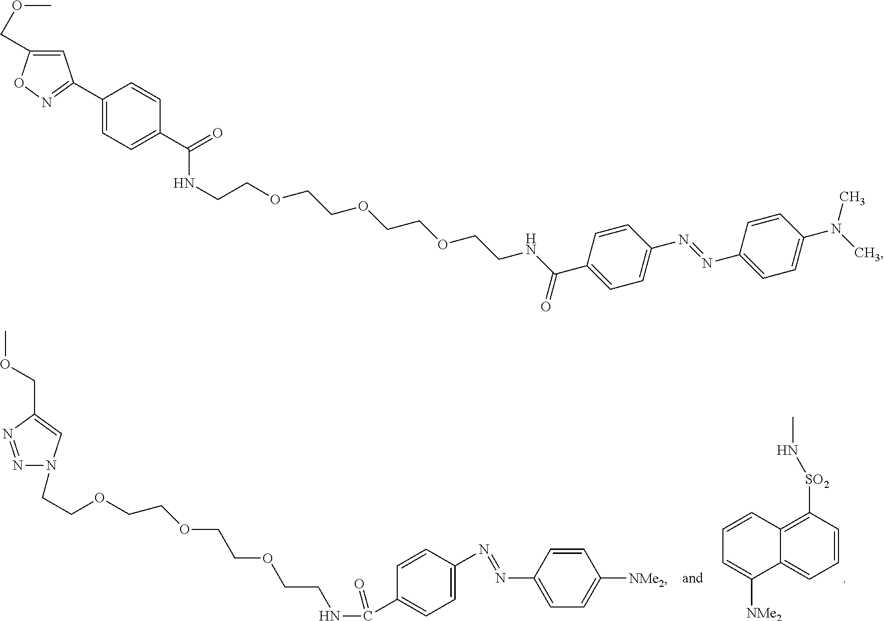 [SMILES: COCc1cc(-c2ccc(C(=O)NCCOCCOCCOCCNC(=O)c3ccc(/N=N/c4ccc(N(C)C)cc4)cc3)cc2)no1 ; COCc1cn(CCOCCOCCOCCNC(=O)c2ccc(/N=N/c3ccc(N(C)C)cc3)cc2)nn1 ; CNS(=O)(=O)c1cccc2c(N(C)C)cccc12]

In some embodiments, B₃ is —OR¹ and B₄ is —OR² wherein R¹ joins with R² such that B₃ and B₄ form -2′,3′-O-isopropylidene; X is —CH₃; and R is H. In some embodiments, B₃ is —OR¹ and B₄ is —OR² wherein R¹ joins with R² such that B₃ and B₄ form -2′,3′-O-isopropylidene; and R is chosen

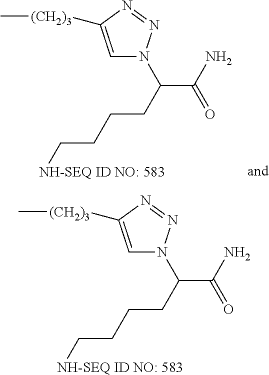

covalently linked to a dye. In some embodiments, B₃ is chosen from —OR¹ wherein R¹ is chosen from propargyl and tert-butyldimethylsilyl; B₄ is —OH; and R is H. In some embodiments, B₃ is chosen from —OR¹ wherein R¹ is chosen from propargyl and tert-butyldimethylsilyl; B₄ is —OH; and R is chosen from

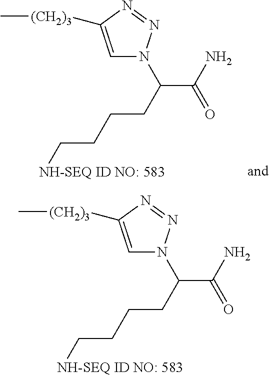

covalently linked to a dye. In some embodiments, each dye is independently chosen from azobenzene dyes, naphthalene containing dyes, cyanine dyes, rhodamine dyes, coumarin, and pyrene dyes. In some embodiments, each dye is independently chosen from

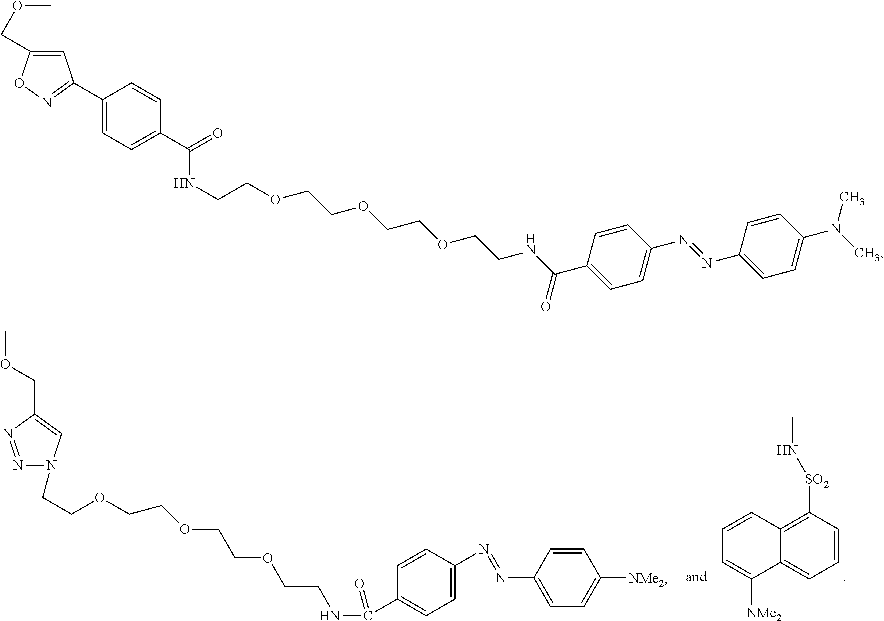 [SMILES: COCc1cc(-c2ccc(C(=O)NCCOCCOCCOCCNC(=O)c3ccc(/N=N/c4ccc(N(C)C)cc4)cc3)cc2)no1 ; COCc1cn(CCOCCOCCOCCNC(=O)c2ccc(/N=N/c3ccc(N(C)C)cc3)cc2)nn1 ; CNS(=O)(=O)c1cccc2c(N(C)C)cccc12]

In some embodiments, B₃ is a dye; B₄ is —OH; and R is H. In some embodiments, B₃ is a dye; B₄ is —OH; and R is chosen from

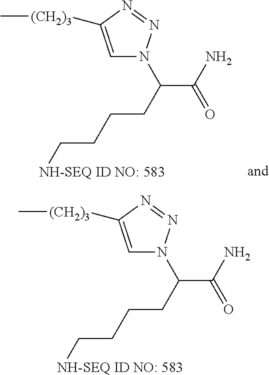

covalently linked to a dye. In some embodiments, each dye is independently chosen from azobenzene dyes, naphthalene containing dyes, cyanine dyes, rhodamine dyes, coumarin, and pyrene dyes. In some embodiments, each dye is independently chosen from

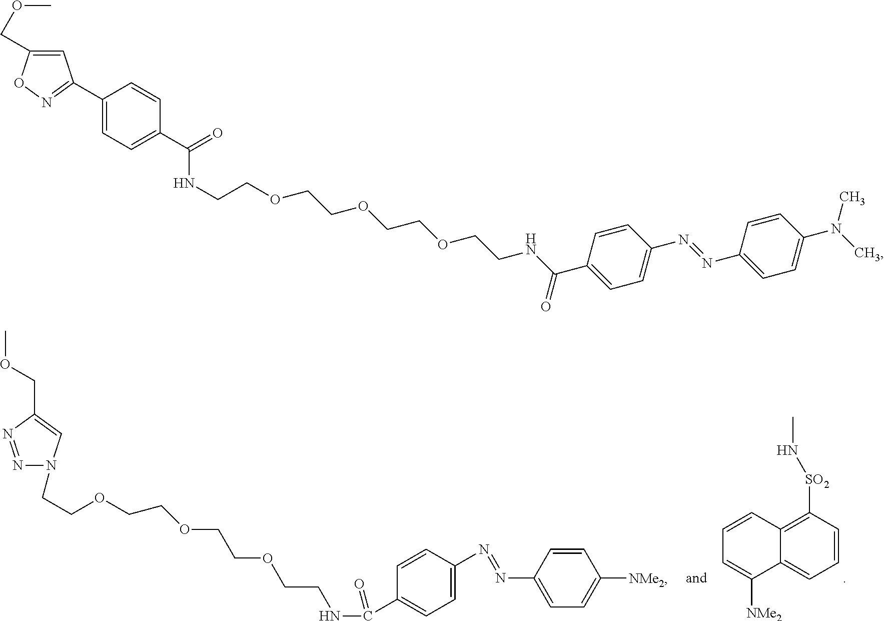 [SMILES: COCc1cc(-c2ccc(C(=O)NCCOCCOCCOCCNC(=O)c3ccc(/N=N/c4ccc(N(C)C)cc4)cc3)cc2)no1 ; COCc1cn(CCOCCOCCOCCNC(=O)c2ccc(/N=N/c3ccc(N(C)C)cc3)cc2)nn1 ; CNS(=O)(=O)c1cccc2c(N(C)C)cccc12]

In some embodiments, B₃ is —OH; B₄ is chosen from —OR² wherein R² is chosen from propargyl and tert-butyldimethylsilyl; and R is H. In some embodiments, B₃ is —OH; B₄ is chosen from —OR² wherein R² is chosen from propargyl and tert-butyldimethylsilyl; and R is chosen from

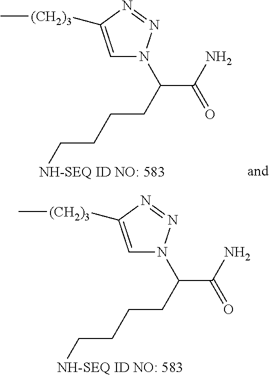

covalently linked to a dye. In some embodiments, B₃ is —OH; B₄ is a dye; R is H; and X=—CH₃. In some embodiments, B₃ is —OH; B₄ is a dye; and R is chosen from

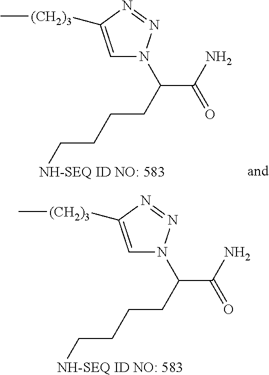

covalently linked to a dye. In some embodiments, each dye is independently chosen from azobenzene dyes, naphthalene containing dyes, cyanine dyes, rhodamine dyes, coumarin, and pyrene dyes. In some embodiments, each dye is independently chosen from

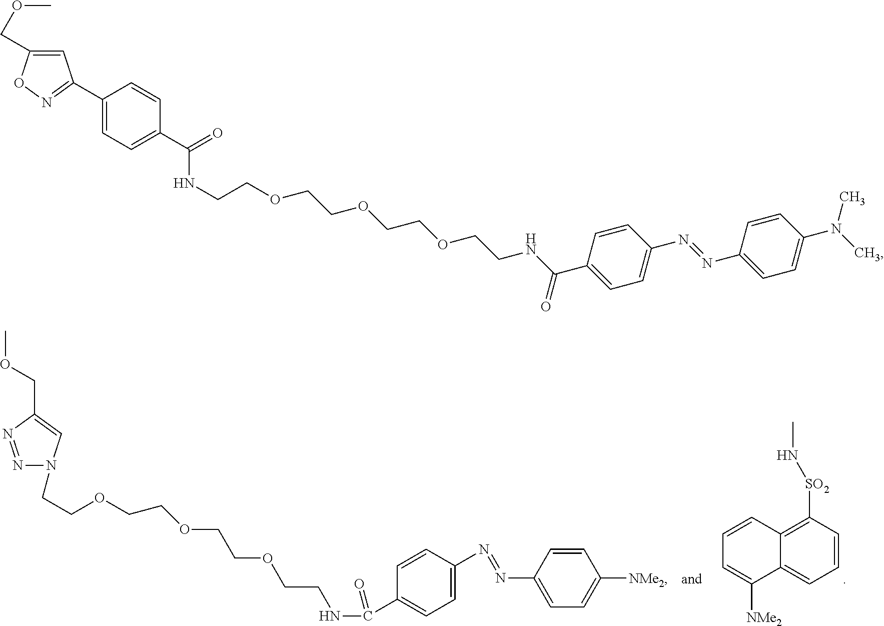 [SMILES: COCc1cc(-c2ccc(C(=O)NCCOCCOCCOCCNC(=O)c3ccc(/N=N/c4ccc(N(C)C)cc4)cc3)cc2)no1 ; COCc1cn(CCOCCOCCOCCNC(=O)c2ccc(/N=N/c3ccc(N(C)C)cc3)cc2)nn1 ; CNS(=O)(=O)c1cccc2c(N(C)C)cccc12]

In some embodiments of the trinucleotide cap analog of Formula (I), B₃ is —OR¹, and R¹ forms a methylene bridge with the 4′C such that the trinucleotide cap analog is Formula (I) is the locked trinucleotide cap analog of Formula (II):

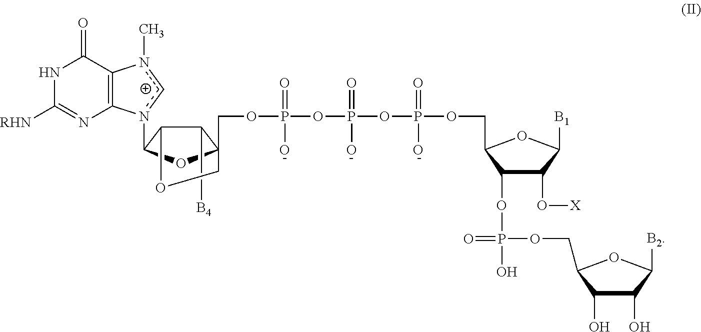

In some embodiments, R is chosen from a linker-bound cell-penetrating peptide, wherein the cell-penetrating peptide is chosen from any of SEQ ID NO:1-583. In some embodiments, R is chosen from a linker-bound cell-penetrating peptide covalently linked to a dye, wherein the cell penetrating peptide is chosen from any of SEQ ID NO:1-583. In some embodiments, B₄ is —OH and R is H. In some embodiments, B₄ is —OH; and R is chosen from

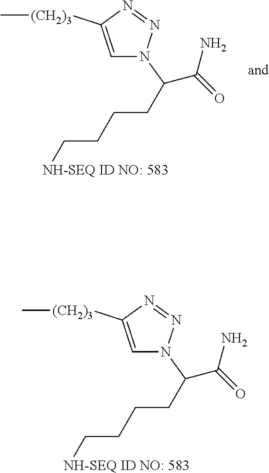

covalently linked to a dye. In some embodiments, B₄ is a dye; and R is H. In some embodiments, each dye is independently chosen from azobenzene dyes, naphthalene containing dyes, cyanine dyes, rhodamine dyes, coumarin, and pyrene dyes. In some embodiments, each dye is independently chosen from

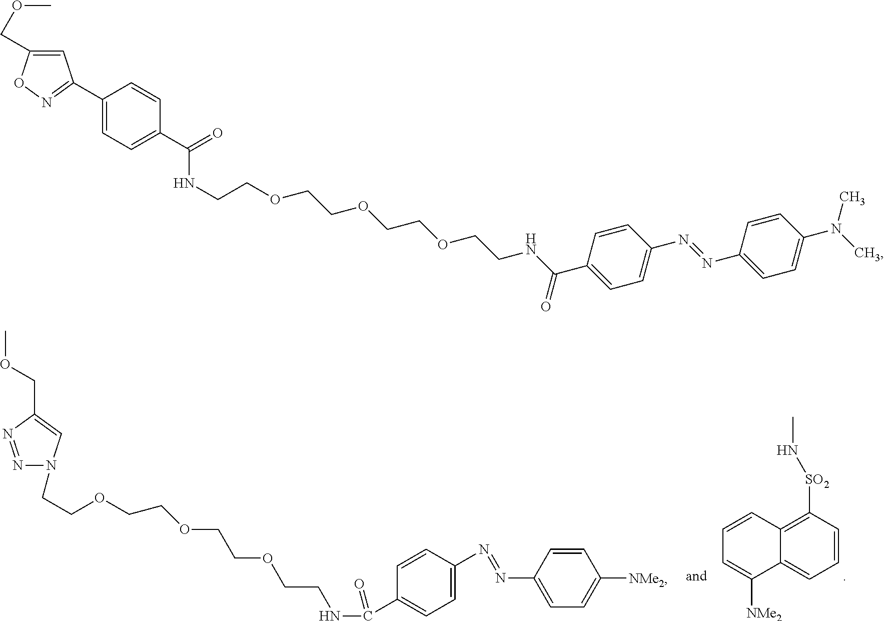 [SMILES: COCc1cc(-c2ccc(C(=O)NCCOCCOCCOCCNC(=O)c3ccc(/N=N/c4ccc(N(C)C)cc4)cc3)cc2)no1 ; COCc1cn(CCOCCOCCOCCNC(=O)c2ccc(/N=N/c3ccc(N(C)C)cc3)cc2)nn1 ; CNS(=O)(=O)c1cccc2c(N(C)C)cccc12]

Another aspect of the present disclosure is a composition comprising a trinucleotide cap analog of Formula (I) or any of the embodiments thereof disclosed herein.

Another aspect of the present disclosure is a method of making a lipid nanoparticle, comprising combining a composition comprising a trinucleotide cap analog of Formula (I) or any of the embodiments thereof disclosed herein with at least one cationic lipid, optionally one or more neutral lipids, and optionally one or more conjugated lipid that prevents aggregation (e.g., PEG lipids, and/or polyglycol lipids) and optionally one or more cell penetrating peptides, or any combination thereof.

Another aspect of the present disclosure is a composition comprising RNA having a trinucleotide cap analog of Formula (I), or any of the embodiments thereof disclosed herein, covalently bonded thereto. In some embodiments, the composition further comprises at least one RNA delivery agent. In some embodiments, the at least one RNA delivery agent comprises at least one cationic lipid. In some embodiments, the at least one RNA delivery agent further comprises at least one neutral lipid. In some embodiments, the at least one RNA delivery agent is chosen from cell penetrating peptides.

In some embodiments, the at least one cationic lipid is chosen from:

2,3-dioleyloxy-N-[2(sperminecarboxamido) ethyl]-N,N-dimethyl-1-propanaminium trifluoroacetate (DOSPA), 1,3-dioleoyloxy-2-(6-carboxy-spermyl) propylamide (DOSPER), dioctadecylamido-glycylspermine (DOGS), tetramethyltetrapalmitylspermine (TMTPS), tetramethyltetrapalmitoylspermin (TMTOS), tetramethlytetralauryl spermine (TMTLS), tetramethyltetramyristyl spermine (TMTMS), tetramethyldioleylspermine TMDOS), N-1-dimethyl-N-1-(2,3-diaoleoyloxypropyl)-2-hydroxypropane-1,3-diamine, N-1-dimethyl-N-1-(2,3-diamyristyloxypropyl)-2-hydroxypropane-1,3-diamine, N-1-dimethyl-N-1-(2,3-diapalmityloxypropyl)-2-hydroxypropane-1,3-diamine, N-1-dimethyl-N-1-(2,3-diaoleoyloxypropyl)-2-(3-amino-2-hydroxypropyloxy)propane-1,3-diamine, N-1-dimethyl-N-1-(2,3-diamyristyloxypropyl)-2-(3-amino-2-hydroxypropyloxy)propane-1,3-diamine, N-1-dimethyl-N-1-(2,3-diapalmityloxypropyl)-2-(3-amino-2-hydroxypropyloxy)propane-1,3-diamine, L-spermine-5-carboxyl-3-(DL-1,2-dipalmitoyl-dimethylaminopropyl-p-hydroxyethylamine, 3,5-(N,N-di-lysyl)-diaminobenzoyl-glycyl-3-(DL-1,2-dipalmitoyl-dimethylaminopropyl-p-hydroxyethylamine), L-Lysine-bis(O,O′-oleoyl-p-hydroxyethyl)amide dihydrochloride, L-Lysine-bis-(O,O′-palmitoyl-p-hydroxyethyl)amide dihydrochloride, 1,4-bis[(3-(3-aminopropyl)-alkylamino)-2-hydroxypropyl)piperazine, L-Lysine-bis-(O,O′-myristoyl-β-hydroxyethyl)amide dihydrochloride, L-Ornithine-bis-(O,O′-myristoyl-β-hydroxyethyl)amide dihydrochloride, L-Ornithine-bis-(O,O′-oleoyl-β-hydroxyethyl)amide dihydrochloride, 1,4-bis[(3-(3-aminopropyl)-oleylamino)-2-hydroxypropyl]piperazine, L-Ornithine-bis-(O,O′-palmitoyl-β-hydroxyethyl)amide dihydrochloride, 1,4,-bis[(3-amino-2-hydroxypropyl)-oleylamino]-butane-2,3-diol, 1,4,-bis[(3-amino-2-hydroxypropyl)-palmitylamino]-butane-2,3-diol, 1,4,-bis[(3-amino-2-hydroxypropyl)-myristylamino]-butane-2,3-diol, 1,4-bis[(3-oleylamino)propyl]piperazine, L-Arginine-bis-(O,O′-oleoyl-β-hydroxyethyl)amide dihydrochloride, bis[(3-(3-aminopropyl)-myristylamino)2-hydroxypropyl]piperazine, L-Arginine-bis-(O,O′-palmitoyl-β-hydroxyethyl)amide dihydrochloride, L-Serine-bis-(O,O′-oleoyl-O-hydroxyethyl)amide dihydrochloride, 1,4-bis[(3-(3-aminopropyl)-palmitylamino)-2-hydroxypropyl]piperazine, Glycine-bis-(O,O′-palmitoyl-β-hydroxyethyl)amide dihydrochloride, Sarcosine-bis-(O,O′-palmitoyl-β-hydroxyethyl)amide dihydrochloride, L-Histidine-bis-(O,O′-palmitoyl-β-hydroxyethyl)amide dihydrochloride, cholesteryl-3β-carboxyl-amidoethylenetrimethylammonium iodide, 1,4-bis[(3-myristylamino)propyl]piperazine, 1-dimethylamino-3-trimethylammonio-DL-2-propyl-cholesteryl carboxylate iodide, cholesteryl-3β-carboxyamidoethyleneamine, cholesteryl-3β-oxysuccinamidoethylenetrimethylammonium iodide, 1-dimethylamino-3-trimethylammonio-DL-2-propyl-cholesteryl-3β-oxysuccinate iodide, 2-[(2-trimethylammonio)-ethylmethylamino]ethyl-cholesteryl-3β-oxysuccinate iodide, 3β[N—(N′,N′-dimethylaminoethane)carbamoyl]cholesterol, and 3β-[N-(polyethyleneimine)-carbamoyl]cholesterol, 1,4-bis[(3-palmitylamino)propyl]piperazine, L-Ornithylglycyl-N-(1-heptadecyloctadecyl)glycinamide, N²,N⁵-Bis(3-aminopropyl)-L-ornithylglycyl-N-(1-heptadecyloctadecyl)glycinamide, 1,4-bis[(3-(3-amino-2-hydroxypropyl)-alkylamino)-2-hydroxypropyl]piperazine N²—[N²,N⁵-Bis(3-aminopropyl)-L-ornithyl]-N,N-dioctadecyl-L-glutamine, N²—[N²,N⁵-Bis(aminopropyl)-L-ornithyl]-N—N-dioctadecyl-L-α-glutamine, 1,4-bis[(3-(3-amino-2-hydroxypropyl)-oleylamino)2-hydroxypropyl]piperazine, N²—[N²,N⁵-Bis(aminopropyl)-L-ornithyl]-N—N-dioctadecyl-L-α-asparagine, N—[N²—[N²,N⁵-Bis[(1,1-dimethylethoxy)carbonyl]-N²,N⁵-bis[3-[(1,1-dimethylethoxy)carbonyl]aminopropyl]-L-ornithyl-N—N-dioctadecyl-L-glutaminyl]-L-glutamic acid, N²—[N²,N⁵-Bis(3-aminopropyl)-L-ornithyl]-N,N-diolyl-L-glutamine, N²—[N²,N⁵-Bis(aminopropyl)-L-ornithyl]-N—N-dioleyl-L-α-glutamine, 4-bis[(3-(3-amino-2-hydroxypropyl)-myristylamino)-2-hydroxypropyl]piperazine, N²—[N²,N⁵-Bis(aminopropyl)-L-ornithyl]-N—N-dioleyl-L-α-asparagine, N—[N²-N⁵-Bis[(1,1-dimethylethoxy)carbonyl]-N²,N⁵-bis[3-[(1,1-dimethylethoxy)carbonyl]aminopropyl]-L-ornithyl-N—N-dioleyl-L-glutaminyl]-L-glutamic acid, 1,4-bis[(3-(3-aminopropyl)-oleylamino)propyl]piperazine, N²—[N²,N⁵-Bis(3-aminopropyl)-L-ornithyl]-N,N-dipalmityl-L-glutamine, N²—[N²,N⁵-Bis(aminopropyl)-L-ornithyl]-N—N-dipalmityl-L-α-glutamine, N²—[N²,N⁵-Bis(aminopropyl)-L-ornithyl]-N—N-dipalmityl-L-α-asparagine, N—[N²—[N²,N⁵-Bis[(1,1-dimethylethoxy)carbonyl]-N²,N⁵-bis[3-[(1,1-dimethylethoxy)carbonyl]aminopropyl]-L-ornithyl-N—N-dipalmityl-L-glutaminyl]-L-glutamic acid, N²—[N²,N⁵-Bis(3-aminopropyl)-L-ornithyl]-N,N-dimyristyl-L-glutamine, N²—[N²,N⁵-Bis(aminopropyl)-L-ornithyl]-N—N-dimyristyl-L-α-glutamine, N²—[N²,N⁵-Bis(aminopropyl)-L-ornithyl]-N—N-dimyristyl-L-α-asparagine, 1,4-bis[(3-(3-amino-2-hydroxypropyl)-palmitylamino)-2-hydroxypropyl]piperazine, N—[N²—[N²,N⁵-Bis[(1,1-dimethylethoxy)carbonyl]-N²,N⁵-bis[3-[(1,1-dimethylethoxy)carbonyl]aminopropyl]-L-ornithyl-N—N-dimyristyl-L-glutaminyl]-L-glutamic acid, 1,4-bis[(3-(3-aminopropyl)-myristylamino)propyl]piperazine, N²—[N²,N⁵-Bis(3-aminopropyl)-L-ornithyl]-N,N-dilaureyl-L-glutamine, N²-8 N²,N⁵-Bis(aminopropyl)-L-ornithyl]-N—N-dilaureyl-L-α-glutamine, N²—[N²,N⁵-Bis(aminopropyl)-L-ornithyl]-N—N-dilaureyl-L-α-asparagine, N—[N²—[N²,N⁵-Bis[(1,1-dimethylethoxy)carbonyl]-N²,N⁵-bis[3-[(1,1-dimethylethoxy)carbonyl]aminopropyl]-L-ornithyl-N—N-dilaureyl-L-glutaminyl]-L-glutamic acid, 3-[N′,N″-bis(2-tertbutyloxycarbonylaminoethyl)guanidino]-N,N-dioctadec-9-enylpropionamide, 3-[N′,N″-bis(2-tertbutyloxycarbonylaminoethyl)guanidino]-N,N-dipalmitylpropionamide, 3-[N′,N″-bis(2-tertbutyloxycarbonylaminoethyl)guanidino]-N,N-dimyristylpropionamide, 1,4-bis[(3-(3-aminopropyl)-palmitylamino)propyl]piperazine, 1,4-bis[(3-(3-amino-2-hydroxypropyl)-oleylamino)propyl]piperazine, N,N-(2-hydroxy-3-aminopropyl)-N-2-hydroxypropyl-3-N,N-diolylaminopropane, N,N-(2-hydroxy-3-aminopropyl)-N-2-hydroxypropyl-3-N,N-dipalmitylaminopropane, N,N-(2-hydroxy-3-aminopropyl)-N-2-hydroxypropyl-3-N,N-dimyristylaminopropane, 1,4-bis[(3-(3-amino-2-hydroxypropyl)-myristylamino)propyl]piperazine, [(3-aminopropyl)-bis-(2-tetradecyloxyethyl)]methyl ammonium bromide, [(3-aminopropyl)-bis-(2-oleyloxyethyl)]methyl ammonium bromide, [(3-aminopropyl)-bis-(2-palmityloxyethyl)]methyl ammonium bromide, Oleoyl-2-hydroxy-3-N,N-dimethyamino propane, 2-didecanoyl-1-N,N-dimethylaminopropane, palmitoyl-2-hydroxy-3-N,N-dimethyamino propane, 1,2-dipalmitoyl-1-N,N-dimethylaminopropane, myristoyl-2-hydroxy-3-N,N-dimethyamino propane, 1,2-dimyristoyl-1-N,N-dimethylaminopropane, (3-Amino-propyl)->4-(3-amino-propylamino)-4-tetradecylcarbamoyl-butylcarbamic acid cholestryl ester, (3-Amino-propyl)->4-(3-amino-propylamino-4-carbamoylbutylcarbamic acid cholestryl ester, (3-Amino-propyl)->4-(3-amino-propylamino)-4-(2-dimethylamino-ethylcarbamoyl)-butylcarbamic acid cholestryl ester, Spermine-5-carboxyglycine (N′-stearyl-N′-oleyl) amide tetratrifluoroacetic acid salt, Spermine-5-carboxyglycine (N′-stearyl-N′-elaidyl) amide tetratrifluoroacetic acid salt, Agmatinyl carboxycholesterol acetic acid salt, Spermine-5-carboxy-β-alanine cholesteryl ester tetratrifluoroacetic acid salt, 2,6-Diaminohexanoeyl β-alanine cholesteryl ester bistrifluoroacetic acid salt, 2,4-Diaminobutyroyl β-alanine cholesteryl ester bistrifluoroacetic acid salt, N,N-Bis(3-aminopropyl)-3-aminopropionyl β-alanine cholesteryl ester tristrifluoroacetic acid salt, [N,N-Bis(2-hydroxyethyl)-2-aminoethyl]aminocarboxy cholesteryl ester, Stearyl carnitine ester, Palmityl carnitine ester, Myristyl carnitine ester, Stearyl stearoyl carnitine ester chloride salt, L-Stearyl Stearoyl Carnitine Ester, Stearyl oleoyl carnitine ester chloride, Palmityl palmitoyl carnitine ester chloride, Myristyl myristoyl carnitine ester chloride, L-Myristyl myristoyl carnitine ester chloride, 1,4-bis[(3-(3-amino-2-hydroxypropyl)-palmitylamino)propyl]piperazine, N-(3-aminopropyl)-N,N′-bis-(dodecyloxyethyl)-piperazinium bromide, N-(3-aminopropyl)-N,N′-bis-(oleyloxyethyl)-piperazinium bromide, N-(3-aminopropyl)-N,N′-bis-(palmityloxyethyl)-piperazinium bromide, N-(3-aminopropyl)-N,N′-bis-(myristyloxyethyl)-piperazinium bromide, N-(3-aminopropyl)-N′-methyl-N,N′-(bis-2-dodecyloxyethyl)-piperazinium bromide, N-(3-aminopropyl)-N′-methyl-N,N′-(bis-2-oleyloxyethyl)-piperazinium bromide, N-(3-aminopropyl)-N′-methyl-N,N′-(bis-2-palmityloxyethyl)-piperazinium bromide, N-(3-aminopropyl)-N′-methyl-N,N′-(bis-2-myristyloxyethyl)-piperazinium bromide, Phospholipids useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE),

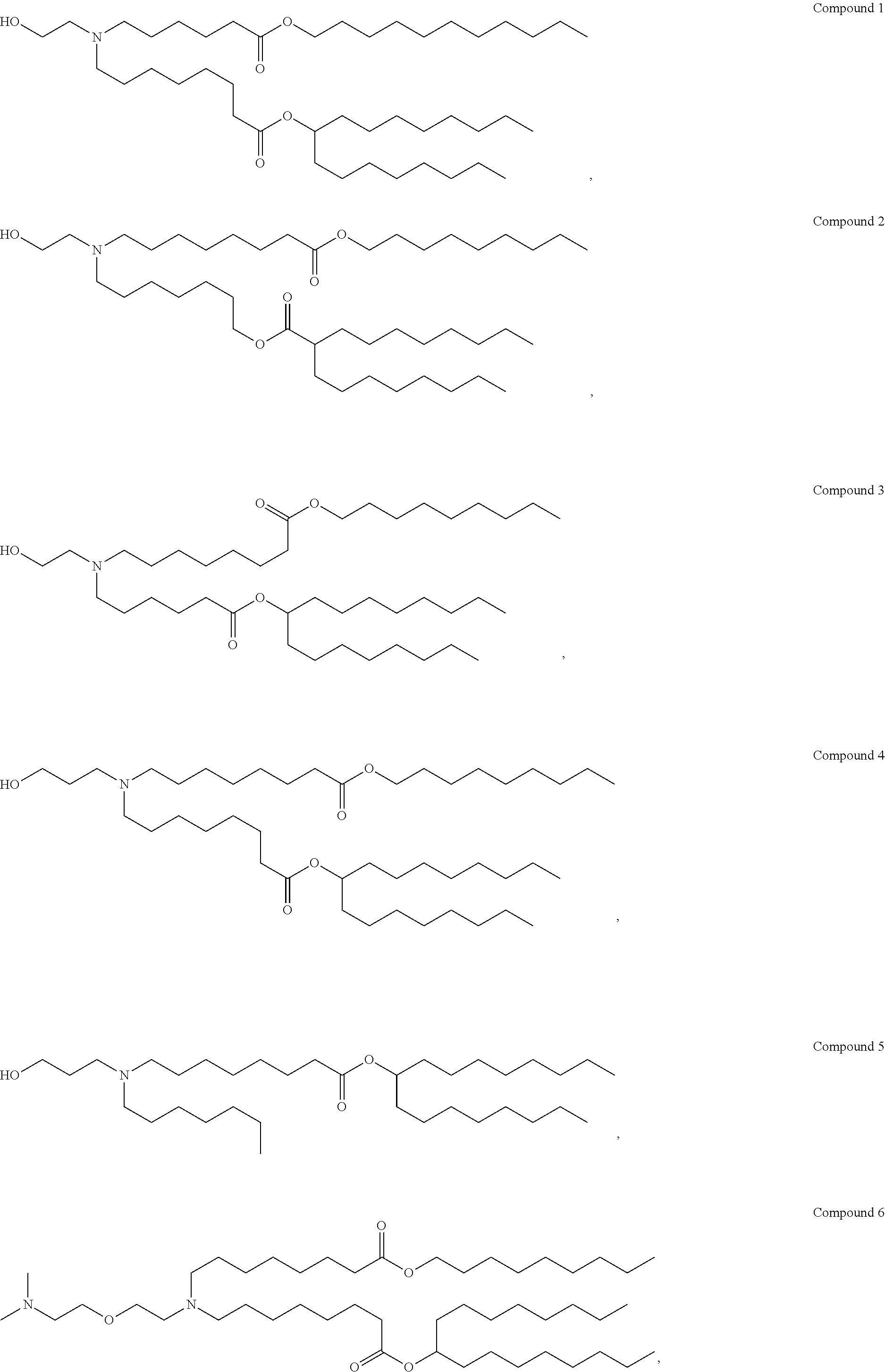

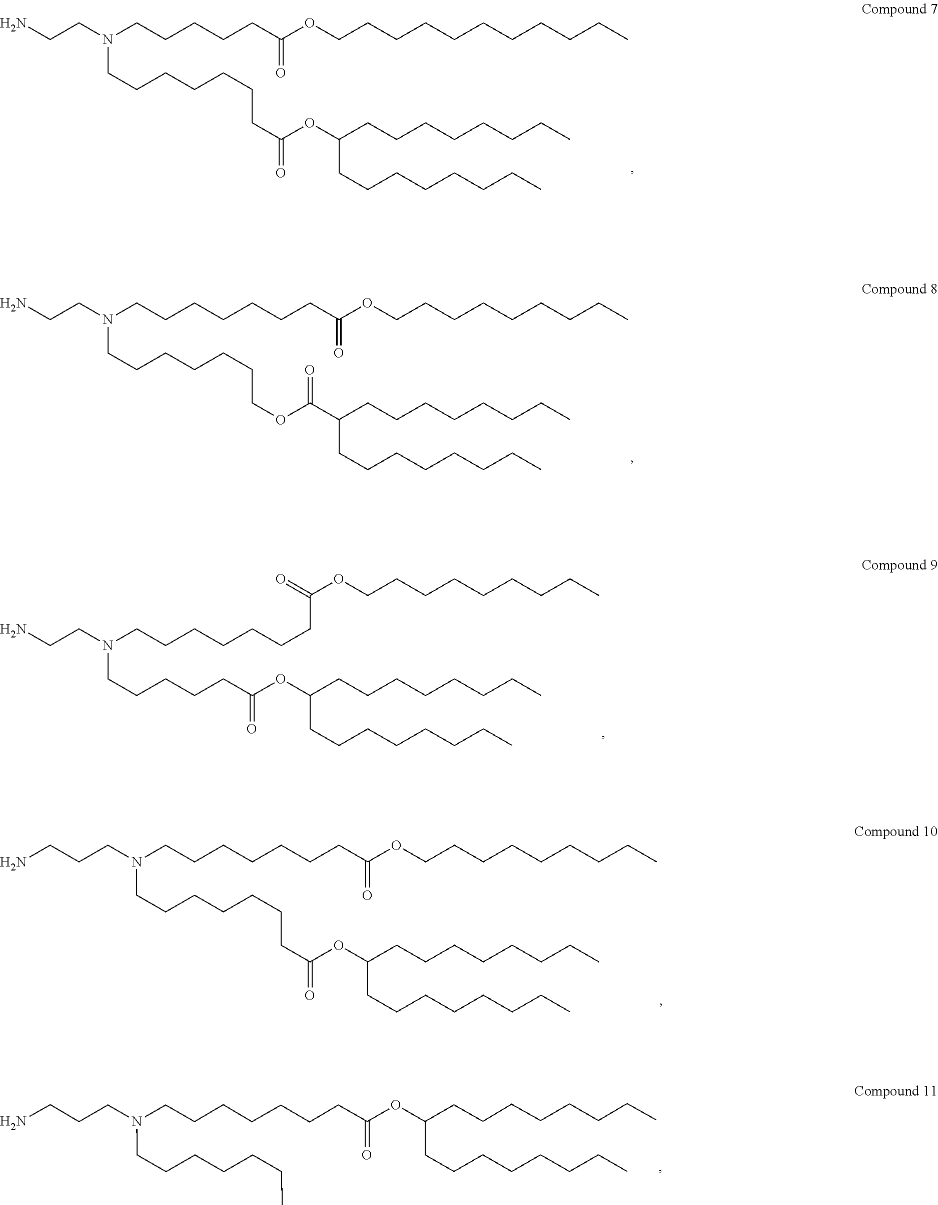 [SMILES: CCCCCCCCCCCOC(=O)CCCCCN(CCN)CCCCCCCC(=O)OC(CCCCCCCC)CCCCCCCC ; CCCCCCCCCOC(=O)CCCCCCCN(CCN)CCCCCCCOC(=O)C(CCCCCCCC)CCCCCCCC ; CCCCCCCCCOC(=O)CCCCCCCN(CCN)CCCCCC(=O)OC(CCCCCCCC)CCCCCCCC ; CCCCCCCCCOC(=O)CCCCCCCN(CCCN)CCCCCCCC(=O)OC(CCCCCCCC)CCCCCCCC ; CCCCCCCCC(CCCCCCCC)OC(=O)CCCCCCCN(CCCN)CCCCCCC]

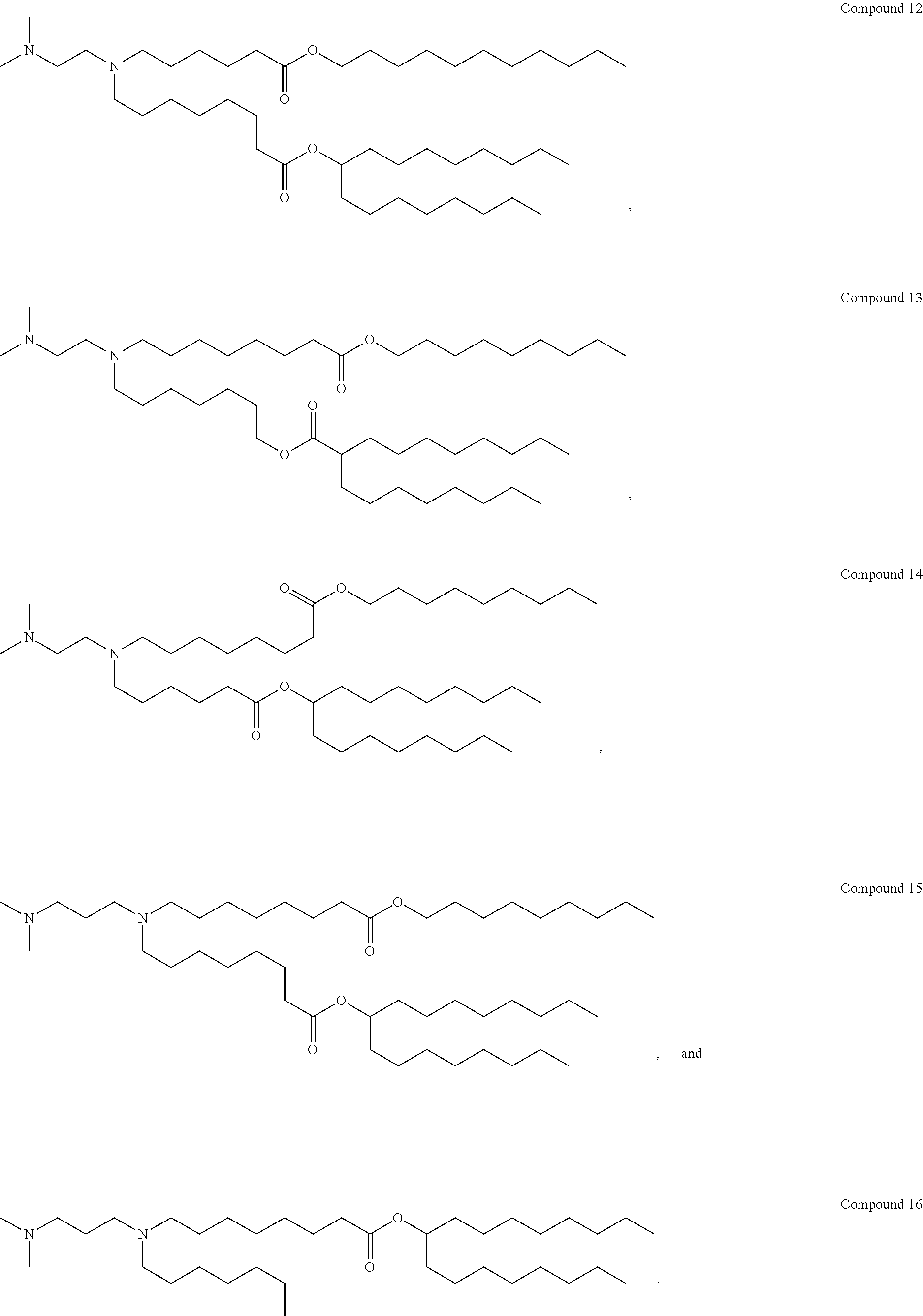

In some embodiments, the at least one neutral lipid is chosen from: cholesterol, dioleoylphosphatidylethanolamine (DOPE), dioleoylphosphatidylcholine (DOPC), and diphytanoylphosphatidylethanolamine (DDhPE).

In some embodiments, the compositions disclosed herein further comprise a pharmaceutically acceptable carrier.

Another aspect of the present disclosure is a kit comprising a trinucleotide cap analog of Formula (I), as well as any components of compositions set out herein (e.g., one or more RNAse inhibitor, etc.); nucleotide triphosphate molecules; and an RNA polymerase.

Another aspect of the present disclosure is a method of producing trinucleotide capped RNA comprising contacting a nucleic acid substrate with an RNA polymerase and a trinucleotide cap analog of Formula (I) or any of the embodiments thereof disclosed herein, in the presence of nucleotide triphosphates under conditions and for a time sufficient to produce a trinucleotide capped RNA.

Further aspects of the present disclosure include a method comprising contacting a cell with the trinucleotide cap analog comprising a trinucleotide cap analog of Formula (I) or any of the embodiments thereof disclosed herein. In some embodiments, the method is for increasing intracellular stability of an RNA, comprising incorporating a trinucleotide cap analog according to Formula (I) or any of the embodiments thereof disclosed herein into the RNA. In some embodiments, the method is for introducing an RNA into a cell, comprising contacting the cell. In some embodiments, the method is for RNA translation inhibition in a cell.

For the purposes of interpreting this specification, the following definitions will apply and whenever appropriate, terms used in the singular will also include the plural and vice versa. In the event that any definition set forth below conflicts with the usage of that word in any other document, the definition set forth below shall always control for purposes of interpreting this specification and its associated claims unless a contrary meaning is clearly intended (for example in interpreting the document where the term is originally used). The use of “or” herein means “and/or” unless stated otherwise or where the use of “and/or” is clearly inappropriate. The use of “a” herein means “one or more” unless stated otherwise or where the use of “one or more” is clearly inappropriate. The use of “comprise,” “comprises,” “comprising,” “include,” “includes,” and “including” are interchangeable and not intended to be limiting.

As used herein, the term “moiety” and “group” are used interchangeably to refer to a portion of a molecule, typically having a particular functional or structural feature, e.g., a linking group (a portion of a molecule connecting two other portions of the molecule), or an ethyl moiety (a portion of a molecule with a structure closely related to ethane).

As used herein, “Me” is equivalent to “CH₃;” “OCH₃” or “OMe” denotes an oxygen atom bound to a methyl group; “CHO” denotes a carbon atom, C, bonded to a hydrogen atom, H, and double-bonded to an oxygen atom, O, (O═CH—); and “Et” denotes “C₂H₅”.

As used herein, the names of bases: adenine, guanine, cytosine, and uracil are interchangeable with their capitalized initials: “A,” “G,” “C,” and “U,” respectively.

As used herein, the term “cap” refers to a non-extendible di-nucleotide (also referred to herein as a “dimer”) that facilitates translation or localization, and/or prevents degradation of an RNA transcript when incorporated at the 5′ end of an RNA transcript, typically having an m7GpppG or m7GpppA structure. Caps generally consist in nature of the modified base 7-methylguanosine joined in the opposite orientation, 5′ to 5′ rather than 5′ to 3′, to the rest of the molecule via three phosphate groups, i.e., PI-guanosine-5′-yl P3-7-methylguanosine-5′-yl triphosphate (m⁷G5′ppp5′G).

As used herein, the term “cap analog” refers to a structural derivative of an RNA cap that may differ by as little as a single element. Cap analogs may be trinucleotides (also referred to herein as a “trimer”), as well as pentamers and longer multimers (e.g., nucleic acid multimers that are five, six, seven, eight or nine nucleotides in length).

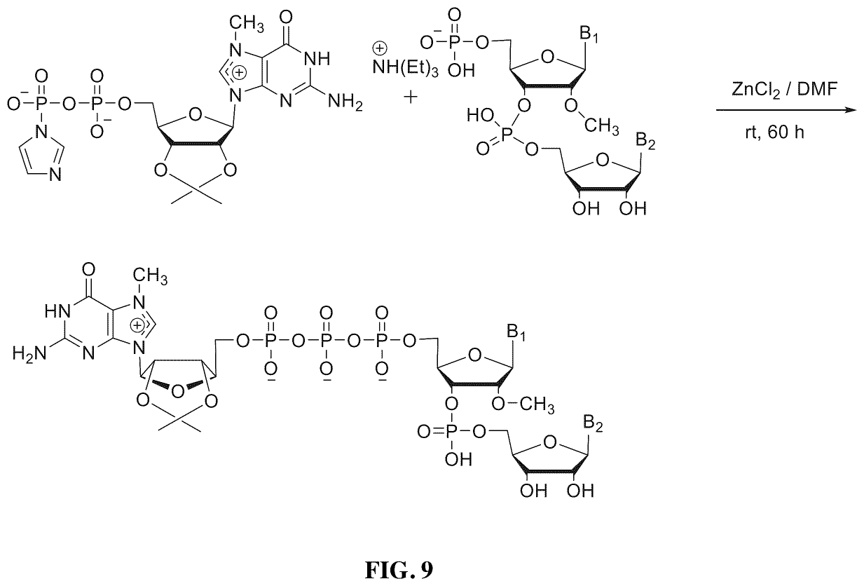

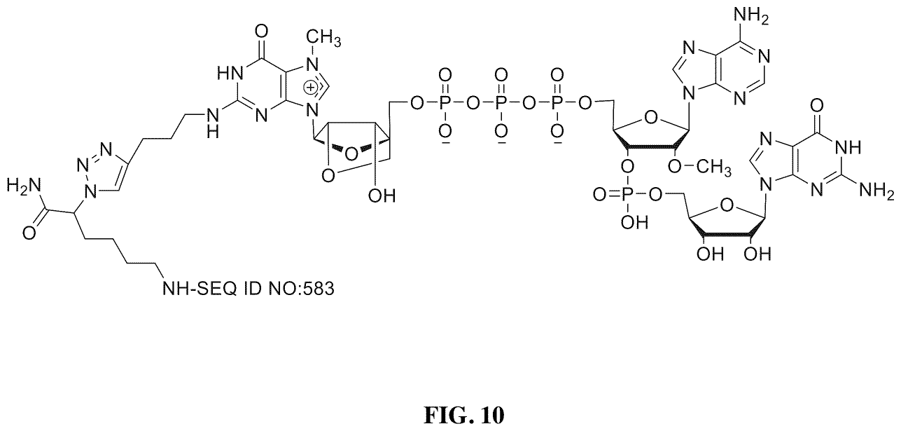

As used herein, the term “capped oligonucleotides” or “capped primers” refer to a transcriptional initiating primer containing a Cap 0, Cap 1, Cap 2 or 2,2,7-trimethylguanosine (TMG)-Cap structure on 5′-end of the primer. The capped primers will generally have an unmodified or open 3′—OH group and it may be extended by RNA polymerase through the incorporation of an NTP onto the 3′-end of the primer. Such oligonucleotides will generally be able to initiate in vitro transcription under the control of a promoter in a transcription system. The term “capped oligonucleotides” or “capped primers” include caps such as those set out herein that can be used to generate capped RNA molecules by transcription (see, e.g., the cap analog generated in the workflow set out in FIG. 9). Also used herein, “initiating primer” or “initiating oligonucleotide primer” refers to an oligonucleotide, carrying a terminal 3′—OH group that can act as a substrate for RNA polymerase in initiation of RNA synthesis (e.g., template directed RNA synthesis). By way of example, naturally occurring caps that may be added to RNA molecules by transcription are cap primers. Thus, cap primers may include naturally occurring caps or cap analogs, such as caps or cap analogs set out herein.

The term “nucleotide”, as referred to herein, includes naturally-occurring nucleotides, synthetic nucleotides and modified nucleotides, unless indicated otherwise.

The term “trinucleotide cap analog” refers to a cap or cap analog that comprises three nucleotides. By way of example, the cap analog generated in the workflow set out in FIG. 9 is a trinucleotide cap analog where the first nucleotide (on the left) is separated from the other two nucleotides (on the right) by three phosphate groups. At least one of the two nucleotides on the right will generally be designed to hybridize to the initiation site and act to primer transcription driven by RNA polymerase. Caps and cap analogs set out and used herein may be longer than three nucleotides. For example, there may be more than two nucleotides analogous to those on the right hand side of FIG. 9. In particular, caps and cap analogs may contain anywhere from four to twenty in which three to nineteen (e.g., from about three to about sixteen, from about three to about twelve, from about four to about sixteen, from about four to about ten, from about five to about ten, etc.) of these nucleotide may be capable of hybridizing to an initiation site and act to primer transcription driven by RNA polymerase.

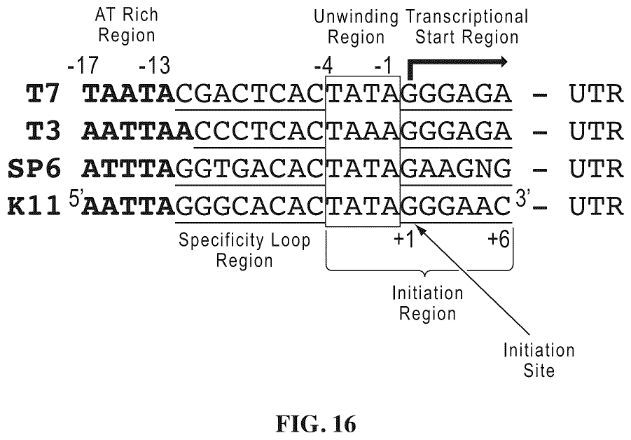

As used herein, the term “promoter” refers to a nucleic acid region that is recognized by RNA polymerase and capable of acting as an initiation template for operably linked nucleic acid region, resulting in transcription of part of all of the operably linked nucleic acid region. Promoters may be of eukaryotic, prokaryotic, viral, or organelle origin. Further, promoters can be natural occurring, modified naturally occurring, or synthetic (e.g., fusions of two naturally occurring promoters or promoters designed from consensus sequences of naturally occurring promoters). One category of promoters is the “T7 like” promoters, such as those set out in FIG. 16.

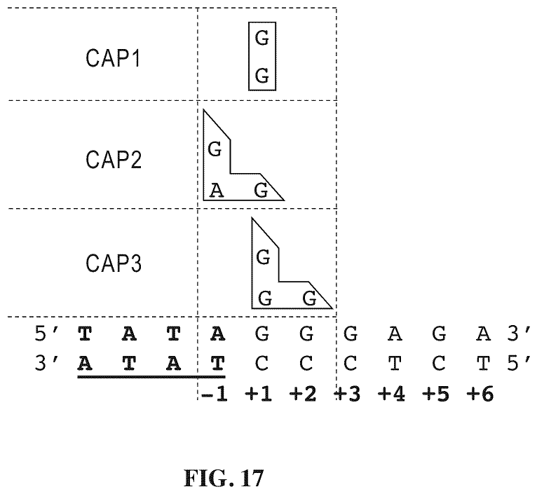

In many instances, promoters will be double stranded nucleic acid composed of a template strand and a non-template strand. FIG. 16 shows non-template strands of four different promoters and FIG. 17 shows both template and non-template strands of a portion of a bacteriophage promoter.

In some instances, promoters will be single stranded nucleic acid composed of a template strand or a nontemplate strand. Examples of such promoters are promoters of RNA and DNA single stranded viruses (e.g., Alphaviruses, Hantaviruses, and Flaviviruses).

Promoters are typically located immediately adjacent to (or partially overlapping with) the nucleic acid to be transcribed. Nucleotide positions in the promoter are generally designated relative to the transcriptional start site, normally referred to as position +1 (see FIG. 16) in wild-type systems. At least one base of the initiating oligonucleotide primer (e.g., mRNA cap or cap analog) is complementary to the template strand of the initiation site of promoter sequence which is used for initiation of transcription (e.g., position +1, −1 and +1, or +1 and +2 as set out in FIG. 16).

As herein, the term “initiation complex” refers to the association of primer and the template strand of a nucleic acid molecule, under conditions in which allow for the initiation of transcription of an RNA molecule by an RNA polymerase. In many instances, the primer will be a cap analog (e.g., a mRNA cap analog provided herein). Exemplary cap analog RNA initiation complex structures are set out schematically in FIG. 17.

As herein, the term “initiation site” refers to the base or bases on the template strand of a promoter where capped primers bind for the initiation of RNA transcription. In many instances, initiation sites will be identified by the nucleotide sequence of the non-template strand (see, e.g., FIG. 16). Numerical values herein for both initiation sites and surrounding nucleic acid are in reference to native initiation sites (see, e.g., FIG. 16). Thus, using FIG. 17 for reference, naturally occurring transcriptional initiation based upon T7 promoters with a GG capped primer (CAP3) is believed to normally begin at position +1. Thus, hybridization of a AG capped primer (CAP2) at position −1 and +1 of FIG. 17 means that the capped primer hybridizes to one base of the ‘TATA box” and the +1 position, the naturally occurring initiation site. In such an instance, transcriptional initiation begins at the −1 position. Further, when the capped primer hybridizes to positions −1 and +1, then the initiation site is located at positions −1 and +1.

As used herein the term “in vitro transcription and translation (IVTT)” refers the generation of messenger RNA (mRNA) molecules and the production of proteins from these mRNA molecules. Typically, IVTT will employ cellular extracts that contain transcription and translation “machinery” (e.g., ribosomes, tRNA synthetases, charged and uncharged tRNA molecules, RNA polymerases, etc.). These are cellular components capable of performing transcription and translation reactions. Together with transcription components that include T7 RNA polymerase and nucleotides, IVTT can be employed transcribe and translate genes that are supplied in the form of a purified DNA molecule. Cellular components used in IVTT reactions may obtained for essentially any cell type and may be supplemented with various reagents (e.g., buffers, amino acids, tRNA molecules, etc.).

IVTT reactions are composed of two sub-components: (1) “in vitro transcription” (IVTr, or IVT) and (2) “in vitro translation” (IVTl). These processes may occur in a single reaction mixture or may be performed in separate reaction mixtures.

As used herein, the term “cationic lipid” refers to a lipid that which under physiological conditions possess at least one positive charge.

The term “ARCA” or anti-reverse cap analog refers to a modified cap analog in which either the 3′ —OH group or the 2′ —OH group of the m⁷G is modified. This modification forces RNA polymerases to initiate transcription with the remaining —OH group in the G residue of the cap and thus synthesize RNA transcripts capped exclusively in the correct orientation. Therefore, use of the cap analog provided herein allows for synthesis of capped RNAs that are 100% functional in contrast to transcription reactions using traditional cap analogs where only half of the cap analog is incorporated in the correct orientation. Capped mRNAs provided herein are used for protein synthesis in reticulocyte lysates, wheat germ lysates, and other in vitro systems, or can be, for example, microinjected, electroporated, or transfected into cells or organisms for in vivo studies. They can also be used in RNA splicing and stability studies.

As used herein, the term “cell-penetrating peptide” refers to a modified peptide or other entity that aides in cellular uptake of an RNA, e.g., by facilitating transfer of a cargo molecule from the membrane to the cytoplasm and nucleus. Non-limiting examples of suitable cell penetrating peptides useful in the embodiments disclosed herein include the peptides listed in Table 2, and the peptides listed in Table 2 optionally covalently linked to a dye:

TABLE 2

| SEQ | |
|---|---|
| ID | |
| No. | Sequence |
| 1 | GYSTPPKKKRKVEDP |
| 2 | GYSTPPKTRRRP |
| 3 | GYSTPGRKKR |
| 4 | GYSTPRRNRRRRW |
| 5 | PDEVKRKKKPPTSYG |
| 6 | PRRRTKPPTSYG |
| 7 | RKKRGPTSYG |
| 8 | WRRRRNRRPTSYG |
| 9 | GYGPPKKKRKVEAPYKA |
| 10 | PAAKRVKLD |
| 11 | RQRRNELKRSP |
| 12 | KRPAATKKAGQAKKKK |
| 13 | VRKKRKTEEESPLKDKDAKKSKQE |
| 14 | RLRRDAGGRGGVYEHLGGAPRRRK |
| 15 | KRKGDEVDGVDECAKKSKK |
| 16 | NQSSNFGPMKGGNFGGRSSGPYGGGGQYFAKPRNQGGY |
| 17 | GGKRTADGSEFESPKKARKVEAYPKAW |
| 18 | GGKRTADGSEFESPKKKRAVEAYPKAW |
| 19 | GGKRTADGSEFESPKKKAKVEAYPKAW |
| 20 | GGKRTADGSEFESPKKKRKVEAPYKAWK |
| 21 | GGKRTADGSEFESPKKKRKVEYKAWK |
| 22 | GYGPAAKRVKLDEAYPKAWK |
| 23 | GGKRTADGSEFEPAAKRVKLDEAYPKAWK |
| 24 | GTGPKKKRKVGGGGYGPKKKRLVG |
| 25 | KRPAATKKAGQAKKKKLEAYPKAWK |
| 26 | ATKGTKRSYEQMETGE |
| 27 | GKWERKPIRCAS |
| 28 | GYGKRTADSQHSTPPKKKRKVEAPYKAWK |
| 29 | KRTADSQHSTPPKKKRKVEAPYKAWK |
| 30 | GYGPPKKKRKVEAPYKAWKWAKYPAMRRAHHRRRRASHRRRTTTG |
| | T |
| 31 | GYGPPKKKRKVEAPYKAWKRGARRYSKMKRRRRRVARRHRRRP |
| 32 | FWGYGYGPPKKKRKVEAPYKAWK |
| 33 | GKPSSDDEATADSQHSTPPKKKERKVED |
| 34 | GKPTADDQHSTPPKKKRKVED |
| 35 | GGKRTADGSEFESPKKARKVEAYPKAK |
| 36 | EKIRLRPGRKKRYRLKHL |
| 37 | PEGTRQARRNRRRRWRKR |
| 38 | PEGTRQPRRNRRRRWRKR |
| 39 | GVKRSYGAARGDDRRRPNVVAPYKAW |
| 40 | KSVPNRTRTYIKLKRLRFKGAPYKAW |
| 41 | EMRRRREEEGLQLRKQKREEQLFKRRN |
| 42 | FEAALAEALAEALA |
| 43 | Ac-LARLLPRLLARL-NHCH₃ |
| 44 | GLLEELLELLEELWEELLEG |
| 45 | GWEGLIEGIEGGWEGLIEG |
| 46 | GLFEALAEFIEGGWEGLIEG |
| 47 | GLFEALLELLESLWELLLEA |
| 48 | GGYCLEKWMIVASELKCFGNTA |
| 49 | GGYCLTRWMLIEAELKCFGNTAV |
| 50 | WEAALAEALAEALAEHLAEALAEALEALAA |
| 51 | GLFGAIAGFIENGWEGMIDGWYG |
| 52 | GIGAVLKVLTTGLPALISWIKRKRQQ |
| 53 | GRKKRRQRRRPPQ |
| 54 | RQIKIWFQNRRMKWKK |
| 55 | GWTLNSAGYLLGKINLKALAALAKKIL |
| 56 | WEAKLAKALAKALAKHLAKALAKALKACEA |
| 57 | GLFKALLKLLKSLWKLLLKA |
| 58 | GLFRALLRLLRSLWRLLLRA |
| 59 | GLFEALLELLESLYELLLEA |
| 60 | GLFEALEELWEA |
| 61 | GLFLLEEWLE |
| 62 | GLFLLEEWLEK |
| 63 | GLFEALLELLESLWELLLEAK |
| 64 | Suc-GLFKLLEEWLE |
| 65 | Suc-GLFKLLEEWLEK |
| 66 | GLFEAIAEFIEGGWEGLIEG |
| 67 | GLFKAIAKFIKGGWKGLIKG |
| 68 | IRFKKTKLIASIAMALC |
| 69 | ALAGTIIAGASLTFQVLDKV1EELGKVSRK |
| 70 | GLFEAIEGFIENGWEGMIDGWYG |
| 71 | GYICRRARGDNPDDRCT |
| 72 | GLFEAIAEFIEGGWEGLIEGCA |
| 73 | GLFHAIAHFIHGGWHGLIHGWWYG |
| 74 | RRRQRRKKRGGDIMGEWGNEIFGAIAGFLG |
| 75 | GLFEAIADFIENGWEGMIDGGG |
| 76 | ALAGTIIAGASLTFQVLDKV1EELGKVSRKK |
| 77 | IRFKKTKLIASIAMA |
| 78 | GLWHLLLHLWRRLLRLLR |
| 79 | KKIMLLLMTLLLVSLPLAQEQ |
| 80 | GLFEALLELLESLWELLLEAWYG |
| 81 | RLLRLLLRLWRRLLRLLR |
| 82 | LLELELLELELLLELELLELELLLEL |
| 83 | GLFEALLELLESLWELLLEARRRRRRRR |
| 84 | GLFEALLELLESLWELLLEARRRRRR |
| 85 | GLFEALLELLESLWELLLEAKKKKKKKK |
| 86 | GLFEALLELLESLWELLLEAKKKKKK |
| 87 | GLFEALLELLESLWELLLEAKK |
| 88 | GLFEALLELLESLWELLLEAKKKK |
| 89 | GLFEALLELLESLWELLLEAEE |
| 90 | GLFEALLELLESLWELLLEAEEEE |
| 91 | GLFEALLELLESLWELLLEAEEEEEE |
| 92 | GLFEALLELLESLWELLL |
| 93 | PLSSIFSRIGDPRGARRYAKMKRRRRRVARRHRRRP |
| 94 | GPFHYFQFLFPPV |
| 95 | GSSSWWQRWWPPW |
| 96 | RRRQRRKKR |
| 97 | KKKK |
| 98 | KKKKKK |
| 99 | KKKKKKKK |
| 100 | KKKKKKKKKK |
| 101 | KKKKKKKKKKKK |
| 102 | KKKKKKKKKKKKKKKK |
| 103 | KKKKKKKKKKKKKKKKKKKK |
| 104 | KKKKKKKKKKKKKKKKKKKKKKKK |
| 105 | RRRR |
| 106 | RRRRRR |
| 107 | RRRRRRRR |
| 108 | RRRRRRRRRR |
| 109 | RRRRRRRRRRRR |
| 110 | RRRRRRRRRRRRRRRR |
| 111 | RRRRRRRRRRRRRRRRRRRR |
| 112 | RRRRRRRRRRRRRRRRRRRRRRRR |
| 113 | YKA |
| 114 | KKKKKKKKWKGGGGACYGLPHLFCG |
| 115 | YKAKKKKKKKKWK |
| 116 | KTPKKAKKPKTPKKAKKP |
| 117 | KKAKKPAATRKSSKNPKKPKTVKPKKVAK |
| 118 | RGARRYSKMKRRRRRVARRHRRRP |
| 119 | TRQARRNRRRRWRERQRGSGSG |
| 120 | KRPRGRPKGSKKNWRRRKRRASRRSPRRR |
| 121 | KRGRGRPRKQPPKEPSEVPTPKRPRGRPKGSKNK |
| 122 | KEKYEKDIAAYRAKGKPAAKKGVVKAEKSKKKK |
| 123 | YKAKKKKKKKKKKWK |
| 124 | KKKKKKKGGC |
| 125 | YRARRRRRRRRWR |
| 126 | YRARRRRRRRRRRWR |
| 127 | KGDPKKPRGKMSSYAFFVQTCREEHKKKHPDASVNFSEFSKK |
| 128 | KKQLKKQLKKQLKQWK |
| 129 | KKSPKKSPKKSPKKSK |
| 130 | KLSKLEKKSKLEK |
| 131 | KLSKLEKKLSKLEKKSKLEK |
| 132 | KSLKKSLKKSLKKSK |
| 133 | KIRRRGKNKVAARTCRQRRTDR |
| 134 | KIRRRGKNKVAAQNCRKRKLET |
| 135 | KRRIRREKNKMAAAKCRNRRRELT |
| 136 | KDRSNLLERHTR |
| 137 | KRPAATKKAGQAKKKL |
| 138 | RRRRRREEEE |
| 139 | RRRRRREEEEEE |
| 140 | RRRRRREEEEEEEE |
| 141 | RRRRRRRREEEE |
| 142 | RRRRRRRREEEEEE |
| 143 | RRRRRRRREEEEEEEE |
| 144 | RRRRRRRRRRRREEEE |
| 145 | RRRRRRRRRRRREEEEEE |
| 146 | RRRRRRRRRRRREEEEEE |
| 147 | KLSKLEKK |
| 148 | SKLEK |
| 149 | KLSKLEKKLSKLEKK |
| 150 | PKKKRKVGGGRGDSP |
| 151 | LPHKSMPCG |
| 152 | GACLQHKSMPCG |
| 153 | YGLPHLFCG |
| 154 | SERSMNFCG |
| 155 | DHYSLYEDLERGTDK |
| 156 | ISLPRTSGAQRASTTR |
| 157 | EKLQTKYGLPHKVEFCG |
| 158 | TRISESQAKPGD |
| 159 | LVFFDY |
| 160 | WGGNGPTTFDCSGYTKYVFAK |
| 161 | INIGTTGWGDHYSLY |
| 162 | YDNIHG |
| 163 | AGWGKFLVGFGRV |
| 164 | SIGYPLP |
| 165 | TTHWGFTL |
| 166 | HLQIQPYPQISG |
| 167 | KLNIVSVNG |
| 168 | RGH |
| 169 | DNRIRLQAKAA |
| 170 | KIKMVISWKG |
| 171 | LPWYSYLYAVSA |
| 172 | WNLPWYYSVSPT |
| 173 | WNL |
| 174 | PWYYSVSPT |
| 175 | SSWESYKSGGGTRL |
| 176 | RDWSSQHPGRCNGETHLK |
| 177 | SLPTLTL |
| 178 | VICTGGDYSFALPVGQWPVMT |
| 179 | DKPSYQFGGHNSVDFEEDTLPKV |
| 180 | RARRRKRASATQLYQTCKASGTCPPD |
| 181 | SGDYSFALPVGQWPWMTG |
| 182 | CTGGDYSFALPVGQWPW |
| 183 | FYYDYDFFFDYWGQG |
| 184 | HLRRLRRRLLREAEG |
| 185 | DYYCAAWDDSLNGYSVF |
| 186 | YYCLQSMEDPYTFGG |
| 187 | YYCARSDGNYGYYYALDYDY |
| 188 | AARSPSYYRYDY |
| 189 | GPYYAMDYD |
| 190 | YYCQQRSSYPYTEGGAYPKAWK |
| 191 | YYCQRYDSDWSFGQGTKL |
| 192 | YYCARSGYYAMDYWGQGT |
| 193 | RVRRGACRGDCLG |
| 194 | RVRRGACRYDCLG |
| 195 | YYCAKGTHWGFWSGYFDYWGQGT |
| 196 | GRENYHGCTTHWGFTLC |
| 197 | VQATQSNQHTPRGGGSK |
| 198 | DPRAPGS |
| 199 | YYCQQRSSYPYTFGG |
| 200 | AARSPSYYRYDYGPYYAMDYD |
| 201 | GPKLTGILISILSLFVES |
| 202 | KYILRWRPKNS |
| 203 | IKVAV |
| 204 | WTPPRAQITGYRLTVGLTRR |
| 205 | AASIKVAVSADR |
| 206 | KLDAPT |
| 207 | NRWHSIYITRFG |
| 208 | PHSRN |
| 209 | SSFHFDGSGYAM |
| 210 | RGDS |
| 211 | IAFQRN |
| 212 | GRGDSP |
| 213 | TWYKIAFQRRK |
| 214 | EDGIHEL |
| 215 | SLVRNRRVITIQ |
| 216 | YRVRVTPKEKTGPMKE |
| 217 | LQVQLSR |
| 218 | SPPRRARVT |
| 219 | RKRLQVQLSIRT |
| 220 | ATETTITIS |
| 221 | NAPFPKLSWTIQ |
| 222 | VSPPRRARVTDATETTITISWRTKTETITGG |
| 223 | WTIQTTVDRGLL |
| 224 | KPDVRSYTITG |
| 225 | DTINNGRDHMILI |
| 226 | ANGQTPIQRYIK |
| 227 | MILISIGKSQKRM |
| 228 | PRARITGYIIKYEKPGSPPREVVPRPRPGV |
| 229 | PPFLMLLKGSTR |
| 230 | WQPPRARI |
| 231 | NQRLASFSNAQQS |
| 232 | WQPPRARITGYIIKYEKPG |
| 233 | ISNVFVQRMSQSPEVLD |
| 234 | YEKPGSPPREVVPRPRPGV |
| 235 | KARSFNVNQLLQD |
| 236 | KNNQKSEPLIGRKKT |
| 237 | KNSFMALYLSKG |
| 238 | EILDVPST |
| 239 | KNSFMALYLSKGRLVFALG |
| 240 | IDAPS |
| 241 | RDSFVALYLSEGHVIFAGLG |
| 242 | VVIDASTAIDAPSNL |
| 243 | KPRLQFSLDIQT |
| 244 | LDVPS |
| 245 | DGQWHSVTVSIK |
| 246 | REDV |
| 247 | FVLYLGSKNAKK |
| 248 | PHSRNRGDSP |
| 249 | LAIKNDNLVYVY |
| 250 | LWVTVRSQQRGLF |
| 251 | AYFSIVKIERVG |
| 252 | GTNNWWQSPSIQN |
| 253 | DVISLYNFKHIY |
| 254 | WVTVTLDLRQVFQ |
| 255 | FFDGSSYAVVRD |
| 256 | RQVFQVAYIIIKA |
| 257 | LHVFYDFGFGFSNG |
| 258 | LTRYKITPRRGPPT |
| 259 | LKKAQINDAKYREISIIYHN |
| 260 | LLEFTSARYIRL |
| 261 | RAYFNGQSFIAS |
| 262 | YIRLRLQRIRTL |
| 263 | SRLRGKNPTKGK |
| 264 | RRYYYSIKDISV |
| 265 | LHKKGKNSSKPK |
| 266 | SINNTAVNQRLT |
| 267 | RLKTRSSHGMIF |
| 268 | GGFLKYTVSYDI |
| 269 | GEKSQFSIRLKT |
| 270 | RDQLMTVLANVT |
| 271 | TLFLAHGRLVFM |
| 272 | ANVTHLLIRANY |
| 273 | LVFMFNVGHKKL |
| 274 | AGTFALRGDNPQG |
| 275 | TLFLAHGRLVFMFNVGHKKL |
| 276 | VLIKGGRARKHV |
| 277 | DFMTLFLAHGRLVFMGNVG |
| 278 | LSNIDYLIKAS |
| 279 | HKKLKIRSQEKY |
| 280 | LQQSRIANISME |
| 281 | GAAWKIKGPIYL |
| 282 | NLLLLLVKANLK |
| 283 | VIRDSNVVQLDV |
| 284 | HRDELLLWARKI |
| 285 | GLIYYVAHQNQM |
| 286 | KRRARDLVHRAE |
| 287 | DYATLQLQEGRLHFMFDLG |
| 288 | SQFQESVDNITK |
| 289 | KKGSYNNIVVHV |
| 290 | PGGMREKGRKAR |
| 291 | ADNLLFYLGSAK |
| 292 | MEMQANLLLDRL |
| 293 | GSAKFIDFLAIE |
| 294 | LSEIKLLISAR |
| 295 | KVSFLWWVGSGV |
| 296 | RDFTKATNIRLRFLR |
| 297 | SYWYRIEASRTG |
| 298 | ISTVMFKFRTFS |
| 299 | YFDGTGFAKAVG |
| 300 | KQANISIVDIDSN |
| 301 | NGQWHKVTAKKI |
| 302 | FSTRNESGIILL |
| 303 | AKKIKNRLELVV |
| 304 | RRQTTQAYYAIF |
| 305 | GFPGGLNQFGLTTN |
| 306 | YAIFLNKGRLEV |
| 307 | NQFGLTTNIRFRG |
| 308 | KNRLTIELEVRT |
| 309 | IRSLKLTKGTGKP |
| 310 | GLLFYMARINHA |
| 311 | AKALELRGVQPVS |
| 312 | VQLRNGFPYFSY |
| 313 | GQLFHVAYILIKF |
| 314 | HKIKIVRVKQEG |
| 315 | NVLSLYNFKTTF |
| 316 | DFGTVQLRNGFPFFSYDLG |
| 317 | SQRIYQFAKLNYT |
| 318 | NIRLRFLRTNTL |
| 319 | EVNVTLDLGQVFH |
| 320 | GKNTGDHFVLYM |
| 321 | GQVFHVAYVLIKF |
| 322 | VVSLYNFEQTFML |
| 323 | HQQDLGTAGSCLRKFSTMFLF |
| 324 | RFDQELRLVSYN |
| 325 | HQQDLGTAGSCLRKFSTMFLFCNI |
| 326 | RLVSYSGVLFFLK |
| 327 | VAEIDGIEL |
| 328 | NWRHISYITRFG |
| 329 | GIIFFL |
| 330 | KRLQVQLRSIRT |
| 331 | ASKAIQVFLLGG |
| 332 | TWYKIAFQRNRK |
| 333 | VLVRVERATVFS |
| 334 | QVFQVAYIIIKA |
| 335 | TVFSVDQDNMLE |
| 336 | GEFYFDLRLKGDK |
| 337 | RLRGPQRVFDLH |
| 338 | GTPGPQGIA |
| 339 | FDLHQNMGSVN |
| 340 | GQRDVV |
| 341 | LRAHAVDVNG |
| 342 | TAGSCLRKFSTM |
| 343 | LFSHAVSSNG |
| 344 | KGHRGF |
| 345 | TAGSCLRKFSTMFLF |
| 346 | TAGSCLRKFSTMFLFCNI |
| 347 | DLGTAGSCLRKFSTM |
| 348 | HQQDLGTAGSCLRKFSTM |
| 349 | RNIAEIIKDI |
| 350 | SIGFRGDGQTC |
| 351 | LNRQELFPFG |
| 352 | RIQNLLKITNLRIKFVK |
| 353 | KKQRFRHRNRKGYRSQ |
| 354 | SINNTAVMQRLT |
| 355 | FRHRNRKGY |
| 356 | RYRVRVTPKEKTGPMKE |
| 357 | SETTVKYIFRLHE |
| 358 | GHRGPTGRPGKRGKQGQKGDS |
| 359 | KAFDITYVRLKF |
| 360 | GDLGRPGRKGRPGPP |
| 361 | YIGSR |
| 362 | RGEFYFDLRLKGDK |
| 363 | LAGSCLARFSTM |
| 364 | LALFLSNGHFVA |
| 365 | ISRCQVCMKKRH |
| 366 | PGRWHKVSVRWE |
| 367 | TDIPPCPHGWISLWK |
| 368 | VRWGMQQIQLVV |
| 369 | TAIPSCPEGTVPLYS |
| 370 | KMPYVSLELEMR |
| 371 | GPAGKDGEAGAQG |
| 372 | VLLQANDGAGEF |
| 373 | GLPGER |
| 374 | DGRWHRVAVIMG |
| 375 | LAGSCLPVFSTL |
| 376 | APVNVTASVQIQ |
| 377 | TAGSCLRRFSTM |
| 378 | KQGKALTQRHAK |
| 379 | TAGSCLRKF |
| 380 | RYVVLPR |
| 381 | TAGSCL |
| 382 | SPYTFIDSLVLMPY |
| 383 | TAG |
| 384 | PDSGR |
| 385 | QQNLGSVNVSTG |
| 386 | SRATAQKVSRRS |
| 387 | DPGYIGSR |
| 388 | GSLSSHLEFVGI |
| 389 | VILQQSAADIAR |
| 390 | RNRLHLSMLVRP |
| 391 | KDISEKVAVYST |
| 392 | APMSGRSPSLVLK |
| 393 | LGTIPG |
| 394 | AFGVLALWGTRV |
| 395 | TDIRVTLNRLNTF |
| 396 | IENVVTTFAPNR |
| 397 | AFSTLEGRPSAY |
| 398 | LEAEFHFTHLIM |
| 399 | TSAEAYNLLLRT |
| 400 | HLIMTFKTFRPA |
| 401 | LNRRYEQARNIS |
| 402 | KTWGVYRYFAYD |
| 403 | SLLSQLNNLLDQ |
| 404 | TNLRIKFVKLHT |
| 405 | RDIAEIIKDI |
| 406 | KRLVTGQR |
| 407 | SHAVSS |
| 408 | GPGVVVVERQYI |
| 409 | ADTPPV |
| 410 | NEPKVLKSYYYAI |
| 411 | LRAHAVDING |
| 412 | YYAISDFAVGGR |
| 413 | DSITKYFQMSLE |
| 414 | LPFFNDRPWRRAT |
| 415 | YTALIIATDN |
| 416 | FDPELYRSTGHGGH |
| 417 | VITVKDINDN |
| 418 | TNAVGYSVYDIS |
| 419 | GLDRESYPYY |
| 420 | APVKFLGNQVLSY |
| 421 | MKVSATDADD |
| 422 | SFSFRVDRRDTR |
| 423 | PQVTRGDVFTMP |
| 424 | TWSKVGGHLRPGIVQSG |
| 425 | KEAEREVTDLLR |
| 426 | RGDV |
| 427 | AAEPLKNIGILF |
| 428 | FALWDAIIGEL |
| 429 | VGVAPG |
| 430 | LWPLLAVLAAVA |
| 431 | PGVGV |
| 432 | VFDNFVLK |
| 433 | TSIKIRGTYSER |
| 434 | TTSWSQCSKS |
| 435 | DPETGV |
| 436 | KRSR |
| 437 | QGADTPPVGV |
| 438 | SVVYGLR |
| 439 | PLDREAIAKY |
| 440 | DGRGDSVAYG |
| 441 | HAVDI |
| 442 | LALERKDHSG |
| 443 | DQNDN |
| 444 | YSMKKTTMKIIPFNRLTIG |
| 445 | QDPELPDKNM |
| 446 | RGDF |
| 447 | LVVQAADLQG |
| 448 | GVYYQGGTYSKAS |
| 449 | NDDGGQFVVT |
| 450 | TAGSCLRKFSCL |
| 451 | YILHVAVTN |
| 452 | CNYYSNSYSFWLASLNPER |
| 453 | TYRIWRDTAN |
| 454 | TGLSCLQRFTTM |
| 455 | GFTCECSIGFRGDGQTCYGIVFWSEV |
| 456 | HHLGGAKQAGDV |
| 457 | SCLPGFSGDGRACRDVDECGH |
| 458 | MAPRPSLAKKQRFRHRNRKGYRSQRGHSRG |
| 459 | KKQKFRHRNRKGYRSQ |
| 460 | KKQKFKHRNRKGYRS |
| 461 | KKQKFRRRNRKGYRSH |
| 462 | TAIPPCPHGWISLWK |
| 463 | KKQKSRHRSRKRYRS |
| 464 | KKQKSRRRSRKGYRS |
| 465 | ISRCTVC |
| 466 | ISRCQVCMKRRH |
| 467 | VSRCTVC |
| 468 | TDIPPCPQGWISLWK |
| 469 | TVKAGELEKIISRCQVMKKRH |
| 470 | TDIPSCPHGWISLWK |
| 471 | TDIPPCPAGWISLWK |
| 472 | TEIPPCPQGWISLWK |
| 473 | TDVPPCPQGWISLWK |
| 474 | RLVSYNGILFFLK |
| 475 | RLVSYSGVIFFLK |
| 476 | RLVSYNGILFFL |
| 477 | RLVSYSGIIFFLK |
| 478 | RFEQELRLVSYSGVLFFLKQ |
| 479 | RLVSYNGIIFFLK |
| 480 | DPAFKIEDPYSPRIQNLLKITNLRIKFVKL |
| 481 | TKRFEQELRLVSYSGVLFFL |
| 482 | GGRLKYSVAF |
| 483 | GGFLRYTVSYDI |
| 484 | GGFLKYTVSYDV |
| 485 | LGNKLTAFGGFLKYTVSYDIPV |
| 486 | GGYLKYTVSYDI |
| 487 | GEIFFDMRLKGDK |
| 488 | GEIYFDLRLKGDK |
| 489 | GEIYLDMRLKGDK |
| 490 | IGQPGAKGEPGEFYFDLRLKGDKGDPGFPG |
| 491 | GEVFFDMRLKGDK |
| 492 | LAGSCLPIFSTL |
| 493 | AHNQDLGLAGSCLARFSTMPFLYCNPGDIC |
| 494 | QEKAHNQDLGLAGSCLPVFSTLPFAYCNIH |
| 495 | LAGSCLPVFSTM |
| 496 | GNKRAHGQDLGTAGSCLRRFSTMPFMFCNI |
| 497 | RAHGQDLGTAGSCLRRFSTMP |
| 498 | RKRLQVQLNIRT |
| 499 | HLVLPLQQSDVRKRLQVQLSIRTFASSGLI |
| 500 | RKRLSVQLRIRT |
| 501 | DLGTAGSCLRRFSTM |
| 502 | RNIAEIIKDI |
| 503 | TAGSCLRKFSTMRRRRRRRRRRRR |
| 504 | FTLTGLLGTLVTMGLLT |
| 505 | APYKAWK |
| 506 | STSKTNRGDDSNWSKRVTNNKPS |
| 507 | STSKRKRGDDSNWSKRVTKKKPS |
| 508 | STSKRKRGDDSNWSKRVSKKKPS |
| 509 | STSKRKRGDDANWSKRVTKKKPS |
| 510 | PLAGSKRKRADEVAWSKRGTKKKPER |
| 511 | PLAGSKRKRADEVAWSKRGTKKKPERTSAARAGPSRRIR |
| 512 | STSKRKRGDDANWSKRTTKKKPSS |
| 513 | STSKRKRGDDANWSKRTTKKKPSSAGLKRAGSKADRPSL |
| 514 | PTTAGKRKRSDDAAWSKRARPKAGRT |
| 515 | PTTAGKRKRSDDAAWSKRARPKAGRTSAARPGTSVRRIR |
| 516 | SSSLGKRKRSDEGAWSKGKSKKKAMR |
| 517 | SSSLGKRKRSDEGAWSKGKSKKKAMRGSSSRRPGPVRGP |
| 518 | PTTAGKRKRTDDAAWSKRARPKAGR |
| 519 | PTTAGKRKRTDDAAWSKRARPKAGRTSAARPGTAVRRVR |
| 520 | PATAGKRKRSDDAAWSKRARPKAGRTSAAR |
| 521 | PATAGKRKRSDDAAWSKRARPKAGRTSAARPGTSVRRIR |
| 522 | SSSLGKRKRSNGGDWSKRSAKKKPA |
| 523 | SSSLGKRKRSNGGDWSKRSAKKKPAGTPSRRAGPGRGPR |
| 524 | SSSLGKRKRSDEGAWSKGKSKKKAMR |
| 525 | SSSLGKRKRSDEGAWSKGKSKKKAMRGSSSRRPGPVRGP |
| 526 | STSKRKRGDDANWNKRPTKKKPSS |
| 527 | STSKRKRGDDANWNKRPTKKKPSSAGLKKAGSKAERPSL |
| 528 | SGALKRKRSDEVAWSRRRPVKKPV |
| 529 | SGALKRKRSDEVAWSRRRPVKKPVRRAPPPRAGPSVRRG |
| 530 | SGALKRKRSDEVAWSRRKPAKKPAR |
| 531 | SGALKRKRSDEVAWSRRKPAKKPARQPPPPRAGPSVRRG |
| 532 | AGALKRKRSDEVAWSRRKPAKKPAR |
| 533 | AGALKRKRSDEVAWSRRKPAKKPARAPPPRAGPSVRRGL |
| 534 | STSKRKRGDDSNWSKRVTKKKPSSAGLKRAGSKADRPSLQIQT |
| | LQHAGTTMITVPSGGVCDLINTYARGSDEGNRHTSETLTYKIAI |
| | DYHFVADAAACRYSNTGTGVMWLVYDTTPGGQAPTPQTIFSYP |
| | DTLKAWPATWKVSRELCHRFVVKRRWLFNMETDGRIGSDIPPS |
| | NASWKPCKRNIYFHKFTSGLGVRTQWKNVTDGGVGAIQRGAL |
| | YMVIAPGNGLTFTAHGQTRLYFKSVGNQ |
| 535 | DPQNALYYQPRVPTAAPTSGGVPWSRVGEVAILSFVALICFYLL |
| | YLWVLRDLILVLKARQGRSTEELIFGGQAVDRSNPIPNIPAPPS |
| | QGNPGPFVPGTG |
| 536 | GSQLVPPPSAFNYIESQRDEFQLSHDLTEIVLQFPSTASQITAR |
| | LSRSCMKIDHCVIEYRQQVPINASGTVIVEIHDKRMTDNESLQA |
| | SWTFPIRCNIDLHYFSSSFFSLKDPIPWKLYYRVSDSNVHQMTH |
| | FAKFKGKLKLSSAKHSVDIPFRAPTVKILAKQFSEKDIDFWHVG |
| | YGKWERRLVKSASSSRFGLRGPIEINPGESWATKSAIVTPNRNA |
| | DLDIEEELLPYRELNRLGTNILDPGESASIVGIQRSQSNITMSM |
| | SQLNELVRSTVHECIKTSCIPSTPKSLS |
| 537 | RTGVKRSYGAARGDDRRRPNVV |
| 538 | SYVKTVPNRTRTYIKLRVR |
| 539 | MYSTSNRRGRSQTQRGSHVRRTGVKRSYGAARGDDRRRPNVV |
| | SKTQVEPRMTIQRVQENQFGPEFVLSQNSALSTFVTYPSYVKTV |
| | PNRTRTYIKLKRVRFKGTLKIERGQGDTIMDGPSSNIEGVFSMV |
| | IVVDRKPHVSQSGRLHTFDELFGARIHCHGNLSVVPALKDRYYI |
| | RHVTKRVVSLEKDTLLIDLHGTTQLSNKRYNCWASFSDLERDCN |
| | GVYGNITKNALLVYYCWLSDAQSKASTYVSFELDYLG |
| 540 | RRRRRRRRRRRRVDYGKWERKPIRCASMSR |
| 541 | RRRRRRRRRRRRGKWERKPIRCAS |
| 542 | KKKKKKKKKKKKKKKKGKWERKPIRCAS |
| 543 | RRRRRRRRRRRRVDFSHVDYGKWERKPIRCASMSRLGLRG |
| 544 | GVKRSYGAARGDDRRRPNVVAPYKAWRRRRRRRRRRRR |
| 545 | KSVPNRTRTYIKLKRLRFKGAPYKAWRRRRRRRRRRRR |
| 546 | RTGVKRSYGAARGDDRRRPNVVRRRRRRRRRRRR |
| 547 | SYVKTVPNRTRTYIKGGGGGRRRRRRRRRRRR |
| 548 | VDIPFRAPTIKILSKQFTEDDIDFWHVGYGKWERKLVRPASLSG |
| | RRGLRR |
| 549 | IDFWHVGYGKWERKLVRPASLSGRRGLRR |
| 550 | IDFWSVEKGETRRRLLNPTPHAHSPRPIAHR |
| 551 | IDFSHVGYGKWERKMIRSASISRLGLHN |
| 552 | VDFSHVGYGKWERKLIRSASTVKYGLPS |
| 553 | IDFSHVDYGKVERKLVKCESSSRLGLHS |
| 554 | IDFWSVGRKAQQRKLVQGPSLIGSRSMRY |
| 555 | IDFWSVGSKPQTRRLVDGSRLIGHSSRSLRV |
| 556 | IDFWSVERGETRRRLLNPTPSAGSNRALSKR |
| 557 | VDFWSVGKPKPIRRLIQNDPGTDYDTGPKYR |
| 558 | VDFWSVEKPKPIRRLLNPGPNQGPYPNTGHR |
| 559 | VDFSHVDYGKWERKLIRSASTSRYGLRS |
| 560 | VDFSHVDYGKWERKTLRSRSLSRIGLTG |
| 561 | IDFWHVGYGKWERRLVKSASSSRFGIRG |
| 562 | VDFFHVDYGRWERKHIRCASMSRVGLRG |
| 563 | GTFQHVDYGKWERKPIRCQSMSRVGYRR |
| 564 | VGYGKWERKLVRPASLS |
| 565 | VEKGETRRRLLNPTPHA |
| 566 | VGYGKWERKLIRSASTV |
| 567 | VEKPKPIRRLLNPGPNQ |
| 568 | VDYGKWERKLIRSASTS |
| 569 | VDYGKWERKTLRSRSLS |
| 570 | VGYGKWERRLVKSASSS |
| 571 | VDYGRWERKHIRCASMS |
| 572 | VERPKPIRRLLTPTPGC |
| 573 | PFRAPTIKILSKQFTEDDIDFWHVGYGKWERKLVRPASLSGRRGL |
| | RR |
| 574 | PFRAPTVKILSKQFTDKDIDFSHVGYGKWERKMIRSASISRLGL |
| 575 | DIAFRAPTVKILSKQFTDRDVDFSHVGYGKWERKLIRSASTVKY |
| | GL |
| 576 | DIRFKPPTINILSKDYTADCVDFWSVEKPKPIRRLLNPGPNQGP |
| | YPNTG |
| 577 | DIPFRAPTVKIHSKQFSHRDVDFSHVDYGKWERKTLRSRSLSRI |
| | GL |
| 578 | DIPFRAPTVKILAKQFSEKDIDFWHVGYGKWERRLVKSASSSRF |
| | GI |
| 579 | DIPFRAPTVKILSKQFTDKDVDFFHVDYGRWERKHIRCASMSRV |
| | GL |
| 580 | DIKYKPPTIKILSKDYTADCVDFWSVERPKPIRRLLTPTPGCG |
| 581 | ARTKQTARKSTGGKAPRKQLATKAARKSAPATGGVKKPHRYR |
| | PGTVA |
| 582 | SGRGKGGKGLGKGGAKRHRKVLRDNIQGITKPAI |
| 583 | GRKKRRQRRR |

As used herein, the term “linker-bound cell-penetrating peptide” refers to a modified peptide or other entity that aides in cellular uptake of an RNA, e.g., by facilitating transfer of a cargo molecule from the membrane to the cytoplasm and nucleus, which is bound to a linking moiety that allows the cell-penetrating peptide to conjugate or attach to the trinucleotide cap analogs described herein. One skilled in the art would understand the linker bound to the cell-penetrating peptide can be chosen from those commercially available, such as biotin, 3′ maleimidobenzoic acid N-hydroxysuccinimide ester, and

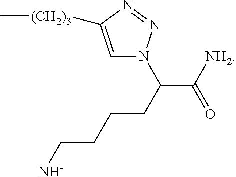

In some embodiments the cell penetrating peptides disclosed herein may be covalently attached to the RNA caps disclosed herein. By way of example, in some embodiments, the cell penetrating peptide may be incorporated into a fluorescent label that is attached to the RNA cap disclosed herein. In some embodiments, cell penetrating peptides are not covalently linked to the RNA caps disclosed herein. For example, in some embodiments, provided are compositions that comprise an RNA molecule with a CAP as described herein, in combination with one or more cell penetrating peptides.

As used herein, the terms “click” or “click chemistry,” as used herein, refer to the Huisgen cycloaddition or the 1,3-dipolar cycloaddition between an azide and an alkyne to form a 1,2,4-triazole.

As used herein, the term “enzymatically incorporatable” means that a nucleotide is capable of being enzymatically incorporated onto the terminus, e.g., 3′ terminus, of a polynucleotide chain, or internally through nick-translation of a polynucleotide chain, through action of a template-dependent or template-independent polymerase enzyme. A nucleotide-5′-triphosphate is an example of an enzymatically incorporatable nucleotide.

As used herein, the term “enzymatically extendable” or “3′ extendable” means a nucleotide or polynucleotide that is capable of being appended to a nucleotide or polynucleotide by enzyme action. A polynucleotide containing a 3′ hydroxyl group is an example of an enzymatically extendable polynucleotide.

As used herein, the term “halogen” refers to nonmetal elements of Group 7A of the Periodic Table of the Elements comprising fluorine, F, chlorine, Cl, bromine, Br, iodine, I, and astatine, At. Halogens are monovalent, readily form negative ions and occur as compounds or ions.

As used herein, the terms “intracellular molecular stability” and “intracellular stability” refers to the ability of RNA to exist in a cell without degradation leading to loss of function. Thus, increasing intracellular stability refers to an increase of the duration that an RNA exists in a cell. By way of non-limiting example, uncapped RNA can exist in a cell an average of 4 to 6 hours, whereas a capped RNA can exist an average of 10 to 48 hours depending on the cap.

As used herein, the term “locked nucleic acid” (LNA) refers to a bridge between the 2′O and 4′C methylene bicyclonucleotide monomers.

As used herein, the term “nucleobase” refers to a nitrogen containing heterocyclic moiety nucleobase. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil, 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(8-aza-7-deazaadenine).

As used herein, the term “nucleoside” refers to a compound consisting of a nucleobase linked to the C-1′ carbon of a ribose sugar or analog thereof. The ribose or analog may be substituted or unsubstituted. Substituted ribose sugars include, but are not limited to, those riboses in which one or more of the carbon atoms, preferably the 3′-carbon atom, is substituted with one or more of the same or different substituents such as R, OR, NRR or halogen (e.g., fluoro, chloro, bromo, or iodo), where each R group is independently H, C₁-C₆ alkyl or C₃-C₁₄ aryl. Particularly, riboses are ribose, 2′-deoxyribose, 2′,3′-dideoxyribose, 3′-haloribose (such as 3′-fluororibose or 3′-chlororibose) and 3′-alkylribose. Typically, when the nucleobase is A or G, the ribose sugar is attached to the N⁹-position of the nucleobase. When the nucleobase is C, T or U, the pentose sugar is attached to the N¹-position of the nucleobase (Kornberg and Baker, DNA Replication, 2^{nd} Ed., Freeman, San Francisco, Calif., (1992)). Examples of ribose analogs include arabinose, 2′-O-methyl ribose, and locked nucleoside analogs (e.g., WO 99/14226), for example, although many other analogs are also known in the art.

As used herein, the term “nucleotide” refers to a phosphate ester of a nucleoside as a monomer unit or within a polynucleotide.

As used herein, the term “nucleotide triphosphate” refers to a nucleotide with a triphosphate ester group at the 5′ position.

As used herein, nucleosides and/or nucleotides of the present teachings can comprise “natural sugars” (i.e., -ribose, 2′-deoxyribose, and the like) or sugar analogs.

The term “reporter moiety” and “reporter” are interchangeable and refer to a moiety that is detectable. In some embodiments, the reporter is specifically bound by an affinity moiety. In some embodiments, the interaction of the reporter moiety and the affinity moiety provides for the isolation of 1,4-triazole-derivatized RNA that is attached to the reporter moiety. Examples include, but are not limited to biotin or iminobiotin and avidin or streptavidin. A sub-class of reporter moiety is an “epitope tag,” which refers to a tag that is recognized and specifically bound by an antibody or an antigen-binding fragment thereof. Other reporters include, but are not limited to tags (with affinity partner), epitope tags (with antibody), and enzyme substrate (with enzyme). The reporter moiety can allow for attachment to a solid support for purification of the capped RNA. The reporter can be, for example, a dye, biotin, or a peptide. Examples of biotin molecules that can comprise the reporter moiety include C₅-C₂₀ O-biotin, SS-biotin, XX-biotin ((6-((6-((biotinoyl)amino)hexanoyl)amino)hexanoic acid succinimidyl ester), and NHS esters. For use in certain methods herein, the reporter includes an azide group to allow use in “click” technology.

As used herein, the term “sugar analog” refers to analogs of the sugar ribose. Exemplary ribose sugar analogs include, but are not limited to, substituted or unsubstituted furanoses having more or fewer than 5 ring atoms, e.g., erythroses and hexoses and substituted or unsubstituted 3-6 carbon acyclic sugars. Typical substituted furanoses and acyclic sugars are those in which one or more of the carbon atoms are substituted with one or more of the same or different R, OR, NRR or halogen groups, where each R is independently H, (C₁-C₆) alkyl or (C₁-C₁₄) aryl. Examples of substituted furanoses having 5 ring atoms include but are not limited to 2′-deoxyribose, 2′-(C₁-C₆)alkylribose, 2′-(C₁-C₆)alkoxyribose, 2′-(C₅-C₁₄)aryloxyribose, 2′,3′-dideoxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C₁-C₆)alkylribose, 2′-deoxy-3′-(C₁—C₆)alkoxyribose, 2′-deoxy-3′-(C₅-C₁₄)aryloxyribose, 3′-(C₁-C₆)alkylribose-5′-triphosphate, 2′-deoxy-3-′-(C₁-C₆)alkylribose-5′-triphosphate, 2′-deoxy-3′-(C₁-C₆)alkoxyribose-5′-triphosphate, 2′-deoxy-3′-(C₅-C₁₄)atyloxyribose-5′-triphosphate, 2′-deoxy-3′-haloribose-5′-triphosphate, 2′-deoxy-3′-aminoribose-5′-triphosphate, 2′,3′-dideoxyribose-5′-triphosphate or 2′,3′-didehydroribose-5′-triphosphate. Further sugar analogs also include so called locked nucleic acids (LNAs) having the structure

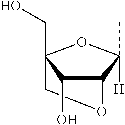

and those described in Wengel et al., WO 99/14226.

As used herein, the terms “polynucleotide”, “oligonucleotide” and “nucleic acid’ are used interchangeably and refer to single stranded and double stranded polymers of nucleotide monomers, including ribonucleotides (RNA) and 2′-deoxyribonucleotides (DNA) linked by internucleotide phosphodiester bond linkages. A polynucleotide may be composed entirely of deoxyribonucleotides, entirely of ribonucleotides or chimeric mixtures thereof.

As used herein, the term “terminator” means an enzymatically incorporatable nucleotide which prevents subsequent incorporation of nucleotides to the resulting polynucleotide chain and thereby halts polymerase-mediated extension. Typical terminators lack a 3′-hydroxyl substituent and include 2′,3′-dideoxyribose, 2′,3′-didehydroribose, and 2′,3′-dideoxy-3′-haloribose, e.g. 3′-deoxy-3′-fluoro-ribose or 2′,3′-dideoxy-3′-fluororibose, for example. Alternatively, a ribofuranose analog can be used, such as 2′,3′-dideoxy-β-D-ribofuranosyl, β-D-arabinofuranosyl, 3′-deoxy-β-D-arabinofuranosyl, 3′-amino-2′,3′-dideoxy-β-D-ribofuranosyl, and 2′,3′-dideoxy-3′-fluoro-β-D-ribofuranosyl (see, for example, Chidgeavadze et al., Nucleic Acids Res., 12:1671-1686 (1984), and Chidgeavadze et al., FEB. Lett., 183:275-278 (1985)). Nucleotide terminators also include reversible nucleotide terminators (Metzker et al., Nucleic Acids Res., 22(20):4259 (1994)).

As used herein, the term “TBDMS” refers to tert-butyldimethylsilyl.

As used herein the term “RNA delivery agent refers to one or more compounds (e.g., lipids, peptides and the like), that facilitate uptake of RNA molecules, such as the capped RNA molecules described herein, by a cell (in vitro or in vivo). Non-limiting examples of RNA delivery agents include cationic lipids and cell-penetrating peptides, optionally in combination with one or more neutral lipids, one or more PEG lipids, or any combination thereof.

Exemplary cationic lipids useful in the embodiments disclosed herein include, but are not limited to, 2,3-dioleyloxy-N-[2(sperminecarboxamido) ethyl]-N,N-dimethyl-1-propanaminium trifluoroacetate (DOSPA), 1,3-dioleoyloxy-2-(6-carboxy-spermyl) propylamide (DOSPER), dioctadecylamido-glycylspermine (DOGS), tetramethyltetrapalmitylspermine (TMTPS), tetramethyltetrapalmitoylspermin (TMTOS), tetramethlytetralauryl spermine (TMTLS), tetramethyltetramyristyl spermine (TMTMS), tetramethyldioleylspermine (TMDOS), N-1-dimethyl-N-1-(2,3-diaoleoyloxypropyl)-2-hydroxypropane-1,3-diamine, N-1-dimethyl-N-1-(2,3-diamyristyloxypropyl)-2-hydroxypropane-1,3-diamine, N-1-dimethyl-N-1-(2,3-diapalmityloxypropyl)-2-hydroxypropane-1,3-diamine, N-1-dimethyl-N-1-(2,3-diaoleoyloxypropyl)-2-(3-amino-2-hydroxypropyloxy)propane-1,3-diamine, N-1-dimethyl-N-1-(2,3-diamyristyloxypropyl)-2-(3-amino-2-hydroxypropyloxy)propane-1,3-diamine, N-1-dimethyl-N-1-(2,3-diapalmityloxypropyl)-2-(3-amino-2-hydroxypropyloxy)propane-1,3-diamine, L-spermine-5-carboxyl-3-(DL-1,2-dipalmitoyl-dimethylaminopropyl-β-hydroxyethylamine, 3,5-(N,N-di-lysyl)-diaminobenzoyl-glycyl-3-(DL-1,2-dipalmitoyl-dimethylaminopropyl-β-hydroxyethylamine), L-Lysine-bis(O,O′-oleoyl-β-hydroxyethyl)amide dihydrochloride, L-Lysine-bis-(O,O′-palmitoyl-β-hydroxyethyl)amide dihydrochloride, 1,4-bis[(3-(3-aminopropyl)-alkylamino)-2-hydroxypropyl)piperazine, L-Lysine-bis-(O,O′-myristoyl-O-hydroxyethyl)amide dihydrochloride, L-Ornithine-bis-(O,O′-myristoyl-β-hydroxyethyl)amide dihydrochloride, L-Ornithine-bis-(O,O′-oleoyl-β-hydroxyethyl)amide dihydrochloride, 1,4-bis[(3-(3-aminopropyl)-oleylamino)-2-hydroxypropyl]piperazine, L-Ornithine-bis-(O,O′-palmitoyl-β-hydroxyethyl)amide dihydrochloride, 1,4,-bis[(3-amino-2-hydroxypropyl)-oleylamino]-butane-2,3-diol, 1,4,-bis[(3-amino-2-hydroxypropyl)-palmitylamino]-butane-2,3-diol, 1,4,-bis[(3-amino-2-hydroxypropyl)-myristylamino]-butane-2,3-diol, 1,4-bis[(3-oleylamino)propyl]piperazine, L-Arginine-bis-(O,O′-oleoyl-β-hydroxyethyl)amide dihydrochloride, bis[(3-(3-aminopropyl)-myristylamino)2-hydroxypropyl]piperazine, L-Arginine-bis-(O,O′-palmitoyl-β-hydroxyethyl)amide dihydrochloride, L-Serine-bis-(O,O′-oleoyl-O-hydroxyethyl)amide dihydrochloride, 1,4-bis[(3-(3-aminopropyl)-palmitylamino)-2-hydroxypropyl]piperazine, Glycine-bis-(O,O′-palmitoyl-β-hydroxyethyl)amide dihydrochloride, Sarcosine-bis-(O,O′-palmitoyl-β-hydroxyethyl)amide dihydrochloride, L-Histidine-bis-(O,O′-palmitoyl-β-hydroxyethyl)amide dihydrochloride, cholesteryl-3β-carboxyl-amidoethylenetrimethylammonium iodide, 1,4-bis[(3-myristylamino)propyl]piperazine, 1-dimethylamino-3-trimethylammonio-DL-2-propyl-cholesteryl carboxylate iodide, cholesteryl-3β-carboxyamidoethyleneamine, cholesteryl-3β-oxysuccinamidoethylenetrimethylammonium iodide, 1-dimethylamino-3-trimethylammonio-DL-2-propyl-cholesteryl-3β-oxysuccinate iodide, 2-[(2-trimethylammonio)-ethylmethylamino]ethyl-cholesteryl-3β-oxysuccinate iodide, 3β-[N—(N′,N′-dimethylaminoethane)carbamoyl]cholesterol, and 3β-[N-(polyethyleneimine)-carbamoyl]cholesterol, 1,4-bis[(3-palmitylamino)propyl]piperazine, L-Ornithylglycyl-N-(1-heptadecyloctadecyl)glycinamide, N²,N⁵-Bis(3-aminopropyl)-L-ornithylglycyl-N-(1-heptadecyloctadecyl)glycinamide, 1,4-bis[(3-(3-amino-2-hydroxypropyl)-alkylamino)-2-hydroxypropyl]piperazine N²—[N²,N⁵-Bis(3-aminopropyl)-L-ornithyl]-N,N-dioctadecyl-L-glutamine, N²—[N²,N⁵-Bis(aminopropyl)-L-ornithyl]-N—N-dioctadecyl-L-α-glutamine, 1,4-bis[(3-(3-amino-2-hydroxypropyl)-oleylamino)2-hydroxypropyl]piperazine, N²—[N²,N⁵-Bis(aminopropyl)-L-ornithyl]-N—N-dioctadecyl-L-α-asparagine, N—[N²—[N²,N⁵-Bis[(1,1-dimethylethoxy)carbonyl]-N²,N⁵-bis[3-[(1,1-dimethylethoxy)carbonyl]aminopropyl]-L-ornithyl-N—N-dioctadecyl-L-glutaminyl]-L-glutamic acid, N²—[N²,N⁵-Bis(3-aminopropyl)-L-ornithyl]-N,N-diolyl-L-glutamine, N²—[N²,N⁵-Bis(aminopropyl)-L-ornithyl]-N—N-dioleyl-L-α-glutamine, 4-bis[(3-(3-amino-2-hydroxypropyl)-myristylamino)-2-hydroxypropyl]piperazine, N²—[N²,N⁵-Bis(aminopropyl)-L-ornithyl]-N—N-dioleyl-L-α-asparagine, N—[N²-N⁵-Bis[(1,1-dimethylethoxy)carbonyl]-N²,N⁵-bis[3-[(1,1-dimethylethoxy)carbonyl]aminopropyl]-L-ornithyl-N—N-dioleyl-L-glutaminyl]-L-glutamic acid, 1,4-bis[(3-(3-aminopropyl)-oleylamino)propyl]piperazine, N²—[N²,N⁵-Bis(3-aminopropyl)-L-ornithyl]-N,N-dipalmityl-L-glutamine, N²—[N²,N⁵-Bis(aminopropyl)-L-ornithyl]-N—N-dipalmityl-L-α-glutamine, N²—[N²,N⁵-Bis(aminopropyl)-L-ornithyl]-N—N-dipalmityl-L-α-asparagine, N—[N²—[N²,N⁵-Bis[(1,1-dimethylethoxy)carbonyl]-N²,N⁵-bis[3-[(1,1-dimethylethoxy)carbonyl]aminopropyl]-L-ornithyl-N—N-dipalmityl-L-glutaminyl]-L-glutamic acid, N²—[N²,N⁵-Bis(3-aminopropyl)-L-ornithyl]-N,N-dimyristyl-L-glutamine, N²—[N²,N⁵-Bis(aminopropyl)-L-ornithyl]-N—N-dimyristyl-L-α-glutamine, N²—[N²,N⁵-Bis(aminopropyl)-L-ornithyl]-N—N-dimyristyl-L-α-asparagine, 1,4-bis[(3-(3-amino-2-hydroxypropyl)-palmitylamino)-2-hydroxypropyl]piperazine, N—[N²—[N²,N⁵-Bis[(1,1-dimethylethoxy)carbonyl]-N²,N⁵-bis[3-[(1,1-dimethylethoxy)carbonyl]aminopropyl]-L-ornithyl-N—N-dimyristyl-L-glutaminyl]-L-glutamic acid, 1,4-bis[(3-(3-aminopropyl)-myristylamino)propyl]piperazine, N²—[N²,N⁵-Bis(3-aminopropyl)-L-ornithyl]-N,N-dilaureyl-L-glutamine, N²-8 N²,N⁵-Bis(aminopropyl)-L-ornithyl]-N—N-dilaureyl-L-α-glutamine, N²—[N²,N⁵-Bis(aminopropyl)-L-ornithyl]-N—N-dilaureyl-L-α-asparagine, N—[N²—[N²,N⁵-Bis[(1,1-dimethylethoxy)carbonyl]-N²,N⁵-bis[3-[(1,1-dimethylethoxy)carbonyl]aminopropyl]-L-ornithyl-N—N-dilaureyl-L-glutaminyl]-L-glutamic acid, 3-[N′,N″-bis(2-tertbutyloxycarbonylaminoethyl)guanidino]-N,N-dioctadec-9-enylpropionamide, 3-[N′,N″-bis(2-tertbutyloxycarbonylaminoethyl)guanidino]-N,N-dipalmitylpropionamide, 3-[N′,N″-bis(2-tertbutyloxycarbonylaminoethyl)guanidino]-N,N-dimyristylpropionamide, 1,4-bis[(3-(3-aminopropyl)-palmitylamino)propyl]piperazine, 1,4-bis[(3-(3-amino-2-hydroxypropyl)-oleylamino)propyl]piperazine, N,N-(2-hydroxy-3-aminopropyl)-N-2-hydroxypropyl-3-N,N-diolylaminopropane, N,N-(2-hydroxy-3-aminopropyl)-N-2-hydroxypropyl-3-N,N-dipalmitylaminopropane, N,N-(2-hydroxy-3-aminopropyl)-N-2-hydroxypropyl-3-N,N-dimyristylaminopropane, 1,4-bis[(3-(3-amino-2-hydroxypropyl)-myristylamino)propyl]piperazine, [(3-aminopropyl)-bis-(2-tetradecyloxyethyl)]methyl ammonium bromide, [(3-aminopropyl)-bis-(2-oleyloxyethyl)]methyl ammonium bromide, [(3-aminopropyl)-bis-(2-palmityloxyethyl)]methyl ammonium bromide, Oleoyl-2-hydroxy-3-N,N-dimethyamino propane, 2-didecanoyl-1-N,N-dimethylaminopropane, palmitoyl-2-hydroxy-3-N,N-dimethyamino propane, 1,2-dipalmitoyl-1-N,N-dimethylaminopropane, myristoyl-2-hydroxy-3-N,N-dimethyamino propane, 1,2-dimyristoyl-1-N,N-dimethylaminopropane, (3-Amino-propyl)->4-(3-amino-propylamino)-4-tetradecylcarbamoyl-butylcarbamic acid cholestryl ester, (3-Amino-propyl)->4-(3-amino-propylamino-4-carbamoylbutylcarbamic acid cholestryl ester, (3-Amino-propyl)->4-(3-amino-propylamino)-4-(2-dimethylamino-ethylcarbamoyl)-butylcarbamic acid cholestryl ester, Spermine-5-carboxyglycine (N′-stearyl-N′-oleyl) amide tetratrifluoroacetic acid salt, Spermine-5-carboxyglycine (N′-stearyl-N′-elaidyl) amide tetratrifluoroacetic acid salt, Agmatinyl carboxycholesterol acetic acid salt, Spermine-5-carboxy-β-alanine cholesteryl ester tetratrifluoroacetic acid salt, 2,6-Diaminohexanoeyl β-alanine cholesteryl ester bistrifluoroacetic acid salt, 2,4-Diaminobutyroyl β-alanine cholesteryl ester bistrifluoroacetic acid salt, N,N-Bis(3-aminopropyl)-3-aminopropionyl β-alanine cholesteryl ester tristrifluoroacetic acid salt, [N,N-Bis(2-hydroxyethyl)-2-aminoethyl]aminocarboxy cholesteryl ester, Stearyl carnitine ester, Palmityl carnitine ester, Myristyl carnitine ester, Stearyl stearoyl carnitine ester chloride salt, L-Stearyl Stearoyl Carnitine Ester, Stearyl oleoyl carnitine ester chloride, Palmityl palmitoyl carnitine ester chloride, Myristyl myristoyl carnitine ester chloride, L-Myristyl myristoyl carnitine ester chloride, 1,4-bis[(3-(3-amino-2-hydroxypropyl)-palmitylamino)propyl]piperazine, N-(3-aminopropyl)-N,N′-bis-(dodecyloxyethyl)-piperazinium bromide, N-(3-aminopropyl)-N,N′-bis-(oleyloxyethyl)-piperazinium bromide, N-(3-aminopropyl)-N,N′-bis-(palmityloxyethyl)-piperazinium bromide, N-(3-aminopropyl)-N,N′-bis-(myristyloxyethyl)-piperazinium bromide, N-(3-aminopropyl)-N′-methyl-N,N′-(bis-2-dodecyloxyethyl)-piperazinium bromide, N-(3-aminopropyl)-N′-methyl-N,N′-(bis-2-oleyloxyethyl)-piperazinium bromide, N-(3-aminopropyl)-N′-methyl-N,N′-(bis-2-palmityloxyethyl)-piperazinium bromide, N-(3-aminopropyl)-N′-methyl-N,N′-(bis-2-myristyloxyethyl)-piperazinium bromide, Phospholipids useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₆ Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE),

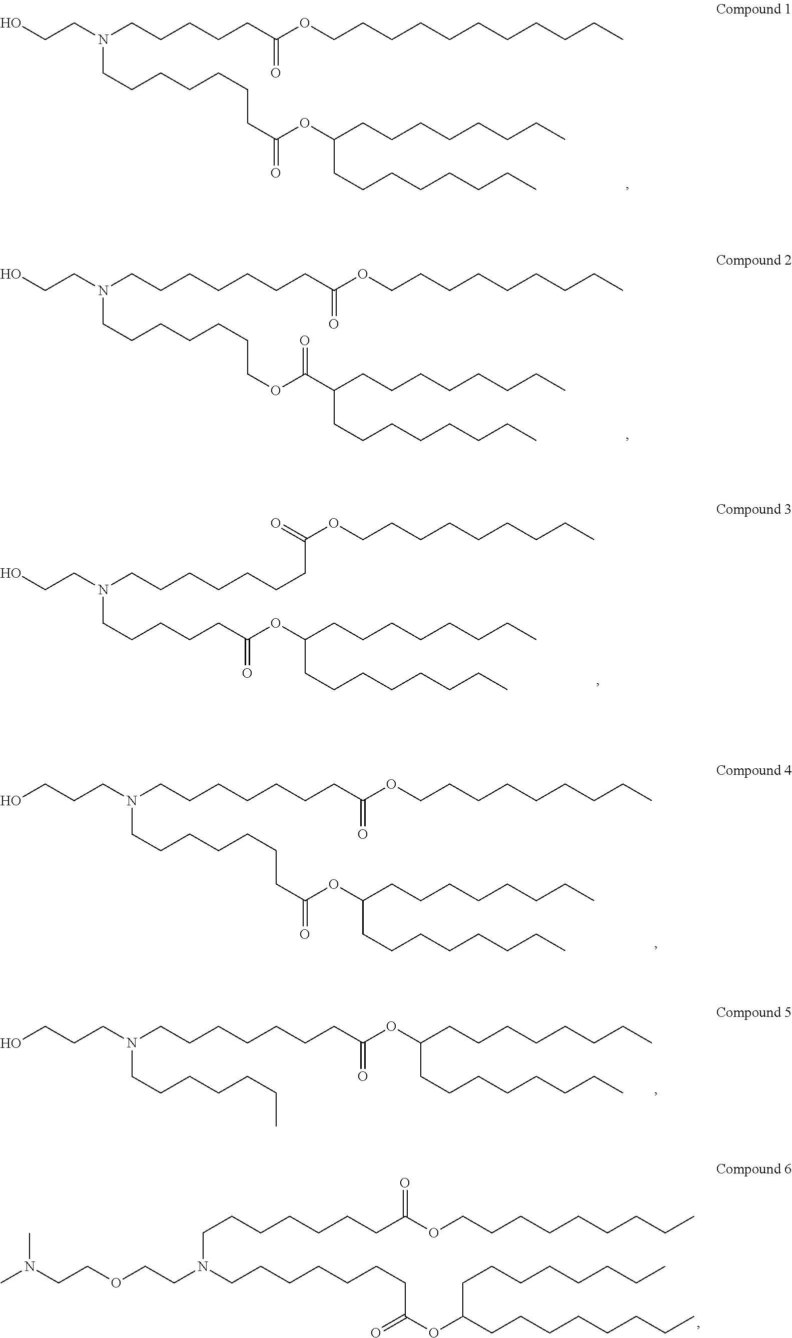

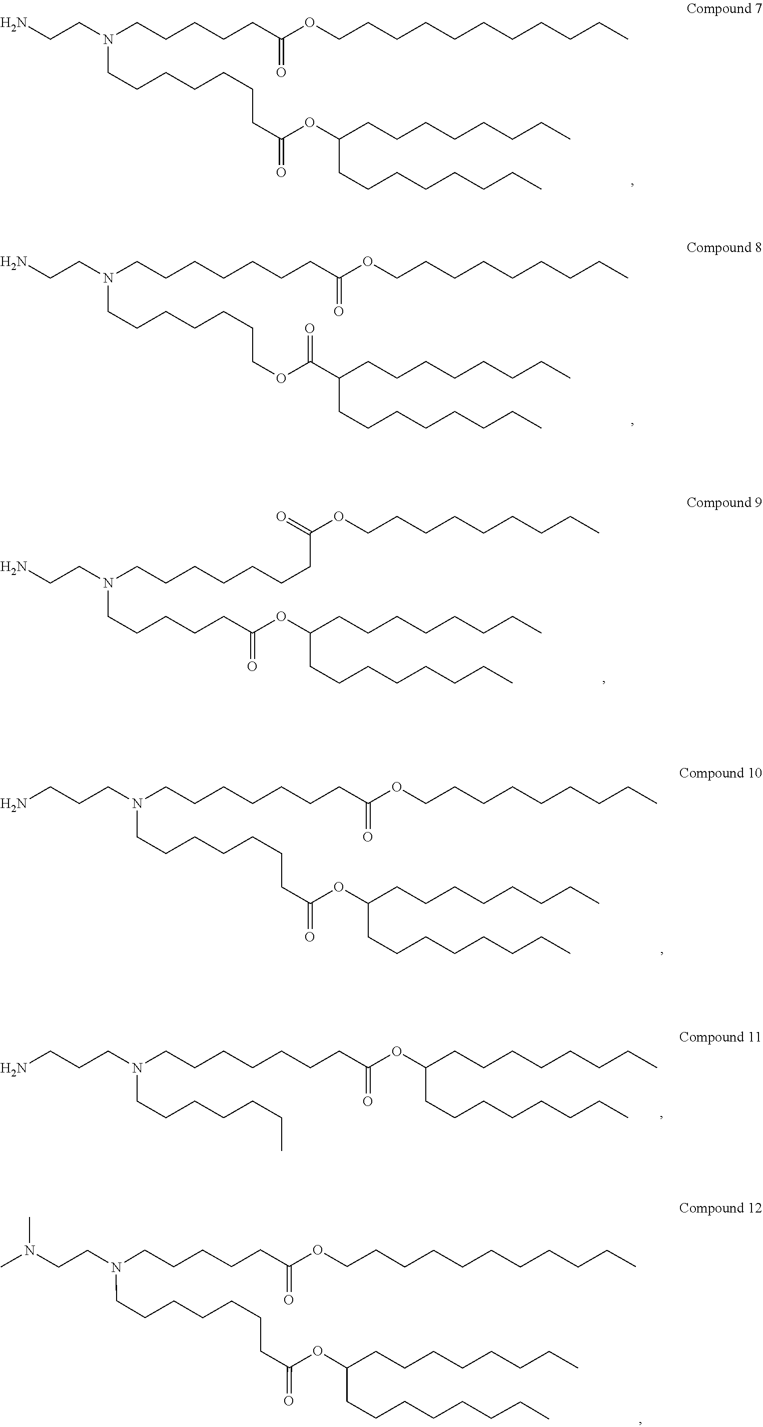

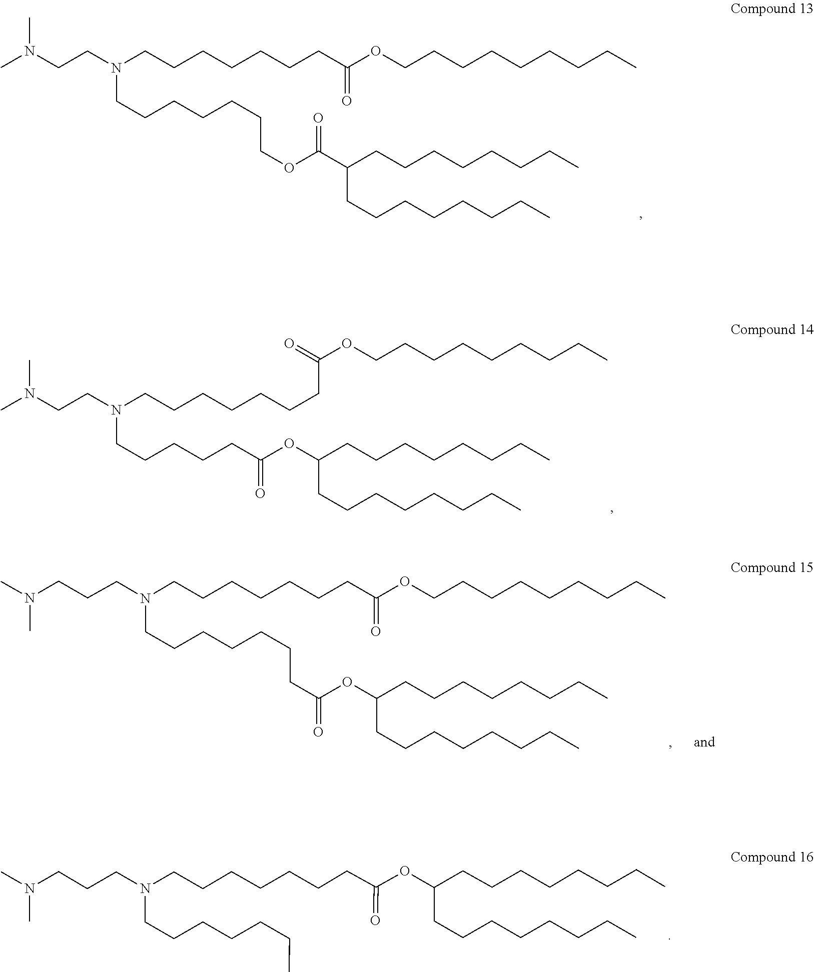

In some embodiments, an RNA delivery agent as described herein can include one or more neutral, or uncharged lipids. Neutral lipids useful in the embodiments described herein include, for example cholesterol, dioleoylphosphatidylethanolamine (DOPE), Dioleoylphosphatidylcholine (DOPC), and diphytanoylphosphatidylethanolamine (DDhPE) fecosterol, sitosterol, ergosterol, campesterol, stigmasterol, brassicasterol, tomatidine, tomatine, ursolic acid, alpha-tocopherol, or combinations thereof.

RNA delivery agent as described herein can include one or more agents that inhibit aggregation of lipid/RNA particles. An exemplary agent that inhibits aggregation of lipid/RNA particles includes PEG lipids, or PEGylated lipids, or polyglyceral lipids, e.g., as described in Fiedl, et al. (2020) Nanomedicine 15(19), 1829-1841.

RNA delivery agents as described herein can include one or more PEG lipids. As used herein, a “PEG lipid” or “PEGylated lipid” refers to a lipid comprising a polyethylene glycol component. A PEG lipid may be selected from the non-limiting group consisting of PEG-modified phosphatidylethanolamines, PEG-modified phosphatidic acids, PEG-modified ceramides (PEG-CER), PEG-modified dialkylamines, PEG-modified diacylglycerols (PEG-DEG), PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

The RNA can be transfected into a cell to be translated intracellularly. Methods of transfection are known to those of skill in the art and include microinjection, electroporation, transfection, chemical treatments and the like. In some aspects, the RNA compositions provided herein are delivered to cells in vitro. In some aspects, the RNA compositions provided herein can be used for ex vivo delivery of mRNA to cells. In other aspects, the RNA compositions provided herein can be used for in vivo delivery of mRNA to cells, e.g., in the context of mRNA vaccines or the like. Cells for use in in vivo translation include any patient cell for which it is desired to express a protein of interest. Non-limiting examples of cells useful in the embodiments disclosed herein include immune cells (e.g., T cells, B cells, NK cells, dendritic cells, macrophages, etc.), liver cells, lung cells, pancreatic cells, bone marrow cells, tissue culture cells, germ cells, stem cells such as induced pluripotent stem cells (iPSCs), human embryonic stem cells (hESCs), mesenchymal stem cells (MSCs), adipose-derived stem cells (ADSCs), and the like.

Dyes that are suitable for use are known to those skilled in the art and include, but are not limited to coumarin, cyanine, benzofuran, a quinoline, a quinazolinone, an indole, a benzazole, a borapolyazaindacene and xanthenes including fluorescein, rhodamine and rhodol as well as other dyes described in RICHARD P. HAUGLAND, MOLECULAR PROBES HANDBOOK OF FLUORESCENT PROBES AND RESEARCH CHEMICALS (11^{th} edition, January 2010.

Fluorescent dyes used herein include, without limitation; a pyrene (including any of the corresponding derivative compounds disclosed in U.S. Pat. Nos. 5,132,432 and 8,039,642), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine (including any corresponding compounds in U.S. Pat. Nos. 6,977,305; 6,974,873; 6,664,047; 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445, 7,446,202; 7,598,390; 7,776,529; PCT International Publication Nos. WO 02/26891, WO 97/40104, WO 99/51702, WO 01/21624, WO 2018/085449; and European Patent Application Publication No. 1 065 250 A1), a benzocyanine (including any corresponding compounds in U.S. Pat. Nos. 9,249,307; 9,751,868; 10,000,467; 10,053,447; 10,125,120; 10,351,551; 10,526,317; and US2017/0158858); a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343 and 6,716,979), an oxazine (including any corresponding compounds disclosed in U.S. Pat. No. 4,714,763) or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs.

When the dye is a xanthene, the dye is optionally a fluorescein, a rhodol (including any corresponding compounds disclosed in U.S. Pat. Nos. 5,227,487 and 5,442,045), or a rhodamine (including any corresponding compounds in U.S. Pat. Nos. 5,798,276; 5,846,737; 6,562,632; 7,256,292; 7,985,602; 8,729,267; 9,040,674; 9,315,859; 9,745,336; 9,783,560; 9,790,544; 10,131,936).

Typically the fluorescent dye contains one or more aromatic or heteroaromatic rings, that are optionally substituted one or more times by a variety of substituents, including without limitation, halogen, nitro, cyano, alkyl, perfluoroalkyl, alkoxy, alkenyl, alkynyl, cycloalkyl, arylalkyl, acyl, aryl or heteroaryl ring system, benzo, or other substituents typically present on fluorescent dyes known in the art.

### Caps/Cap Analogs and Promoters

Provided herein are compositions and methods in which (1) RNA caps and cap analogs and (2) promoters function to result in the production of capped RNA, such as messenger RNA (mRNA). In some instances, these compositions and methods relate to mRNA caps or cap analogs that interact with specific promoters (e.g., modified naturally occurring promoters) in manner that allows for the production of capped RNA.

In some instances, compositions and methods provided herein will be designed interact in a manner that result in the high yield production of mRNA with high capping efficiency. As used herein, high yield production of mRNA refers to 3 mg/ml (60 μg/20 μl) or higher of RNA. As used herein, RNA capping efficiency refers to the percentage of RNA present in a composition that contains caps. By way of example, if an uncapped population of RNA molecules is subjected to a process by which caps or cap analogs are added to these molecules, capping efficiency would be determined by the percentage of RNA molecules in the resulting composition containing caps. Similarly, when capped mRNA is formed by transcription, capping efficiency would also be determined the percentage of RNA molecule in the resulting composition containing caps. High capping efficiency refer to the production of RNA population where the percentage of the capped RNA molecules (e.g., target RNA molecules) compared to uncapped RNA molecules (e.g., target RNA molecules) is greater than or equal to 70% (e.g., from about 75% to about 99%, from about 80% to about 99%, from about 85% to about 99%, from about 90% to about 99%, from about 95% to about 99%, from about 75% to about 95%, from about 75% to about 90%, from about 80% to about 95%, from about 85% to about 95%, from about 75% to about 100%, from about 80% to about 100%, from about 90% to about 100%, from about 95% to about 100%, etc.). The term “target RNA molecules” refers to RNA molecules which are the desired subject of capping processes. By way of example, if a coupled transcription/translation system is used, then ribosomal RNA molecules present for translation would not be considered to “target RNA molecules”. Said another way, capping efficiency relates to the RNA molecule that are intended for capping (e.g., RNA molecules coding for specific proteins) and does not include other RNA molecules in a reaction mixture.

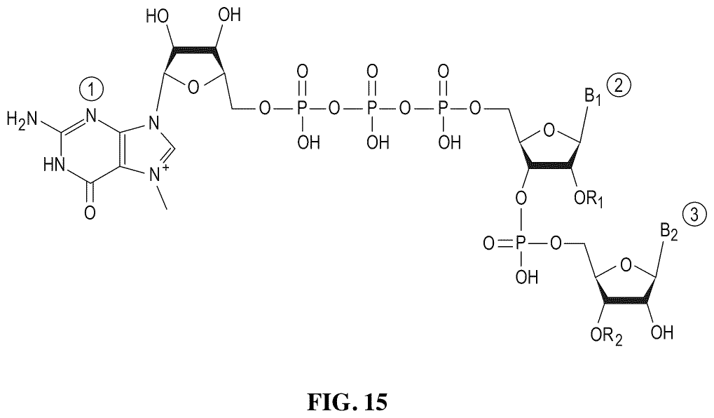

FIG. 15 shows the structure of an exemplary 7-methylguanosine trinucleotide cap analog, that in many instances set out here may function as a capped primer. This cap analog schematic indicates where the bases can be located at B₁ and B₂. The three bases of this cap analog are labeled with the numbers 1, 2 and 3 in circles. Of course, any number of cap analog variations may be present in compositions and used in methods set out herein.

Caps and cap analogs may vary substantially in terms of nucleotide sequences. Using the cap analog structure set out in FIG. 15 for purposes of illustration, the first bases, labeled with circled number 1, will generally be G. The other two bases, labeled with circled numbers 2 and 3, will generally be complementary to one or more bases at initiations sites of nucleic acid molecules in reaction mixture for which transcription is desired. Further, the initiation site may vary from the positions marked off as such in FIG. 16.

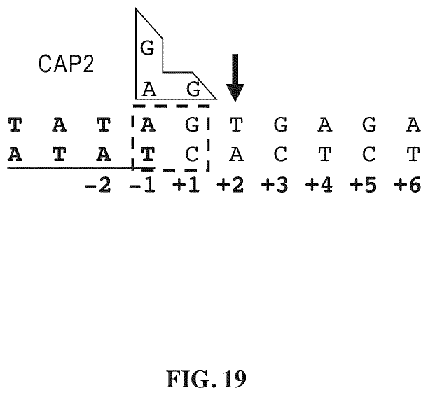

The nucleotide sequence of one exemplary capped primer is shown in FIG. 19 as GAG. This AG portion of this capped primer (cap analog positions 2 and 3) are complementary to positions −1 and +1 of the initiation region of the template strand shown. In this instance, positions −1 and +1 of the initiation regions are referred to herein as the initiation site. Exemplary capped primer nucleotide sequences and initiation sites they are complementary to are set out in

TABLE 3
Table 3: Exemplary Cap/Initiation Site Specifications

| | Capped Primer | Capped Primer | Initiation | Promoter Positions | |
|---|---|---|---|---|---|
| No. | Sequence | Type | Site | (FIG. 16) | |
| 1 | GAG | Trimer | TC | −1 to +1 | +2 to +3 |
| 2 | GAU | Trimer | TA | −1 to +1 | +2 to +3 |
| 3 | GAGG | Tetramer | TCC | −1 to +2 | +2 to +4 |
| 4 | GAGGG | Pentamer | TCCC | −1 to +3 | +2 to +5 |
| 5 | GAGGGU | Hexamer | TCCCA | −1 to +4 | +2 to +6 |
| 6 | GGG | Trimer | CC | +1 to +2 | +2 to +3 |
| 7 | GGGG | Tetramer | CCC | +1 to +3 | +2 to +5 |
| 8 | GAA | Trimer | TT | −1 to +1 | +2 to +3 |
| 9 | GGA | Trimer | CT | −1 to +1 | +2 to +3 |

RNA caps and cap analogs that may be contained in compositions and used in methods set out herein include those set out herein and specifically include commercially available trimer caps and cap analogs, such as those sold by TriLink Biotechnologies (San Diego, CA) (e.g., CLEANCAP® Reagent GG, cat. no. N-7133; CLEANCAP® Reagent AU, cat. no. N-7114; CLEANCAP® Reagent GG (3′ OMe), cat. no. N-7433; CLEANCAP® Reagent AG (3′ OMe), cat. no. N-7413; and CLEANCAP® Reagent AG, cat. no. N-7113).

As shown in Table 3, capped primers containing more than three nucleotides may be used in the practice of subject matter set out herein. Such caps and cap analogs may contain from about three to about twenty bases (e.g., from about three to about nineteen, from about three to about eighteen, from about three to about fifteen, from about three to about twelve, from about three to about ten, from about three to about eight, from about three to about six, from about three to about four, from about four to about ten, from about four to about eight, etc.). Further, such caps and cap analogs may have sequence complementarity to the template strand of a promoter initiation site.

FIG. 16 shows a comparison of four bacteriophage promoters of T7, T3, SP6 and K11 phages. Each of the bacteriophage promoters shown in FIG. 16 are 19 nucleotides in length and include a ten nucleotide initiation region at positions −4 to +6. Nucleotides +1 to +6 are referred to herein as the transcription start region, with initiation typically beginning at position +1. In bacteriophage promoters, the +1 position is conserved as a G and positions +2 to +5 are conserved as purines (A and G). Thus, pyrimidines (T and C) are typically not found at these positions. Further, FIG. 16 shows only purine bases present from positions −1 to +5. Also the only promoter shown to contain a purine (i.e., C) at position +6 is the K11 promoter.

Promoter positions −5 to −12 interact with a T7 RNA polymerase structural domain located near the carboxyl terminus of the protein. The AT rich region (positions −17 to −13) is believed to interact with a T7 RNAP structural domain located near the amino terminus of the protein.

FIG. 17 shows three different caps designed to have sequence complementarity at or near the transcriptional initiation region. CAP1 is a dinucleotide cap with sequence complementarity to the +1 position of the transcriptional initiation region. CAP2 is a trinucleotide cap with sequence complementarity to the −1 and +1 positions of the transcriptional initiation region. CAP3 is a trinucleotide cap with sequence complementarity to the +1 and +2 positions of the transcriptional initiation region.

Ishikawa et al., “Preparation of eukaryotic mRNA having differently methylated adenosine at the 5′-terminus and the effect of the methyl group in translation”, Nucleic Acids Symposium Series No. 53, pages 129130 Oxford University Press (2009), performed a study using several different mRNA cap similar to CAP2 shown in FIG. 17 and showed that these caps could initiate transcription on template with 2′-deoxycytidine residues at template positions +1 and +2 (“CC” template; Nucleic Acids Symposium Series No. 53: 129 (2009)). The authors state, “The different result from the case of using ^{m7}G5′pppG may be caused from base pairing between additional adenosine (N1) in ^{m7}G5′pppN1pG and 2′-deoxythymidine in T7 promoter at −1 position.”

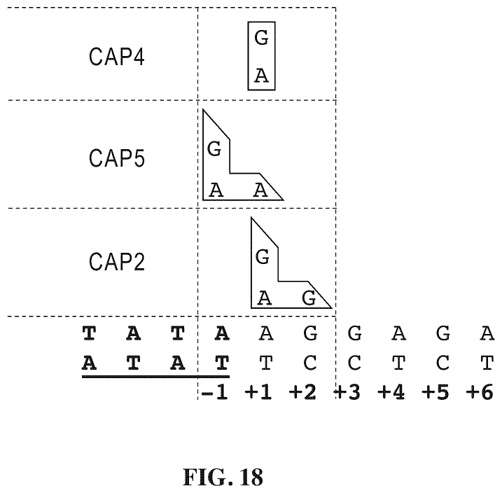

FIG. 18 is similar to FIG. 17 but the base pairs at +1 position of the initiation region have been changed from G/C to A/T. Also, the CAP4, CAP5 and CAP2 are positioned above their cognate complementary hybridization initiation sequences.

FIG. 19 is similar to FIGS. 17 and 18 but it shows only a single mRNA cap, CAP2, and above its cognate complementary hybridization initiation sequence at positions −1 and +1. Further, the initiation region is structured so that neither of the two bases of the cap that hybridized to the initiation region are complementary to the immediate flanking bases at positions −2 and +2.

A number of promoters and modified promoters may be present in compositions and used in methods provided herein. Using the schematic of FIG. 16 for reference, promoters present in compositions and used in methods provided herein may be wild-type promoters or maybe be modified in some manner. Such modifications include (1) 5′ and 3′ truncations and/or (2) internal substitutions and/or deletions.

When transcriptional initiation at the −1 or +1 position is desired, promoters may be designed to facilitate such initiation. For example, when a trinucleotide capped primer is used for initiation at the −1 position, then promoters having the following non-template strand nucleotide sequences may be used: TATY₁ Y₂Z. In this context, Y₁ is at the −1 position, Y₂ is at the +1 position, and Z is at position +2, which is “adjacent” to +1 end of the initiation site. By “adjacent” is meant that a base is located as the first base before and/or after the initiation site. Further, when a trinucleotide capped primer is used for initiation at the +1 position, then promoters having the following non-template nucleotide sequences may be used: TATA Y₁Y₂Z. In these instances, Y₁ and Y₂ are the same base as the second and third bases of the trinucleotide capped primer. Thus, the template strand would contain bases at positions corresponding to Y₁ and Y₂ that are complementary to the bases of the trinucleotide capped primer. Further, Z is transcriptional blocking nucleotide, the base of which may independently be A, T or C, as well as a chemically modified nucleotide.

By way of illustration, when a GAG primer is used for initiation at the −1 position, then suitable promoters include those comprising the following nucleotide sequences: (1) 5′-TATA GA-3′, (2) 5′-TATA GT-3′, and (3) 5′-TATA GC-3′.

When transcriptional initiation at the +2 position is desired, promoters may also be designed to facilitate such initiation. For example, when a trinucleotide capped primer is used for initiation, then promoters having the following non-template strand nucleotide sequences may be used: TATA X₁Y₁Y₂X₂, where Y₁ and Y₂ (located at positions +2 and +3) are the same as the second and third bases of the trinucleotide capped primer. Further, X₁ and X₂ are transcriptional blocking nucleotides (located at positions +1 and +4), the bases of which may independently be A, T or C, or chemically modified nucleotides. In this context, Y₁ is at the +2 position, Y₂ is at the +3 position, and X₁ and X₂ at positions +1 and +4 are said to be “adjacent” to each end of the +2/+3 initiation site. Further, in some instances, X₁ may be a transcriptional blocking nucleotide and X₂ is not a transcriptional blocking nucleotide

By way of illustration, when a GAG primer is used for transcriptional initiation at the +2 position, then suitable promoters include those comprising the following nucleotide sequences: (1) 5′-TATA TAGA-3′, (2) 5′-TATA TAGT-3′, (3) 5′-TATA TAGC-3′, (4) 5′-TATA AAGA-3′, (5) 5′-TATA AAGT-3′, (6) 5′-TATA CAGC-3′, and (7) 5′-TATA CAGA-3′.

Provided herein are compositions, as well as methods for using such compositions, for the production of RNA in which transcriptional initiation occurs at a position other than the natural +1 transcriptional initiation position. As examples, initiation may occur at the −2/−1, −1/+1, +2/+3, −1/+1/+2, −1/+1/+2/+3, or +2/+3/+4 positions. In many instances, the bases A, T or C may be located at one or both positions adjacent to one or both termini of initiation sites. Further, other transcriptional initiation blocking nucleotides may be located at the same positions.

Also, provided herein are compositions, as well as methods for using such compositions, comprising multimeric capped primers comprising three of more nucleotides (e.g., from about three to about ten, from about three to about eight, from about three to about seven, from about three to about five, from about three to about four, from about four to about eight, etc., nucleotides) and promoters comprising transcriptional initiation sites in which the template strand is complementary to bases of the capped primers. In many instances, the capped primers will be designed to hybridize to transcriptional initiation sites located in positions other than at, or in addition to, the +1/+2 positions. In many additional instances, the bases A, T, or C will be located at one or both position adjacent to initiation sites (e.g., at position +1 and +4, when a +2/+3 initiation site is used). Further provided herein are compositions, as well as methods for using such compositions, comprising trimeric caps and promoters comprising +1/+2 transcriptional initiation sites where the base at position +3 is A, T or C.

Further provided herein are compositions, as well as methods for using such compositions, for transcriptional initiation using multimeric capped primers greater than three nucleotides in length. An exemplary tetrameric capped primers primer has the nucleotide sequence GAGG. When this capped primer is used to initiate transcription at the +1/+2+3 position, the promoter used for transcriptional initiation may comprise one of the following nucleotide sequences: (1) 5′-TATA AGGA-3′, (2) 5′-TATA AGGT-3′, (3) 5′-TATA AGGC-3′, (4) 5′-TATA GAGGT-3′, (5) 5′-TATA GAGGA-3′, and (6) 5′-TATA GAGGC-3′.

In some aspects, provided herein are promoters that contain a transcriptional initiation site, flanked by transcriptional initiation blocking nucleotides. “Transcriptional initiation blocking nucleotide” are nucleotides that are not preferred for transcriptional initiation at the position they are located in. By way of example, with respect to T7 RNA polymerase promoters, the base thymine in the non-template strand may be used to increase capping efficiency when placed at position +2, proceeded by the sequence AG, and when a GAG capped primer is used for RNA capping. In this instance, thymidine at position +2 would be a transcriptional initiation blocking nucleotide.

Transcriptional initiation blocking nucleotides may be any nucleotide that is disfavored for transcriptional initiation while not significantly effecting transcriptional initiation at the desired initiation site. Transcriptional initiation blocking nucleotides function in conjunction with the promoter, capped primer, and reaction conditions being used. In some instances, transcriptional initiation blocking nucleotides may be deoxythymidine, thymidine, cytidine, adenosine, guanosine, and/or uridine. Transcriptional initiation blocking nucleotides may also be chemically modified. Further, such chemical modifications may be of the bases, the sugars, the phosphate linkages, or a combination of these.

The use of transcriptional initiation blocking nucleotides may increase capping efficiency by at least 20% (e.g., from about 20% to about 200%, from about 20% to about 180%, from about 20% to about 150%, from about 20% to about 120%, from about 20% to about 100%, from about 20% to about 80%, from about 20% to about 60%, from about 20% to about 40%, from about 30% to about 100%, from about 40% to about 90%, from about 50% to about 150%, from about 30% to about 60%, etc.). One exemplary assay for measuring increased capping efficiency is by comparing the capping efficiency under two different conditions. Under these exemplary conditions a GAG capped primer is used to produce capped mRNA with two different promoters. The non-template strand of one promoter comprises the nucleotide sequence TATA AGG and the other promoter comprises the nucleotide sequence TATA AGT, the difference being the presence of T at the +2 position.

Transcriptional initiation blocking nucleotides may be used in a number of different ways. Along these lines, the position and number of transcriptional initiation blocking nucleotides may vary. For example, more than one (e.g., one, two, three, etc.) transcriptional initiation blocking nucleotide may be adjacent to one or both termini of transcriptional initiation sites. One exemplary promoter sequence is as follows: 5′-TATA TAGTT-3′, where AG is the initiation site. In this instance, one transcriptional initiation blocking nucleotide is adjacent to the 5′ end of the initiation site and two transcriptional initiation blocking nucleotides are adjacent to the 3′ end of the initiation site.

### Transcription Reaction Mixtures

Variables in addition of caps and cap analogs to RNA molecules and promoters that can affect capped mRNA yield and capping efficiency include the composition of reaction mixtures used in the RNA production process (e.g., mRNA production process).

Some prior methods for generating capped mRNA through the use of capped primers use reagent mixtures in which the amount GTP present is lower amount than the amount of cap and the other three NTPs. This is so because if high concentrations of GTP are used with dimeric caps that initiate transcription at the +1 position with a G, then the GTP competes efficiently with the dimeric caps for initiation from the +1 nucleotides at NTP concentrations closer to the Kd (2 mM), producing large proportion of RNA that starts with pppG. While decreasing the GTP concentration results in a higher capping efficiency, it also results in lower capped mRNA yields. Provided herein are compositions and methods for the production of capped RNA molecules with both high yields and high capping efficiency.

Some aspects provided herein relate to IVT reaction mixtures that contribute to the production of mRNA populations in which a high percentage of the RNA molecules present are capped (high capping efficiency). In some aspects, IVT reaction mixtures and methods set out herein may be designed to result in high yield production of RNA. In additional aspects, IVT reaction mixtures and methods set out herein may be designed to result in both high capping efficiency and high yield production of RNA.

Reaction mixtures that may be employed to result in both high capping efficiency and high yield production of RNA may comprise chemically modified RNA components designed, for example, to enhance the production of mRNA and/or to stabilize RNA present in the reaction mixture and/or increase translation or reduced immunogenicity.

IVT reaction mixtures will generally contain the following components: (1) One or more RNA polymerase, (2) one or more cap (e.g., one or more capping primer), (3) all four standard nucleotide triphosphates (i.e., GTP, ATP, CTP, and UTP), and (4) one or more nucleic acid template (e.g., one or more DNA templates, one or more RNA templates, a combination of one or more DNA templates and one or more RNA templates, etc.).

IVT reaction mixtures used in methods set out herein may also contain one or more of the following components: (1) One or more buffer (e.g., phosphate, histidine, citrate, maleate, tartrate, acetate, tris-(hydroxymethyl)-aminomethane (tris), and bicarbonate, etc.), (2) one or more divalent metal ion (e.g., Ca²⁺, Mg²⁺, Mn²⁺, Co²⁺, Ni²⁺, etc.), (3) one or more chemically modified nucleotide triphosphate (e.g., pseudouridine (ψ) triphosphate, 1-methylpseudouridine (m¹ψ) triphosphate, 5-methoxyuridine (mo⁵U) triphosphate, 5-methylcytidine (n⁵C) triphosphate, α-thio-guanosine triphosphate, α-thio-adenosine triphosphate, etc.), (4) one or more polyamine (e.g., spermidine, spermine, tris(2-aminoethyl)amine, diethylenetriamine, etc.), (5) one or more reducing agent (e.g., DTT (2,3 dihydroxybutane-1,4-dithiol/, also referred to as “dithiothreitol”), DTE (2,3 dihydroxybutane-1,4-dithiol), thioglycolate, cysteine, sulfites, bisulfites, sulfides, bisulfides, TCEP (tris(2-carboxyethyl)phosphine), 2-mercaptoethanol, etc.), (6) one or more non-ionic detergent (e.g., octylphenoxypolyethoxyethanol (nonidet P-40); polyoxyethylene glycol sorbitan alkyl esters, such as Polysorbate 20 or Polysorbate 80; block copolymers of polyethylene glycol and polypropylene glycol (Poloxamers), such as Poloxamer 407; polyethoxylated tallow amine (POEA) salt; nonoxynols, such as Nonoxynol-9; Triton X-100; Tween 80, etc.), (7) one or more crowding agents (e.g., polyethylene glycol, dextran and ficoll, etc.) and/or (8) one or more RNAse inhibitors (e.g., one or more vanadyl ribonucleoside complex (VRC), one or more nucleotide analog, SUPERASE-IN™ (Thermo Fisher Scientific, cat. no. AM2696), RNASEOUT™ (Thermo Fisher Scientific, cat. no. 10777019), inorganic pyrophosphatase (e.g., Thermo Fisher Scientific, cat. no. EF0221), etc.).

The concentrations of nucleoside triphosphates and cap analog present in an IVT reaction mixture may vary. In some embodiments, NTPs and cap analog are present in the reaction at equirnolar concentrations. In some embodiments, the molar ratio of cap analog (e.g., trinucleotide cap) to nucleoside triphosphates in the reaction is greater than 1:1. For example, the molar ratio of cap analog to any one nucleoside triphosphate (e.g., ATP) in the reaction may be from about 1.1:1 to about 25:1, from about 2:1 to about 25:1, from about 3:1 to about 25:1, from about 5:1 to about 25:1, from about 1,1:1 to about 15:1, from about 2:1 to about 15:1, from about 4:1 to about 15:1, from about 6:1 to about 15:1, from about 8:1 to about 15:1, from about 1.1:1 to about 10:1, from about 2:1 to about 10:1, from about 3:1 to about 10:1, from about 4:1 to about 10:1, or from about 2:1 to about 6:1. In many instances, the molar ratio of cap analog to any one nucleoside triphosphate (e.g., ATP) in the reaction may be from about 1:1 to about 10:1 (e.g., from about 1,5:1 to about 8:1, from about 2:1 to about 8:1, from about 2:1 to about 6:1, from about 2:1 to about 5:1, from about 1.5:1 to about 5:1, from about 2:1 to about 4:1, etc.).

In some embodiments, the molar ratio of cap analog (e.g., trinucleotide cap) to any one nucleoside triphosphate in the reaction is less than 1:1. For example, the molar ratio of cap analog (e.g., trinucleotide cap) to nucleoside triphosphates in the reaction may be from about 1:1.1 to about 1:25, from about 1:2 to about 1:25, from about 1:4 to about 1:25, from about 1:5 to about 1:25, from about 1:1,1 to about 1:10, from about 1:2 to about 1:10, from about 1:4 to about 1:10, from about 1:2 to about 1:6, or from about 1:3 to about 1:6.

The concentrations of individual NTPs (e.g., the “standard” NTPs (ATP, UTP, CTP, GTP)) present in an IVT reaction may also vary. Further, such variances may be due to factors such as cap nucleotide sequences, the initiation site sequence, and/or the presence of “non-standard” NTPs (e.g., pseudouridine (ψ) triphosphate, I-methyl pseudouridine (m1ψ) triphosphate, 5-rnethoxyuridine (mo5U) triphosphate, etc.).

For purposes of illustration, when a dimer cap is used in an IVT reaction, then the concentration of the NTP capable of hybridizing at position +1 of the initiation site may be lower than the other NTPs in the reaction mixture. Thus, compositions and methods are provided herein where three standard NTPs are present in equimolar amount and one standard NTP is present in a lower amount. Using the promoter sequence in FIG. 17 for specific illustration, where the base at position +1 of the template strand is C. Thus, in some such instances, IVT reaction mixtures will contain equimolar amounts of ATP, UTP, and CTP but a lower amount of GTP. Further, the ratio of the three other NTPs to GTP may be from about 1:0.1 to about 1:0.95 (e.g., from about 1:0.1 to about 1:0.9, from about 1:0,2 to about 1:0.9, from about 1:0.25 to about 1:0.9, from about 1:0.3 to about 1:0.9, from about 1:0.4 to about 1:0.9, from about 1:0.2 to about 1:0.7, from about 1:0.25 to about 1:0.6, from about 1:0.15 to about 1:0.6, etc.). Further, if, for example, the dimer cap has the nucleotide sequence G-A, then the amount of ATP present in a transcription reaction mixture may be lower than for the other three NTPs.

In some instances, GTP, CTP and UTP may be used in excess of ATP in transcription reaction mixtures. As a non-limiting example, an IVT reaction may include 7.5 millimolar GTP, 7.5 millimolar CTP, 7.5 millimolar UTP, and 3.75 millimolar ATP. The same IVT reaction mixture may include 3.75 millimolar cap analog (e.g., trimer cap). In some instances, the molar ratio of G:C:U:A:cap may be 1:1:1:0.5:0.5, 1:1:0.5:1:0.5, 1:0.5:1:1:0.5, 0.5:1:1:1:0.5, 0,9:0.9:1:1:0.5, 0.9:0,9:1:0.5:0,5. In some instances, the ratio of one or both of the NTPs that form three hydrogen bond with their cognate bases (GTP and CTP) may be in lower ratios compared to the NTPS that form two hydrogen bonds (ATP and UTP), Further, the ratio ATP/UTP to CTP/UTP present in IVT reaction mixtures may be from 1.5 to 1 to 1.1 to 1.

RNA Molecules with Chemical Modifications

In some instances, it may be desirable to generate RNA molecules comprising one or more chemical modifications. In this context a chemical modification refers to a chemical alteration not normally found in RNA generated in IVT systems containing the four standard NTPs. Thus, chemical modifications include the well over 100 naturally occurring RNA chemical modifications, such as N 6-methyladenosine (m6A), pseudouridine, 3-methylcytidine (m3C), and 2-O-methyl modifications.

Examples of naturally-occurring nucleotides used for the production of RNA, e.g., in an IVT reaction, as provided herein include adenosine triphosphate (ATP), guanosine triphosphate (GTP), cytidine triphosphate (CT P), uridine triphosphate (UTP), and 5-methyluridine triphosphate (m⁵UTP). In some embodiments, adenosine diphosphate (ADP), guanosine diphosphate (GDP), cytidine diphosphate (CDP), and/or uridine diphosphate (UDP) are used. One method for generation RNA molecules (e.g., mRNA molecules) containing chemical modifications is by the inclusion of chemically modified nucleosides or other components in IVT reaction mixtures.

Examples of nucleotide analogs include that can be used in IVT reactions using the compositions described herein include, but are not limited to, antiviral nucleotide analogs, phosphate analogs (soluble or immobilized hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5 moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, a RNA transcript (e.g., mRNA transcript) of the present disclosure may include a modified nucleobase selected from pseudouridine (ψ), 1-methylpseudouridine (m¹ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudo uridine, 4-thio-1-methyl-pseudouridine, 4-thio-pseudouridine, 5-aza-uridine, dihydropseudouridine, 5-methyluridine, 5-methoxyuridine (mo⁵U) and 2′-—O-methyl uridine. In some embodiments, a RNA transcript (e.g., mRNA transcript) includes a combination of at least two (e.g., 2, 3, 4 or more) of the foregoing modified nucleobases.

The nucleoside triphosphates (NTPs) as provided herein may comprise unmodified or modified ATP, modified or unmodified UTP, modified or unmodified GTP, and/or modified or unmodified CTP. In some embodiments, NTPs of an IVT reaction comprise unmodified ATP. In some embodiments, NTPs of an IVT reaction comprise modified ATP. In some embodiments, NTPs of an IVT reaction comprise unmodified UTP. In some embodiments, NTPs of an IVT reaction comprise modified UTP. In some embodiments, NTPs of an IVT reaction comprise unmodified GTP, In some embodiments, NTPs of an IVT reaction comprise modified GTP. In some embodiments, NTPs of an IVT reaction comprise unmodified CTP. In some embodiments, NTPs of an IVT reaction comprise modified CTP.

In some embodiments, a RNA transcript (e.g., mRNA transcript) includes a modified nucleobase selected from pseudouridine (ψ), 1-methylpseudouridine (mV), 5-methoxyuridine (mo⁵U), 5-methylcytidine (m⁵C), a-thio-guanosine and a-thio-adenosine. In some embodiments, a RNA transcript (e.g., mRNA transcript) includes a combination of at least two (e.g., 2, 3, 4 or more) of modified nucleobases, such as modified nucleobases set out herein.

In some embodiments, an RNA transcript (e.g., mRNA transcript) includes pseudouridine (ψ). In some embodiments, an RNA transcript (e.g., mRNA transcript) includes 1-methylpseudouridine (m¹ψ). In some embodiments, an RNA transcript (e.g., mRNA transcript) includes 5-methoxyuridine (mo⁵U). In some embodiments, an RNA transcript (e.g., mRNA transcript) includes 5-methylcytidine (m⁵C). In some embodiments, a RNA transcript (e.g., mRNA transcript) includes a-thio-guanosine. In some embodiments, a RNA transcript (e.g., mRNA transcript) includes a-thio-adenosine.

In some embodiments, the polynucleotide (e.g., RNA polynucleotide, such as mRNA polynucleotide) is uniformly modified (e.g. fully modified, modified throughout the entire sequence) for a particular modification. For example, a polynucleotide can be uniformly modified with 1-methylpseudouridine (m¹ψ), meaning that all uridine residues in the mRNA sequence are replaced with 1-methylpseudouridine (m¹ψ). Similarly, a polynucleotide can be uniformly modified for any type of nucleoside residue present in the sequence by replacement with a modified residue such as any of those set forth above. Alternatively, the polynucleotide (e.g., RNA polynucleotide, such as mRNA polynucleotide) may not be uniformly modified (e.g., partially modified, part of the sequence is modified).

### Capped RNA Preparations

Without wishing to be bound by a particular theory, the use of capped RNA (e.g., capped mRNA molecules) preparations where with a high ratio of capped/uncapped RNA may result in increased expression compared to preparations with a lower capped/uncapped RNA ratio. Thus, in many instances, it will be desirable to separate capped RNA (e.g., capped mRNA molecules) from uncapped RNA (e.g., uncapped mRNA molecules) prior to introduction of the capped RNA molecules into cells. Such separation may occur by any number methods, including purification of capped RNA (e.g., capped mRNA molecules) by methods such as high performance liquid chromatography (HPLC) or electrophoresis and/or selective degradation of uncapped RNA molecules.

Methods and compositions described herein provide for methods of generating RNAs incorporating cap analogs as described herein. The efficiency of mRNA produced in IVT reactions using the cap analogs as described herein can be at least 40%, at least 50%, at least 60%, at least 70%, at least 80%, at least 90%, at least 95% or more, or any value in between, of the yield of mRNA produced in an IVT reaction under identical conditions, except for the inclusion of a cap analog as described herein.

The methods and compositions provided herein provide for method of generating RNAs wherein at least 50%, at least 60%, at least 70%, at least 80%, at least 90%, at least 95% or more, or any value in between, of the total mRNA produced in an IVT is capped with the cap analogs described herein. In other words, the capping efficiency is at least 50%, 55%, 60%, 65%, 70%, 75%, 80%, 85%, 90%, 91%, 92%, 93%, 94%, 95%, or more (e.g., from about 50% to about 99%, from about 60% to about 99%, from about 70% to about 99%, from about 80% to about 99%, from about 85% to about 99%, from about 85% to about 95%, from about 90% to about 96%, etc.).

Methods provided herein for making RNA preparations incorporating the caps provided herein can advantageously include the step of degrading RNA that is not capped, thereby purifying or enriching the capped RNA species. Removal of uncapped RNA can be accomplished by any means known to those skilled in the art, including but not limited to enzymatic digestion. For example, RNA preparations can be treated with RNA 5′ polyphosphatases, which removes pyrophosphate from 5′ triphosphorylated RNA, leaving a monophosphate 5′ end. The preparation can subsequently be treated with a 5′→3′ exoribonucleases, which requires a 5′ monophosphate RNA as a substrate. One category of such enzymes are the XRN 5′→3 exoribonucleases (see Nagarajan et al, “XRA 5′→3′ exoribonucleases: Structure, mechanisms and functions”, Biochim Biophys Acta., 1829:590-603. (2013)). Thus, treatment with these two enzymes will selectively degrade uncapped RNAs, leaving capped RNAs intact.

Methods set out herein include those where RNA molecules (e.g., mRNA molecules) that are not capped are preferentially degraded over capped RNA molecules (e.g., capped mRNA molecules), as well as compositions used to perform such methods. Such methods may be performed with or without treatment of RNA present in the reaction mixture prior to preferential degradation of uncapped RNA. When reaction mixtures are treated to prepare a subpopulation of RNA molecules (e.g., uncapped RNA molecules) for degradation this preparation may occur before or at the same time as degradation of RNA with a 5′->3′ exoribonuclease. Thus, both preparation of RNA molecules for degradation by a 5′-+3′ exoribonuclease and degradation of the RNA molecules may occur in the same reaction mixture at different times or at the same time. In some instances, the 5′ termini of RNA molecule for which degradation is desired may need to be modified so that a 5′-+3′ exoribonucleases will act upon the termini. One example of this is when an XRN1 exoribonuclease is used to degrade uncapped RNA molecules.

XRN1 is a progressive XRN1 exoribonuclease that degrades termini of RNA molecules that contain a single 5′ phosphate group. One commercially available XRN1 exoribonuclease is available from New England Biolabs (cat. no. M0338S). In instances where some or all of the RNA molecules present that one seeks to degrade contain more than one 5′ phosphate group, it will normally be desirable to reduce the number of phosphate groups down to one. A number of methods of methods may be used to remove 5′ phosphate groups from RNA, including methods that employ phosphatases for enzymatic removal of these groups, One category of such enzymes are the RNA 5′ polyphosphatases (e.g., Lucigen, cat. no. RP80921H).

Methods set out herein include those where the amount of capped RNA is increased over uncapped RNA by at least 50% (e.g., by at least from about 50% to about 500%, from about 100% to about 500%, from about 150% to about 500%, from about 200% to about 500%, from about 250% to about 500%, from about 300% to about 500%, from about 50% to about 1,000%, from about 150% to about 1,000%, from about 300% to about 1,000%, from about 400% to about 1,000%, etc.). The following is an example of how percent increase may be calculated, Assume that there are 100 mg of capped RNA and 50 mg of uncapped RNA in a sample. If the amount of uncapped RNA is decreased to 25 mg, then the total amount of uncapped RNA is decreased by half. Further, the ratio of capped RNA to uncapped RNA would go from 2:1 to 4:1 and the amount of capped RNA would increase over uncapped RNA by 21%.

Compositions comprising trinucleotide capped RNA as described herein can be used for in vitro transcription, in vitro translation, and in vivo translation, for example. Current biotechnology efforts for in vitro, in situ, and in vivo protein production will also benefit from these methods and compositions. Further, compositions provided herein are useful for therapeutic purposes. For example, the present technology may be useful for generating vaccines against infectious diseases or cancers, protein replacement therapies, and the like. The skilled artisan will readily appreciate that the capping technology and the compositions described herein can be used generally in mRNA vaccines. For example, the RNA caps described herein can be incorporated into RNA sequences useful in vaccines, including but not limited to sequences described in US20180318409A1, US20190351040, US20180271970, US20190054112, US20190336595, US20180311336, US20180303929, WO2017/070601, WO2019/202035, WO2020/002525, WO2019/193183, WO2019/115635, WO2019/038332, WO2019/008001, WO2018/167320, WO2018/115527, WO2018/115525, WO2018/115507, WO2018/104538, WO2018/104540.

Alkyne-derivitized capped RNA can be used to produce non-infectious particles of a virus containing an RNA encoding immunogen. These non-replicating viral particles can be injected into humans where they can enter host cells. Once in the host cell, the viral particles dissociate and the mRNA encoding the immunogen is translated into protein. These proteins can induce an immune response.

RNA-based vaccines may be used to vaccinate against infectious agents such as viruses, e.g., corona viruses (such as MERS, SARS-CoV and SARS-CoV-2), human immunodeficiency virus (HIV), feline immunodeficiency virus, human papilloma virus type 16, tumors, lassa virus, Ebola virus, Marburg virus, anthrax toxin from Bacillus anthraces, and botulinum toxin. Accordingly, non-limiting examples of viruses for which an RNA vaccine could be used for include: Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolaviurs, Echovirus, Encephalomycarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus Human herpesvirus 1, s, Hepatitis delta virus, Horsepox virus, Human adenovirus, Human astrovirus, Human coronavirus, Human cytomegalovirus, Human herpesvirus 1, Human herpesvirus 2, Human herpesvirus 6, Human herpesvirus 7, Human herpesvirus 8, Human immunodeficiency virus, Human papilloma virus 1, Human papilloma virus 2, Human papilloma virus 16,18, Human parainfluenza, Human parovirus B19, Human respiratory syncytial virus, Human rhinovirus, Human SARS coronavirus, Human spumaretrovirus, Human T-lymphotropic virus, Human toroviurs, Influenza A virus, Influenza B virus, Influenza C virus, Isfahan virus, JC polyomavirus, Japanese encephalitis virus, Junin arenavirus, KI polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polymavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus Sagiyama virus, Salivirus A, Sandfly fever Sicilian virus, Sapporo virus, SARS coronavirus 2, Semliki forest virus, Seoul virus, Simian foam virus, Simian virus 5, Sindbis virus, Southampton virus, St. louis encephalitis virus, Tick-borne Powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitits virus, WU polyomavirus, West Nile virus, Yaba monkey tumor virus, Yaba-like disease virus, Yellow fever virus, Zika virus.

These vaccine strategies can require large quantities of capped RNA. The present methods facilitate such synthesis and subsequent purification of capped RNA so as to make these vaccines commercially feasible. As well, strategies to increase the percentage of full-length capped RNA in a transcription reaction leading to a more homogenous product will be preferred in the vaccine industry as highly pure components are usually required for human use. In addition, researchers prefer to use products that are as pure as possible to minimize the number of variables in an experiment. As well, the purer the product, the more potent it is.

An additional embodiment relates to the administration of a composition which generally comprises an active ingredient (e.g., trinucleotide capped RNA) formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may include novel cap analogs, antibodies to novel cap analogs, and mimetics, agonists, antagonists, or inhibitors of novel cap analogs.

In various embodiments, the compositions described herein, such as pharmaceutical compositions, may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Embodiments of the present disclosure can be further understood in light of the following examples, which should not be construed as limiting the scope of the present disclosure in any way.

Those having ordinary skill in the art will understand that many modifications, alternatives, and equivalents are possible. All such modifications, alternatives, and equivalents are intended to be encompassed herein.

### EXAMPLES

The following examples provide methods of producing trinucleotide cap analogs.

Reagents: Reagents and solvents are used as such without further purification, unless otherwise stated. 3′-O-propargyl guanosine was purchased from Chemgenes, USA, ¹H NMR and ³¹P NMR spectra were recorded in D₂O on a Burker 400 MHz instrument. ESI mass spectra were recorded on an Applied Biosystems/Sciex API 150 model. HPLC was run on a waters 2996 (Waters Corporation) using anion exchange column. Ion exchange chromatography was performed in an AKTA purifier (Amersham Biosciences, GE Healthcare) using a DEAE Sepharose column. The gel shift assay is performed by using a pTri β actin template and the IVT reaction uses linearized AmbLuc poly(A) DNA template and a MEGASCRIPT kit (Thermo Fisher Scientific). Radiation in the gel bands of interest is quantified by a phosphorimager (GE Healthcare). Purifications of the RNA from these transcription reactions are done by using the MEGACLEAR Kit (Life Technologies Corporation) as per manufacturer's protocol. Luminometer (POLARstar OPTIMA. BMG Labtech) in 96-well plates is used for the luciferase assay readings as per manufacturer's protocol.

### Example 1: Intermediate Synthetic Schemes

Exemplary synthetic routes to obtain the intermediates used in the trinucleotide synthesis are set forth below

### Intermediate Scheme A: Synthesis of Imm⁷(LNA)GDP (8):

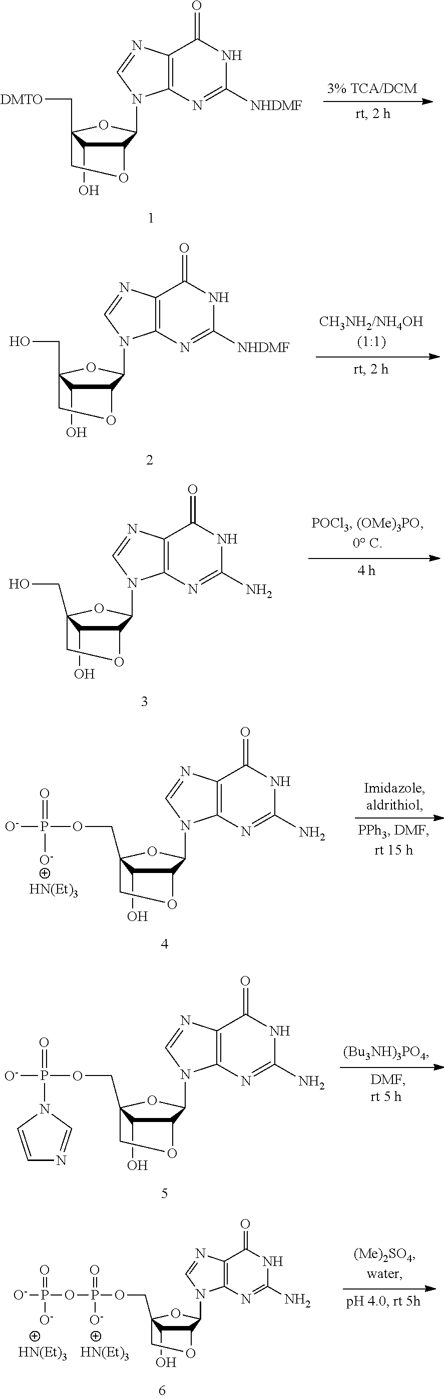

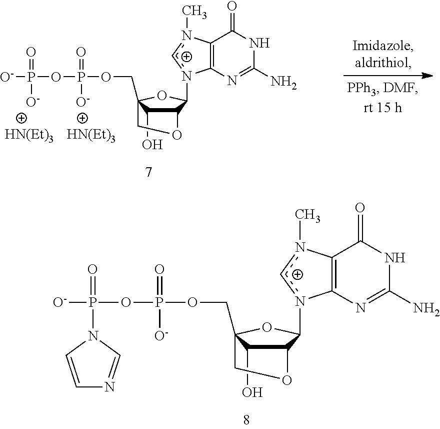

### Intermediate Example 1: Synthesis of DMF-Protected LNA Guanosine (2)

To a stirred solution of 50 mL 3% trichloroacetic acid in dichloromethane, 5′-DMT-N-DMF LNA guanosine 1 (5.00 g, 7.48 mmol) was added and the reaction mixture was stirred for 2 h at room temperature. The reaction mixture was evaporated to dryness under rotary evaporator. To the resulting orange solid, 50 mL diethyl ether was added and allowed to stir at room temperature for 30 min. The resulting mixture was filtered and dried under vacuum to get a white colored solid 2 (Yield: 2.67 g, 95%). This crude material was used for next step without further purification.

### Intermediate Example 2: Synthesis of LNA Guanosine (3)

To a stirred solution of 40 mL 1:1 mixture of aqueous 40% methyl amine and 28% ammonium hydroxide, DMF-protected LNA guanosine 2 (2.67 g, 7.04 mmol) was added and the reaction mixture was stirred at room temperature for 2 h. After 2 h, the reaction mixture was evaporated under rotavapor to get a white colored solid 3 (Yield, 1.98 g, 95%). This crude material was used for next step without further purification.

### Intermediate Example 3: Synthesis of LNA-GMP (4)

To a stirred solution of POCl₃ (1.69 g, 11.19 mmol) and (MeO)₃P (15.0 mL) at 0° C. under argon atmosphere, LNA guanosine 6 (1.10 g, 3.72 mmol) was added and the reaction mixture was stirred for 4 h at 0° C. After 4 h, 50.0 mL water was added to the reaction mixture. The resulting reaction mixture was washed with ethyl acetate (2×50 mL) to remove phosphorylating agent. The collected aqueous solution was adjusted to pH 1.5 and allowed to stir at 4° C. for 15 h. After 15 h, the aqueous solution was adjusted to pH 5.5 and loaded on a DEAE Sepharose column. The desired product was eluted using a linear gradient of 0-1M TEAB (triethyl ammonium bicarbonate, pH 7.5) and the fractions containing the product were pooled, evaporated and dried in vacuum desiccator over phosphorous pentoxide to give a fine white powder 4 (Yield: 1.43 g, 78%). Data for 4. ¹H NMR (D₂O, 400 MHz) δ 8.01 (s, 1H), 5.91 (s, 1H), 4.63 (s, 1H), 4.58 (s, 1H), 4.16 (m, 3H), 4.03 (d, J=8.4 Hz, 1H), 3.20 (q, J=7.6 Hz, 6H), 1.28 (t, J=7.2 Hz, 9H); ³¹P NMR (D₂O, 162 MHz) δ −5.40 (s, 1P); MS (m/z): 374 [M-H]—.

### Intermediate Example 4: Synthesis of ImLNA-GMP (5)

To a stirred solution of LNA-GMP TEA salt 4 (1.35 g, 2.84 mmol) in 20 mL dry DMF, imidazole (0.97 g, 14.24 mmol), triphenyl phosphine (1.50 g, 5.70 mmol), aldrithiol (1.25 g, 5.70 mmol) and triethylamine (0.29 g, 2.84 mmol) were added. The reaction mixture was stirred under argon atmosphere at room temperature for 15 h. To a solution of sodium perchlorate (2 g) in 100 mL acetone in a centrifuge tube at 0° C., the above reaction mixture was added slowly for 5 minutes. The resulting mixture was centrifuged, and the supernatant liquid was removed. The solid was ground with a new portion of acetone (100 mL), cooled, and centrifuged again. This process was repeated for two more times, and the resulting solid was dried in a vacuum desiccator over P₂O₅ to give a white powder 5 (Yield: 1.05 g, 83%). MS (m/z): 424 [M-H]⁻.

### Intermediate Example 5: Synthesis of LNA-GDP (6)

To a stirred solution of ImLNA-GMP 5 (1.00 g, 2.23 mmol) and zinc chloride (0.61 g, 4.46 mmol) in 10.0 mL dry DMF, 15 mL of 1M tris(tributylammonium) phosphate in DMF was added under argon atmosphere. The reaction mixture was stirred at room temperature for 5 h. After 5 h, the reaction mixture was diluted with 50.0 mL of water. The resulting reaction mixture was washed with ethyl acetate (2×50 mL) to remove phosphorylating agent. The collected aqueous solution was adjusted to pH 5.5 and loaded on a DEAE Sepharose column. The desired product was eluted using a linear gradient of 0-1M TEAB and the fractions containing the product were pooled, evaporated and dried in vacuum desiccator over phosphorous pentoxide to give a fine white powder 6 (Yield 1.10 g, 75%). Data for 6. ¹H NMR (D₂O, 400 MHz) δ 8.00 (s, 1H), 5.95 (s, 1H), 4.63 (s, 2H), 4.37 (m, 2H), 4.14 (d, J=8.4 Hz, 1H), 4.04 (d, J=8.4 Hz, 1H), 3.20 (q, J=7.2 Hz, 12H), 1.28 (t, J=7.6 Hz, 18H); ³¹P NMR (D₂O, 162 MHz) δ-8.94 (d, J=20.9 Hz, 1P), 9.99 (d, J=21.3 Hz, 1P); MS (m/z): 454 [M-H]—.

### Intermediate Example 6: Synthesis of m^{7(LNA)}GDP (7)

To a stirred solution of LNA-GDP 6 (1.00 g, 1.52 mmol) in 20.0 mL of water, acetic acid was added slowly to adjust the pH of the solution to 4.0. To this mixture, dimethyl sulfate (2.0 mL) was added drop wise over a period of 30 min. and the reaction mixture was allowed to stir at room temperature for 5 h. As the methylation proceeds, the pH drops down to around 2.0 and the pH was readjusted back to 4.0 using 1M NaOH solution. After 5 h, the reaction mixture was extracted with ethyl acetate (3×50 mL) to remove unreacted excess dimethyl sulfate. The collected aqueous solution was adjusted to pH 5.5 and loaded on a DEAE Sephadex column. The desired product was eluted using a linear gradient of 0-1M TEAB and the fractions containing the product were pooled, evaporated and dried in vacuum desiccator over phosphorous pentoxide to give a fine white powder 7 (Yield 0.70 g, 68%). Data for 7. ¹H NMR (D₂O, 400 MHz) δ 6.05 (s, 1H), 4.73 (s, 1H), 4.55 (s, 1H), 4.42 (m, 1H), 4.32 (m, 1H), 4.13 (s, 3H), 4.11 (d, J=6.0 Hz, 1H), 4.00 (d, J=8.8 Hz, 1H), 3.20 (q, J=7.2 Hz, 12H), 1.28 (t, J=7.2 Hz, 18H); ³¹P NMR (D₂O, 162 MHz) δ −6.18 (d, J=23.0 Hz, 1P), −9.56 (d, J=22.8 Hz, 1P); MS (m/z): 468 [M−H]⁻.

### Intermediate Example 7: Synthesis of Imm^{7(LNA)}GDP (8)

To a stirred solution of m^{7(LNA)}GDP TEA salt 7 (0.65 g, 0.96 mmol) in 15 mL dry DMF, imidazole (0.33 g, 4.84 mmol), triphenyl phosphine (0.51 g, 1.93 mmol), aldrithiol (0.43 g, 1.93 mmol) and triethylamine (0.10 g, 0.96 mmol) were added. The reaction mixture was stirred under argon atmosphere at room temperature for 15 h. To a solution of sodium perchlorate (2 g) in 100 mL acetone in a centrifuge tube at 0° C., the above reaction mixture was added slowly for 5 minutes. The resulting mixture was centrifuged, and the supernatant liquid was removed. The solid was ground with a new portion of acetone (100 mL), cooled, and centrifuged again. This process was repeated for two more times, and the resulting solid was dried in a vacuum desiccator over P₂O₅ to give a white powder 8 (Yield: 0.42 g, 80%). MS (m/z): 517 [M−H]⁻.

Intermediate Scheme B: Synthesis of Dinucleotide pAmpG (13):

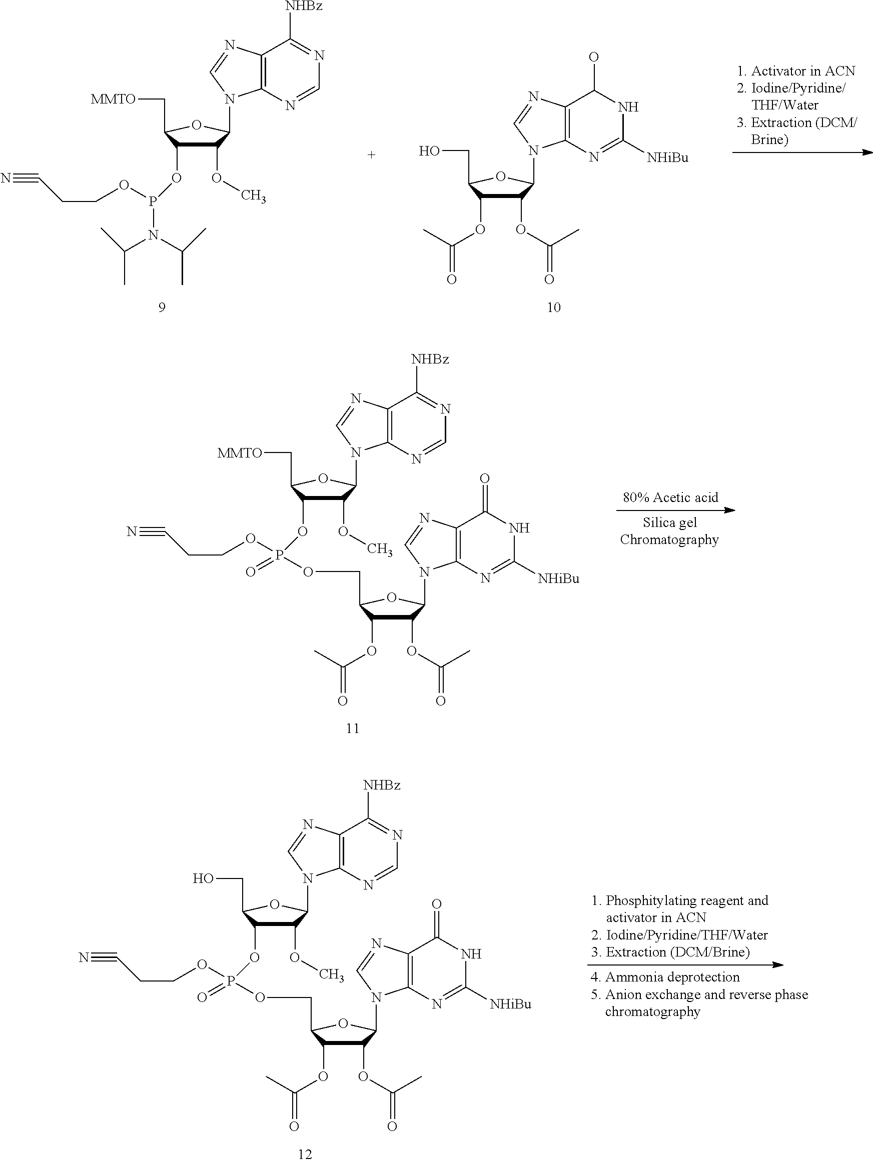

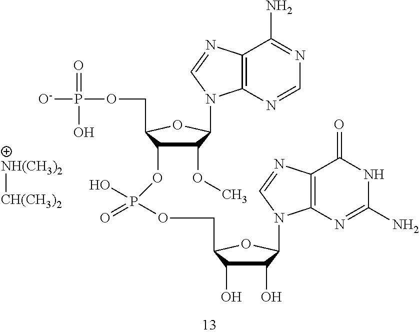

### Intermediate Example 8: Synthesis of Dinucleotide pAmpG (13)

In a typical reaction, MMT-2′-O-Methyl Adenosine (n-bz) CED phosphoramidite 9 (2.0 mmol) and 2′,3′-Diacetyl Guanosine (n-ibu) 10 (2.0 mmol) are reacted in 20 mL of acetonitrile containing 4.5 molar equivalents of activator (tetrazole in acetonitrile). After 2 hours of stirring at room temperature the intermediate product is oxidized from the P(III) to P(V) state with Iodine/Pyridine/THF/Water and extracted with dichloromethane (400 mL) and brine (400 mL). The resulting organic layer is dried with sodium sulfate and is evaporated to solid form intermediate 11.

To remove the 5′-MMT group, intermediate 11 is dissolved in 20 mL of 80% acetic acid and resulting reaction mixture is stirred at room temperature for about 2 to 3 hours. After reaction is completed, the mixture is evaporated and co-evaporated with methanol (6×60 mL) to remove acetic acid. The crude 5′-OH dimer 12 is isolated and purified by silica gel chromatography using 5% methanol in dichloromethane as an eluent.

The 5′-OH dimer 12 (2.0 mmol) is phosphitylated with four equivalents of bis-cyanoethyl-N, N-diisopropyl-phosphoramidite and four equivalents of activator (tetrazole in 20 mL acetonitrile). After 45 minutes of stirring at room temperature the 5′-phosphitylated dimer is oxidized from the P(III) to P(V) state with Iodine/Pyridine/THF/Water and extracted with dichloromethane (300 mL) and brine (300 mL). The organic layer is evaporated to an oily residue, co-evaporated with methanol (2×60 mL), and dissolved in 25 mL of methanol and concentrated ammonia (25 mL) was added. The resulting mixture was kept at room temperature for over 48 hours until deprotection of the pAmpG dimer 13 is complete. The mixture is evaporated and co-evaporated with methanol and resulting dimer is characterized by LC/MS (MS (m/z): 705 [M−H]⁻) and used for further conjugation to synthesize trinucleotide cap analog.

### Example 2: Synthesis of LNA Trinucleotide Cap Analog (14)

The General Trinucleotide Scheme below illustrates the combination of the intermediates from Intermediate Scheme (A) and Intermediate Scheme (B) to arrive at an exemplary trinucleotide cap analog described herein. In this illustration, the trinucleotide analog of the present disclosure (14) is a locked cap analog.

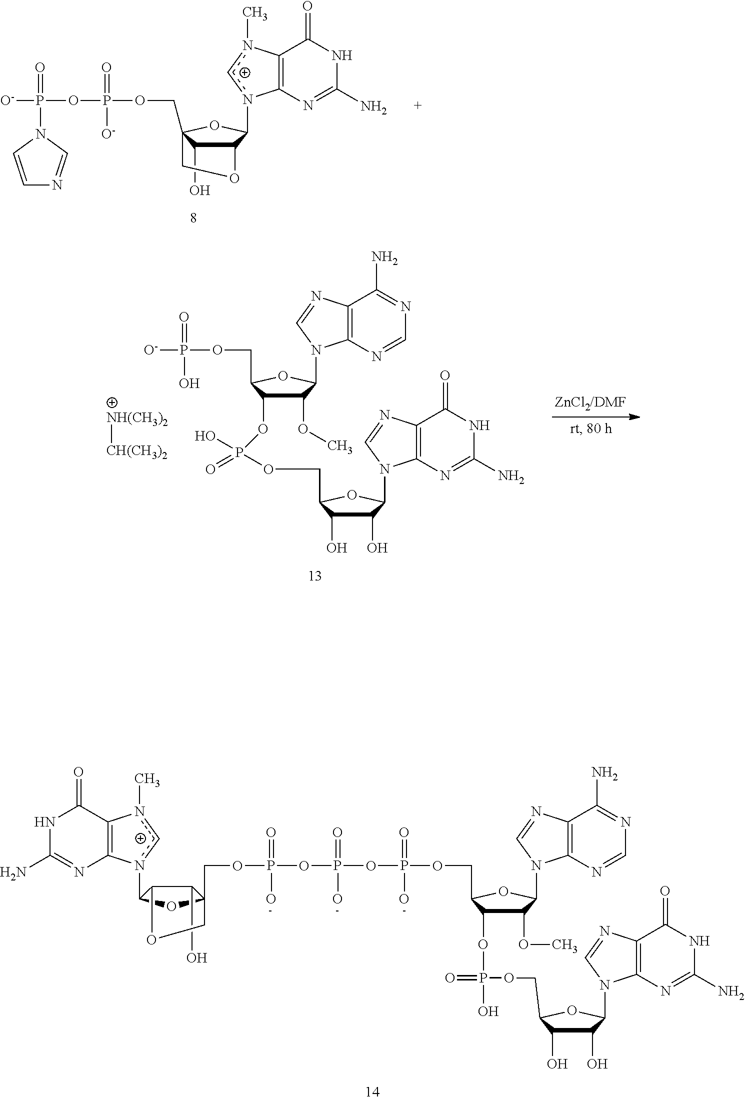

### Trinucleotide Cap Analog Example 1—Synthesis of LNA Trinucleotide Cap Analog (14):

To a stirred solution of Imm⁷(LNA)GDP 8 (0.10 g, 0.18 mmol) and pAmpG N,N-dimethyl isopropyl ammonium salt 13 (0.14 g, 0.18 mmol) in 10.0 mL dry DMF, zinc chloride (0.15 g, 1.10 mmol) was added under argon atmosphere and the reaction mixture was stirred at room temperature for 80 h. The reaction mixture was added to a solution of EDTA disodium (0.55 g, 1.48 mmol) in 100.0 mL of water at 0° C. The resulting aqueous solution was adjusted to pH 5.5 and loaded on a DEAE Sephadex column. The desired product was eluted using a linear gradient of 0-1M TEAB and the fractions containing the product were pooled, evaporated and concentrated to 10.0 mL TEA salt of 14. The TEA salt of the product was dissolved in water (5 mL) and then poured into a solution of sodium perchlorate (2.0 g) in acetone (50 mL). The resulting mixture was centrifuged, and the supernatant liquid was discarded. The solid obtained was washed with acetone (2×50 mL) and dried in vacuum to give a sodium salt of LNA trinucleotide cap Analog 14. (Yield: 0.12 g, 55%) Data for 14. ¹H NMR (D₂O, 400 MHz) δ 8.39 (s, 1H), 7.94 (s, 1H), 7.84 (s, 1H), 6.01 (d, J=6.0 Hz, 1H), 5.87 (d, J=6.0 Hz, 1H), 5.55 (s, 1H), 4.83 (m, 1H), 4.43 (m, 4H), 4.31 (m, 2H), 4.23 (m, 4H), 4.10 (m, 3H), 3.99 (m, 1H), 3.93 (m, 1H), 3.91 (s, 3H), 3.29 (s, 3H); ³¹P NMR (D₂O, 162 MHz) δ-0.92 (s, 1P), −11.11 (d, J=19.4 Hz, 1P), −10.42 (d, J=17.8 Hz, 1P), −22.91 (t, J=17.8 Hz, 1P); MS (MALDI, m/z): 1156 [M−H]⁻.

### Example 3: In Vitro Transcription with LNA Trinculeotide CAP Analog

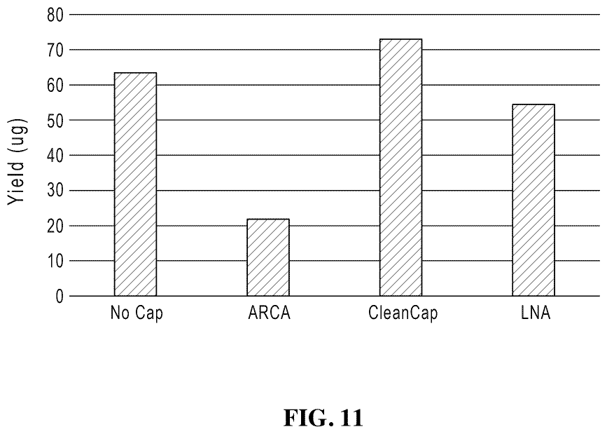

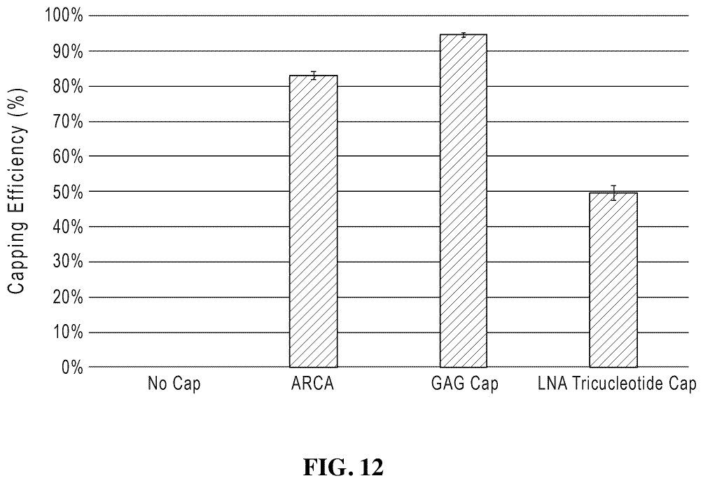

The following example demonstrates that the synthetic CAPs described herein can be incorporated into mRNA in vitro. Briefly, linearized DNA including the coding sequence for GFP under control of a T7 promoter was used in an in vitro transcription reaction using the buffer and enzymes from the MMESSAGEMMACHINE™ T7 Kit (Thermo Fisher Scientific, cat. no. AM1344), and a NTP/cap mixture. The NTP/cap mixture contained a mixture of NTPs and either no cap, ARCA cap analog (Thermo Fisher Scientific, cat. no. AM8045), GAG Cap analog) (CLEANCAP™ AG, Trilink Biotechnologies, cat. no. N-7113), or Compound (14) as described herein. Reactions with ARCA or no cap were performed using a DNA template containing the wild-type T7 promoter which contains a GGG start. A DNA template containing a modified T7 promoter containing an AGG start was used for reactions with GAG Cap cap analog or LNA-modified GAG Cap cap (Compound 14). In all reactions except for those with ARCA, the concentration of each NTP and cap was 5 mM. The reactions with ARCA contained the following cap/NTP concentrations: 6 mM ARCA, 1.5 mM GTP, and 7.5 mM ATP, CTP, and UTP each. Reactions were processed according to the manufacturer's instructions. Each reaction generated full-length RNA transcripts that are approximately 1000 nucleotides, including −120 nt poly(A) tail. RNA transcripts were purified using the MEGACLEAR™ RNA purification kit (Thermo Fisher Scientific, cat. no. AM1908) according to the manufacturer's instructions, and RNA yield was quantified by measuring absorbance at 260 nm in a NANODROP™ 2000C spectrometer (Thermo Fisher Scientific, cat. no. ND-2000C). As shown in FIG. 11, the RNA yield in the reactions containing the LNA Cap analog was superior to the ARCA cap reaction.

### Example 4: Capping Efficiency with LNA Cap Analogs

The capping efficiency of LNA cap analogs was compared to no cap control, ARCA cap analogs, and CLEANCAP™ cap analogs. Briefly, in vitro transcription reactions were performed as described Example 3. In order to be able to resolve uncapped vs. capped mRNAs, e.g., on a Bioanalyzer, 1-2 μg RNA sample from the in vitro transcription reaction was treated with a DNAzyme oligonucleotide to trim the RNAs to 30 nucleotides long from 5′ end (no cap), 31 nucleotides long (ARCA, GAG-cap (GpppAG)), LNA Cap Analog (LNA-modified GAG cap) in a reaction containing 5 μl of 200 mM Tris-HCl, pH 7.5, 1 μl of 10 μM DNAzyme oligonucleotide TTGAGGTTGCTAGTGAAGGCTAGCTACAACGAACAGTTGTGTCAGAAGC (SEQ ID NO: 584) and water to a total volume of 16 μl. The mixture was preheated at 85° C. for 30 seconds, and equilibrated at 37° C. for 5 minutes. 4 μl of 50 mM MgCl₂ was added to the mixture, which was allowed to incubate at 37° C. for one hour. To stop the reaction, 2 μl TURBO™ DNAse (Thermo Fisher Scientific, cat. no. AM2238) was added and incubated at 37° C. for 30 minutes. 1-2 μl of the reaction was loaded onto a Bioanalyzer chip (Agilent, San Jose, CA) using the small RNA Analysis kit (Agilent, Cat. No. 5067-1548), according to the manufacturer's protocol. Uncapped mRNA molecules were 1 base shorter than mRNA species that were successfully capped with ARCA or LNA-cap analogs, respectively. The capping efficiency was calculated as amount capped mRNA/total mRNA, by measuring the area under the peaks corresponding to capped or uncapped species

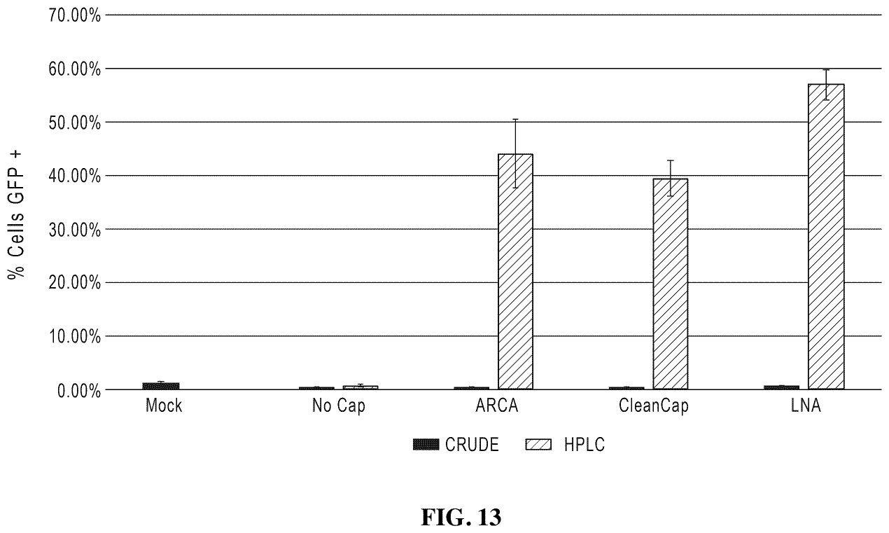

As shown in FIG. 13 the capping efficiency of the LNA cap analog is lower than the ARCA cap analog and the GAG cap analog.

### Example 5: Transfection Efficiency and Expression Efficiency of mRNA Capped with LNA Cap Analogs

The transfection efficiency and expression efficiency of LNA cap analogs was compared to uncapped mRNA, ARCA cap analogs, and GAG cap (CLEANCAP™) cap analogs. The JAWSII murine immortalized dendritic cell line were used for the analysis. Transfections were done with “crude” mRNA or “HPLC Purified mRNA.” For “crude” mRNA, transcripts were used directly after purification using the MEGACLEAR™ RNA purification kit as described in Example 3. For “HPLC Purified” mRNA, uncapped mRNA was removed by mixing 10 ug mRNA with 20 units of RNA 5′ Polyphosphatase (Lucigen, cat. RP8092H), which dephosphorylates uncapped, but not capped mRNAs leaving monophosphate 5′ ends, and incubating for an hour at 37° C. RNA transcripts were purified using GeneJET RNA Cleanup and Concentration Micro Kit™ (Thermo Fisher Scientific, cat. no. K0842) according to the manufacturer's instructions. This purified mRNA was added to 2 units of XrnI (New England Biolabs, cat. M0338S, a processive 5′->3′ exoribonuclease, that requires a 5′ monophosphate as a substrate, and incubated at 37° C. for an hour. The mRNA transcripts were purified using GeneJET RNA Cleanup and Concentration Micro Kit™ (Thermo Fisher Scientific, cat. no. K0842) according to the manufacturer's instructions. Removal of uncapped transcripts was confirmed by performing the capping assay as described in Example 4. Double stranded RNA (dsRNA) was removed from the above treated samples using an Agilent Technologies Series 1260 Infinity HPLC equipped with a Clarity® 5 μm Oligo-RP 150 ×4.6 mm column set to 65° C. A linear gradient of buffer B (0.1 M triethylammonium acetate pH 7.0 and 25% acetonitrile) from 38% to 70% in buffer A (0.1 M triethylammonium acetate pH 7.0) over 10 min at 1 ml/min was applied. RNA was recovered from collected fractions using the GeneJET RNA Cleanup and Concentration Micro kit. Concentration of the recovered mRNA was determined by Nanodrop, and depletion of dsRNA was confirmed by performing a dot blot with the anti-dsRNA J2 antibody. The quality of the transcripts was also checked using the Bioanalyzer with the RNA Nano 6000 kit.

Crude and HPLC purified mRNAs were then used to transfect JAWS II cells. The cells were cultured according to protocols outlined by American Tissue Cell Collection (ATCC) organization. 50,000 cells were seeded onto a 96 well plate so that cells are 70-90% confluent the next day (day of transfection). Cells were transfected using LIPOFECTAMINE™ MESSSENGERMAX™ transfection reagent (Thermo Fisher, Cat. LMRNA001). 25 ng crude or HPLC purified mRNA mixed with 0.3 μL MESSENGERMAX™ transfection reagent per manufacturer's protocols, and the mix was added to the cells and incubated at 37 C. 24 hours after transfection, media containing suspension cells was removed and added to a clean plate, and the adherent cells were detached with 50 μL TRYPLE™ Express Enzyme (Thermo Fisher Cat. No. 12604013) according to the manufacturer's protocol. The detached cells were transferred to the plate containing the suspension cells. This cell mixture was run through an ATTUNE NxT™ flow cytometer (Thermo Fisher Cat. No. A29004), and the GFP fluorescence for 10,000 cells were measured for each sample. Cells were gated on live single cells based on forward and side scattering. The gating for GFP was determined by using cells with no GFP. Transfection efficiency was measured by determining the percentage of cells in the GFP-positive gate, and GFP expression was quantified by taking the median fluorescence intensity (MFI) for each sample in the GFP-positive gate. Data were analyzed using FLOWJO™ software.

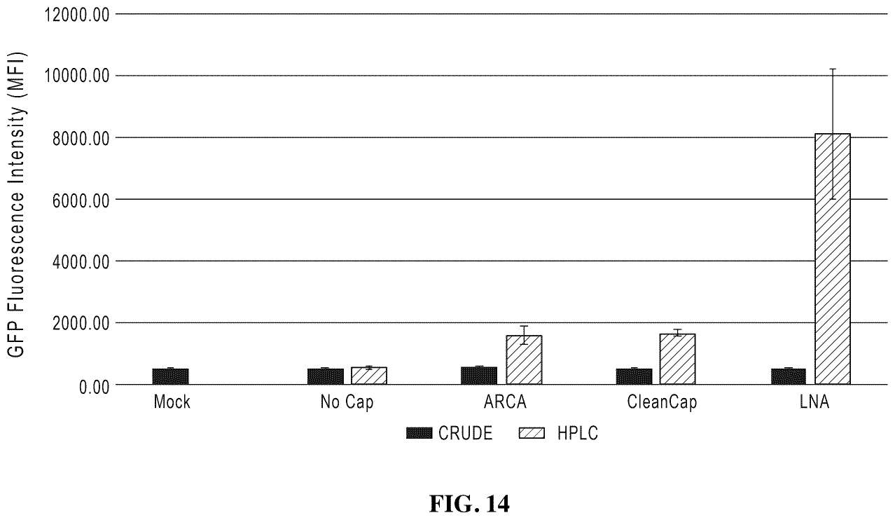

As shown in FIG. 13, mRNA transcripts capped with the LNA cap analog showed significantly higher transfection efficiency, when compared to uncapped mRNA, mRNA capped with ARCA cap analog, and mRNA capped with GAG-cap (CLEANCAP™ AG) cap analog. Furthermore, FIG. 14 shows that the expression efficiency of mRNA capped with the LNA cap analog was more than 4 fold greater than compared to uncapped mRNA transcripts, mRNA transcripts capped with the ARCA cap analog, and mRNA transcripts capped with GAG-cap (CLEANCAP™ AG) cap analog.

### Example 6

### Creation of In Vitro Transcription Templates

The various in vitro transcription (IVT) templates were created by PCR with a DNA plasmid containing the wildtype (WT) T7 promoter, 5′-UTR, eGFP, and 3′-UTR sequences using the PLATINUM™ SUPERFI™ II Green PCR Master Mix (Thermo Fisher Scientific, cat. no. 12369010). Modified T7 promoters were added by site-directed mutagenesis via the forward primer (Table 4). The PCR products were purified using the PURELINK™ PCR Purification kit (Thermo Fisher Scientific, cat. no. K310001) and diluted to 100 ng/μL in water. In vitro transcription reaction

Twenty microliter (20 μL) IVT reactions were performed with 500 ng DNA template, 10 μL 2×NTP/cap mix (described below), 2 μL 10×T7 Reaction Buffer, and 2 μL T7 Enzyme Mix. The reaction buffer and enzyme mix were from the MMESSAGE MMACHINE™ T7 Ultra kit (Thermo Fisher Scientific, cat. no. AMB13455). Reaction mixtures were incubated at 37° C. for 2 hours to synthesize the RNA transcripts. At the end of the reaction, 1 μL TURBO™ DNase (Thermo Fisher Scientific, cat. no. AM2239) was added and incubated at 37° C. for 15 minutes to degrade the DNA template. The RNA transcripts were purified by the MEGACLEAR™ Transcription Clean-Up kit (Thermo Fisher Scientific, cat. no. AM1908). A NANODROP™ spectrophotometer (Thermo Fisher Scientific, cat. no. ND-2000C) was used to measure the concentration of the purified RNA samples.

The 2×NTP/cap mixture was prepared depending on the cap used. The ARCA mixture, which has a 4:1 ARCA:GTP ratio, consisted of 3 mM GTP, 15 mM ATP/CTP/UTP each, and 12 mM ARCA. The CLEANCAP® mixture contained 10 mM GTP/ATP/CTP/UTP each and 10 mM CLEANCAP® (TriLink Biotechnologies, cat. nos. N-7113). For the testing of the different CLEANCAP®:NTP ratios, the CLEANCAP® concentration was changed with respect to the NTP concentration (e.g., 4:1 CLEANCAP®:NTP ratio means that the 2×NTP/cap mixture contained 10 mM GTP/ATP/CTP/UTP each and 40 mM CLEANCAP®). The no cap reactions used a 2×NTP mixture containing 10 mM of each NTP.

### Capping Efficiency Assay

A 10-23 DNAzyme was designed to cut the RNA transcripts 30 nucleotides from the expected transcription start site (Cairns et al., “Optimisation of the 10-23 DNAzyme-substrate pairing interactions enhanced RNA cleavage activity at purine-cytosine target sites”, Nucleic Acids Res. 31(11):2883-2889 (2003)). The DNAzyme reaction was performed in a 20 μl reaction containing 1-2 μg RNA and 0.5 μM DNAzyme in 50 mM Tris-HCl pH 7.5. The reaction mixture was preheated at 85° C. for 30 seconds and equilibrate at 37° C. for 5 minutes and added magnesium chloride to 10 mM so that the total volume was 20 μl. The reaction mixture was incubated at 37° C. for 1 hour and stopped by adding 2 μL TURBO DNase and incubating at 37° C. for 30 minutes.

The DNAzyme reaction mixture was prepared for gel analysis by mixing with 2× NOVEX™ TBE-Urea sample buffer (Thermo Fisher Scientific, cat. no. LC6876) and heating at 70° C. for 2 minutes. The sample was loaded onto a NoVEX™ 15% TBE-Urea gel (Thermo Fisher Scientific, cat. no. EC68855BOX) and ran at 15 V, 15 mA for 1 hour and 40 minutes or until the bromophenol blue dye migrated to the bottom of the gel. The gel was removed from the cassette, washed in water, and stained with SYBR™ Gold Nucleic Acid Gel Stain (Thermo Fisher Scientific, cat. no. S11494) for 5-10 minutes. Following a brief wash in water, the gel was visualized over an UV light and an image was captured using an IBRIGHT™ system (Thermo Fisher Scientific). Capping efficiency was determined using ALPHAVIEW™ software (ProteinSimple) to measure band intensities of the fast and slow migrating transcripts (capped transcripts are longer than uncapped transcripts; therefore uncapped migrates faster).

### Results

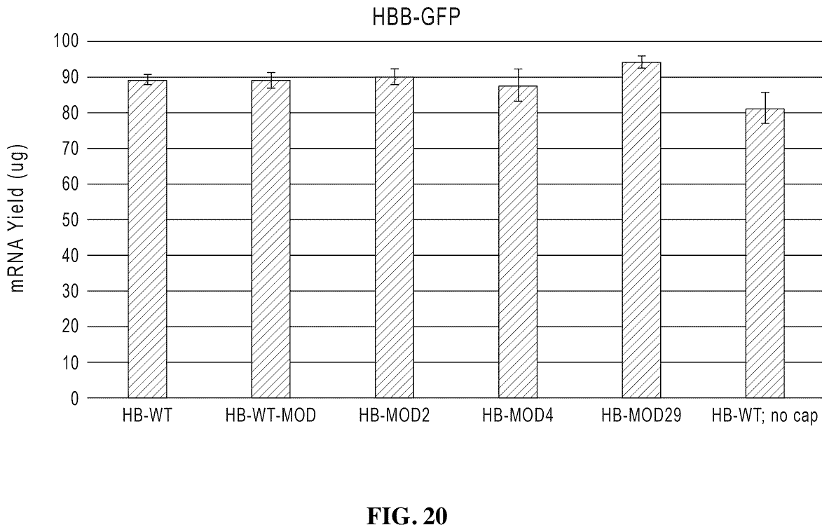

Results from a number of experiments are set out in FIG. 20 and in Tables 6 through 8.

TABLE 4
Forward Primers used for Template Generation

| ID | Sequence | SEQ ID NO: |
|---|---|---|
| HB-WT | AGTAATACGACTCACTATAGGGAGA | 585 |
| | ACATTTGCTTCTGACACAAC | |
| HB-WT-MOD | AGTAATACGACTCACTATAAGGAGA | 586 |
| | ACATTTGCTTCTGACACAAC | |
| HB-MOD2 | AGTAATACGACTCACTATAGTGAGA | 587 |
| | ACATTTGCTTCTGACACAAC | |
| HB-MOD4 | AGTAATACGACTCACTATAGCGAGA | 588 |
| | ACATTTGCTTCTGACACAAC | |
| HB-MOD29 | AGTAATACGACTCACTATAGAGAGA | 589 |
| | ACATTTGCTTCTGACACAAC | |

Reverse primer for all PCR reactions:
GCCCTCTAGATCAACCACTTTGGCCCTCT (SEQ ID NO: 590)

Table 4 lists the PCR forward and reverse primers used to create GFP templates with different promoter modifications

TABLE 5
Promoter Modifications and Designations

| No. | Promoter Designation | Sequence |
|---|---|---|
| 1 | Wild Type | TATA GGG |
| 2 | Wild Type Mod | TATA AGG |
| 3 | MOD2 | TATA GTG |
| 4 | MOD4 | TATA GCG |
| 5 | MOD26 | TATG GTG |
| 6 | MOD27 | TATA TTG |
| 7 | MOD28 | TATT GTG |
| 8 | MOD29 | TATA GAG |

Table 5 lists modified promoters and their sequences. The base after the space in the sequence is the natural +1 position.

TABLE 6
mRNA Yield and Capping Efficiency Using −1/+1 Initiation
Site and 1:1 NTP to Trinucleotide GAG Cap Ratio (FIG. 20)

| Template | Avg (ug) | St Dev | Avg % capped | St dev |
|---|---|---|---|---|
| HB-WT | 89.15 | 1.48 | 53.765 | 2.74 |
| HB-WT-MOD | 89 | 2.12 | 91.415 | 0.90 |
| HB-MOD2 | 89.95 | 2.05 | 82.3 | 2.28 |
| HB-MOD4 | 87.65 | 4.45 | 73.75 | 0.11 |
| HB-MOD29 | 94.15 | 1.63 | 77.5 | 1.20 |
| HB-WT; no cap | 81.25 | 4.31 | 0 | 0 |

| | | |
|---|---|---|
| HB-WT Template, ARCA Cap (data to right) | 71.93 | 0.38 |

Table 6 lists the IVT yields and capping efficiencies for various modified promoters when using a 1:1 NTP to cap ratio. The −1 start promoters, HB-MOD2, HB-MOD4, and HB-MOD29, give high yields and high capping efficiencies.

TABLE 7
IVT yield of using HB-MOD2 promoter with Different Trinucleotide GAG Cap:NTP Ratios

| Promoter | Yield (ug) | | | | | | | | |
|---|---|---|---|---|---|---|---|---|---|
| Cap:NTP | Mix 1 | Mix 2 | Mix 3 | Mix 4 | Mix 5 | Mix 6 | Mix 7 | Mix 8 | Mix 9 |
| ratio | 6:1 | 4:1 | 2:1 | 1.5:1 | 1.1:1 | 1:1 | 0.8:1 | 0.5:1 | No cap |
| HB-WT | 52.9 | 83 | 86.4 | 84.5 | 86.8 | 91.7 | 85.1 | 86.4 | 82.8 |
| HB-MOD2 | 28.8 | 44.6 | 89.8 | 70.2 | 92.5 | 97.7 | 89.9 | 85.4 | 80.5 |

TABLE 8
IVT capping efficiency of HB-MOD2 promoter with
Different Trinucleotide GAG Cap:NTP Ratios

| Promoter | Capping Efficiency (%) | | | | | | | | |
|---|---|---|---|---|---|---|---|---|---|
| Cap:NTP | Mix 1 | Mix 2 | Mix 3 | Mix 4 | Mix 5 | Mix 6 | Mix 7 | Mix 8 | Mix 9 |
| ratio | 6:1 | 4:1 | 2:1 | 1.5:1 | 1.1:1 | 1:1 | 0.8:1 | 0.5:1 | No cap |
| HB-WT | 85.89 | 83.14 | 65.75 | 58.53 | 51.99 | 53.11 | 51.83 | 41.56 | 1 |
| HB-MOD2 | 94.43 | 95.78 | 90.7 | 85.89 | 80.99 | 83.55 | 77.86 | 69.89 | 0 |

The cap:NTP ratio with the modified promoters influences IVT yield and capping efficiency as shown in Tables 7 and 8, respectively.

All of the following documents are individually incorporated by reference here in their entirety: US Patent Publication 2018/0318409A1; US Patent Publication 2019/0351040; US Patent Publication 2018/0271970; US Patent Publication 2019/0054112; US Patent Publication 2019/0336595; US Patent Publication 2018/0311336; US Patent Publication 2018/0303929; PCT Publication WO 2002/26891; PCT Publication WO 1997/40104; PCT Publication WO 1999/51702; PCT Publication WO 2001/21624; PCT Publication WO 1999/14226; PCT Publication WO 2018/085449; PCT Publication WO 2017/070601; PCT Publication WO 2019/202035; PCT Publication WO 2020/002525; PCT Publication WO 2019/193183; PCT Publication WO 2019/115635; PCT Publication WO 2019/038332; PCT Publication WO 2019/008001; PCT Publication WO 2018/167320; PCT Publication WO 2018/115527; PCT Publication WO 2018/115525; WO 2018/115507; PCT Publication WO 2018/104538; PCT Publication WO 2018/104540; U.S. Pat. Nos. 5,132,432; 8,039,642; 5,227,487; 5,442,045; 4,603,209; 4,849,362; 5,696,157; 5,459,276; 5,501,980; 5,830,912; 5,798,276; 5,846,737; 6,562,632; 7,256,292; 7,985,602; 8,729,267; 9,040,674; 9,315,859; 9,745,336; 9,783,560; 9,790,544; 10,131,936, 6,977,305; 6,974,873; 6,664,047; 4,774,339; 4,810,636; 4,714,763; 5,187,288; 5,248,782; 5,274,113; 5,433,896; 4,981,977; 5,268,486; 5,569,587; 5,569,766; 5,486,616; 5,627,027; 5,808,044; 5,877,310; 6,002,003; 6,004,536; 6,008,373; 6,043,025; 6,162,931; 6,130,101; 6,229,055; 6,339,392; 5,451,343; 6,716,979; 6,127,134; 6,130,094; 6,133,445, 7,446,202; 7,598,390; 7,776,529; 9,249,307; 9,751,868; 10,000,467; 10,053,447; 10,125,120; 10,351,551; 10,526,317; and RICHARD P. HAUGLAND, MOLECULAR PROBES HANDBOOK OF FLUORESCENT PROBES AND RESEARCH CHEMICALS (11 edition, January 2010).

1-68. (canceled)

69. A method of producing trinucleotide capped RNA comprising:
contacting a nucleic acid substrate with an RNA polymerase and a trinucleotide cap analog, wherein the mRNA cap analog of Formula (I) 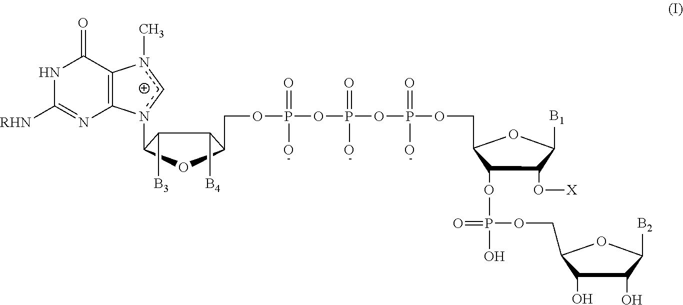
wherein
B₃ is chosen from —OH, halogen, dyes, —OR¹,
wherein R¹ is chosen from propargyl, tert-butyldimethylsilyl, and a methylene bridge with the 4′C;
B₄ is chosen from —OH, dyes, and —OR², wherein R² is chosen from propargyl and tert-butyldimethylsilyl;
or R¹ joins with R² such that B₃ and B₄ form -2′,3′-O-isopropylidine;
on the condition that B₃ and B₄ cannot both be —OH
X is chosen from —H and —CH₃;
B₁ and B₂ are each independently chosen from adenine, guanine, cytosine, and uracil;
R is chosen from H, a linker-bound cell-penetrating peptide, a linker-bound cell-penetrating peptide covalently linked to a dye, and a linker-bound dye, wherein the contacting is performed in the presence of nucleotide triphosphates, and wherein the contacting is performed under conditions and for a time sufficient to produce a trinucleotide capped RNA.

70. The method of claim 69, wherein B₃ is —OR¹, and R¹ forms a methylene bridge with the 4′C such that the trinucleotide cap analog is of Formula (II):

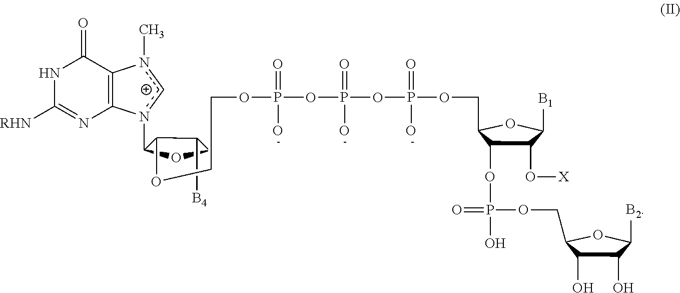

71. The method of claim 69, wherein each dye is independently chosen from azobenzene dyes, naphthalene containing dyes, cyanine dyes, rhodamine dyes, coumarin, and pyrene dyes.

72. The method of claim 69, wherein R is chosen from
(a) a linker-bound cell-penetrating peptide, wherein the cell-penetrating peptide is chosen from any of SEQ ID NO:1-583;
(b) a linker-bound cell-penetrating peptide covalently linked to a dye, wherein the cell penetrating peptides are chosen from any of SEQ ID NO:1-583; and
(c) a linker-bound dye.

73. The method of claim 69, wherein
B₃ is —OR¹ and B₄ is —OR² wherein R¹ joins with R² such that B₃ and B₄ form -2′,3′-O-isopropylidene;
X is —CH₃; and
R is H.

74. The method of claim 69, wherein
B₃ is chosen from —OR¹ wherein R¹ is chosen from propargyl and tert-butyldimethylsilyl;
B₄ is —OH; and
R is H.

75. The method of claim 69, wherein
B₃ is chosen from —OR¹ wherein R¹ is chosen from propargyl and tert-butyldimethylsilyl;
B₄ is —OH; and
R is chosen from 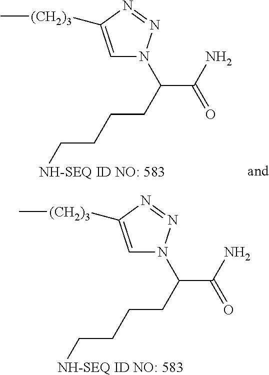
covalently linked to a dye.

76. The method of claim 69, wherein
B₃ is a dye;
B₄ is —OH; and
R is H.

77. The method of claim 69, wherein B₃ is —OR¹, and R¹ forms a methylene bridge with the 4′C such that the trinucleotide cap analog is of Formula (II):

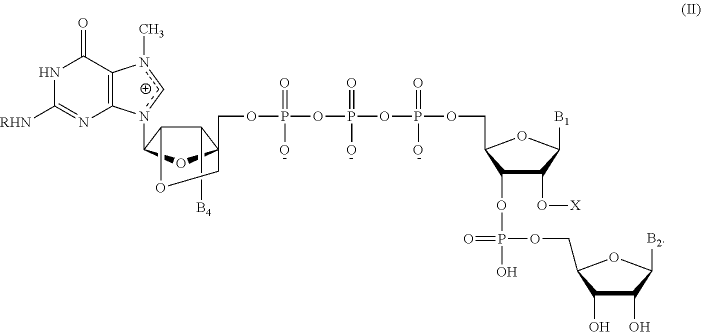

78. The method of claim 69, wherein each dye is independently chosen from

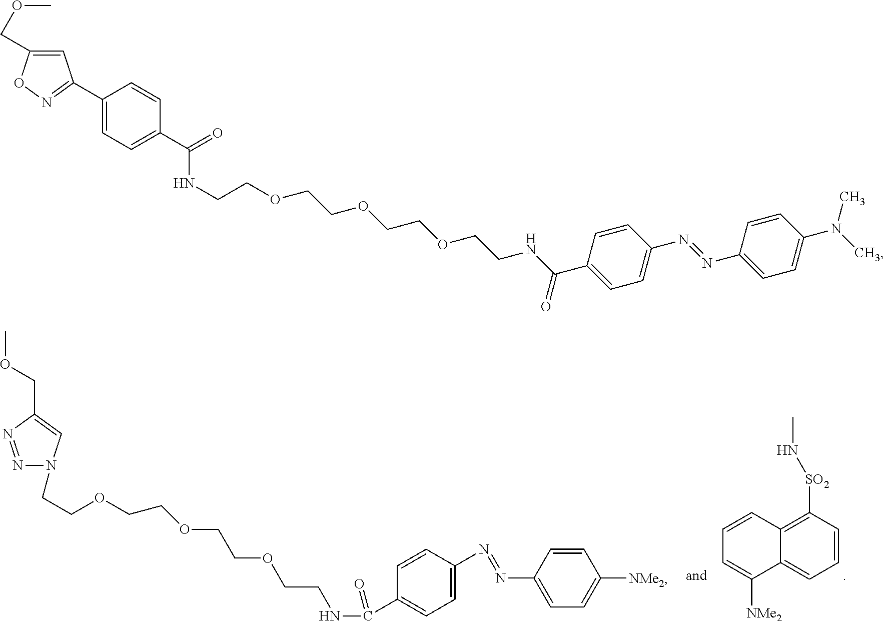 [SMILES: COCc1cc(-c2ccc(C(=O)NCCOCCOCCOCCNC(=O)c3ccc(/N=N/c4ccc(N(C)C)cc4)cc3)cc2)no1 ; COCc1cn(CCOCCOCCOCCNC(=O)c2ccc(/N=N/c3ccc(N(C)C)cc3)cc2)nn1 ; CNS(=O)(=O)c1cccc2c(N(C)C)cccc12]

79. The method of claim 69, wherein
B₄ is —OH and R is H.

80. The method of claim 69, wherein
B₄ is —OH; and R is chosen from 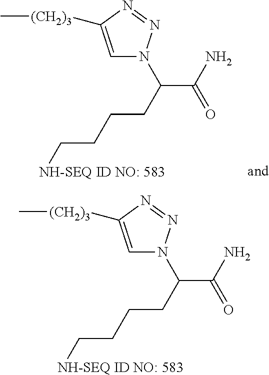
covalently linked to a dye.

81. The method of claim 69, further comprising contacting the trinucleotide capped RNA with an RNA delivery agent.

82. The method of claim 81, wherein the at least one RNA delivery agent comprises a at least one cationic lipid.

83. The method of claim 82, wherein the at least one RNA delivery agent further comprises at least one neutral lipid.

84. The method of claim 81, wherein the RNA delivery agent comprises a cell penetrating peptide.

85. The method of claim 81, wherein the RNA delivery agent comprises a PEGylated lipid.

86. The method of claim 69, wherein the nucleotide triphosphates comprise modified nucleotides.
